SEQUENCE LISTING (47 sequences)

SEQ ID NO: 1 - DNA, 7797 bp, Artificial sequence
  Nanopore PCR Sequences for Event 1
ctacttcgttcagttatagccctagactttggtcaaggtacagtcactgtagcttcaaggtccatcaacggttggaatttgaccctcattaacctcagcaagatataaggcaaaccaaaccaaggtgtacaacagccaccccagaatgctatctcattcaaagattttgcatgctagaatagtcaaactcaagagctcaagatatcaaggtcacaacaaatatcaaagattaaggctttggctccgagcctcaagaagataaggaggtcatatagtgccgacaagattgcgtatccccggtgctcaggcctcaggcctcaagaaatccaagggtttaaaacgcgatggttgctgaaccacccaaatcaagttttggggtcggtgccgtaaagcactgatcggaacctaaaggaatttgatttaggcaaacaggaaagccagcagacaatggcgaaggaaggaagaagcgaagggcaggcgccattcgatgcgcaactgggaagggcgatcggtgggcctcttcgctaaccgccagctagctgaaagagggatgtgctgcaaggcggtcagttaggtaaccagggttttccagtcgacgttcaaaggcgacggccagtgggtgttaattaagaattcgctggctatcactgaaagacaactacagacagtggtgtctcgatgcgcagaaccatctttgcagcagatgtaggcaatagatggtccgggcctccagaaagcgtcttcgacacagaaaagacaaccacagctcatcatgtatatggagctgtcgtgccatcggctgagataagactgactgagccagcactaaagaaataatgcaagtagtctagctccgctggctttaataattatatttcattattattctctgcttttactctctatgaggcttgtattttcattgaaggccaggcgaacacacacagaacctcgcttacaaacagatcttaaaccatggctcacgctggttttgtacctcttgctattcttactgctgctgctgttgctgtaagctactttaagttctttcttaattcagcaattaggccagtgactgatgagctgcttctactcttactcaattggatctatgtctgatagaccaaggaaggatcagttaatccagaagttgcttatgattgcatggctactgctgagacaggataccttagattccttggagtgctgagttcttcaacttcactggactcttggacttgaagaatggaggaggaaatgtactagaactattggaggaggacagaagtaggatgaagcagaaagagatgatacgctgtgaaggacaggtgtatatttcattggattcttacgatgtagttcttgctggatctcaataggcttttgaaggctacgttcagtctggatcgactactctctgctgggtcttttgttgtagagtagggacttgctgacaatatcaggaaattaggaactggagattcctgctcctggaggattcattggattcgctattctgggtgtgaggcggtggagtacaggatgacgatgatgatcagcttttcactgtttacatgaatcaggacgggagattgtcgccaaccttgatcacaagtctggttccagaaccttaacggtgctctccagtggtggttatacttagaagttcaatggagcaggtagggattagaacgtgaagcgatactcttctattgtggtgcatagaacggaccaacaaggctgtaaccttgaaaccattccaaacggatggactcagaaggaggatgctggattctgcaatcagatggagaactcttcaggaggacaaccacacaagagttttccttgctgtattcgttagacatgctccattccttccagattcttcagagcttcttcactggattaccgcagatgggtaacgcttttccttccttagcagttttccgacacattatttagaccccgcggacagcttcggatgtttctctgctgtgaagttggttggtcagagaagctctttctcaggaagctagggatatggctatggatttgtaggcaggatcaagtgcagttctacttctcttgatgctgatgctgtgctactaactggactcttaggattcatgatgagaacaggaaagtggtgctcaatgctttctaggcggggctagtctaatttctgattatctttctcctgatgagtactacgctagatcagaggactaagtagggcttattcagagaagagtaggagttgggcgttcttatatttctcgaacgtgattggggagatactctggatgggcttcacagaggatgttttctctggatctggtactggtcagatggcttctgcaagtcaccagggataaggaatttccttcacgctgtgtaggttcggaagattgctacttcaagatacgatactgggcacctgtctgatcttttggattttcagtagcagtggttggaaggcagtacattcacgagaactactctagggctcttgaagggaaggaattgtggagcaaccatgcccagatattttactggttcccatgctgaatatattacgtgggcttgttgctggatggggcagtacggacaatggtggaggtagacgcgggattctaaacgctggaggtgcgagaacgttcaactgtagatattccatgaagcaagtgggatacggatcaatggctggcagcacccaggacttatgggactgtgtggctgagtctcttttcccaggatacactaaggctagggctgtatagacttcgttgtggtatcgtccagatgagcagccatctcttaagccacaccacggtcttctttcctctggctcttaaccacagggacttgattatgagaggatgccgttccttagatacagttgcgtgatttcttaccaagaagggatgggctcttcttcatccaggaaaacactcattaccacggggactttaactacttgagactagatataatgggtatctttctggatccatggctgctttaatgagatatgcgggacgcctatgaccgcatgatgttgctttcaattctgttgtgcacgttgccaaaacacaggcatgtggctcggtcttaccgccggacagttcattaatgaatatatcaccgttactatcgtattttatgaataatattccgttcaatttactgattgtccaggtcaggctcggtacggggatcctctgagatctagtaacatagatgacaccgcgcgcgataatttatcctaatttgcacttgctatattttatttctatcgcgtaaatataattgggactctaatccataaaacccatcataaataacatcatacattgcatgtggagtattacatatagcatgtaattcatagaaattataccagtaatcatcgcaagaccggcagcagttcaatcagaaactttgttgcaaatgttgaacgatcaattcaggctctccacggccttcagtcatcatcctcctccatatcccaaaccactcaactgcccaatctcaagatcatcatcatctccagctccatcttatcgccagactcaaggaactgccagatcatcaaagtcctgttataatcaatcacagtcctatcagcaagtaggaagaacttcaaagttagaggaaagtgaaccttacagcctccacctcattagcaatgaatcatacttaatcacaatgttctcagtccttataaatctctcccaaccttgctcccagaattgggcaagctgcttcggtagccaccaggtgcgtagaactccacgaacgttcttcttctcgtaagcccacatcctcaagttttaccacctttttgccaactggctgtatcccaatcctctgggaagctcttgagactgaggtgtggcaatctcctcaaagtctggcagaactccagtaatcctctcagcggtagttttcaccggactctgacatgtgatcatctcctcaccgagtatggcctaacatttaggctcttattgcttaagtccgaagactcaaagtcctctggttatcaattgtagtcagaatcagtagaaatgaaagatcttgtgaagactcagcagtcttgaagttggagcaactttccatcgtaatcagacacagactttggaaagaatgcgagtcttaatctctcctccgaaatctggaatagtctatagtgaactcaatcacaagtgaagctggttgtacttaatgaaatcaagaaggttctcctttatgcactctcctcagaattgttcagctatgttcttgaaagcaccctccatcattatcaagctggtacttagagaacacatcagagttagaagtaatttcagatggaatataaatattagcctctccagcagcttgaaggaactgcccttgaatcagactccatcatgaaagtcaatcactgcctcagatgactcacaagagattccagcagcagctcatctgaaagtagtagctggtcagttctcttggccagttctacatgctcatcagcctcctttggcagtatactcctttggaagcagtatctcgtctccgttcctgaagaacttaatggttgacatcccctaactccgtactgtgagcaagatcggactcctcagtggcatcactttagcaaactaatttcagatcctcaactggcaccagcggccttaacgctctgggcaagagcttttgatgtccgcgcaagggcataagagctccacaaggaggtgcgcgcaggcggcgggaagccttcagcgaagttggaacctagctgggcgtgatcctcctcttctggggcatccatgtcgacaatttaggtaaggtgacggctctatggtcttcgccctaattaagttagaatgtgaagatgaaggccaccacggcaaaaagcagcggtaagcaagacagcgggcaaagcaaagccctgcgcgcgggccatggtttactcgactgtgattatgtcgtaggccataatgttgttgtttgttgtgacgagaagtagtgtatttgaaggatctcaggggcctatcctctcgaatgaaatgcgactttatatatgaggaaggtcttgcgagaaggatagtggatgtcgtcatccgcccgtcggaatatccatcaatccgcggcacagggcaatggactgggacgtgctaacaccacgatgttcctcagtgggtgggaattccatcttgggacccactgctcggtgaggcatcttagacgtggcctttcctttacctcactgtgatggcatttgccgagctacgcccttttctactgtcctttcggtaaagtgacgaatggctgggcaatggaatccgaggaagtttcgatgtaccgctttgttgaaagtctcaatggccctctggtgtacaggatcctctgagtcgacctgcattgaaacggcgcgccagcttggcgtaatcatggtcatagctgtttcctgtgtggtattgtcatcacaattccacaacatacgaaaccaggcgtaaagtgtaaacctacctgggtgcctaatggtgactaactcacattaattgcgtgcgctcctgcccgctttcagtcgaaacctgtcattggctgcgttaatgatcagccaacgcgcgggaggcggtttgcgtattgggccaaagacaagggcgacgttcagccagttggggagggtaaatattattgggtattcggtaaggtgaattatccgtcaccgacgatgcatttaggattaaattggcgaatcgcagataaccattaccattagcaagggccgagcgtcgcagtataagaaccatcgatggcacaccgtagtgatggcggcagatcaatttacctttagcgtcggactggcgcgttttcatcggcattttcggtcatggcccttattggcgtttgccatgccataatcaaatctggaaccgaccaccgcagaccgcctcctcaggccgccaccctcagaacccgccacctcagggccaccacctcaggccgccaccagggccaccaccaggccgccgccagcgtgtgaggctgatctagtaacatagatgtaccgcgcgataatttatcctagtttgcgctatatatttcatttctatatagcgtattaaatatagtgcaggactctaatcataaaacccatctcataaataacgtcatgcattacatatgaattgtacatgtagcgtaattcaagcagaaattatgataatcatcgcaagaccggcaacaggattcaatcttaagagctttatttgcaaatgtttgaacgatgagatcatcggtctgtgaactcacgaaaatatcggagcgcagcagatatcgcggtgcatctcgggtcttgcctgggcagtcgccgccgacgccgttagtgggctgggccgatcatgtgtcgctcaggatcgtggcgttgtagcatcggccattgctgtcgtaatgatactcagcgccttcgaccgcctgttcgcagagatcgtgggcgaagggctccagcggatcccgcgctggaggatcatccagcccggcgtcggaaaacgattccgaaatgacgctgccagaaggcggcggtaggtcgaaatcgtgatggcaggttgggcgtcgctcagtcggtcattcagacccagagtcccgctcagaagaactcgtcaagaaggcgatgaaggcgtgcgctgcagatcgggcagcgatgcagcaagcacgaggaagcgtcggccgatttcgccgccagctcttcagcaatatcacgggtagtaacgctatgtcacaatggcggtccgccacgcagccattacggtcgatgaatcagaaagcggcatttcaccgccgagtatttcggcgacgggcatcgccataggtcatgggatcatcgccgtcgggcatgtgcgcgccttgagcctggcgaacagttcggctggcgcaggctattatcttcgtcagatcatcctgatctgacaggaccggcttccatccgagtacgtgctcgctcgaccgatgtttcgcttggtagtcgaatgggcggtggcggatcaagcgtatgcagccgccgcattgcatcagccattggatacactttctcggcaaggcaggtaggatgtgcggagatcctgctggccgccccgctggcagcggccaagtgcccttccagtgacatgtcggctgcctgcaaggaacgccatcgtggcagccgataataccgcgctaccctcatcctgcagttcattcggggctggacaggtggtcttgacgaaagaccaggcgcctgcgcgtggccaggtgcggcggcatcaggcagccgattgtctgtgctgcttgaatcatagccagataacctccccaagcggcggagacctgcatgcagtcatcttgttcaatcatgcaggcagtcggtcggtgcaggtgttggttgagtagatgggataattggataccgaagttggaacgtcgtggagcatttttgacaaagtatttactggctggtggtgatatgggcgacactgggcctttagctggccatttctgacgtatgtacttgctcattagctcaaggccgcgtgatggctccttcaacgtgattcgccagttccaagagcatgcaaggcagcatcccgcgtcatcggcaaagtcatagcagcaatcgcaattctccgctcatgatcagattgtcgtttgccttcggttggtatcgcctgatagaccgttttcgccttggcgacaggtgttctttgtgatggactctttatttaaactggaacaacataaacctatcaaactcttcttttgatttatgag

SEQ ID NO: 2 - DNA, 11796 bp, Artificial sequence
  Nanopore PCR Sequences Event 1
ccgctggttcagttacgtattgctcaagaaattcgatgaagaacaatttcgaggaaggtgtaaaggagaaccttcttgatttcattagcacaaccagcttccacttgtgattgagttcactgagcagactgctcaaagattttcagaggagattaaagactcacattcttcttttccttcaaagtctgtgtctgattacgatggaaagttgtctaacttcaagactgctgctggatactcgggaagattcttttcatttcattgattctgatcacactgataacagagttcttgagttcttcggacttaagaaggaagagtgccaactgttaggcttgtactcttgaggagaatgactaagtacaagccagagtctgaaaatttactgctgagaggatggttctgcagattccttgaggaagattaagccacaccttatgtctcaaactttgaagttgggataagcagccggattaaggtgttggtgggtaaaaacttccggatgtggctttcgatgaggaaagaagaacgtgttcgtgtgggttctacgcaccttggtgtgaagtcactgccagcagcttgctccaggttggataagttgggagacttacaaggatgcgagaacattgattgctaagatggattctactgctaacgagtggagaggctgttaaggttcctctttagcagttcttccagcttctgctgatgggactgtgattgattacaacggagaaagtttcttgatggattcaagaagttcttgagtctggaggacaagatggagctggagatgatgatgatcttgaggatttggaagctgaggcagatatggaggatgatgatcaggctgtgatgagtgattgatcaaacggcaataaggttctgggtgggtctgttgcggtcttgatgattatcgcctgttctgttgaattacattagcgtaataattaacatgtaatgcgccggcgttatttatagaatgggtttatgattgggagtccgcattatacatttaatgcgcgatgaaaacaaaaatatagcgcaaactaggataaattatatggcgcgcggtgtcatctatgttactagatctagaggtccccggtaccgaagagaaaactcaattctggacagtagtaagtggtggagaatgtattcataaaaatacgatagtaacgggtattcttcattagaatgaaccgaaaccggtaggatctgagctacatgctcaggttttaacgtgcacagcaaattgacaaatatcatgcgatcatgggcatcgcatatcattaagcagtcatggatcacgaacaccgccatctagttcccaagtagttgaagtcctcgtggtaataggtaagccttcctggatgaaggccatccctttctgtagtgaagaaatcacgcaatcgtatctaagagcagcatcctcctcataatcaattacttgtggtttaagagccgcgacaagtgaaagtagaagaatcgtggtgtggcctagagatggttgctcatctggacgatacctcacagcaagttcgcagcctaaccttagtgtggtatcctgggaaaagaataatcattggtcccataagtctaagctgaagcctcgtatcccactttaggccatgtgaatatccacagttggaacgttctcgtatcctccagcaagctagaatcctcatgtctacctcctccattgtcgtaatatatctcaaccttaagctcatgcacatttgctcagaaaagtagaacagtaaacatctgggcatggttgctcctaattccttctattcagagcctaagtagttctcgtgaatgtactgctccttcaatcctgggttatcaatctgccaaagtcaggatgaaggtgctcagtatcgtatcttgatagcaagcaatcttcgaactcatgctggttagaaaggtgaaggaaaattcatatcctgaacttgcgaaagccatatctggatcagtatcagatcagaaaacatcctctgggctcctctaagagtatctcccctaatcacgtaagcctgaaatataagaacgttccaagctccctgctcgcagacaaatactctgatctagctagtataaatgggagaaaggcacacagtagacacaacttcccgtgctagaaagcattgttaatcactttcctgttctcctcaatgatcctaagagtctgaaggttgataagcacagcatggcatcaagagaagtagaactcactctggatcctgcctgcaaatcataaccatatctagcttctctggagaaaggcttcttctggaccaaccaacttcacagcaaatgatcctgaagctgtggcaagaatcaacaatggctcatggaaaacctcgttgttgtgaagaaaagtatacctatctggtagataatccaaagaaactctgaagaaatcttggaaggagttggctgttccatagtatgaaaactcttggtggttgtcctcctggaaagttctctatctgattgcagaatcccatctcttctggagtcatccgtttagacgtagtttcgagtagttaagctggagttttgttggttccatgccacaatgggaaaatttatcgtaagccgttctaatcctcactctgttctatcgaacagcacaacctcatcaaaaccgttaaggttctggaaaatctagacttgtgatcaggttagaaccttctcctaagtctggatcagtaaagcctagtgtgaaagctgatcatcgtcgtcgccttgctgcctcacaatctggtgaatagtagcggaatcagtagatcctccaggttaaagaatctttcagttcccgcttctggtgttatcagcaagtcccattctggccaacaaaagactcagcagaaaagcctgcctagatccagactggtgttcttcaaaagctcgttggagtccggcaagaaatcacatcgtaagaatccacgaacgcaataatcatatcctctccagcgtacttctcatctctttcttagccatctcactttcctgtcctctccagcagttctagcatcctccctccttcttctccaggtcaaggtcctcacggtggtgggctcagcactctaagagatctaggtatccttccagtctcagcagtagccacattaatctggcaactaccagttaactggatcgctcatggtctcgggcatagatctcaattgggcaaaggtaaacgctctatcggtcttggccctaagtgggttcgtcagcgaagagcgaaagccacagcaacatggcaatgtaagaacagccgagcaagcaaaaccctagcgtggtatggtttagatctggtttgccagcagggaggttctgtgtgtgtgttcgcctctgcactgccgtgaaaatacaagctctttatagaaaccaaagcagagaataataatgaaacatggtaatgcgtagacggagctggaccacttgcattgtatttcttcttcgtgctttgctttggggtcagtctttatcttcagcaaacatagcaactgatctcacatgttggatgagctgtggtgtccttttctatgtcgtgagatgcctttctggtgcgtcttgtggacctctggctgcttcatctgctgcaaaatggttctgattatcgaacaccattgtgctgctgtcttttcagtagctagtgtaattaacaattgtcactggccgtcgtttaacgtcgtgactgaaacctggcgttaccagcaatcgccttacctttatcccctttcgccagctggcgtaatagcagtacaccgatcgcccttcccaacagttgcgcagcccattaaccactcctttaactcttccttcctttctcgcccacgttcacgccggctttttcccgtcaagctcgtcggggctcgctggggttcgatttatttaaaacacctcgacaaaagcgatttggtgatggttcctgaaactatcagtgtttaaaccgctggatttcgcccgaggtttgaacgtcattttaggacatgcaattgtttggtataattatattactgatttcttggttttagacaaagttttaatctgatatatatttgttgtggacactttgatatcttgagttttaggagtttgttattttggggtacaaaattcttgaatgaatatgaagtattctggggtggttgttgtgtatcccaatcttgtttggttttggttttgtagtcttgttggggattaataagggtcaaattccaaccggttgatggaccttttaagcgaacactgcggttgggctggggcatgctgttggttgccagacgcagtcaagttcaaggactgttaaagtttgagctgaacgaatatctgttccctgtatgactctgtttagttctagcggattacaggtaaacttgctaaagttttcttcacccaacctaatagtttgaggttcaaattttgaaggggccttttcgtaacttgtaaggttgctcatcttgttaaaggcttgtggattaagcgggtaaacaggcttcttgcaatctgatgaggcgcatacaactagacccttgtggtaaaacatcacggtgatatagagcaggtgcacgggcctttattagattgagtttgaatgttagcttgaagaattggtgctttatcaattccagttaatcacactgtagcacagtgcgggttactatttctttcgcaatactatatggtatacttgagaagctatttatgatatcaatgttcatcgtctaacagagtatgttaagattcagttatgtcacaaatttgagttgataaaatgggaagtttgttaaggttgtatactttttagtcatggccaacttctcaattcatagcgataggcatattatagcatatgcagatattcttcattggtctttgcaattttaaattgtgtgagatgaaatatattttattaactggttaggggccgtaacattcttcatctgggatgcttggtttagatatgcatttactgtatttgcaggaatatcaaattggtgacaacattaaaccgtggatcaagtaaagttggcggtgttgttcaatatggaaagataaatcctcgttaaggctgaaacggtgtgatccttatttataatcgctttatccattttgaaggacttcagaaccacttctggaatcgccaccatgttacaggtatgcttatagaaactggtagcagctcctcttaatacagctgaattcattctctgctgagttgatgtttagtacggtctgaatctcctttagtaaacactgcttttgaatatagcaaactatttgttcattcgtcagctttactctttatctgcaaagaactgaatgtttgaactagatgtcagtgttaacttgacaccatggacaaagtagtcgtacactaagtagagtcgccatcacacagttactattgaatcaaatttagaatgttgcctctattaattgatagtaatgtagaaatatcttcactacagtttcacttgctgctgttaggcctctctacaactgcaaagcctgcttagtttggtttctgcggaagcgtgtatatagctcatgctttgaggcggtttgacgctggttgacttccttgggtctttgtacaactgcaacaagaatcttttcaagtctcattcacaatttcatctactgaatttcttttgggggtggtgttggaccttcacaatggattaataatgcaagtctcttatgtaatgatttgtttctcataatttgatttgatcatctggttttaattttagattctgcaggacaaaaccaatacattatactattgctcgatgtggagatcgcttctataccagctatgagtactatcaccatttcaagaccgcgtactatttcttctccaaagagctacccgagaatatagcagtgccagggactttcggtctaacgtgcaataccacttcctgttgtatttgatggagaacaatcagatcttgttctattggttgatgtcacatatgctaattgtctcgagcggaactggttcagattatattcgtgctcattcacggcttactccgataccacgagacccatcagcctgggtgggactgtgggaaggtgagcttgactcctttgcccattcaaatttgattacacatcgcgataagtaataaaatgtgatcgtgagcaaatggatagttcaactactgtactgagagttcaggttcagcgagataacacctgatcattcacttaaaaagaccagttttagatttctgtcgacgaaacacatgtatacttcactaatttaatttgtgcttctgttttcgacattgtaaatagatataaccccggcgctggtcaaaattcctatcatggtggaggaggaaccatgagatagaagacaagctgaaaatcagacatttgcagcctatcgttctcgcacgcctgcccatcaaagtgatcatcaccccatttactattcttttaatgcgggtagcgcacttcatcatgctcggagatataataagcctataatagatcaggtaaaaacctctcaaccttcagccaatcctcttctcccatttcacatgcttgcggtgaactttgaaatttgaaatctcaaatcatcgatcggtgtcgcttagttttcatatctttgcgttctgctccatctagtaaactgattcactaagcttctgattacagatgatcaatacatggttgaagggacgctgtgttgataggagagttactccattggctggtgccacttggcatccaccttggtactctacttacggcacgcaagctaagatgtatggtggcgatggaagatttgctatgcagtggtgtcgatttagtcttcatggacgtagtacaggattaacatacgttagcctagagcacgccagctatttgtatattccattccattgcacatttatgtatcaaatactgataacgaagaaatctatggttcatgcttatgaaggtcaaacagagtctataaactacactggatccttgtggtccgtctatataactatcggtggaaacggcgaagatggcaattgaacatgctgatgagccgaaatcttcaaattagatacttcccggacaaattgtaggcagttctgtgcctataattttacatcaggtcagctgctggtaactttgttgggatcaacctgattataagcatgcagtagccggccatgccagatactagaggtaaatatttatttctttcattgcgatatttatcaattcttactcctacagacaacagcagaagcagaattttggtatacctcgcgcagaccttcttataccttagattaagataattattttctagcaatctctgagcttttgttgtctagtggtaaaactcattgatgtgatggcgcacatgcagttcgaactctacatgaaggcgggtcgaaattgtccattaggtctcatactgtgtgccaactggcctcagttcttgattataaaaagcgttttgtagcacaagattattttgaaagtcagtctattgaacggcgaggtcttaggaaacatctccttctgagaggtgaagctgtgatgcatactaccctcccggaatccacttgtgggaatatgtattgggtatgttgttattatttgttaatcataaaccttcagatcacagaacatcaagtagcctataaaccaaaacagcagcgacaacatgatgtccgcaagtgaatctgaaaatgatatctggatacctacattaagaaagccagaggttgtttccgaccaactcgctcaaaagttaagttttcataaagtaggaatgaaggcgtgtcatgttatgcgttacagttcttgcaactttagttgctcgacttattgccgctaaatgccataacgtcaccttttgttcatagtacagaaatttgatggcaattttgttggtgcaggtgaaaaatgagacacatgctcatggacatggcataggaatcaggatatgtacaagctggagatataatttacatagtcgaacaaccctgaaatgccctgttaaactaggtaattaaacctggccgaattaaaataccaatttaattggatttaaagatactcgaccccaattataatttagctgagaggaaaaagaataggaaaaataatctattctttggaaataatgatttataaattatgattgacaacattaatatttttaatatattaaaagtgcacatattataatcaagaaccctccatttgatgtatatttggagagctaatcacaacgaattttaatattttctagcaatttatatatgatttgttgtgaataaactccatttccttcatctaattattattctttttatcaagtaaagtggtcatcttttcttttattatcttctacatttatagtttttcccgtggattagataactgaaattgtgaattaaatcaagaaaaacctttattgttattacaggcatcaggtggttttagttttatatttttgaatttatttgtgattcgttttgaatgaatgtcaaataaactcatcttttcctttcctttttaatcctcttcatttgtaaggttttctattaaaatggaaattatgtaatttcacgaagatgtcatcacgaagcagatgataaggagacgaagcaaatgggattagagacaatgtaccaacttcgaaagcagtaatcataaataataaaaatataaatttcagaaatatacatagctatttccattcaaataaaatattaagttggtttggacataaagaaaataagacctctaaaaccattggtcttaagtagaaataaataagttaattacttcattttgattataatttagaatctaaatttaaataacgtctggtacggacaacatgagtaaatacagatttccttttaaacaatgaatagttgtcatattttcctcacaaagcgactggcgcactaagctccgttatggtgaattggagaagaccaaaccaaagattacattatgcgtggcgcttatcgccgttctggtattcgtggctcggtcaggtgacctcatgacaacaactttgattctgcaaggctcctctcgtaccaagaacaaagcaaaaatataatcaaagaaaagcagtcaaaatgttaattttgttaattatcaatatgaaataatgatacaaagcataaataaatgaaatgggtatgcaagacacaaaatggtagaacttgggcatgctaacagatgaaatggttttaacataacaatgaacctagacttgtcattgttataatgagaaattcgaacatgttcgaaactcagtgaataatgaacctgctaatcctctccattttaaaggctaggttttgataagaattcaaactcgcaataagctaactcacacattacatgttgcactcatgccactagatcagcacttgttatcaacatggcagacaattgatattatgggacaatatatttactctaatgaacaagttttcatatgtaaaatgaaacttccttttctaacaaaattgtattgtcctagtctttgaaactagcataaagtatgggtttactctgcaaatgcctacaatagttgaaaaatatgaatgaagagcataaaaatgcaggaactaaatttgagcgaagctttagattctctggtgggaatatcaggaccaacgcgcggtgttgttcactccattaccatgaaagttgcagcgcctggtggtgtgtggatgaagttgacgtcgtgaccggtgtagtcgtccctccagtcattgccaccaccagggtgctcaggtaaatagagccaccagcagtgctgaaattacagaatccaagtttggtcagaatgccattttactacagaattgactacgaataatagcatgcatgatagtatgcttgtccatatacatgttggttgaacgagcaaatgatctttaatacagatactagcagattgattgatgcattagcatgcatgtctaaaactagtgtttggcagatcaaaatttaggcgtggggtactaaaatgcatatctaagaaattagtttgaatgcgagcgataagggataagtaatcgagattaaatttaaaatgagtttatcggttgatttgggataagtggtaagtataataattcgggattagttgtgcaggattgtagtgcttttatcccatgggagagtgggataactaatgtcgaatagtgtcatgagataatttatttcccagcaaacaacctaagtgtatctatgcttattttctgcacatatggccataagctacgcactgtatggttttctcaaaatataggtagcaaaaagttgcaaacatatataatgccacaatagtggcgttatacgatgcgttaagcgcacgttcgtatggcgtagctattgtggcgttatctgacacataaccgtgtagtggcattatactactaatgcagcccttaatatttacggatttaataatttaatcgtgtaaagccacttttgtggctatatacaaaaatccggctagccatttctatataaacataagtagaatagccctgacttcgttcaaatttttaactcttgttggtttctattgttgttaaattctcggcatttttcattatgtttagcgataggaataataatatatcgttatgatattggggtgaggttgtgatgaagaataataagtagactctacagtttacacgctgagtgtaatgttaagtgccactcaatggagtctgaaacattgatgtcgttgttatgcaaaaatgagtgtaagaaagcgttaagtgatactcaaagtagcaggaagatatcaggtgttaaattcgcataaaggagttacttttactcgggagtttaacatgcgacgatgatgacactttgatggaatttcttggggactcacgggataaatttcttgtaatcacaatgttggagatatgcgtgaaggtgaaggcgttcaaaaatgagttgtcgtggtaaagataaccccggtcatccgggtggttattacggatcattttagcgtattagaatgaaaagatttttcttgatttaaatttatccgtcggcaaatgaagcggccgagaaaataatttataccacctttccataattcacaagaaagtggtagcttcaattttcatttgtgttaattatgtatattttcttgcattaattaattttagcagctcatttatttaatagggagatgccggtaggatatagatttacaagtagcccatcggtgagtcatcacctaagcgaaagcgtccatcatggaatgtcatcacattacgacttgtaaagtggctacagccatatataagcatttgataatttagtaacccatatttttgttgaagtaatggtgaaggcgagcaagttgaactaccgtgccatggtgcgaaagcggcgtattacatgggatcacggcggatgagcagtgaggaaagtgccgattagataacaatgcagatgaatcccggggaagtgtgcggtggactgctgtaatgcactctttgattttataccttatgggcggccgatgtcgcggaatagtacagaatatcaagtcaatatccaagttgatgattccgttgaaagtgttcatttaagctgtgtgggtgggaatgtatttacccgcatgtcgtgtcgttataacttcctcactatacagctgctggtgcagtgcgttactgtatgcgccactatttgtggcgttatatatggatttggcttagctttttacacttatttggtattttgggtaaaaac

SEQ ID NO: 3 - DNA, 687 bp, Artificial sequence
  Border PCR Sanger Sequence for Event-1, Left border
ccgggcgaattcgaatgcctttagatactttttgggaaaccagcgtggacctcttgctgcaactctctcagggccaggcggtgaagggcaattagctgttgcccgtctcactggtgaaaagaaaaaccaccccagtacattaaaaacgtccgcaatgtgttattaaccacaagaaaatcaaaaggaaatttgcatgccctaaataatcaaactccaaacttcaagaaaccaaaagggtttacaaataatatatcaaagattaaaacttttgcaaaatagcccaaagaaaatcaaaatggatcatataactagagaatatgctcctaataaccaaattctaggcaaagaacttcaaaaaaccaaaagggtttacaaaaagtatatcaaagattaaaactttagcacaattacaccaagaaaatcagaaggggccaccaagaaaatcagaaggggaccacaaaaaaattaaaaggggccccacaaaaaaataaaggggggccccccaaaaaaaattagggggggccccccaaaaaaatttaggggggccccccaaaaaaattttggggggccccccaaaaaaatttggggggcccccaaaaaataagggggggccccccaaaaaaaaatgggggggccccgtaaaaaaggggggggggggggggtattatgggagggggggctaggtag

SEQ ID NO: 4 - DNA, 602 bp, Artificial sequence
  Border PCR Sanger Sequence for Event-1, Right border
caaaaggtagggtggactgaggcggcgctggtggaaggcttagagccccggagacatcaagatactgacagttacttattttcttaggcgaaacgcagctctttgaataagaactcacttcctctatattatccgcctctgagcctaattcaccagtataattttcaggggagggaatataaaagatccgactaggcctaaggatcaaaacaaatagctccaaacctcaagaaaatcaaaaggggtcatataattataccaaacaaatttgcatgtcccaaaattatcaaactccaaacctcaagaaatccaaagggtttaaaacactgatagtttgtgaaccatcacccaaatcaagttttttggggtcgaggtgccgtaaagcactgaatcggaaccctaaagggagccccctatttagagcttgacggggaaagccggcgaacgttgcgagaaaggaagggaagaaagcgaaaggagcgggtccattcaggctgctcgactgtggggaagggcgatcggtgcgggcctctacgctatccgccagctggcaaaagggggatgtgctgcaaggcgattaagttgctagcccaacgaaatga

SEQ ID NO: 5 - DNA, 5164 bp, Artificial sequence
  Nanopore PCR Sequences for Event 2 Left Junction
atcctttagttcggtgcatgtgctaggttaataggaaacacacgatagaatccgaacggcactaattgttctgtgaaggcggggcatacaccggatatggactcgacggatgaaggaaacaagtttactacgcttgggtgggtaactggcaaagccaatgtccggagtcaatgtgcatggccattctggccaatccgtgcggaccacaaacgccgccgcctttggtgcttccaaacgtggcgatgttggagtggacggcatggtaatcaacagcaacagacaatacagcgttaatgctgtagcctgagacccgggcacgttgttcttggcggttatcgcccagggcccacagcaacgcaccattagaagctgtgtctgctgcagaaggatattggccccaggtcgggcactttcccgggttccaggtagagacgttggttgataaaaaaactgattgcttcgctacaatgcggaagacggagcagcggcaactgattgcccttcaccgcctggcctggcgagttgcggcaagcggtccacgctggtttgcccagcagagccgaaaatcctgtttgatgttgtagtttccgaaatcgagccaaaatcccttataaatcaaaagaatagcccgagatagggttgagtgttgttccagttgaacaagagtccgcaataagaacgtggactccaacgtaaggcagcaatctatcagggcagcgtggccctacaagcgcaggcgagaaacgacaatgatcataaggggtacgttactgaccgccgatgacgccaggttaaaccgtttacgtttagattgacaagaaccaccgcaacatttgaaggagccactcagccgcgggtttcctggagtttcacaatgagctagtatacacgtccagaaaccattattgcatagatttcaagtcacctaaggtcactatctcagctagcaaatattcttaataaaatgctccactgacgtttccgccaagtgattcccctcagtatccagtagagtctcatattcactctcaatccaaataataatctgcaccggatctggatcgtttcgcatgattgaacaagatggaattgcacgcaggttctcggcgcttgggtggagagctactaaactatgactgggcacaacaaaccaatcggctgctctgataccgccgtgttcagctgtccagcgcaggggcgcccgttctttgtcagaaccgacctgtcggtgcctgggtgtgaactaccggacgaggcagcgcatgactatcgtggctggccacgacgggcgttccactgcgcggctgtgctctgacgttgtcactgaagcggggaaggactggctgctattgggcgaagatgccgggaagggcaggatctcctgtcatctccaccttgctcctgccagaaagtatccatcatggctgatgtgcaatgcggcggctgcatacgcttgatcgactacctgcccattcaccgaccaccaagcaacatcgcgcatcggcgaaacagcacgtactcggatgaagccggtcttgtcgatcagtgatctggacggcgaaaggcatcaggggctcgcgccgaaagccagactgttcatgtgggctcagcacgcatgccgacggcgatgatcgtcgtgacccatggcgtgcctgctttgcgccgaataacatggtggaaaaatatcgccgctttctgggattaatcgacctgacccggctgggtgcggcggaccgctatcaggttgctggcgttggctacccattgatattgctatgaagagcttggcggcgaatgggctgaccgcttcctcgtgctttacggtatcgccacgctttccgattcgcagcgcatcgccttctatcacgccttcttgacagttcttctgagcaggactctggggttcgaaaatggccgaccaagcgacgctcaacctgccatcacgaatttcagattccaccgccttctgaaggactgggcttcggaatcgttttccgggtacgccggctggatgatcctcagcgcgaggatcaataactggagttcttcgcccacgggatctctgcagacaggcggtcgaaagtgcccgatatcgtacgacatggcagccattccgaagcacaacgctacgatcctgagcaggcatactctgatcgggccagcgtcccgtaacggcgtcggcggcgacctgccaaggtaagaccgagatgcgccgcgatgccgtaatatctttgctgcgttccggatgactttagtgtaccaggttccgccacagacccggatgatccccgatcgttcaaacattttggcaataagtttcttagattgaatcctgttgccggtcttgcgatgattatcatactgtttctgttgattacgttaagcatagcaataattaacatgtgtaatacatgacgttatttatgagatggttttatgattagggatccgcaattatacatttgtacacgcgatgaaaacaaatatagcgcgcaagctaggataaattatcgcgcgcggtggctcatctatgttactagatcaggcctcctgtcaatgccaatggcagcgttgctctggtggattctggtggcagctctgaggtgtggctctgagaggtggcagagttccacagggtggcggctctgagggaaggcagtttcggtggtggcgctctctggttccgtgattttgatgaaagaatggcagaagccttaaagaggctatgatagaaaatgctacgatgaaaacgctacagtctgacgctaaaggcaaaccttgattcgcagtagctactgattgcgggtgctgctatcaggtggtttcattgacgttttccggccttgcttcaattgtgtgttgcctttactttggtgattttacggctctaattccccaatgttcagatcattcaggtgacggtgataattcaccgcacaatgaagatgggctgatcaatgttaccttcctctatcaatcaggttgaatgtcgcccttttgtctttattttaatgcactgagctacgcctctctcccgcgttgtacaatgggattcgtggcttatctacagctggcacggaacacaggcttaccaggaagcgggcgattgagcgcaacgcaattaatgtgggttaagctcactcattaggcacccagcttttacgctatgctccagctcgtatgttgtgtgtggaattgtggacaacaatttcacacgggaaacagcaggcatcatgattacgcaagctggcgccaaacatgcctgcaagaagttaggcttccaggaatctaaagtaacatagacttttgcgcgataatttatcctagttttacgctatatttttttgttctatcagcatggtaaatgtcttatattatagtgcgggactcaatcataaaaaagccatctcataaataacgtcatgtatgcatttacatgttacatgaagctggcatgattcaacagaaattatatgagtaatcatcgcagagaccgacagcaggattcaatcttaaggaaacttttattgccggcaatgtttgaacaggtagtccagagctcatcactcaagaagctcagaaagagtgcatggccttcctaaactcttgtccctctctgaagccatatttttttgacaccacttgtttctgcccgtaagaactgggcaagcaacatgcctagtagaggtaatctccctctccagaagcagaaaaggttgtgggaactgggttccatttgggcagggagcaaaagctgggaaggcaggcatcagtcacttgggttcgagtgtggtctgcttgaaatcgaaattgtggctcataaacgtgtcctccaactccacgtagttagcaacttgaagctctcctctagcagtagagcacactcagagtcagtaagatcctgaatctcatgttaatcctagacaccactgggttcccgtatccgaaagccaagcagacttagaaatctgtgcacagtctcaagatctgccaatgacagggttgaaaatagtagcctctgcgtcttggcttgccaaagatccttcacaatctccagtagcactcaagaaatatatcgtggggaacctaataatcacttggcttgctccactcatcttcttgcttaagctgggcaagaatgaacttcaggttcctgtttccatcaccgtgatacacagaacagcttcttctgccccttcagcaggtcatcttaaccaccctctccccctacacaacacatctcatcacttcacttgcttgcctctcagggaagataatcaacagccactccttcttctttgggagtgtctttttagtcagactgatcatccagaagcagacttgttcacatccttttccttagccataatgtactcagtacttaaggtttccgaattagcccctctgatgttctggatcaagctcaaccttcttagtaagcaagagccttatcaggagatcaccctgctgtggtacacagcgtaagaagagatgaaatcagcactgacactgcttatcaatgaagtagaaatctctccctctaaataatttgctaagagcttgttcctgtccaaagctcagtgtgttggtaatcggctactcccattgtatgcaacacccaagctcccagaagcaatcctcagcagcaggaagacttgttgcttaactcctggaaggtttcccttaagacaactaacaataaaatgtatcaagtttgtaagtatctgtgacctagaaggagcagcaacttagctcccacttgatcctcttcatcatttggaagtactacttacctctctgaatagtaggttagaaactaagatccatcagacatatcctcatacttttgaggttctccagctcactcactcggttaagcctcttcatcaacttgaaacttcactggatgaccaacggaatccctctggatctttgtagcagtagaggcaaaaagcctatccaactataagcccacttcttaatctgctccaacttagctccatctctcagccttaatgtaatcacttagagaagtcacaagattaagctactcagtgtgaataaagatcagtcatctgtccaatagaagtgaagaatcctgggtgagtgctgttggattctatcttatcatttcccctaatgccttagcaatacatggcgtaaa

SEQ ID NO: 6 - DNA, 2834 bp, Artificial sequence
  Nanopore PCR Sequences for Event 2 Right Junction
aattgaataatgaatgttttggtgagaggtcgctcattatattttgctgcctttatgctgaatttgttgttgtataaggatgtagtctactaccttgatggtgctaatttcttatcatattataacggattcagactgactttttcaagttgtatagcttaattgtgtttcaagccaatcgcgacggtcaggatttggtacttagaatgagtcatcttgccggccatattattgaacaaaatttcaactgtatgcagtgtacaatacgttatagaaaaacataggagatctttaaatcaactttattaattttcagaaaggagaaacatacatgtgaaattttgtagaatccatttatttctcagcagggtctgttctttcatgaatacaggctatttacgcaaattcaatcgaacgacaaatttttgaaatttcagcatcaaaatgtcaataattcacactagtgtttgcaagttgaggttttgggtcccatagcggaagcaaatattttctgatgcatcaaacgctgatagtttgtgaaccatcacccaaattaagttttggggtcgaggtgcgtaagcactaaatcggaacctaaagaattttgatttaggcttgacgggaaagccgagaacgtggcgagaagaaggaagaaagcgaaaggcgggcgccattcaggctgcgcaacattgggaagggcgatcggtgggcctcttcgtattacgccagctggcgaaaggggatgtgcaaggcgattgagttggtaactgggaacaccagtcacgacgttaaaacgacggccagtggtgttggtaagaattcgagctcggtacggggatctctagaggatcccctcagagcagagggctattgaacttttcaacaaagggtgtatcgaaacatatagttcattgccttatctcacttcatcgaaaggacagtagaaggtggctcctacaaatgccatcattgcgataaggaaggctatcgttcaagatactctaccgacagtggtcccaaagatggttaccgaacatcgtggaaagacgttcaaccacgtctttaaagcaagtggattgatgtgatatctccactgacgtgagatgacgcacaatcccactatccttcgcaagaccttcctctatatggaagttcatttcattggaaggacaggctgcccgagatccttcaacagtaccaacaacaacaaacaacaacacaattactgtacaattgcagtcgagtaaaccatggctcatgctagggttttgcttttggctcttgtgttcttgctactgctgttgctgtggcttcttcttcatctttcgctgattcttagtgggccagtgactgactactcttgctcaattggtcgacatgcaggattcttcacttctattggacagatgactgatcttattcacgcaggaaggatcttgacttcttaagttacattagtgaggaggataagttgggcagattaagtgggctgaagttggataggcttacttctactgccacaagatcagagttcgttggtcatcagtgacgctttcaagttgatgaagaagcaacactgagtgggtgagcttgaaccttgtgcttaggatatgactgatggattcatttctaaccttactattcagaggtgagtacttaaatgatgagagtcaagtgggagctgctaaggctcttcttaggctttgggatacttacaaccttgatactgatacaatttctgaaaccttccggggttaagcacaagtctttccttactgctgaggattgcttcgagcttggaaggttgcatacactgaggctgattgctaccacactgactttgagatggaacaagctcttaggcagcgatgaggagatttctactattgataaggtgtcaattagcgattacctttcttacgctgtgtgtaccagcagggtgatcttgataaggctcttttgcctactaagttgcttgagcttgatcgaacatcagagggctaacggaaaccttaagcgctggtacattggctaaggaaaggataacaagtctgcttctgatgatcagtctgatcaaagactactcaagaaggggtggctgttgattatcttcctgagaggcagaagtatgagatgttgtgtgggagaggtattaagatgactcaaggaggcagaagttgttctgcaggctcacgatgaagcaaggcaaagttcattcttactcaataagcaagaagatgagtgggataagccaagttattaggttccacgatatttctgatgctggggtggtgtgaggatcttgccaaggcttaggaggcctacaacctattactggtgatcttgagactgtgcactacaggttctaagtctgcttggctttctggatacgagaacccggtggtgtcacaagttaacatgattcaggatcttactggacttgatgtgtctactgtgaggcttcggttgctaactacgagttggagacaatatgagccacacttcgatttcgctgggaaggatgaccagatgcttttaaggagcttggaactgaagcaggattgctacttggctttcatgtccgatgtttctgctggaggagctactgttttcccagaagtgaacttctgtttggccaagaaggaactgctgtgttctggtacagcctcttcatctggaggaaattactctactaggcatgctgcttgcccagttcttgtgaaacaagtgggtgtcaaacaagtatttcatgagaggacaaaagtttagaaggccatgcactctttctgagcttggtgatgagctcgaattgatcgttcaaacatttgtatgct

SEQ ID NO: 7 - DNA, 407 bp, Artificial sequence
  Border PCR Sanger Sequence for Event-2; Left border Sequence 2-3F (1825F)
cgtttgactatgtcggttatgtgcatggcattctataagctatcgtcggacagaacgccgtgttagatgtcatcatgccatgttgcagtggaccgtaaggttttcaaccaagcacagttttgcgtgtacagtaacgaacccatttgatggcggatatcacccgggcccaactacgcatcattggaagctgtgtctccttggccgggatatttgggtcgggcgctttccccaggttcccgggtcagacgttggttgacaaaaaaactggtgctagctaccatgcggacgggcaacagctgattgcccttcaccgcctggccctgagagagttgcagccagccgtccacgctggtttgccccccaggcgaaaatcctgtatgatggtggtccgaaatcggcaaatcttt

SEQ ID NO: 8 - DNA, 477 bp, Artificial sequence
  Border PCR Sanger Sequence for Event-2; Left border 2-4F (forward sequence_9R)
ccgggttggaacaacggtggctgagggacaagtttgcataccttgggtgggtaactgggcaagccaatgtccgggtcaatgtgatggccattctataagccaatcgtaggaccacaaacgccgcctttggttgctccaaatggcgatgttggagtggacggcatggtgatcaacctagcaacagtattagcgtgtactgtaacgaacccgtttgatggcggatatcaccagggcccagctaacgcaccattagaagctgtgtctgcttgcaccgggatatttgggtcgggcgctttccccgggttcccgggagagaaattggttgataaaaaaactggtgctagctacaatgcggacgggcaacagctgattgcccttcaccgcctggccctgagagagttgcagcaagcggtccacgctggtttgccccagcgggcgaaaatcctgtttgaggcggttccaaaataaagaattaaa

SEQ ID NO: 9 - DNA, 889 bp, Artificial sequence
  Border PCR Sanger Sequence for Event-2; Right border
gcgaaagccttggttcatcaggttgtaacatcacgaccgtagagtgttttttttagtaagaggcgccctttctccctatttttgcataccctttaacttcttttgattttgttgttgtataaaggatgttagttactaccttgatagcaccccagtttcttatccattaataacggattcagataacttttttcaagtttgtatagcttaattgtgtttcagaaaccaatcatgacggtcaagagtttggtacttagaatatgaaaaggtcatcttgccggccatattattggaacaaaatttcaactgtatgcggtgtacaatacgttatagaaaaacataggagatctttaaaatcaactttattaattttcagaaaggagaaacatacatgcaataaagaattttgtagaatccattttatttctcagcagggtctgttctttcatgaatacaggctatacacgcaaattcaatcgaacgaacgaattttttttgaaatttcatatcaaaatgtatcaataattcacactagtgttttgcaagttgaggttttttgggtcccacatagccggaagcaaatcttttctacgatgcatcacacactgatagtttgtgaaccatcacccatattaagtttttgtggggtcgaggtgccggaaaagcactatatcggaaccctaagggagccccctatttagagcttgacggggaaagccggccaacgtgcgagaaggagggaagaaagcgaaggagcggcccaataggctgccacttgggaaggcgatcggtgcagtctctcgctatcctccagctgcgaaggggagtgctgcagcgattaattggggacaccattgtttactacaccccccccgcgctagatataagg

SEQ ID NO: 10 - DNA, 10063 bp, Artificial sequence
  Nanopore PCR Sequences for Event 3 Left border
tgtgttgtacttcggtttcagttacgtattactaaaaggttaataggaaacacgatagaatcagggcagcacctggttgtaacttcctccagattccactttagagtgattttgttgttttacacatattaaaaaatttaccttttagcgatagtgtatagtgagccatattaactatcaatttgtttgaatacaataaatactcttaaatcttgatattttaaaaaattaaatatttatcgaccacaaaaaaaatcaaaaaatcacttaagtggagggcttgtaaagtattaataaggaatcataaaatattttaataatacagatttttttgtaattttactagcaaaaaactagtactcttgcgtaaaattaacctttgttttaattaacttagatgcttaattaattgagaaaaaataattacatgggttattaccacagcaggtcagttgagacattattgtttgaaacaatgatagattacaaaccagagtttttctgacaaaagtagttcctaggatgtctgtccagagtgcatcattagacaaacacaggaggagcttctaaaatctagtgttgttgttgaacagtccttttctttgctccttcgcagccaatggagtccgacgattcaactcctcttaaactatgaagctggtgaagatcggttccagttgagtcatcagggacctgcgttcaaaacaaagttatcataaaattaacctttttttattttaaaataacctttcgttgatgatatggtgacgtttctttgtccaccgtagagaaagtctcataatagagcactttgtatctttgtaacaccaaaactctgtaatcctataattatttaacatcgtacaaaaatttcaaacaatttcggctgcgctgaagaaccgagcaggacttctacattattttttgtgcacataaaaaaaaaaaaagaataaaacatttcctttaaatttgctttgttatttatttcctcttgcacaactcaatcacctgtcaattttatttgacctaaggtcccattatatttaggctgtggccttcattaagaagagaaaaattaatacttttcttttaacgcggttttgtttaggaacttataaacaaagccctaccaatatttattttatcctcttttttatttatcaaagcagattaagatttctctgaaaaacaaggtatctcttttgttttttttttgaaacaaaggacaatttatattttggaaatagtagttgttttacatttaaaatgaaatagagattatcaaacttaagaaatttatcaccagaaataaatcaaactctagactggggtttgatgtgattggatgatgttctgtcccaaattctcccatggattcatgggatatccatataaatcaaaatagcgaagatagggttgagtgttattccagatttggaacagagtccatattaagaacacggactccaacgtcaaaggcgaaaagcaaatctatcagaggcgtggcccacaaactgaaaggcgaggcggcaatctgatcatgaagcggagaattaaagggaagtcacattatgaccccaccgatgacgcaggacaaccatttacgtttagacagcagaacacaacgttgaagagccactcagcctgctgcaaggtttaatgagctaactttatacgtcagaaacccattattgcgcgttcaaaagtcgcctaaggtcactatcagctatataaatgtttcattcaaaaaatgctccactgacgttccataaattcccctcggtatcaattagagtctcatattcactctcaatccaaataatctgcggatctggatcgtttcgcatgattggtaagatagttgcacaccaggttctcagccgcccaggtgcagagaggctattcggctatgactgagctgcaacagacaatcagctgctctgatgccgccgtgttccagctgttcagcgcaggggcgcccagttccttttgtcaagaccgacctgtccggtgcctatgaatgaactgcaggacgaaggcagcgcggctatcgtggctggccgacgggcgttcttgcgcagctgtgctcacgctatcactgaagcggaaggacggctgctattgggcgcgaagtgccggggcaggatctcctgtcatctcaccttgctcctgccgagaagtatccatctcatggctgatgcaatgcggcggctgcatgcgcttgatcggctacctgcccattcgaccaccaacgaaacatcgcatcaggcgagcacggctactcggatggaaacttgatcttgtcgatcagggatgatctggacgaagaagagcatctggggctcgcgccagccgaactgttcgccaggctcaaggcgcgcatgcccgacggcgatgatctcgtcgtgaccatagcgataccctgcttgccggaatgtcgcgggtggaaaatggcgcttttctggattcatcgactggcccggctgggtgtggcggaccgctatcaggacgcaagcgttggctacccgtgatgctgctgaagagcttggcggcgaatggggcgcgacgcttcctcgtgcttttacggtatcttagctcgattcgctgcagcatcgcgccttctatcgccgcttcttgacgagttcttctgagcagggactctggggttcgaaatgaccaaaccaagcgacgcccaacctgccatcacgagatttcaattccaccgccgccttctatgaaggttgggcttggaatcgtttcaggacgccggctggatgatcctctccagcgcggggatctcatgctggagttcttcgcccacacgggatctcgcggaacaggcggtcgaaggtgccgatatcattacgacagcaacggcccggcaaagcacaacgcccacgatcctgagcgacaaccgagtcgggccagcgtccacatcaacggcgtcggcggcagctgcgagcaagaccgagatgcaccgcagtaataacgcaccagatttcggatattttcgtggagttcccgccacaggcggatgatccccgatcgtttcaagaagcatttggcaataaagttttgctaagattgaatcctgttgccggtctttacgtgaattatcgcctgtttctgttgaattacgttaagcatgtaataattaacatacttaatacgcggtatttatgaatgagttttatgattagagtcccgcaattatacgactggcatacgcgatagaaacaaaatatggcgcgcgcaaactaggatgataaccgcgcgcgaattgtgcatcatgttactgaatcgggcctcatgtcaatgctggcggcagctctggtggtggttctggtggcagctctgaggtgagtagctctacgaggtagcggttctgaggtggcggctctgaggagagcagtttccggtggtggctctctggttccagtgattttgattatgaaagatggcaaacgctaataaggggctatgaccgaatacgtgaaaacgcgctacggtcgacgctaaaacagaaaacttgattctgtcgctactgattacgaattgctgctatcgatagttttcattagtgacatttccagcactttaatgaggcaatggtgctactggtgattttgctggctctagtcccaaatggctcaagtcggtgacggtgataattcacctttaatgaatgatttccgtcaatatttacagccactccctcaatcgaggtttgaatgtcatggcggcacgtctttggcccaatgcgcaaaccacatacctctcccgcgcgttggccgattcacattaatgcagctggcacgacaggtttcccgacttctggaaagcgggcagtggaagcgcctggtcaatgggattgaagctcactcattaggcacaggctttacactttaataagcttccggctcattatgttgtgtggaattgtgggcggataacaatttcacacggaaacaactatgaccatgattacgccaagctggcgcgccaatagacaaacacccttgttatacaaagaatttgctacaaaatcaaattcgagaaaataatatatgcactaaataagattattctggatcaatctaaccaaattacgatacgctttgggtacacttgattttttgtttcagtagttacatatatcttgttttatatgctaatctttaaggatcttcactcaagacctttatttgttgatgttcttgatggggctcacggaagatttgatatgatacactctaatctttaggaataccagccaggattatattcaatgaataatcaaatttacgtgttcaaactcgttatcttttcatttaatggatgagccaagtctctatagaatgattgcagtcgagaatatgttcggccgatatcccgctttgttggcttcaatattctacatatcacacagaatcgaccgtattatgccctctttccataaaggaacacacagtatacgaatgcttttccatgcagtaacataggtattcaaaaatggctaaaagaagttggataacaaattgaaaacagctatttccatttctgttatataaatttcacaacacacaaaagcccgtaatcaagagtctgcccatgtacagtaacttcttattatttggtattgggcctaagcaactcagagtacgtgggggtacccacatataggaaggtaacaaatactgcaagatagccccataacgtaccagcctcgctccttaccacgaagagataagatataagacccaccctgccacgtgtcacatcgtcatggtggttaatgataaggaagttacatccttctatgtttgtggacatgatacatgtaatgtcataaaaccacatgatccaatagccacaaggaacgtaagaatatagatagatttgattttgtccgttagatgtacaaaacaacattataaaggtgtgctatccaatacgaactaattcactcattggattcatagaagtccattcctcctaagtatctaaaccatggctcacgctctgatggtactctcctcctcgctctcgctgtttgtagcaagctgctgtggctgtggcttcaagttctggttttgctgatttcaaccccaattcgtccagttactgataaggcagcttccatgactccaattgcttcaaaagaagaaactgtgaggaagggcccaccagctggctgatgggggaccctagggccgaaaggtcaccaggaccactcaggcagggatggcgaagatggtccaactggccctcctggacctccctggccctcaagggccgccttgacaggcggaaacttcgcagctcaatacacgatggcagggtgttggtcttggtctggtcctatgggcttgatgggacctaggaggcctggtgctgctgattgctcctggaccacagggttttcagggacaactgacccaggagagccaggccaaaccaggaccagctggtgcaaggccctgctggacccctaccggctggtgaagagatgcaaattactttagggcaggccacgtgcaaggggggtgggcaatttgttggacccacaaggcgctgggggatttccaggagtacacctggtgccagttttaagggcattcaatggtcataacttggcctctctgatagttgaaggacagcctggcgcacctggcgttaaggagcagacctaggcaccaaggagtggcaagtactctcctgaccagactggtgcaagaggactcccaggtgaaagggtagagtgggtgctcctgtggacctgctggaggagctaggggtagtgatggtgagtgttggtcctgtgggccctgctggtccaatcggttcccgctggcccacctggattaggcgctccaggacctaagaggagaaatcggtgctgtgggtaacgcaggtcctactagtccagcaggtcctcatggagaggtaagattgccaggactctggtcagtgggcctcagggagcaaccctggagctaacacggcttgacaggagctaagagcgcagcttaggactccctggggtggctggcgcaccaggattacctggtcagggtatcccaggcccctgtggcacccagctggagctactagtgcacgtggacctgttggcagacagagcccctactggatcaaggcgagtctgggaaataagggaggagcctggttctgctggacctcaaggtcctcctggacctttctggagaaggaaaaaagggaggaccaaatggcgaggctgagatcagcaggtccaccagagaccacctgaacttcgtggatccctggtagtgagaggacttccaggcgctgatagtagagcaggcgttatgggaccaccaggaaatagagacatcaggagtccagcaggagttaggggtcctagcacagaatgctggtagaccagaaccagtcttatgggcccaaagggcactcccagatgatccaggaaatatcggcctgctgaaaaggccctgttggacttccagtattgatggacatcctggccctattagcccagcaggtgcagaggaagacctagcaatattggatttccaggaccaaaagggtccaacaggcgatcctggaagaaagaatgaagtcaggtcatgctggattagcaggcaccaagggcactcctggtagatggaaacaacggcacacaggagtccacctggcccctcagggtgttcaaaggcggaaaaggcaggagctgttaggcccagctggaccaccaggctttcaaggcttgcagggccaagtagtccagcaggtgaggtgtggcaaaccaggcagcgtggacttcatggcgagtttggactccctggaccagcaggaccaagggtgagccctcctggaaagtggcgctttcctgctggaccagcacccaatcagtggcagagtctagtaggacctcaggcccagatggaaataaggggtgaactaggagttgtgggcactgttacggaacagctggtccttcaggaccagctcaggactcccaggcgagaggcgctgctggcattcctggagaaaaaaaggtgaaaaaaggcgaacctggcactcccgtggcgaaatcctggacgtgatggtgctcgtggtgcacggcgctgtaggcactttccaggccctgctagtgctactggtgatagaggagaggctggcgcagctggcccagcaggtcacaccccagccccaagaaggcaatccctggtgaggcagattggacctgctggctagcagctgctggccctgctggaacaacagaatcaaccctggcgctaaaggtgaaaagggcagaaggagcccaaaaagattggctcagcgttacaagaccaactggtcagtgggcgcagctggacctgtagtcccaaatggaccaccaggacaacaggtagtagagggagatgggtgagacctccaggaatgacaggttttcagagtgctgctggtagagaacagggacctccctggtcacaagtggtattctggtccaccaggaccactcaggtcctgctgaaaaaaaagttaggggtccacgtggtgatcagggattccagtgggcagaactggtgagattggcgcagtggggacccacctggttttaactggagaaagggcccttctgggaggcagggaacggctgggtcactcctggtacacctggacctcaagggactggtacctattctcggtgccaggagagtaggggcaggcattgttcctggcaggagcagttggagaactggcctcaggaatgccccaccaggcaccgagaccaccagagctgttggatcaccaggcgtgaatggtactggcaggctggtcgtgaaaccaggaaatgatggcccaccaaggaagagatggccaacctggacacaaaaggctgaaggctgccaggaaatattggcccgaaagttggtgctgctggcgcaccaggcccacagtgagtggggccatagggaaaatacagtaatcgtggcgaaacaggcccttcaggcccagtgggacctactggtgctgttggccagaaggaccatctggacctcaggcattagaaacgcgacaaggagagcctggcgaaaaggacctagaggcttgctggttttaagacacacgcttggtctccaaggacttccaggtatcgctgggtcatcgcggaatcagggtgctcctggatcagtggtccagcagtccacaagaggcccagcaggcccttccggtccagcaggaaaggatggacatactggccacccctgctgggctgtgggcctgctggagtagaggtctcaggtcatcagagctactggccctccaaggtccaccacccagagtcaccaagcccaccaggagtttcaggtggtttgcgattttggttgatgattttaccattgctgatcaacctaagtgctccttctctccgtctaaagattatgaagttgatactttgaaatcataacagcagattgagactcttcctcacctgagggaatcgaaaagtccagcacgtatgcggtgatctcagacttaatcacccaaagtattcagagtggctattattggattgaagtctaatcaggggttgtacaatgagagctatcaagttgtgatttttccaactggaggaacatgtattgggggcacagcaagaacattccggcgctaaaggtggtatcgttctctaaggtcagaaacatgtttggctcgggctattaacgcttccagttctcagttcgagtataatgttggggggcgttacttctacgagagatggcagctcgccaactttttagagattgctcgctaactacatccaaaacttatccttatcttcaccaggaattccattacatacatggatgaaggagactaaggaaatttgaagaaagcaattattctcaaagtgtataacgatgttgggcttgtaaactgaaataaagtagattcactacagttttaattggattggatgctcaaaactaataaggtgggaaccattaatcagtaggctaagacagactctaaggctcccataggtgcactattcggattccgtgtttccctaatcaacaacagccagcctcggacttgagaaggccacataagttttactatttaccaagacttttgaatattaaccttcttataacttgagtcagttaaatttgattgtttagggttttgtattattttttttggtcttttaattcatcactttaattcccctaataattgtcctgattcatttcgttgtttgtttccggatcgacaatgaggaagccatgagatatcatatataaataataagtgtcgtttcatatttgcaatctttttttttacaaacctctaattaattgtatgtatatgactgttttcttcttgttatatattagaaataatattaataaaggcaaagataaacaaatacatcgaccttcttgaataaattacctagccaaaaaagaaagtacacctttcatatacttctacatgagagcattttcaacatttcaaataaggagtgtcaccacaaccgcataataacatcacagaatttttttataaaataacgcttatatcagacagtgattgaaagattttattgtagtttcgttatcttctttttctcattaggcgaatcactactaacacgtcattttgtaggaaaatattttttggatatgttttatatagttaagcattcctctttgatcagggtttgtttgaggatagcatttcagccggttcatacaacttaaagcatatactctaatgctggaaaaagactaaaaatcttgtaagttggcacaagaatattgttaattaatatacatgaccccactatataaggaataattacacttttaaccactaataataattattactgtattataacgtactaattaaacttggcaggttttgctggaattattaccataccatatactacctaagcgagacataggcctttggcatgtagcaactacagtagactgattaattgtgtctataattgacgcattaattagccaaaatgcctctt

SEQ ID NO: 11 - DNA, 657 bp, Artificial sequence
  Border PCR Sanger Sequence for Event-3; Left border Sequence 3-2F (Forward_14731F)
ctgccatctgctatggacgataagggtcggtcatcagggggctaaaccagttatggtagcactttgtatgcgacgctttcagggccaggcggtgaagggcaatcagctgttgcccgtctcactggtgaaaagaaaaaccacccctgtgcattaaaaacgtccgcaatgtgttattaagttgtctaagcgtcaatttgtttacaccacaaaataaagatatatacatgtatttggacatatatatttatttaatcataatcaattatgaaatatatgacctgataaataatttaactatgaaaaggttagatagttaggatggcagttttttaccgctttgaccccttctcaaaaacaacttgaatcccatgcattttctgtcttgtcaagctaaactcctcggttcaagagtatacgacaacttctatgtctgctacactgcaaccaatagacgacttaatagttaatagtaatcgtctaatacctaagaaagaaacaaatttaccaaaatgaattatattttttggcaagattagtggatataagacacgacaaagtataagcaggtttttcataacataggagatacttttttcgtgctgaatatcacagttcaatatattaagttacaaattttacaacaccccaaaaaaaaaa

SEQ ID NO: 12 - DNA, 657 bp, Artificial sequence; identical to SEQ ID NO: 11
  Border PCR Sanger Sequence for Event-3; Left border Sequence 1-3R (Reverse_RP1)

SEQ ID NO: 13 - DNA, 1213 bp, Artificial sequence
  Border PCR Sanger Sequence for Event-3; Left border 3-1F (forward Sequences_MP_Col_5R
cgggggtttacccattaggtatcttacaacgttcggtcattttgagagctgtcgccatgtccccccatgtaatgctcatttgttgcccgaggtttctgatgcggctttcgggtaccactctgttaacgaccggtccccagcgggatttgcctaaataccgattcttttgggaatacaggagatttatccaaaatgtcaatcacaacaagggcatctgatgcagcatcctatatagtttctactgaagtatcctaattagtaagttatggacaaaccatccatatgtcttattttaggagaagtgaaataaccattgttgccggcttaggatgaattttgatgccagagaatggtaaatacttctatataaaatcagaagcatgccactgttttttaaatagccgtgacgactttattcaacattaattgttttctttttcaagtttcaagtgatattagtccttcttgataaggacatgattcagcaagctgttcagtatattatcttgtgagaaatacaaacaaaacaacttaaggaagaagatcaaataagaagctggcgacagtgatcttgtgataaagaaaagaagaaaagacgcggaagatgttttttcaaaagatctttgtgagtatatcaaataagaatatgttattacttgtctatgataatgaaatcatctgaaagtgagtacggactctaacatttgtatttaaacttacagcttagtcattgctgtattatctttgacttattcaccacagtcacatcacctgtatatcttccccttgacgggacgagtctcacattagttgttaaccgtaactaatactaacgatagtgattactaattgtttaacccttaagaagtttgcggacaagtaaggcctttcttaggagttacttgcgagtactacctaatgtgatgaactgaccccgcctaacgataggtaggagtcgtagtgtcgttggtattaatgatgaaaattaatcatctagttcaaaaactgtatgattcactactagaatagcttatcggaggtcactgcgagactcaattcaagaaccgcttatcatcgacttaagtcatagctcgcaagcactgcgtgatgccgtatcgcgtgtactcgaatacagactaagccaatgttccaggcacatggttgatcacatcgacgcttaaggttgcatctctcgaattgtgactatgaaaag

SEQ ID NO: 14 - DNA, 1262 bp, Artificial sequence
  Border PCR Sanger Sequence for Event-3; Left border 3-3R (reverse sequence_ RP2)
cgccgcgccttatcgtcgtgggtgtttttaagtcaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatattaatatatatacatgtatttggacatatatttttatttaatcataattaattattatatattttacctcataaatcatttaactatgaaaagtttatatagtttggtgtggataaatttttaccgctttgaccgcgtctcatctcctacgtaaattccatgcatttgcagtgttgttgaagaaaaatactcctccggtgtcaagttattttcgacaacgtaactatatctagctttcactgttaatttatctagtacgtacttcatttgtttcatagtaaaaatctctgcgttattttccaaattgaaaagaaatgcaacattttaaacggaccaaatggtaattaacactattcttttagacataggagtatatgctgttattctaaagtacaacgtaatacgagcaattaaaggctaatgcttttctgctaggaattaaatttggaatgattaagcgtttctttatattgagtttaatttgcaaattactccataagtatttctaagtaagtgattataaaattatttaaccaaaagattaaatatcattatatatacaaaccaacaacaacgactcctaacctatagtttgactggtcattatttcattattagtacttctcaagtaatctcagaaagccttatatgtcgcaaacttttagggttaaaatttagtaatcattatgtgtttagttacggttaaaaactaatgtgagactctcccgtttaggggaaaaaatacatgtgatgtgacttggtataatgtcaaagataatacagcagtactaagctgtagatcaatacaataataagagtcgtactcactttcagatgattttatttcatagacagctataacatattatatttgtcatagctcacaaagatttttgtaaatactcgtccggctttcttcttaccatttcaaaagcacaactgtcccagcacgtagttgattcctccggacggttaaggtggcattctccgagatgactgagcgggtgtttatactgacctctcaggagcatcagatatccttgacactgtgaggagtacgcagat

SEQ ID NO: 15 - DNA, 5324 bp, Artificial sequence
  Nanopore PCR Sequences for Event 4 Left border
cccacttaagcagacaaccacgcgattagctttaagctataatcacaagcaatattgagcccacaatgattccttcttctcgaaaccctgatcccgcaccgttaacccgccaaatttcttgaagtgttttgtcaaatctcttcatgaacattctgaatcatcagccacaatctcatatccgacctctttccatgacgttgtatacgttcgttccttccccattaacatatcaactgagagttcaacaatatcatccaaactcaagtcatatcgcatctgtaaacgatagtcaaacctttacacccatcttcatcccaagcaaactcctttctgcaatattcattctttttccatacggcaaccatcattccaagaaaatacgtataccgaatcaccttctatcaggattcccaaacccttctacaatttcgtaaggtatgtggctatgattaccatagaatccagatgctactcggcctctcccacagaatcggtatgatactcatccgcatattgtggtgtaaaacaaattgacgcttagaaataacttagacaacacattgcggacgttttaatgtactggggtggtttttctttcaccagtgagatcggaagtaacagctgattgcccttcaccgcctggccctgagagagttgcagcaagtccacgctggtttgccccagcaggcgaaaatcctgtttgatggtggttccgaaatcggcaaaatcctcataaatcaaaagaagagtttgataggtttatgttgttccagtttaaataaagagtccatcacaaagaacacctgtcgccaaggcgaaaacggtctatcaggcgataccaaccgaaggcaaacgacaatctgatcatgagcggagaattgcgattgctgctatgactttgccgatgacgcgggatgctgccttgcatgctcttggaactggcgaatcacgttgaaggagccatcacgcggccttgagctaatgagcaagtacatacgtcagaaatggccagctaaaggcccagtgtcgcccatatcaccaccagccagtaaatactttgtcaaaaatgctccacgacgttccaacttcggtatccaattatcccatctactcaaccaacacctgcaccgaccgactgcctgcatgattgaacaagatgactgcatgcaggtctccgccgcttggggaggttatctggctatgattcaagcagcacagacaatcggctgcctgatgccgccgcacctggccacgcgcaggcgcctggtctttcgtcaagaccacctgtccagccccgaatgaactgcaggatgagggtagcgcggtattatcggctgccacgatggcgttccttgcaggcagccgacatgtcactggaagggcacttggccgctgccagcggggcggccagcaggatctccgcacatcctacctgccttgccgagaaagtgtatccaatggctgatgcaatgcggcggctgcatacgcttgatccgccaccgcccattcgactaccaagcgaaacatcggtcgagcgagcacgtactcggatggaagccggtcctgtcagatcaggatgatctgacgaagataatagcctgcgccagccgaactgttcgccaggctcaaggcgcgcacatgcccgacggcgatgatcccatgacctatggcgatgcccgtcgccgaaatactcggcggtgaaatgccgctttctgattcatcgaccgtaatggctgcgtggcggaccgccattgtgacatagcgttactacccgtgatattgctgaagagctggcggcgaaatcggccgacgcttcctcgtgcttgctgcatcgctgcccgatctgcagcgcacgccttcatcgccttcttgacgagttcttctgagcgggactctgggtctgaatgaccgactgagcgacgcccaacctgccatcacgatttcgacctaccgccgccttctggcagcgtcatttcggaatcgttttccgacgccgggctggatgatcctccagcgcgggatccgctggagcccttcgcccacgatctctgcgaacaggcggtcgaaggcgctgagtatcattacgacagcaatggccgatgctacaacgccacgatcctgagcgacacatgatcggcccagcccactaacggcgtcggcggcgactgcccaggcaagacccgagatgcaccgcgatatctgctgcgctccgatattttcgtgagttcacagaccgatgatctcatcgttcaaacatttgcaaataaagctcttaagattgaatcctgttgccggtcttgcgatgattatcataatttctgcttgaattacgctacatgtacaattcatatgtaatgcatgacgttatttatgagatgggttttatgattagagtcctgcactatatttaatacgctatatagaaatgaaatatatagcgcaaactaggataaattatcgcgcggtacatctatgttactagatcagcctcacacgctggcggcggcctggtggtggccctggtggcggcctgaggtggtggccctgaggtggcgggttctgagggtggcggcctgaggaggcggtctgcggtggtcggttccagatttgattatggcatggcaaacgccaataagggccatgaccgaaaatgccgatgaaaacgcgccagtccgacgctaaaggtaaattgatctgccgccatcactacggtgtgccatcgatggttcttatactgcgacgctcggcccttgctaatggtaatggttatctgcgattcgccaatttaatcctaaatgcatcgtcggtgacggataattcaccttaccgaatacccaataatatttaccctccccaactggctgaacgtcgcccttgtctttggcccaatacgcaaaccgcctcccgcgcgttggctgatcattaacgcagccaatgacaggtttcgactgaaagcgggcaggagcgcacgcaattaatgtgagttagtcaccattaggcacccaggtaggtttacactttacgcctggtttcgtatgttgtggaattgtgatgacaataccacacaggaaacagctatgaccatgattacgccaagctggcgcgccgtttcaatgcaggtcgactcagaggatcctgtacaccagagggccattgagactttcaacaaagcggtacatcgaaacttcctcggattccattgcccagccattcgtcactttaccgaaaggacagtagaaaagggcgtagctcggcaaatgccatcacagtgaggtaaaggaaaggccacgtctaagatgcctcaccgagcagtgggtcccaagatggaattcccacccactgaggaacatcgtggtgttagcacgtcccagtccattgccctgtgccgcggattgatggatattccgacgggcggatgacgacatccactatccttctcgcaagaccttcctcatatataaagtcgcatttcattcgagaggataggcccctgagatccttcaaatacactacttctcgtcacaacaaacaacaacattatggcctacgacataatcacagtcgagtaaaccatggcccgcgcgcagggctttgctttgcccgctgtcttgcttaccgctgctttttgccgtggtggccttcatcttcacattctaacttaattagggcgaagaccatagagccgtcaccttacctaaattgtcgacatggatgccccagaagaggaggatcacgcccagctaggttccaacttcgctgaaggcttcccgccgcctgcgcgcacctccttgtggagctcttatgcccttgcgcggacatcaaaagctcttgcccagagcgttaaggccgctggtgccagttgaggatctgaaattagtttgctaaagtgatgccactgaggagtccgatcttgctcacagtacggagttaggggatgtcaaccattaagttcttcaggaacggagacgagatactgcttccaaaggagtatactgccaaaggaggctgatgagcatgtagaactggccaagagaactgaccagctactactttcagatgagctgctgctggaatctcttgtgagtcatctgaggcagtgattgactttcatgatggagtctgattcaagggcagttccttcaagctgctggagaggctaatatttatattccatctgaaattacttctaactctgatgtgttctctaagtaccagcttgataatgatggagggtgctttcaagaacatagctgaacaattctgaggagagtgcataaaggagaaccttcttgatttcattaagtacaaccagcttcacttgtgattgagttcactatagactattccagatttcggaggagagattaagactcgcattctttccaaagtctgtgtctgattacgatggaaagttgctccaacttcaagactgctgagtcttcacaagatctttcatttctactgattctgactacaattgataaccagaggactttgagtcttcggacttaagcaataagagcctaaatgttaggccatactcggtgaggagatgatcacatgtcagagtccggtgaaaactaccgctgagaggattactggagttctgccagactttgaggagattgccacacctcagtctcaagagcttcccagaggattgggatacagccagttggcaaaaaggtggtaaaacttgaggatgtgggcttacgagaagaagaacgttcgtggagttctacgcacctggtggctaccgaagcagcttgcccaattctgggagcaaggttgggagagatttataaggactgagaacattgtgattaagtatgattcattgctaatgaggtggaggctgtaaggttcactttcctctaactttgaagttcttcctacttgctgataggactgtgattgattataacaggactttgatgatctggcagttccttgagtctggcgataagatggagctggagatgatgatgatcttgagattgggcagttgagtggtttgggatatggaggaggatgatgactgaaggccgtggagagcctgaattgatcgttcaacatttgcaacaaagtttctgattgaactgctgccggtcttgcgatgattactggtataatttctatgaattacatgctatatgtaatactccacatgcaatgtatgatgttatttatgatgggttttatggattagagtcccaattatatttacgcgatagaaataaaatatagcaagtgcaaattaggataaattatcgcgcgcggtgtcatctatgttactagatctcagaggatccccgtaccgagcc

SEQ ID NO: 16 - DNA, 465 bp, Artificial sequence
  Border PCR Sanger Sequence; Left border
gtgggggctcccacttagctttaagctataatcacaaaatattgagcccacaatgattccttcttctcgaaaccctgatcccgcaccgttaacccgccaaatttcttgaagtgttttgtcaaatctcttcatgaacattctgaatcatcagccacaatctcatatccgacctctttccatgacgttgtatacgttcgttccttccccattaacatcaactgagagttcaacaatatcatccaaactcaagtcatatcgcatctgaaacgatagtcaaacctttacacccatcttcatcccaagcaaactcctttctgcaatattcattctttttccatacggcaaccatcattccaagaaaatacgtataccgaatcaccttctatcaggattcccaaacccttctacaatttcgtaaggtatgtggctatgattaccatagaatccagatgctactcggcctct

SEQ ID NO: 17 - DNA, 6535 bp, Artificial sequence
  Nanopore PCR Sequences for Event 5 Left border
atcggtgtgcttcgttcagttacgtattgctaaggttaatagggaaacacgatagaatcaggaacagcaccttggatcaacttagcgggagttaagaataaaattagagcttcgtgctgataacgtgttataaaataaaaagcaatgcagtaaaatgtaaacaagaagatagagaaatgaagaatattttcttctttcactttagatatattttcattgcaattacatggcctttatagacataaaaaagtaaagatgatggacataaaatagaatttaatctttaagttattcacatttttttttttttttttttttttttttttttttttttttttttttttttttaacatgggcatccaatacttggcatcaatagtatcaatgtagtatccaaattagtatctaatgtacaaactattcatatgtcaaattttaggagaagtgaaataagttgcttttgccagcttaggatgaatattgaggacaaagaatagaagaaaaatacttctatatataaaatcagaagcatgccactgtttttaaataatacgtgatgactttattcaacatttattgttttctttccaagtttcttagagattatattagtcatgcttgataaggacatgattcaacaaactgttcagtatattatcttgtggaaatacaaacaaaactaacaggaaagaagatcaaataggctagtgacagttatcatcttgtgaaaagaaagaaagaaaagacgcggaaggtcataattttcaaaagatctttgtagtattatcaaataataatatgttattacttgtctatgaaaataaaatcatctgaaattaatttttggactctaatatttgtgtttaaaacttttacaacttagtaattgctgtattatctttgacaattataaccaaagtcacatcacatgtattttttccctaaacgggaagagtctcacattagtttttagccgtaactaaaaacaacataatgattactaaatttttaaccctaaaaagtttgcgacatatataaggcttttctaggggttacttgaagtactaatagtgaaataatgaccagtccaaactataggttagagtcgttgttgttggtttgtatgctcaatgatatttaatcttttggttaaaataattttataatcttacttagaaatgcttatggagtaatttgcaaattaaactcaatataaaagaaacgcttaatcattccaaaatttaattcctagcagaaaagcattaatacctttaattgctcgtattacgttattgctttagaataacagcatatactcctatgtctacaaaagaatagtgttaattaccattttggtccgtttaaaatgttgcattttcttttcaatttggaaaataacgcagagacttttactatgaaacaaatgaagtacgtactagataaattaacagtgactagatatggttacgttgtcgaaaataacttgacaccggaggtatttttcttcaacaacactgcaaatgcgtggaatttacgtaggagatagagacgcggtcaaagcagtaagaattatccacaccaaactatataaacttttcatagttaaatgatttatgaggtaaaatatataataattaactatgattaaataaaaatatatattagaatatccacatgtatatataataatattgtggtgtaaactagattgacagcttggagccataataactagggacgtttttaatgtgctgagattggtttctttcacaccaggagtgaggacagaccctggctccagctgatgattgcccttcaccactctggcccctgaggagtttacactgacaagcggtccacgctgaggttttgccccagcttgagcgaaatcctgtttgatggtggttccagaaatctggcaaaatcacttataaatcaaaagaatagcccgagatagggttgagtgttgttccagtttggaacagagtccataataaaagaacgtggactcaacgtcaaaggcgaaaaaaaccgtctatcaggggcgtggccacaaactgaaggcaggaaacgacaatctgatctatgagcggaggtaaagtcacgttactgaccccgccgatgacgccaaggacaagccgttttacgtttggaactgacagaaccgcaacgttggaaggagccactcagccgcgggtttctgaggtttgcaatagagctaagcacagctgcgtcagaaaccattattgcgcgttcaagtcgcctaaaggtcactatcagctagcaaatatttcttgtcaaaaaatgctccactgacgttccataaattcccctcggtatccagtaccagagtctcatattcctctcaatcaaaataatcttccaccggatctggatcgtttcgcatgattgaacaagatggattgcacaggttctccgagccacttgggtggaggctattcagctatgactgggcacaacagacaatcggctgctctgagtaccgccgtgttcagctgtcagcgcagggcgccagttctttttggtcaagaccgacctgtcggtgcctagaccgaactgcagagacctgaggcagcgcagctatcgtggctggccgacaggcgttccttgcgcagctgtgtgctcgacgttgtcacgcaagcaggaagggctggctgctgtgggcggcgagaagtgccggggcagaatctcctgtcatctcaccttgctcctgccgaagtatccatcatggctgatgcaatgccggcggctgcatgcgcttgatccggctaccgccttgttcgaccaccaaacaggcatcgcatcagcgagcacgtactcggatgggaagccggtcttgtcgatcaggatgatctggacgaagagcatcaggggctcgcgccaacccgaactgttcgccgagagctcaaggcgcacccggcgatgatctcgtcgtgacccatggcgatgcctgcttgccggaatatcatggtggaaaatggccgctttctggattcatcgactgtggcccggctgggtgtggcggacccgctatccaggacatagcgttatttacccgtgatattgctgaaagagcttggcgaatgggctgaccacgcttcctcgtgctttacggtatcgccgctcccgattcgcagcatcgccttctatcgccttcttgacgagttcttctgagcaggactctggggttcgaaatgaccgaccaagcgacatagcctgccatcacgagatttccgattccaccgccgccttctatgaaggttgggcttcggaatcgttttccggacgccagctggatgatcctccagcgcgggatctcatgctgggattcttcgcccacgggatctctgcggaacaggcggtcgaaggtgccgcgtatcattacgacagcaacggccgacaagcacaacgccacgatcctgagcgacaatatgatcgggcccggcgtcacatcagcagcgtcggcggcgactgcccagagcaagaccgagatgcaccgcgatatcttgctgcgttcggggatattttcgtaggttccgccacgaacccggatgatcgatcgtttcaaacatttggcaataaagtttcttaggtgaatcctgttgccggtcttgcgatgattatcatataatttctgttgaatacgttaagcatgtaatgagtggcatgcttaatgcatgacgttatttatgagatgggttttatattagttataaggtcctggtatatacatttaatacgcgatagaaaacaaatatagcgcgcaaactaggataaattattatcacgcgcgcggtgtcatctatgttactagatcgggcctcctgtcgagatgctggcagcggcggctctggtggtagttctggtggcggctctgaggggtggtggctctgaaattgtagcagtttctgagggtggcagctctggggaggcagtttccggtaattagctctggttcggtgattttgttgattatgaaagaatggcaaacgctaataaggggctatatgaccgaaaatgccgatgaaacgcgcgctacagtctgacgctaaaggcaaacttgattctgtcgctactgattacggtgctgctatcgatggtttcgtggtgacgtttcatgcttactaatggtaatggtgctgcaacgattttgctggctctgaattccaaatggctcaagtcggtgacagtgataattcacctttaatgaataatttcgtcaatatttaccttcctccctcaatcggttgaatgtcgcccttttgtctttggcccaatacgcaaaccgcctctccccgcttgcgttggccggtctcattaatgctggctggcacgacaggtttcccgactggaaagcgggcagtgagcgcaacgcaattaatgtgagttagctcactcattaggcacccaggctttacactttatgcttccaggctcgtatgttgtattaggtgtgagcggataacaatttcacacgaaacagctatgaccatgattacgccaagctggcgcgccaagcttcagaagaaggggaaaggcggttttggttaattaatgcgtccactatagacaattaatgcgtctaccgtagttgctacagtaccggggcaatgtttgcgttcctgccttttagtatatatgataataattctagcaaaaaaaactcacagaagtttaattggtagatgttataatacagtaataattattagtggttaaaagtgtaattagtctctataataggggtcatgtgtgtatataattttgggtcaatattttctgcgctaacttacaagatttttttgatctttttttcagcattagagtagcatacttttaagttgtatgaaccggctgaaatgctatctcaaacaaaccctaatttgaaaaaagagatgctacaactatataaaacttattcaaaaatatttcctttcaaaatggccgtgttaggtgatgattcagcaatgaaggaaagaagaagatagcagaaactataataaaatcttttcaatcactttgtctgacgtgttattttataaaaaaatttgtggatgttattacgcggttgtagcattccttattttgaaaatgttgaaaatgccttcatgtaggaatatatactcatggaagaaggtgtgcttgcagtacaggtaatttattcagtacgatgtgctgtagtttatttaatacttttatttaacattatttcccctaatataacaaaagaaaatgtcatacatacaattaattaaaagtttgtaaaagattgcaaatatgaaacgacaatttattatttatatgatatctcttacatttcattatcgatccggaaacaaacaacttgaaatgaacagacaattagggaattaaagtgatgaattaaaaagaccaaaaaaaaaataatacaaaaccctaaacaatcacaaatttaaccaaactcgttacagaaaggttaatattcaaaaaatcttggtaaatagtaaaacatgcggccgcaggctcattagagaaacaaactgggcccacatcaaaaccgaactcttgatccaggacggcgccagcatcgagagggagcaacatcaatgattgggagtcttgatgttttgtagtcttatactcgataactgttttaccccaagcgccagtatgtgatgcagccatcacttagtaactgaataagtaaaccttgagttgccctctgctctaatctccaatctcgtttgagccttcaaagaaagagctagccttcttaaggtttcctgtttgttgatccatataagccacactgttttgcagtgtagtaagtgatattttgtattgatgcctctgtggacataagacgcaaatgtaggttgaatagccacatcagctggatcataccttgaccgccgtactcaaattgaaatccatcagtcacatactttccgcagacaacgtgcctcttatctttaggatttgagatgctctggattttctgagccacggaaggttgagtaggataaacgcatgtttcgcctgtttccatgttacgagaaaactttgatagcatcaagagattacagccactgatttggatcagcagtattcaccacttttccaatcaggtgacacattttcaaatcacgacatgttctagctgggttttacgtgaaccttctggaagtctaatgttttcaatttgctgggagagaggatttccaaagttgtatcacttggaaatctctatcctaacaacgttgcatcatcaacacggtaatatctgccgccatcgtgagccttctcttgtagtggctgtggaaataatcagatccagctgatggggggccaggtggacctggtggaccaggaggtcctggaggagccaacaggagccagcatctcctgttcttcctctaggtcacagaggccggtggtcctaaggggaggccattaagaccatctttttcaggaggtgcaccagctgatcctggaggtcctcttgaacccagctgggcaagcacagatggtccct

SEQ ID NO: 18 - DNA, 1287 bp, Artificial sequence
  Border PCR Sanger Sequence; Left Border Sequence 5-2F (MP_Col_3R)
gggaaccggcctgcattttatttaatacaatagggaaccagcgtgggccgcaatgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatattaatatatatacatgtatttggacatatatttttatttaatcataattaattattatatattttacctcataaatcatttaactatgaaaagtttatatagtttggtgtggataaatttttaccgctttgaccgcgtctcatctcctacgtaaattccatgcatttgcagtgttgttgaagaaaaatactcctccggtgtcaagttattttcgacaacgtaactatatctagctttcactgttaatttatctagtacgtacttcatttgtttcatagtaaaaatctctgcgttattttccaaattgaaaagaaatgcaacattttaaacggaccaaatggtaattaacactattcttttagacataggagtatatgctgttattctaaagtacaacgtaatacgagcaattaaaggctaatgcttttctgctaggaattaaatttggaatgattaagcgtttctttatattgagtttaatttgcaaattactccataagtatttctaagtaagtgattataaaattatttaaccaaaagattaaatatcattatatatacaaaccaacaacaacgactcctaacctatagtttgactggtcattatttcattattagtacttctcaagtaactctcagaaagccttatatgtcgcaaactttttagggttaaaaatttagtatcattatgttgttttagttacggttaaaactaatgtgagactctcccgtttaggggaaaaatacatgtgatgtgactttggttataattgtcaagataatacagcaattactaagctgtagtttgaatacaatatcagagtccgtactcactttcagatgattttattcatagacaagtataactatattattgatatactccaaagatctgtggaaatacatcttcccggcgtgctcattccttcacaagaaaacttgtacggctctaatttaacctctcctaaagtgttcgctgtgtcccagatagtaaccgaacgcgtctgatctgttcctatcaggaaggcgatctaatgtgtactttgaatctagtcgtgatacgctaacgcagagcacgcacctagatg

SEQ ID NO: 19 - DNA, 1130 bp, Artificial sequence
  Border PCR Sanger Sequence; Sequence 5-1F (2_ MP_Col_4R)
gcggccggaggcacgcgtgcgtatacgtgtgttgataataaaagctatgcagtaaatgtaaactagaagagatagagagaatgaagaagtgttttcttctttcactttggtgtaatttttcattgcaattacatggccttttatagacataaaaagtaaagatgatggacataaaaaaggaaatttaatctttaagttattcacaacatgggcatcccatgtagcatcccactagtatccaatggagtatccaaattagtatctaatgtacaaactattcatatgtcaaattttaggagaagcgaacaggattgctgccggcttgggaaaatattgaggacaaagaatagaaaatacttctatataaaatcagaagcatgccactgttttttaaatagccgtgacgactttattcaacattgattgttttctttttcaagtttcaagtgatattagtccttcttgataaggacatgattcagcaagctgttcagtatatgatcttgtgagcatacaaacgaaacaacttaagggagaagatcatataagaagctggtgacagttatcttgtgaaaagaaaagaagaaagacgcggaagatgtatttttcaaaagatctttgtgagtatatcaaataataatatgttattacttggtctatgagataaaatcatctaaaagtcagtcggactctatatttgtatttaaacttacagcttagaattctgtttatcttgacaatatcaccaagtcacacaatgtattttttcccctgaacggagagtctacattaggttttaaccgtactagacaacagaatcatactaaatttttaccctggaaagttgcgacaaacaaggctttttagtattacttgagaagtactaatgatgggattattccccgtcggacgataagttagagtcgttgtgtttgttgtgtattgttgaaggaaattcaacctctggttaataatttattgatccctaacttaggaaagacctgctcgcgctattgcaattagagcgcgcgtagaaagaaaccgcctaagctttccgtattgttaatgtccgggccgaaggaagcctttggctcgttaattctcccgggcttacgtttgttgactttttg

SEQ ID NO: 20 - DNA, 4416 bp, Artificial sequence
  Synthetic sequence containing the coding regions of the vacuolar signal sequence of barley gene for Thiol protease aleurain precursor fused to the human Collagen alpha 1(I) chain
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggtggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctctaatgagct

SEQ ID NO: 21 - DNA, 4176 bp, Artificial sequence
  Synthetic sequence containing the coding regions of the vacuolar signal sequence of barley gene for Thiol protease aleurain precursor fused to the human Collagen alpha 2(I) chain
atggctcacgctcgtgttctcctcctcgctctcgctgttttggcaacagctgctgtggctgtggcttcaagttctagttttgctgattccaacccaattcgtccagttactgatagagcagcttccactttggctcaattgcttcaagaagaaactgtgaggaagggccctgctggcgataggggccctaggggcgaaaggggtccaccaggacctccaggcagggatggcgaagatggtccaactggccctcctggacctcctggccctccagggccacccggcttgggcggaaacttcgcagctcaatacgatggcaagggtgttggtcttggtcctggtcctatgggcttgatgggacctagaggcccacctggtgctgctggtgctcctggaccacagggttttcagggaccagctggcgagccaggagagccaggccaaacaggaccagctggtgcaaggggacctgctggacctcctggaaaagctggtgaagatggtcacccaggcaaaccaggacgtcctggcgaaagaggtgttgttggaccacaaggcgctaggggatttccaggtacacctggattgccaggttttaagggcattcgtggtcataacggcctcgatggattgaagggacagcctggcgcacctggcgttaagggtgaacctggagcaccaggtgaaaacggtactcctggccagactggtgcaagaggactcccaggtgaaaggggtagagttggtgctcctggacctgctggagctaggggtagtgatggtagtgttggtcctgtgggccctgctggtccaatcggttccgctggcccacctggattcccaggcgctccaggacctaaaggagaaatcggtgctgtgggtaacgcaggtcctactggtccagcaggtcctcgtggagaagtgggattgccaggactttctggtccagtgggccctccaggcaaccctggagctaacggcttgacaggagctaaaggcgcagcaggactccctggagtggctggcgcaccaggattgcctggtccaaggggtatcccaggccctgttggcgcagctggagctactggtgcacgtggacttgttggcgaaccaggccctgctggatcaaaaggcgagtctggaaataagggagaacctggttctgctggacctcaaggtcctcctggaccttctggagaagaaggaaaaaggggaccaaatggcgaggctggatcagcaggtccaccaggaccacctggacttcgtggatcccctggtagtagaggacttccaggcgctgatggtagagcaggcgttatgggaccaccaggaagtagaggagcatccggtccagcaggagttaggggtcctaacggagatgctggtagaccaggtgaaccaggtcttatgggcccaaggggcctcccaggtagtccaggaaatatcggccctgctggaaaagaaggccctgttggacttccaggtattgatggacgtcctggccctattggcccagcaggtgcaagaggagaacctggcaatattggatttccaggaccaaagggtccaacaggcgatcctggaaaaaatggagataagggtcatgctggattggcaggcgcaaggggcgctcctggtccagatggaaacaacggcgcacagggtccacctggccctcagggtgttcaaggcggaaaaggcgaacaaggcccagctggaccaccaggctttcaaggcttgccaggaccaagtggtccagcaggtgaagttggcaagccaggcgagcgtggacttcatggcgagtttggactccctggaccagcaggaccaaggggtgaaagaggccctcctggagagagtggcgctgctggaccaacaggcccaatcggtagtagaggtcctagtggacctccaggcccagatggaaataagggtgaaccaggagttgtgggcgctgttggaacagctggtccttcaggaccatcaggactcccaggcgagagaggcgctgctggcattcctggaggaaaaggtgaaaaaggcgaacctggcctccgtggcgaaatcggaaatcctggacgtgatggtgctcgtggtgcacacggcgctgtgggcgctccaggccctgctggtgctactggtgatagaggagaggctggcgcagctggcccagcaggtcctgctggcccaaggggtagtcctggtgaaagaggcgaagttggacctgctggccctaacggctttgctggccctgctggagcagcaggtcaacctggcgctaaaggtgaaaggggcggaaagggcccaaaaggtgaaaatggcgttgtgggaccaactggtccagtgggcgcagctggacctgctggtccaaatggaccaccaggaccagcaggtagtagaggagatggtggacctccaggaatgacaggttttccaggtgctgctggtagaacaggacctcctggtcctagtggtatttctggtccaccaggaccaccaggtcctgctggaaaagaaggattgaggggtccacgtggtgatcaaggaccagtgggcagaactggtgaagttggcgcagtgggaccacctggttttgctggagaaaagggcccttctggagaggcaggaacagctggtcctcctggtacacctggacctcaaggacttttgggtgcacctggtattctcggattgccaggaagtaggggcgaacgtggacttcctggcgtggcaggagcagttggagaacctggccctctcggaatcgcaggcccaccaggcgcaagaggaccaccaggagctgttggatcaccaggcgtgaatggtgcacctggcgaggctggtcgtgatggaaacccaggaaatgatggcccaccaggaagagatggtcaacctggacacaaaggcgagaggggctacccaggaaatattggcccagttggtgctgctggcgcaccaggcccacacggtccagttggaccagcaggaaaacacggtaatcgtggcgaaacaggcccttcaggcccagtgggacctgctggtgctgttggcccaagaggaccatctggacctcaaggcattagaggcgataagggagagcctggcgaaaaaggacctagaggcttgcctggttttaaaggacacaacggtctccaaggacttccaggtatcgctggtcatcatggagatcagggtgctcctggatcagtgggtccagcaggtcctagaggcccagcaggcccttccggtccagcaggaaaggatggacgtactggccaccctggaactgtgggccctgctggaattagaggtcctcaaggtcatcagggccctgctggccctccaggtccaccaggtcctccaggcccaccaggagtttcaggtggtggttacgattttggttacgatggtgatttttaccgtgctgatcaacctagaagtgctccttctctccgtcctaaagattatgaagttgatgctactttgaaatcacttaacaaccagattgagactcttctcacacctgagggatcaagaaagaatccagcacgtacatgccgtgatctcagacttagtcacccagagtggtcaagtggctattattggattgatcctaatcagggttgtacaatggaggctatcaaagtttactgtgattttccaactggagagacatgtattagggcacaacctgagaacattccagctaaaaattggtatcgttcctctaaagataagaaacatgtttggctcggagagactattaacgctggttctcagttcgagtataatgttgagggcgttacttctaaagagatggcaactcagctcgcttttatgagattgctcgctaactacgcatcccaaaacatcacttatcactgcaaaaattccattgcatatatggatgaggagacaggaaatttgaagaaagcagttattctccaaggtagtaacgatgttgagcttgtggctgagggaaatagtagattcacttacacagttttggtggatggatgctcaaagaaaactaatgagtggggcaagacaatcattgagtacaagacaaataagccttctaggctcccatttctcgatattgcacctcttgatatcggaggagctgatcacgagttttttgttgatatcggacctgtttgttttaagtaatgagct

SEQ ID NO: 22 - DNA, 1711 bp, Artificial sequence
  Synthetic sequence containing the coding regions of the vacuolar signal sequence of barley gene for Thiol protease aleurain precursor fused to the human Prolyl 4-hydroxylase alpha-1 subunit
atggctcatgctagggttttgcttttggctcttgctgttcttgctactgctgctgttgctgtggcttcttcttcatctttcgctgattctaacccaattaggccagtgactgatagagctgcttctactcttgctcaattggtcgacatgcacccaggattcttcacttctattggacagatgactgatcttattcacactgagaaggatcttgtgacttctcttaaggattacattaaggctgaggaggataagttggagcagattaagaagtgggctgagaagttggataggcttacttctactgctacaaaagatccagagggattcgttggtcatccagtgaacgctttcaagttgatgaagaggcttaacactgagtggagtgagcttgagaaccttgtgcttaaggatatgtctgatggattcatttctaaccttactattcagaggcagtacttcccaaatgatgaggatcaagtgggagctgctaaggctcttcttaggcttcaggatacttacaaccttgatactgatacaatttctaagggaaaccttccaggagttaagcacaagtctttccttactgctgaggattgcttcgagcttggaaaggttgcatacactgaggctgattactaccacactgagctttggatggaacaagctcttaggcaacttgatgagggagagatttctactattgataaggtgtcagtgcttgattacctttcttacgctgtgtaccagcagggtgatcttgataaggctcttttgcttactaagaagttgcttgagcttgatccagaacatcagagggctaacggaaaccttaagtacttcgagtacattatggctaaggaaaaggatgtgaacaagtctgcttctgatgatcagtctgatcaaaagactactccaaagaagaagggagtggctgttgattatcttcctgagaggcagaagtatgagatgttgtgtaggggagagggtattaagatgactccaaggaggcagaagaagttgttctgcaggtatcacgatggaaacaggaacccaaagttcattcttgctccagctaagcaagaagatgagtgggataagccaaggattattaggttccacgatattatttctgatgctgagattgagattgtgaaggatcttgctaagccaagacttaggagggctactatttctaaccctattactggtgatcttgagactgtgcactacaggatttctaagtctgcttggctttctggatacgagaacccagtggtgtctaggattaacatgaggattcaggatcttactggacttgatgtgtctactgctgaggagcttcaagttgctaactacggagttggaggacaatatgagccacacttcgatttcgctaggaaggatgagccagatgcttttaaggagcttggaactggaaacaggattgctacttggcttttctacatgtctgatgtttctgctggaggagctactgttttcccagaagtgggagcttctgtttggccaaagaagggaactgctgtgttctggtacaaccttttcgcttctggagagggagattactctactaggcatgctgcttgcccagttcttgttggaaacaagtgggtgtcaaacaagtggcttcatgagaggggacaagagtttagaaggccatgcactctttctgagcttgagtgatgagctc

SEQ ID NO: 23 - DNA, 1618 bp, Artificial sequence
  Synthetic sequence containing the coding regions of the vacuolar signal sequence of barley gene for Thiol protease aleurain precursor fused to the human Prolyl 4-hydroxylase beta subunit.
atggctcatgctagggttttgcttttggctcttgctgttcttgctactgctgctgttgctgtggcttcttcttcatctttcgctgattctaacccaattaggccagtgactgatagagctgcttctactcttgctcaattggtcgacatggatgctccagaagaggaggatcacgttcttgtgcttaggaagtctaacttcgctgaagctcttgctgctcacaagtaccttcttgtggagttttatgctccttggtgcggacattgcaaagctcttgctccagagtatgctaaggctgctggaaagttgaaggctgagggatctgaaattaggcttgctaaagtggatgctactgaggagtctgatcttgctcaacagtacggagttaggggatacccaactattaagttcttcaggaacggagatactgcttctccaaaggagtatactgctggaagggaggctgatgatattgtgaactggcttaagaagagaactggaccagctgctactactcttccagatggagctgctgctgaatctcttgtggagtcatctgaggtggcagtgattggattcttcaaggatgtggagtctgattctgctaagcagttccttcaagctgctgaggctattgatgatattccattcggaattacttctaactctgatgtgttctctaagtaccagcttgataaggatggagtggtgcttttcaagaaattcgatgagggaaggaacaatttcgagggagaggtgacaaaggagaaccttcttgatttcattaagcacaaccagcttccacttgtgattgagttcactgagcagactgctccaaagattttcggaggagagattaagactcacattcttcttttccttccaaagtctgtgtctgattacgatggaaagttgtctaacttcaagactgctgctgagtctttcaagggaaagattcttttcattttcattgattctgatcacactgataaccagaggattcttgagttcttcggacttaagaaggaagagtgcccagctgttaggcttattactcttgaggaggagatgactaagtacaagccagagtctgaagaacttactgctgagaggattactgagttctgccacagattccttgagggaaagattaagccacaccttatgtctcaagagcttccagaggattgggataagcagccagttaaggtgttggtgggtaaaaacttcgaggatgtggctttcgatgagaagaagaacgtgttcgtggagttctacgcaccttggtgtggtcactgtaagcagcttgctccaatttgggataagttgggagagacttacaaggatcacgagaacattgtgattgctaagatggattctactgctaacgaggtggaggctgttaaggttcactctttcccaactttgaagttcttcccagcttctgctgataggactgtgattgattacaacggagaaaggactcttgatggattcaagaagttccttgagtctggaggacaagatggagctggagatgatgatgatcttgaggatttggaagaagctgaggagccagatatggaggaggatgatgatcagaaggctgtgtgatgag

SEQ ID NO: 24 - DNA, 2299 bp, Artificial sequence
  Synthetic sequence containing the coding regions of the vacuolar signal sequence of barley gene for Thiol protease aleurain precursor fused to the human Lysyl hydroxylase 3.
atggctcacgctagggttttgcttcttgctcttgctgttcttgctactgctgctgttgctgtggcttcttcaagttctttcgctgattctaacccaattaggccagtgactgatagagctgcttctactcttgctcaattgagatctatgtctgatagaccaaggggaagggatccagttaatccagagaagttgcttgtgattactgtggctactgctgagactgaaggataccttagattccttaggagtgctgagttcttcaactacactgtgaggactcttggacttggagaagaatggaggggaggagatgttgctagaactgttggaggaggacagaaagtgagatggcttaagaaagagatggagaagtacgctgatagggaggatatgattattatgttcgtggattcttacgatgtgattcttgctggatctccaactgagcttttgaagaaattcgttcagtctggatctaggcttcttttctctgctgagtctttttgttggccagaatggggacttgctgagcaatatccagaagtgggaactggaaagagattccttaactctggaggattcattggattcgctactactattcaccagattgtgaggcagtggaagtacaaggatgacgatgatgatcagcttttctacactaggctttaccttgatccaggacttagggagaagttgtctcttaaccttgatcacaagtctaggattttccagaaccttaacggtgctcttgatgaggttgtgcttaagttcgataggaacagagtgaggattaggaacgtggcttacgatactcttcctattgtggtgcatggaaacggaccaacaaaactccagcttaactaccttggaaactacgttccaaacggatggactccagaaggaggatgtggattctgcaatcaggataggagaactcttccaggaggacaaccaccaccaagagttttccttgctgtgttcgttgaacagccaactccattccttccaagattccttcagaggcttcttcttttggattacccaccagatagggtgacacttttccttcacaacaacgaggttttccacgagccacacattgctgattcttggccacagcttcaggatcatttctctgctgtgaagttggttggtccagaagaagctctttctccaggagaagctagggatatggctatggatttgtgcaggcaggatccagagtgcgagttctacttctctcttgatgctgatgctgtgcttactaaccttcagactcttaggattcttattgaggagaacaggaaagtgattgctccaatgctttctaggcacggaaagttgtggtctaatttctggggtgctctttctcctgatgagtactacgctagatcagaggactacgtggagcttgttcagagaaagagagtgggagtttggaacgttccttatatttctcaggcttacgtgattaggggagatactcttaggatggagcttccacagagggatgttttctctggatctgatactgatccagatatggctttctgcaagtctttcagggataagggaattttccttcacctttctaaccagcatgagttcggaagattgcttgctacttcaagatacgatactgagcaccttcatcctgatctttggcagattttcgataacccagtggattggaaggagcagtacattcacgagaactactctagggctcttgaaggagaaggaattgtggagcaaccatgcccagatgtttactggttcccacttctttctgagcaaatgtgcgatgagcttgttgctgagatggagcattacggacaatggagtggaggtagacatgaggattctaggcttgctggaggatacgagaacgttccaactgtggatattcacatgaagcaagtgggatacgaggatcaatggcttcagcttcttaggacttatgtgggaccaatgactgagtctcttttcccaggataccacactaaggctagggctgttatgaacttcgttgtgaggtatcgtccagatgagcaaccatctcttaggccacaccacgattcttctactttcactcttaacgtggctcttaaccacaagggacttgattatgagggaggaggatgccgtttccttagatacgattgcgtgatttcttcaccaagaaagggatgggctcttcttcatccaggaaggcttactcattaccacgagggacttccaactacttggggaactagatatattatggtgtctttcgtggatccatgactgc

SEQ ID NO: 25 - protein, 1469 aa, homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 26 - protein, 1389 aa, homo sapiens
MAHARVLLLALAVLATAAVAVASSSSFADSNPIRPVTDRAASTLAQLLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPTGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

SEQ ID NO: 27 - DNA, 20 bp, Artificial sequence
  Single strand DNA oligonucleotide
gtcttatcttcagccgacgc

SEQ ID NO: 28 - DNA, 20 bp, Artificial sequence
  Single strand DNA oligonucleotide
acacaacaaccaccccagaa

SEQ ID NO: 29 - DNA, 22 bp, Artificial sequence
  Single strand DNA oligonucleotide
ccccttctgattttcttggtgt

SEQ ID NO: 30 - DNA, 20 bp, Artificial sequence
  Single strand DNA oligonucleotide
tcccctgaaactttggtcca

SEQ ID NO: 31 - DNA, 24 bp, Artificial sequence
  Single strand DNA oligonucleotide
tgatttataagggattttgccgat

SEQ ID NO: 32 - DNA, 20 bp, Artificial sequence
  Single strand DNA oligonucleotide
aattgttctgtgaaggcggg

SEQ ID NO: 33 - DNA, 21 bp, Artificial sequence
  Single strand DNA oligonucleotide
cacccaggattcttcacttct

SEQ ID NO: 34 - DNA, 20 bp, Artificial sequence
  Single strand DNA oligonucleotide
aaccctggcgttacccaact

SEQ ID NO: 35 - DNA, 20 bp, Artificial sequence
  Single strand DNA oligonucleotide
tgtgtttgggggttgaggat

SEQ ID NO: 36 - DNA, 20 bp, Artificial sequence
  Single strand DNA oligonucleotide
gtttgcatacgcttgggtgg

SEQ ID NO: 37 - DNA, 20 bp, Artificial sequence
  Single strand DNA oligonucleotide
aattgttctgtgaaggcggg

SEQ ID NO: 38 - DNA, 20 bp, Artificial sequence
  Single strand DNA oligonucleotide
attttgccgatttcggaacc

SEQ ID NO: 39 - DNA, 21 bp, Artificial sequence
  Single strand DNA oligonucleotide
tcatcaaggacctgcgttcaa

SEQ ID NO: 40 - DNA, 20 bp, Artificial sequence
  Single strand DNA oligonucleotide
agactcgccttttgatccag

SEQ ID NO: 41 - DNA, 20 bp, Artificial sequence
  Single strand DNA oligonucleotide
aggagtcgttgttgttggtt

SEQ ID NO: 42 - DNA, 22 bp, Artificial sequence
  Single strand DNA oligonucleotide
ataagggattttgccgatttcg

SEQ ID NO: 43 - DNA, 20 bp, Artificial sequence
  Single strand DNA oligonucleotide
taagcagacaaccacgcgat

SEQ ID NO: 44 - DNA, 20 bp, Artificial sequence
  Single strand DNA oligonucleotide
taaggttcgccggtgctatg

SEQ ID NO: 45 - DNA, 20 bp, Artificial sequence
  Single strand DNA oligonucleotide
tggatcaacttagcgggagt

SEQ ID NO: 46 - DNA, 22 bp, Artificial sequence
  Single strand DNA oligonucleotide
cacatcaaaaccgaactcttga

SEQ ID NO: 47 - DNA, 22 bp, Artificial sequence
  Single strand DNA oligonucleotide
acggttttaaagtcttgcaacc

(19) United States
(12) Patent Application Publication
(10) Pub. No.: US 2021/0310082 A1
(43) Pub. Date: Oct. 7, 2021

(54) TOBACCO TRANSGENIC EVENT AND METHODS FOR DETECTION AND USE THEREOF

(71) Applicant: CollPlant Ltd., Rehovot (IL)

(72) Inventors: Oded SHOSEYOV, Shoham (IL); Daphna MICHAELI, Jerusalem (IL); Itamar LUPO, Moshav Elad (IL)

(73) Assignee: CollPlant Ltd., Rehovot (IL)

(21) Appl. No.: 17/263,917

(22) PCT Filed: Jul. 31, 2019
(86) PCT No.: PCT/IL2019/050861

§ 371 Date: Jan. 28, 2021

Related U.S. Application Data
(60) Provisional application No. 62/712,289, filed on Jul. 31, 2018.

(51) Int. Cl. C12Q 1/6895; A01H 5/12; A01H 6/82

(52) U.S. Cl. CPC C12Q 1/6895; C12Q 2600/13; A01H 6/823; A01H 5/12

(57) ABSTRACT

Collagen producing plant events, DNA molecules for detecting same and uses thereof in plant breeding methods are provided.

### RELATED APPLICATION

This application claims the benefit of priority from U.S. Provisional Patent Application No. 62/712,289 filed on 31 Jul. 2018, which is hereby incorporated by reference in its entirety.

### SEQUENCE LISTING STATEMENT

The ASCII file, entitled 78292 Sequence Listing.txt, created on 30 Jul. 2019, comprising 131,072 bytes, submitted concurrently with the filing of this application is incorporated herein by reference.

### FIELD AND BACKGROUND OF THE INVENTION

The present invention, in some embodiments thereof, relates to a tobacco transgenic event and methods for detection and use thereof.

Collagens are the main structural proteins responsible for the structural integrity of vertebrates and many other multicellular organisms. Type I collagen is the predominant collagen component of bone and tendon and is found in large amounts in skin, aorta, and lung. Type I collagen fibers provide great tensile strength and limited extensibility. The most abundant molecular form of type I collagen is a heterotrimer composed of two different alpha chains [alpha 1 (I)]₂ and alpha 2(I). All fibrillar collagen molecules contain three polypeptide chains constructed from a repeating Gly-X-Y triplet, where X and Y can be any amino acid but are frequently the imino acids proline and hydroxyproline.

Fibril forming collagens are synthesized as precursor procollagens containing globular N- and C-terminal extension propeptides. The biosynthesis of procollagen is a complex process involving a number of different post-translational modifications including proline and lysine hydroxylation, N-linked and 0-linked glycosylation and both intra- and inter-chain disulfide -bond formation. The enzymes carrying out these modifications act in a coordinated fashion to ensure the folding and assembly of a correctly aligned and thermally stable triple-helical molecule. In nature, the stability of the triple-helical structure of collagen requires the hydroxylation of prolines by the enzyme prolyl-4-hydroxylase (P4H) to form residues of hydroxyproline within a collagen chain.

Each procollagen molecule assembles within the rough endoplasmic reticulum from the three constituent polypeptide chains. As the polypeptide chain is co-translationally translocated across the membrane of the endoplasmic reticulum, hydroxylation of proline and lysine residues occurs within the Gly-X-Y repeat region. Once the polypeptide chain is fully translocated into the lumen of the endoplasmic reticulum the C-propeptide folds. Three pro-alpha chains then associate via their C-propeptides to form a trimeric molecule allowing the Gly-X-Y repeat region to form a nucleation point at its C- terminal end, ensuring correct alignment of the chains. The Gly-X-Y region then folds in a C-to-N direction to form a triple helix.

Lysyl hydroxylase (LH, EC 1.14.11.4), galactosyltransferase (EC 2.4.1.50) and glucosyltransferase (EC 2.4.1.66) are enzymes involved in posttranslational modifications of collagens. They sequentially modify lysyl residues in specific positions to hydroxylysyl, galactosylhydroxylysyl and glucosylgalactosyl hydroxylysyl residues. These structures are unique to collagens and essential for their functional activity (Wang et al, 2002). A single human enzyme, Lysyl hydroxylase 3 (LH3) can catalyze all three consecutive steps in hydroxylysine linked carbohydrate formation.

WO2006/035442 and WO2009/128076 describe the production of human procollagen in transgenic tobacco plants by expressing all 5 transgenes that constitute the collagen chains as well as the enzymatic units responsible for modifying same (as described above, P4H and LH3). The resultant human Type I procollagen exhibits superior biological function when compared to any tissue-derived collagen, whether from animal or human tissues as described in Stein et al. (2009) Biomacromolecules10:2640-5 2009.

The expression of foreign genes in plants is known to be influenced by their location in the plant genome, perhaps due to chromatin structure (e.g., heterochromatin) or the proximity of transcriptional regulatory elements (e.g., enhancers) close to the integration site (Weising et al. (1988) Ann. Rev. Genet 22: 421-477). At the same time, the presence of transgenes at different locations in the genome influences the overall phenotype of the plant in different ways. For this reason, it is often necessary to screen a large number of events in order to identify an event characterized by optimal expression of an introduced gene of interest. For example, it has been observed in plants and in other organisms that there may be a wide variation in levels of expression of an introduced gene among events. There may also be differences in spatial or temporal patterns of expression, for example, differences in the relative expression of a transgene in various plant tissues, that may not correspond to the patterns expected from transcriptional regulatory elements present in the introduced gene construct. It is also observed that the transgene insertion can affect the endogenous gene expression. For these reasons, it is common to produce hundreds to thousands of different events and screen those events for a single event that has desired transgene expression levels and patterns for commercial purposes. An event that has desired levels or patterns of transgene expression is useful for introgressing the transgene into other genetic backgrounds by sexual outcrossing using conventional breeding methods. Progeny of such crosses maintain the transgene expression characteristics of the original transformant. This strategy is used to ensure reliable gene expression in a number of varieties that are well adapted to growing conditions that may ensure high yields year-round.

Additional Related Art:

WO2006/035442

WO2009/128076

### SUMMARY OF THE INVENTION

According to an aspect of some embodiments of the present invention there is provided a recombinant DNA molecule detectable in a sample containing tobacco DNA, wherein the nucleotide sequence of the molecule is:
a) at least 99% identical to SEQ ID NO: 6 or 9; or
b) a nucleotide sequence completely complementary to (a), wherein the presence of the recombinant DNA molecule is diagnostic for tobacco event A3-29-305-17-09-18 DNA or progeny thereof in the sample.

According to an aspect of some embodiments of the present invention there is provided a DNA molecule comprising a polynucleotide segment of sufficient length to function as a DNA probe that hybridizes specifically under stringent hybridization conditions with a recombinant DNA of tobacco event A3-29-305-17-09-18 or progeny thereof in a sample, wherein hybridization of the DNA molecule under the hybridization conditions is diagnostic for tobacco event A3-29-305-17-09-18 or progeny thereof in the sample.

According to some embodiments of the invention, the recombinant DNA molecule comprises:
a) a nucleotide sequence at least 99% identical to SEQ ID NO: 6 or 9; or
b) a nucleotide sequence completely complementary to (a).

According to an aspect of some embodiments of the present invention there is provided a pair of DNA molecules comprising a first DNA molecule and a second DNA molecule functioning as primers when used together in an amplification reaction with a sample comprising a recombinant DNA of tobacco event A3-29-305-17-09-18 or progeny thereof to produce an amplicon diagnostic for the recombinant DNA of the tobacco event A3-29-305-17-09-18 or progeny thereof in the sample, wherein the amplicon comprises a nucleotide sequence at least 99% identical to SEQ ID NO: 6 or 9.

According to an aspect of some embodiments of the present invention there is provided a method of detecting the presence of a recombinant DNA diagnostic for tobacco event A3-29-305-17-09-18 or progeny thereof DNA in a sample, the method comprising:
(a) contacting the sample with the DNA molecule under stringent hybridization conditions; and
(b) detecting hybridization of the DNA molecule to the recombinant DNA, wherein hybridization is diagnostic for the presence of the recombinant DNA of the tobacco event A3-29-305-17-09-18 or progeny thereof in the sample.

According to an aspect of some embodiments of the present invention there is provided a method of detecting presence of a recombinant DNA of tobacco event A3-29-305-17-09-18 or progeny thereof in a sample, the method comprising:
(a) contacting the sample with the pair of DNA molecules;
(b) performing an amplification reaction sufficient to produce a DNA amplicon using the pair of DNA molecules; and
(c) detecting the presence of the DNA amplicon in the reaction,
   wherein the DNA amplicon comprises a nucleotide sequence at least 99% identical to SEQ ID NO: 6 or 9, and wherein presence of the amplicon is diagnostic for the recombinant DNA of tobacco event A3-29-305-17-09-18 or progeny thereof in the sample.

According to some embodiments of the invention, the method further comprises detecting at least one of a nucleotide sequence at least 99% identical SEQ ID NOs: 1-5, 7-8, 10-19.

According to an aspect of some embodiments of the present invention there is provided a tobacco plant, plant part, or cell thereof comprising a nucleotide sequence at least 99% identical to SEQ ID NOs: 6 or 9.

According to some embodiments of the invention, the method or plant further comprises detecting presence and/or orientation of LH3, P4Hb, collagen alpha 1 and/or collagen alpha 2.

According to some embodiments of the invention, the presence and/or orientation is at least 99% identical to that of event A3-29-305-17-09-18.

According to some embodiments of the invention, the presence and/or orientation is identical to that of event A3-29-305-17-09-18.

According to some embodiments of the invention, the tobacco plant is a progeny of any generation of a tobacco plant comprising the tobacco event A3-29-305-17-09-18.

According to some embodiments of the invention, the tobacco plant, plant part, or cell thereof comprises at least one of a nucleotide sequence at least 99% identical SEQ ID NOs: 1-5, 7-8, 10-19.

According to some embodiments of the invention, the progeny is an inbred or a hybrid tobacco plant.

According to some embodiments of the invention, the progeny is listed in any one of Tables 20, 21, 21a and 22.

According to some embodiments of the invention, the recombinant DNA molecule is derived from a tobacco event or progeny thereof listed in any one of Tables 20, 21, 21a and 22.

According to some embodiments of the invention, the nucleotide sequence is as set forth in SEQ ID NOs: 34 and 35.

According to an aspect of some embodiments of the present invention there is provided a method of producing procollagen, the method comprising:
(a) growing the plant as described herein; and
(b) isolating the procollagen from the plant.
   According to an aspect of some embodiments of the present invention there is provided the procollagen obtainable as described herein.

According to an aspect of some embodiments of the present invention there is provided a method of processing procollagen, the method comprising:
(a) providing a protein preparation of the plant as described herein; and
(b) contacting the protein preparation with an effective amount of an enzyme capable of processing to procollagen to collagen.

According to some embodiments of the invention, the enzyme comprises ficin.

According to an aspect of some embodiments of the present invention there is provided a tobacco seed comprising a detectable amount of a nucleotide sequence at least 99% identical to SEQ ID NOs: 6 or 9, or complete complements thereof.

According to some embodiments of the invention, the tobacco seed comprises a detectable amount of a nucleotide sequence at least 99% identical to SEQ ID NOs: 1-5, 7-8, 10-19, or complete complements thereof.

According to an aspect of some embodiments of the present invention there is provided a nonliving tobacco plant material comprising a detectable amount of the recombinant DNA molecule.

According to an aspect of some embodiments of the present invention there is provided a tobacco plant, tobacco plant part, comprising DNA functional as a template when tested in a DNA amplification method producing an amplicon diagnostic for the presence of event A3-29-305-17-09-18 DNA.

According to an aspect of some embodiments of the present invention there is provided a method of determining the zygosity of a tobacco plant or tobacco seed comprising event A3-29-305-17-09-18 comprising:

contacting a sample comprising tobacco DNA with a primer set capable of producing a first amplicon diagnostic for event A3-29-305-17-09-18 and a second amplicon diagnostic for native tobacco genomic DNA not comprising event A3-29-305-17-09-18;
i) performing a nucleic acid amplification reaction with the sample and the primer set; and
ii) detecting in the nucleic acid amplification reaction the first amplicon diagnostic for event A3-29-305-17-09-18, or the second amplicon diagnostic for native tobacco genomic DNA not comprising event A3-29-305-17-09-18; wherein the presence of only the first amplicon is diagnostic of a homozygous event A3-29-305-17-09-18 DNA in the sample, and the presence of both the first amplicon and the second amplicon is diagnostic of a tobacco plant heterozygous for event A3-29-305-17-09-18 allele;
or contacting a sample comprising tobacco DNA with a probe set which contains at least a first probe that specifically hybridizes to event A3-29-305-17-09-18 DNA and at least a second probe that specifically hybridizes to tobacco genomic DNA that was disrupted by insertion of the heterologous DNA of event A3-29-305-17-09-18 and does not hybridize to event A3-29-305-17-09-18 DNA,
i) hybridizing the probe set with the sample under stringent hybridization conditions, wherein detecting hybridization of only the first probe under the hybridization conditions is diagnostic for a homozygous allele of event A3-29-305-17-09-18, and wherein detecting hybridization of both the first probe and the second probe under the hybridization conditions is diagnostic for a heterozygous allele of event A3-29-305-17-09-18.

According to an aspect of some embodiments of the present invention there is provided a method of producing a plant having an improved agricultural trait, the method comprising:
(a) subjecting the plant as described herein to a breeding program and/or transgenesis and/or genome editing; and
(b) selecting a plant exhibiting an improved agricultural trait.

According to some embodiments of the invention, the progeny comprises A3-29-305-17-09-18 hybrid with Samsun.

According to an aspect of some embodiments of the present invention there is provided a recombinant DNA molecule detectable in a sample containing tobacco DNA, wherein the nucleotide sequence of the molecule is:
a) at least 99% identical to SEQ ID NO: 1-19; or
b) a nucleotide sequence completely complementary to (a), wherein the presence of the recombinant DNA molecule is diagnostic for tobacco event A3-29-305-17-09 DNA or progeny thereof in the sample.

According to an aspect of some embodiments of the present invention there is provided a DNA molecule comprising a polynucleotide segment of sufficient length to function as a DNA probe that hybridizes specifically under stringent hybridization conditions with a recombinant DNA of tobacco event A3-29-305-17-09 or progeny thereof in a sample, wherein hybridization of the DNA molecule under the hybridization conditions is diagnostic for tobacco event A3-29-305-17-09 or progeny thereof in the sample.

According to some embodiments of the invention, the recombinant DNA molecule comprises:
a) a nucleotide sequence at least 99% identical to SEQ ID NO: 1-19; or
b) a nucleotide sequence completely complementary to (a).

According to an aspect of some embodiments of the present invention there is provided a pair of DNA molecules comprising a first DNA molecule and a second DNA molecule functioning as primers when used together in an amplification reaction with a sample comprising a recombinant DNA of tobacco event A3-29-305-17-09 or progeny thereof to produce an amplicon diagnostic for the recombinant DNA of the tobacco event A3-29-305-17-09 or progeny thereof in the sample, wherein the amplicon comprises a nucleotide sequence at least 99% identical to SEQ ID NO: 1-19.

According to an aspect of some embodiments of the present invention there is provided a method of detecting the presence of a recombinant DNA diagnostic for tobacco event A3-29-305-17-09 or progeny thereof DNA in a sample, the method comprising:
(a) contacting the sample with the DNA molecule as described herein under stringent hybridization conditions; and
(b) detecting hybridization of the DNA molecule to the recombinant DNA, wherein hybridization is diagnostic for the presence of the recombinant DNA of the tobacco event A3-29-305-17-09 or progeny thereof in the sample.

According to an aspect of some embodiments of the present invention there is provided a method of detecting presence of a recombinant DNA of tobacco event A3-29-305-17-09 or progeny thereof in a sample, the method comprising:
(a) contacting the sample with the pair of DNA molecules as described herein;
(b) performing an amplification reaction sufficient to produce a DNA amplicon using the pair of DNA molecules; and
(c) detecting the presence of the DNA amplicon in the reaction,
   wherein the DNA amplicon comprises a nucleotide sequence at least 99% identical to SEQ ID NO: 1-19, and wherein presence of the amplicon is diagnostic for the recombinant DNA of tobacco event A3-29-305-17-09 or progeny thereof in the sample.

According to an aspect of some embodiments of the present invention there is provided a tobacco plant, plant part, or cell thereof comprising a nucleotide sequence at least 99% identical to SEQ ID NO: 1-19.

According to some embodiments of the invention, the tobacco plant is a progeny of any generation of a tobacco plant comprising the tobacco event A3-29-305-17-09.

According to some embodiments of the invention, at least one of a nucleotide sequence at least 99% identical SEQ ID NO: 1-19.

According to some embodiments of the invention, the progeny is an inbred or a hybrid tobacco plant.

According to some embodiments of the invention, the progeny is listed in any one of Tables 20-30.

According to some embodiments of the invention, the recombinant DNA molecule is derived from a tobacco event or progeny thereof listed in any one of Tables 20-30.

According to an aspect of some embodiments of the present invention there is provided method of producing procollagen, the method comprising:
(a) growing the plant as described herein; and
(b) isolating the procollagen from the plant.

According to an aspect of some embodiments of the present invention there is provided a procollagen obtainable according to the method as described herein.

According to an aspect of some embodiments of the present invention there is provided a method of processing procollagen, the method comprising:
(a) providing a protein preparation of the plant as described herein; and
(b) contacting the protein preparation with an effective amount of an enzyme capable of processing to procollagen to collagen.

According to some embodiments of the invention, the enzyme comprises ficin.

According to an aspect of some embodiments of the present invention there is provided a tobacco seed comprising a detectable amount of a nucleotide sequence at least 99% identical to SEQ ID NO: 1-19, or complete complements thereof.

According to some embodiments of the invention, the tobacco seed comprises a detectable amount of a nucleotide sequence at least 99% identical to SEQ ID NOs: 1-19, or complete complements thereof.

According to an aspect of some embodiments of the present invention there is provided a nonliving tobacco plant material comprising a detectable amount of the recombinant DNA molecule as described herein.

According to an aspect of some embodiments of the present invention there is provided a tobacco plant, tobacco plant part, comprising DNA functional as a template when tested in a DNA amplification method producing an amplicon diagnostic for the presence of event A3-29-305-17-09 DNA.

According to an aspect of some embodiments of the present invention there is provided a method of determining the zygosity of a tobacco plant or tobacco seed comprising event A3-29-305-17-09 comprising:
contacting a sample comprising tobacco DNA with a primer set capable of producing a first amplicon diagnostic for event A3-29-305-17-09 and a second amplicon diagnostic for native tobacco genomic DNA not comprising event A3-29-305-17-09;
i) performing a nucleic acid amplification reaction with the sample and the primer set; and
ii) detecting in the nucleic acid amplification reaction the first amplicon diagnostic for event A3-29-305-17-09, or the second amplicon diagnostic for native tobacco genomic DNA not comprising event A3-29-305-17-09; wherein the presence of only the first amplicon is diagnostic of a homozygous event A3-29-305-17-09 DNA in the sample, and the presence of both the first amplicon and the second amplicon is diagnostic of a tobacco plant heterozygous for event A3-29-305-17-09 allele;
or
contacting a sample comprising tobacco DNA with a probe set which contains at least a first probe that specifically hybridizes to event A3-29-305-17-09 DNA and at least a second probe that specifically hybridizes to tobacco genomic DNA that was disrupted by insertion of the heterologous DNA of event A3-29-305-17-09 and does not hybridize to event A3-29-305-17 DNA,
i) hybridizing the probe set with the sample under stringent hybridization conditions, wherein detecting hybridization of only the first probe under the hybridization conditions is diagnostic for a homozygous allele of event A3-29-305-17-09, and wherein detecting hybridization of both the first probe and the second probe under the hybridization conditions is diagnostic for a heterozygous allele of event A3-29-305-17-09.

According to an aspect of some embodiments of the present invention there is provided a method of producing a plant having an improved agricultural trait, the method comprising:
(a) subjecting the plant as described herein to a breeding program and/or transgenesis and/or genome editing; and
(b) selecting a plant exhibiting an improved agricultural trait.

According to some embodiments of the invention, the progeny comprises A3-29-305-17-09 hybrid with Virginia K358.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a scheme showing F1 to F5 pedigree in breeding event A3-29-305-17-09. Green fill represents the selected lines and pedigrees.

FIG. 2 is a graph showing pools procollagen (PC) yield analysis of all F5 pedigrees. Pools PC-ELISA consistently suggest that A3-29 F4 lines 305-09 and its various derivatives at F5 are the highest yielding relatively to the other tested F4 lines progenies (segregating to F5 populations). n=2 in all F5 lines and n=3 in controls. Error bars represent the range of PC concentration in the consecutive analysis. Selected plants are in red fill; controls are in blank fill.

FIG. 3 is a graph showing individual plants PC yield analysis for selected F5 lines/pedigrees. PC concentration in F5 A3-29-305-17-09 individual plants showing the isolation of individual best-yielding plants. 7 best plants were selected for “winners” ELISA (red fill).

FIG. 4 is a graph showing PC concentration in A3-29-434-19-15 F5 individual plants showing the isolation of individual best-yielding plants. 3 best plants were selected for “winners” ELISA (red fill).

FIG. 5 is a graph showing PC concentration in A3-29-305-17-17 F5 individual plants showing the isolation of individual best-yielding plants. 3 best plants were selected for “winners” ELISA (red fill). Note that the tests are always done on seeds of the specific plants, therefore the results represent the subsequent generations.

FIG. 6 is a graph showing PC concentration in A3-29-305-17-02 F5 individual plants showing the isolation of individual best-yielding plants. None of these plants was selected for “winners” ELISA.

FIG. 7 is a graph showing PC concentration in A3-29-353-04-19 F5 individual plants showing the isolation of individual best-yielding plants. 3 best plants were selected for “winners” ELISA (red fill).

FIG. 8 is a graph showing PC concentration in A3-29-353-04-42 F5 individual plants showing the isolation of individual best-yielding plants. The best plant was selected for “winners” ELISA (red fill).

FIG. 9 is a graph showing PC concentration in A3-29-353-04-72 F5 individual plants showing the isolation of individual best-yielding plants. None of these plants were selected for “winners” ELISA.

FIG. 10 is a graph showing PC concentration in A3-29-305-13-18 F5 individual plants showing the isolation of individual best-yielding plants. None of these plants were selected for “winners” ELISA.

FIG. 11 is a graph showing “Winners” F5 individual plants comparative PC yield analysis. Two consecutive analysis of winners ELISA's (1^{st} in dark colors and 2^{nd} in pale colors). Results are given as a percentage of control line (A3-29- F1). All samples showed higher PC yields compared to the control. In two cases, one of the two ELISAs had values very close to the control (305-17-17#16 and 353-04-19 #8). Each color represents different pedigree.

FIG. 12 is a graph showing pools PC yield analysis of all F6 pedigrees. Bulks PC concentration in F6 seedlings (n=1). Consistent relative high PC levels were found at progenies of A3-29 F6 lines especially in A3-29-305-17-09 and A3-29-305-17-17 pedigrees. Each color represents different pedigree.

FIG. 13 is a graph showing pools PC concentration in full-developed selected F6 lines (n=3). Top PC yielding lines were progenies of F5-305-17-09. PC levels were increased by ˜50% compares to A3-29 F1 line and by up to 3.5 folds from Z1 production line. Four best lines were taken for individual plants analysis. Each color represents different pedigree.

FIG. 14 is a graph showing individual plants PC yield analysis in selected F6 lines. PC concentration in A3-29-305-17-09-10 F6 individual plants showing the isolation of individual best-yielding plants. 1 high PC yielding plant was selected for “winners” ELISA (red fill).

FIG. 15 is a graph showing PC concentration in A3-29-305-17-09-18 F6 individual plants showing the isolation of individual best-yielding plants. 2 high PC yielding plants were taken to “winners” ELISA (red fill).

FIG. 16 is a graph showing PC concentration in A3-29-305-17-09-25 F6 individual plants showing the isolation of individual best-yielding plants. 3 high PC yielding plants were selected for “winners” ELISA (red fill).

FIG. 17 is a graph showing PC concentration in A3-29-305-17-09-37 F6 individual plants showing the isolation of individual best-yielding plants. 4 plants were selected for “winners” ELISA (red fill).

FIG. 18 is a graph showing PC concentration in A3-29-305-17-09-15 F6 individual plants showing the isolation of individual best-yielding plants. 1 plant was selected for “winners” ELISA (red fill).

FIG. 19 is a photograph of Western Blot (WB) analysis. Anti-COL immunoblot analysis showed that all tested plants had higher PC levels than A3-29 F1 control (red arrow).

FIG. 20 is a photograph of Western Blot (WB) analysis. Anti P4Hα immunoblot analysis. Among candidates tested, plant 37-01 and 18-25 (red arrows) showed lower P4Hα expression, while plants 37-31 and 25-05 showed higher expression of P4Hα (blue arrows).

FIG. 21 is a photograph of Western Blot (WB) analysis. Anti P4Hβ immunoblot analysis. Among candidates tested, plant 25-05 and 37-01 (red arrows) showed decrease while plants 18-33, 37-10, 18-25, 15-13 and 25-04 showed increase in P4Hα (blue arrows).

FIG. 22 is a schematic illustration of insert characterization. EP-Event primer, Gp-Gene primer, LBP-Left border primer, RBP-Right border primer.

FIG. 23 is a schematic illustration of event 1 position in the genome;

FIGS. 24A-B show event 1 characterization on a Gel PCR. FIG. 24A—Insert characterization using event-1 specific primers for right junction, FIG. 24B—Insert characterization using event-1 specific primers for left junction. Primers are set forth in Table 35, Event #1 and Tables 36-37.

FIGS. 25A-B show the results of a border junction PCR: FIGS. 25A-B—Left border PCR using genome prime and border primers, amplicon size,1-400bp, FIG. 25B—Right border PCR using genome prime and border primers, 1-˜500bp. Primers are set forth in Table 35, Event #1 and Tables 36-37.

FIG. 26 shows PCR products and Sanger sequencing of event 1 (SEQ ID NOs: 1-4).

FIG. 27 is a schematic diagram of event-2 (P4H alpha) position in the genome.

FIGS. 28A-B show Event 2 characterization. FIG. 28A—Insert characterization using specific primers for event-2 left junction. FIG. 28B—Insert characterization using specific primers for event-2 right junction. Primers are set forth in Table 35, Event #2 and Tables 36-37.

FIGS. 29A-C show event characterization via Sanger analysis. Border junction PCR: FIGS. 29A-B Left border PCR using genome primers and border primers, amplicon size,1- 400bp, 2-4 500pb respectively. FIG. 29C, Right border PCR using genome primer and border primers, 1-˜800bp. Primers are set forth in Table 35, Event #2 and Tables 36-37.

FIG. 30 shows PCR products and Sanger sequencing of event 2 (SEQ ID NOs: 5-9).

FIG. 31 is a schematic diagram of event-3 position in the genome.

FIG. 32 shows insert characterization using event-3 left junction primers (see Table 35).

FIGS. 33A-B shows border PCR: FIGS. 33A-B, Left border PCR using genome prime and border primers, amplicon size,1-800bp, 2-˜2Kb. Primers are set forth in Table 35, Event #3 and Tables 36-37.

FIG. 34 shows the results of Nanopore-based sequencing and Sanger sequencing (SEQ ID MOs: 10-14).

FIG. 35 is a schematic diagram of event-4 position in the genome.

FIG. 36 shows insert characterization using event-4 left border primers. Primers are set forth in Table 35, Event #4 and Tables 36-37.

FIG. 37 shows the results of Nanopore-based sequencing and Sanger sequencing (SEQ ID NOs: 15-16).

FIG. 38 shows a schematic diagram of event-5 position in the genome.

FIG. 39 shows Insert characterization using event-5 left junction primers. Primers are set forth in Table 35, Event #5 and Tables 36-37.

FIG. 40 shows the border junction PCR: Left border PCR using genome primer, amplicon size 2-˜3Kb, 3-2Kb. Primers are set forth in Table 35, Event #5 and Tables 36-37.

FIG. 41 shows the results of Nanopore-based sequencing and Sanger sequencing (SEQ ID NOs: 17-19).

FIG. 42 is a bar graph showing PC concentration (mg/Kg leaves), Leaves biomass (gr/plant) and total PC (dg) in selected Fl plants. All transgenes in Fl are hemizygous, therefore PC concentration is expected present only half of its potential. Biomass yield (leaves weight) potential in many plants seems to be very high (1000 gr/plant and higher).

FIG. 43 is a bar graph showing PC concentration (mg/kg leaves) in selected F3 plant and 3 controls of production line “A3-29-305-17-09-18 F6 Bulk” (red bars). At least 14 different plants have better yield of PC in comparison to the current production line (red arrow).

FIG. 44 is a bar graph showing PC concentration (mg/kg leaves, blue bars) and leaves weight (gr, orange dots) in selected F4 plants. All plants either have much higher PC concentration and/or much higher biomass compared to production line “A3-29-305-17-09-18 F6 Bulk”.

FIG. 45 shows expected sizes. P4hB+LH3 and P4Ha: MW (ladder III), A3-29-305-17-09-18 F5, WT, NTC. Cola2: MW: A3-29-305-17-09-18 F5, 2-300, WT, NTC, Colal MW, A3-29-305-17-09-18 F5, 2-272, WT, NTC. All lines were assessed in the following sample order: 1: MW (PCRBIO ladder III) 2: A3-29 Fl, 3: A3-29-305-17-09 F4, 4: A3-29-305-17-09-F4, 5: A3-29-305-17-09-18 F6*, 6: A3-29-305-17-09-18 F6**, 7: A3-29-305-17-09-18 F6***, 8: Samson WT*, 9: Samson WT**, 10: Virginia K358 WT*, 11: Virginia K358 WT**, 12: [K358 x A3-29-305-17-09]-35-19-21-18-13 F6*, 13: K358 x A3-29-305-17-09]-35-19-21-18-13 F6**. Asterix refers to duplicates.

FIG. 46 shows top panel P4Hb and LH3 right border. Bottom panel P4Ha right border. All lines were assessed in the following sample order: 1: MW (PCRBIO ladder III) 2: A3-29 Fl, 3: A3-29-305-17-09 F4, 4: A3-29-305-17-09-F4, 5: A3-29-305-17-09-18 F6*, 6: A3-29-305-17-09-18 F6**, 7: A3-29-305-17-09-18 F6***, 8: Samson WT*, 9: Samson WT**, 10: Virginia K358 WT*, 11: Virginia K358 WT**, 12: [K358 x A3-29-305-17-09]-35-19-21-18-13 F6*, 13: K358 x A3-29-305-17-09] 35 19 21 18 13 F6**.

FIG. 47 shows top panel Cola2 left border. Bottom panel Colal left border primer MP_Col_3R and RP2. All lines were assessed in the following sample order: 1: MW (PCRBIO ladder III) 2: A3-29 Fl, 3: A3-29-305-17-09 F4, 4: A3-29-305-17-09-F4, 5: A3-29-305-17-09-18 F6*, 6: A3-29-305-17-09-18 F6**, 7: A3-29-305-17-09-18 F6***, 8: Samson WT*, 9: Samson WT**, 10: Virginia K358 WT*, 11: Virginia K358 WT**, 12: [K358 x A3-29-305-17-09]-35-19-21-18-13 F6*, 13: K358 x A3-29-305-17-09] 35 19 21 18 13 F6**.

FIG. 48 shows Colal left border primers MP Col 4R and RP2.

FIG. 49 shows left top: MW PCRBIO ladder III P4Hb+LH3 right border expected size 800bp. Right top: MW PCRBIO ladder III P4Ha right border, expected size 800bp. Left bottom: MW PCRBIO ladder III Cola2 left border, expected size 800bp. Right bottom: MW PCRBIO ladder II Colal left border expected size 3kb.

FIG. 50 shows MW PCRBIO ladder II, Colal left border expected size 2kb.

### DESCRIPTION OF SPECIFIC EMBODIMENTS OF THE INVENTION

The present invention, in some embodiments thereof, relates to a tobacco transgenic event and methods for detection and use thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have previously generated transgenic plant lines that can be used to produce human procollagen. These transgenic lines express 5 transgenes including human Collagen 1 alpha 1 (Colal), human Collagen 1 alpha 2 (Cola2); human P4H alpha (P4Ha) and P4H beta (P4Hb), as well as human LH3, as described in WO2006/035442 and WO2009/128076.

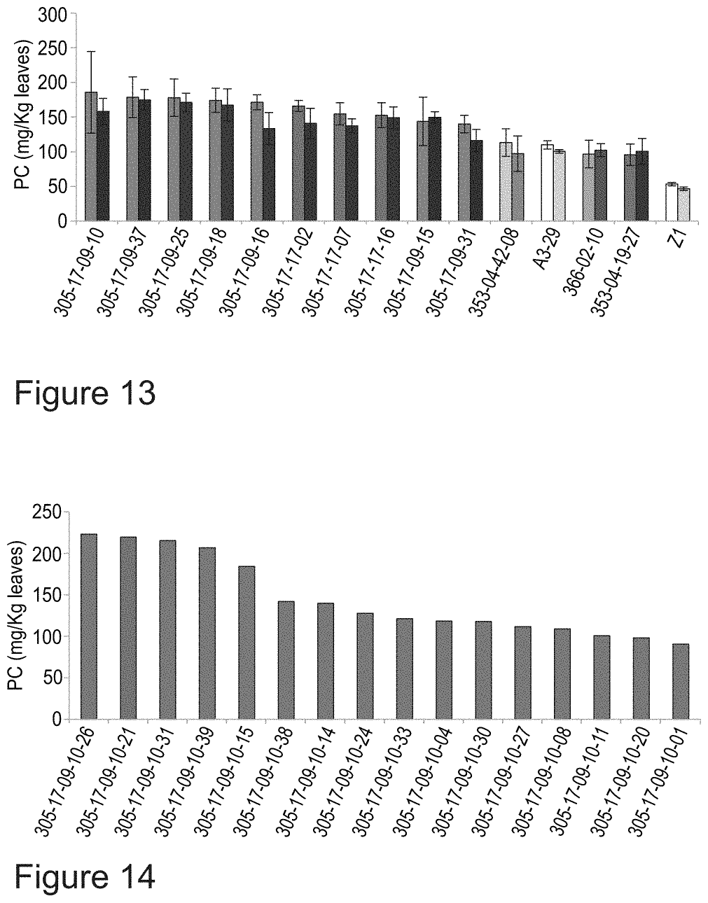

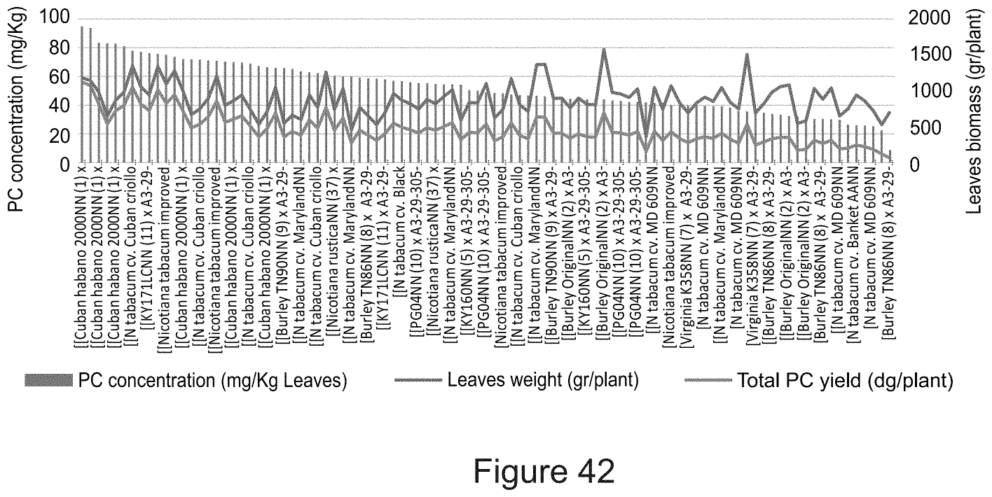

Whilst reducing embodiments of the invention to practice, the present inventors have developed transgenic tobacco plants lines with high yields of human type I procollagen (PC). The breeding program is based on repeated cycles of self-crosses and selection of high yield progenies, which eventually lead to enhanced homozygocity. Homozygous lines are preferred both for their demonstrated higher procollagen yields and the option for propagation via seeds. Seed-based propagation should significantly reduce plantlet costs and shorten the cycle to achieve plantlets for commercial production. The results are based on comparing the F4, F5 lines to the Z1, a hemizygous Samsun line Fl, resultant of crossing of 20-279 (P4Hα; P4Hβ; LH3) with 2-372 (Col1; Col2) (see FIG. 13). The superiority is demonstrated from F4 (A3-29-305-17-09) and progeny such as F5 (A3-29-305-09-18) as well as self-crossing or hybrids thereof, e.g., with different genetic backgrounds (FIGS. 42-44 as well as the Examples section which follows and Tables therein).

The present inventors have then realized that it would be advantageous to be able to detect the presence of the event and in this case, a plurality of integration sites in order to determine whether a progeny of a sexual cross contain a transgene of interest along with its location in the chromosome. In addition, a method for detecting a particular event would be helpful for complying with regulations requiring the pre-market approval and labeling of products derived from recombinant crop plants, or for use in environmental monitoring, monitoring traits in crops in the field, or monitoring products derived from a crop harvest, as well as, for use in ensuring compliance of parties subject to regulatory or contractual terms. Thus, the present inventors have identified molecular junctions that can be used as valuable markers for detecting the presence of the winning event (A3-29-305-17-09 or A3-29-305-17-09-18) or progeny thereof or hybrids thereof. The event is characterized by specific unique DNA segments that are useful in detecting the presence of the event in a sample.

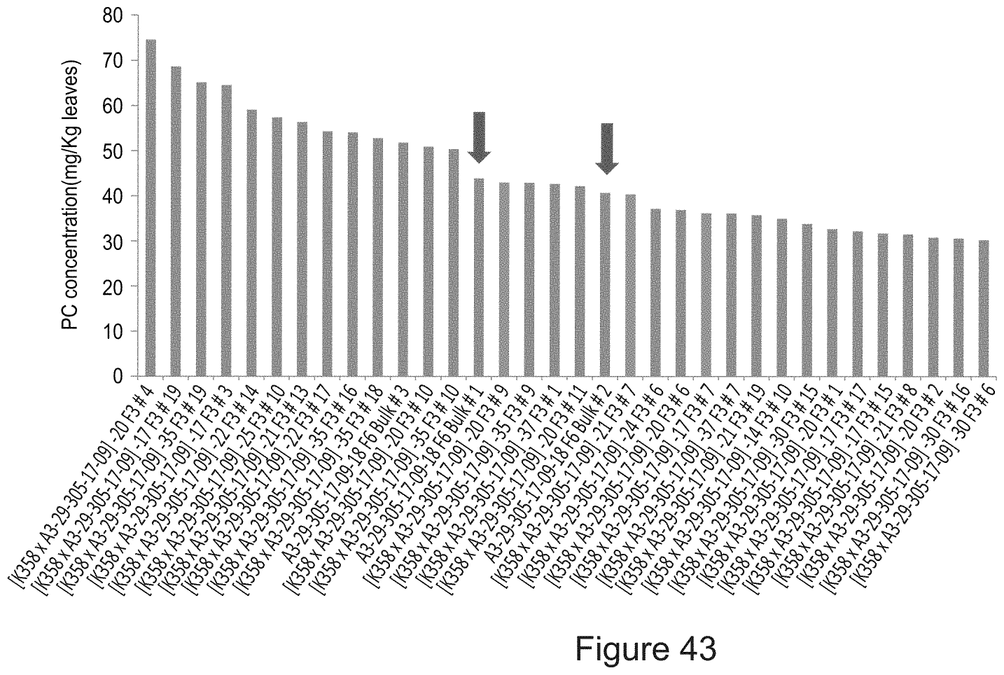

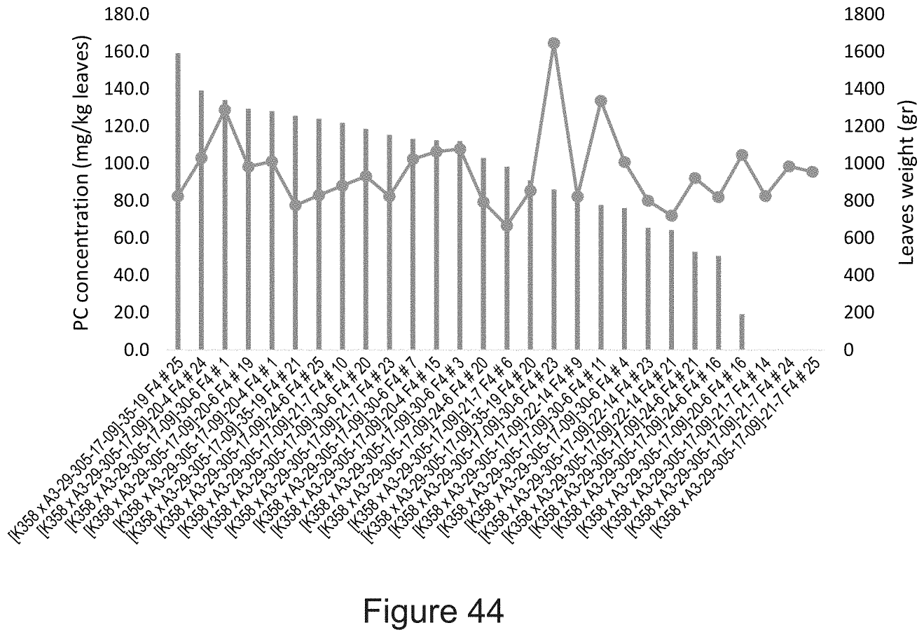

A3-29-305-17-09 or A3-29-305-17-09-18 plants were also used in breeding programs that aim at introducing the event into wild type tobacco cultivars background so as to produce better agriculture performing production lines as well as increasing the yield of the procollagen in the plant (see Examples 3 and 4). For example hybrids (F1), FIG. 42, SHOW CROSS OF A3-29-305-17-09-18 with Samsun lines. Hybrids K358 X A3-29-305-17-09 (F4) are shown in FIGS. 43-44. Further description of such hybrids is provided in Tables 20-30.

As used herein “procollagen” refers to a human collagen molecule that comprises the N-terminal propeptide and the C-terminal propeptide. Human procollagen amino acid sequences are set forth by SEQ ID NOs: 25 and 26.

These sequences are encoded by nucleotide sequences NOs: 20 and 21.

As used herein “P4H” refers to the human P4H enzyme capable of hydroxylating the collagen alpha chain(s) [i.e. hydroxylating only the proline (Y) position of the Gly-X-Y triplets]. P4H is composed of two subunits, alpha and beta as set Genbank Nos. P07237 and P13674. Both subunits are necessary to form an active enzyme, while the beta subunit also possesses a chaperon function. The sequences are encoded by SEQ ID NO: 22 and 23.

As used herein “LH3” or “Lysyl hydroxylase 3” refers to the enzyme set forth in Genbank No. O60568, which can catalyze all three consecutive modifying steps as seen in hydroxylysine-linked carbohydrate formation.

LH3 is encoded by SEQ ID NO: 24.

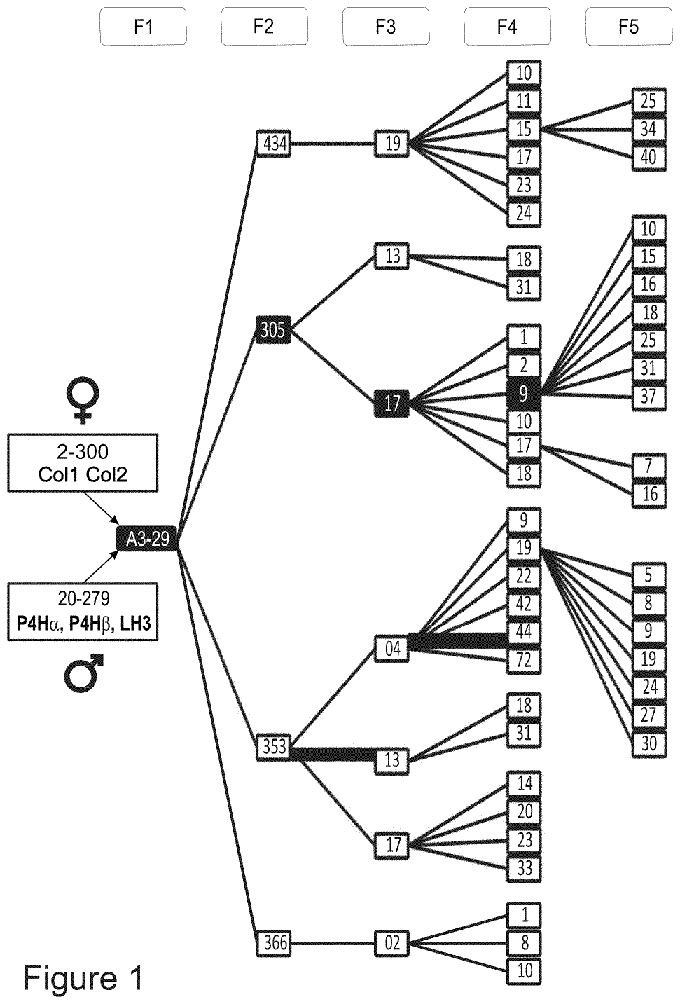

The expression cassettes that were used to transform the initial lines (see FIG. 1) are provided in WO2006/035442 and thus represent the source of the recombinant DNA sequences, which compose the event.

As mentioned, molecular characterization of the integration event was done on the line and A3-29-305-17-09-18 F5, a superior product of self-pollination of the “winning” event A3-29-305-17-09 F4.

Thus, according to an aspect of the invention, there is provided a tobacco plant, plant part, or cell thereof comprising a nucleotide sequence at least 99% identical to SEQ ID NO: 1-19 (e.g., 6 or 9) or complete complement(s) thereof.

A nucleotide sequence(s) at least 99% identical to SEQ ID NOs: represents the “event”.

As used herein the term “event” refers to DNA from the transgenic plant comprising the inserted DNA (recombinant DNA), and flanking genomic sequence (5′ or 3′) of tobacco immediately adjacent to the inserted DNA, also referred to herein as “junctions”. Such DNA is unique and would be expected to be transferred to a progeny that receives the inserted DNA including the transgene of interest as the result of a sexual cross of one parental line that includes the inserted DNA (e.g., A3-29-305-17-09-18 F5) and a stable parental line that does not contain the inserted DNA.

Anywhere in this document, analysis or presence of DNA event being at least 99% identical to SEQ ID NOs: 6 or 9 or complete complements thereof, may be accompanied by analysis or presence of the nucleotide sequences being at least 99% identical to SEQ ID NOs: 1-5, 7-8, 10-18 and/or 19 or complete complements thereof.

Alternatively, analysis can be done on any of nucleotide sequences at least 99% identical to SEQ ID NO: 1-19 (e.g., 6 or 9) or complete complement(s) thereof.

As defined herein, the phrase “stable parental lines” refers to open pollinated, inbred lines, stable for the desired plants over cycles of self-pollination and planting. According to a specific embodiment, 95% of the genome is in a homozygous form in the parental lines of the present invention.

Thus, the event can be transferred to the next generations (progeny) by crossing or self-pollination. Even after repeated backcrossing to a recurrent parent, the event is present in the progeny of the cross at the same chromosomal location.

In this case, the event comprises 10 DNA junctions represented by SEQ ID NOs: 1-19 or a nucleotide sequence at least 99% (e.g., at least 99.1, 99.2, 99.3, 99.4, 99.5 99.6, 99.7, 99.8% e.g., 100%) identical thereto.

An event can be identified by determining the presence of at least 1, 2, 3, 4, 5, 6, 7, 8, 9 or even all (10) junctions.

According to a specific embodiment, each of the transgenes is present in at least single copy in the genome of a heterozygous plant or in at least two copies (e.g., at least 3 copies, at least 4 copies) in a homozygous plant. (e.g., P4Hb-LH3 are present in two locations).

The DNA of the event may be present in each cell and in each genome on one chromosome of the tobacco plant, tobacco seed, and tobacco tissues containing the event. As the tobacco genome is transmitted to progeny in Mendelian fashion, if a tobacco plant were homozygous for the event insertion, each progeny tobacco plant and cell would contain the event DNA on each allele of the parental chromosome containing the event insertion and inherited by the progeny from the parent(s). However, if the tobacco genome containing the event DNA is a heterozygous or hybrid parent, then about fifty percent of the pollen and about fifty percent of the ovules engaged in mating from hybrid parents will contain the tobacco event DNA, resulting in a mixed population of progeny that contain the event DNA, and the percentage of such progeny arising from such crosses with hybrids can range anywhere from about fifty to about seventy five percent having the event DNA transmitted to such progeny.

As used herein, “sequence identity” or “identity” or grammatical equivalents as used herein in the context of two nucleic acid sequences includes reference to the residues in the two sequences, which are the same when aligned.

Identity can be determined using any homology comparison software, including for example, the BlastN software of the National Center of Biotechnology Information (NCBI) such as by using default parameters.

According to a specific embodiment, the plant comprises the event as described herein.

The term “plant” as used herein encompasses whole plants, a grafted plant, and progeny of the plants and plant parts, including seeds, shoots, stems, roots, rootstock, scion, and plant cells, tissues and organs. The plant may be in any form including suspension cultures, embryos, meristematic regions, callus tissue, leaves, gametophytes, sporophytes, pollen, and microspores. According to a specific embodiment, the plant part is a leaf of a seed.

According to a specific embodiment, the plant part comprises DNA (e.g., DNA of the event).

As used herein “tobacco” refers to any plant of the Nicotiana genus, including but not limited to N. tabacum, N. glauca, N. rustica and N. glutinosa.

According to a specific embodiment, the tobacco is of a cultivar belonging to N. tabacum.

According to a specific embodiment, the tobacco is of a cultivar belonging to N. glauca.

According to a specific embodiment, the tobacco is of a cultivar belonging to N. rustica.

According to a specific embodiment, the cultivar is selected from the group consisting of N. tabacum cv. Cuban habano 2000, N. tabacum cv. Burley Original, N. glauca Blue tree, N. tabacum cv. Virginia, N. tabacum cv.KY 160, N. tabacum cv. Virginia K326, N. tabacum cv. Virginia K358, N. tabacum cv. Burley TN86, N. tabacum cv. Burley TN90, N. tabacum cv. PG04, N. tabacum cv. KY 171LC, N. tabacum cv. Maryland, N. tabacum cv. Samsun NN, N. tabacum cv. MD 609, N. tabacum cv. Tukish izmir, N. tabacum cv. Virginia gold 1, N. tabacum cv. Narrow leat Madole, N. tabacum cv. Banket AA, N. tabacum cv. Lizard tail Orinoco, N. tabacum cv. Virginia k346, N. tabacum cv. Black mammoth, N. tabacum cv. Cuban criollo 98, N. tabacum cv. Cuban criollo 98, N. tabacum cv. Cuban criollo 98, N. tabacum perique, N. tabacum little wood, N. tabacum little wood N. and tabacum cv. Burley Hampton.

Additional examples of specific tobacco cultivars which may be used include, but are not limited to brightleaf, burley, cavendish, corojo, criollo, oriental, petite Havana, SR1, thuoc lao, type 22, wild tobacco, Xanthi, and Y1.

As used herein the word “progeny” refers to an offspring or the first (i.e., A3-29-305-17-9-18) and all further descendants from a cross of a plant of the invention that comprises the event with any other plant whether it comprises the event or not. Progeny of the invention are descendants of any cross of a plant of the invention that carries the event.

“Progeny” also encompasses plants that carry the event of the invention which are obtained by vegetative propagation or multiplication.

Thus, according to a specific embodiment, the tobacco plant refers to a tobacco plant which comprises the event A3-29-305-17-9-18 (as described above) or any progeny thereof (as a result of selfing or crossing with an identical background or a different cultivar) or vegentative propagation or multiplication.

According to a specific embodiment, the progeny is F5, F6, F7, F8, F9 or F10.

According to a specific embodiment, the plant is a hybrid plant (e.g., a hybrid seed).

According to a specific embodiment, the plant is an inbred plant.

Examples of such progeny include, but are not limited to A3-29-305-17-09-18 F6; A3-29-305 17 09 18 33 2 F7; A3-29-305 17 09 18 33 10 F7; A3-29-305 17 09 25 04 19 F7; A3-29-305 17 09 37 28 31 F7, as well as the hybrids (as shown e.g., in Tables 20, 21, 21a and 22) described in Examples 3 and 4, as long as they comprise the event.

Progeny plants may be self-pollinated (also known as “selfing”) to generate a true breeding line of plants, i.e., plants homozygous for the transgenes. Selfing of appropriate progeny can produce plants that are homozygous for the transgenes (at least one, 2 3, 4 or 5 transgenes).

Alternatively, progeny plants may be out-crossed, bred with another unrelated plant, to produce a varietal or a hybrid seed or plant. The other unrelated plant may be transgenic or non-transgenic. A varietal or hybrid seed or plant of the invention may thus be derived by sexually crossing a first parent that comprises the specific and unique DNA of the tobacco event A3-29-305-17-9-18 with a second parent comprising an agriculturally valuable trait (e.g., tolerance to biotic and/or abiotic stress, vigor, yield, biomass etc, see e.g., Examples 3-4), resulting in a hybrid comprising the specific and unique DNA of the tobacco event A3-29-305-17-9-18 and having the agriculturally valuable trait which is the result of the crossing and selection and could be the result of heterosis. Each parent can be a hybrid or an inbred/varietal, so long as the cross or breeding results in a plant or seed of the invention, i.e., a seed having at least one allele containing the DNA of tobacco event A3-29-305-17-9-18.

Back-crossing to a parental plant and out-crossing with a non-transgenic plant are also contemplated, as is vegetative propagation. Descriptions of other breeding methods that are commonly used for different traits and crops can be found in one of several references, e.g., Fehr, in Breeding Methods for Cultivar Development, Wilcox J. ed., American Society of Agronomy, Madison Wis. (1987).

According to a specific embodiment, the plant is characterized by a growth temperature 12-36 ° C.

The following measurements are taken at harvest e.g., at harvest stage e.g., 60 days.

According to a specific embodiment, the plant (e.g., hybrid) is characterized by vigor higher than the A3-29 line as manifested by higher biomass by at least 10%, 30%, 50%, 70%, 100 %, 200 %, 250 % or 300 %.

According to a specific embodiment, the plant (e.g., hybrid) is characterized by higher yield than the A3-29 line or Z1 line as manifested by at least 30%, 50%, 70%, 100%, 200%, 250%, 300%, 350%, 400% 500% or more increase in procollagen yield mg/kg leaves.

According to a specific embodiment, the plant (e.g., hybrid) is characterized by leaf weight higher than the A3-29, e.g., at least 450 g/plant. According to a specific embodiment, the leaf weight is higher by at 30%, 40%, 50% than that of A3-29 (e.g., at least 50% for a hybrid of A3-29-305-17-9 F4 or A3-29-305-17-9-18 F5).

According to a specific embodiment, procollagen (e.g., F4, F5 or hybrids thereof) concentration is high e.g., higher than 60 mg/Kg wet leaves.

According to a specific embodiment, procollagen yield is 60-200 mg/plant.

As mentioned, the present inventors have characterized the event and provide herein molecular tools for identifying the event.

Thus, according to an aspect of the invention there is provided a recombinant DNA molecule detectable in a sample containing tobacco DNA, wherein the nucleotide sequence of the molecule is:

a) at least 99% identical to SEQ ID NO: 1-19 (e.g., 6 or 9); or

b) a nucleotide sequence completely complementary to (a),

wherein the presence of the recombinant DNA molecule is diagnostic for tobacco event A3-29-305-17-09-18 DNA or progeny thereof in the sample.

According to an aspect of the invention there is provided a DNA molecule comprising a polynucleotide segment of sufficient length to function as a DNA probe that hybridizes specifically under stringent hybridization conditions e.g., as in Table 31, with a recombinant DNA of tobacco event A3-29-305-17-09-18 or progeny thereof in a sample, wherein hybridization of the DNA molecule under the stringent hybridization conditions is diagnostic for tobacco event A3-29-305-17-09-18 or progeny thereof in the sample.

According to an aspect of the invention there is provided a pair of DNA molecules comprising a first DNA molecule and a second DNA molecule functioning as primers when used together in an amplification reaction with a sample comprising a recombinant DNA of tobacco event A3-29-305-17-09-18 or progeny thereof to produce an amplicon diagnostic for the recombinant DNA of the tobacco event A3-29-305-17-09-18 or progeny thereof in the sample, wherein the amplicon comprises a nucleotide sequence at least 99% identical to SEQ ID NO: 1-19 (e.g., 6 or 9) (e.g., at least 99.1, 99.2, 99.3, 99.4, 99.5 99.6, 99.7, 99.8% e.g., 100% identical thereto).

According to an aspect of the invention there is provided a method of detecting the presence of a recombinant DNA diagnostic for tobacco event A3-29-305-17-09-18 or progeny thereof DNA in a sample, the method comprising:

(a) contacting the sample with the DNA probe molecule as described herein under stringent hybridization conditions; and

(b) detecting hybridization of the DNA molecule to the recombinant DNA,

wherein hybridization is diagnostic for the presence of the recombinant DNA of the tobacco event A3-29-305-17-09-18 or progeny thereof in the sample. Exemplary stringent hybridization conditions are provided in Table 31 but one of skills in the art would know how to modify them within the ranges that still provide for distinct hybridizations.

According to an aspect of the invention there is provided a method of detecting presence of a recombinant DNA of tobacco event A3-29-305-17-09-18 or progeny thereof in a sample, the method comprising:

(a) contacting the sample with a pair of DNA molecules that can serve as primers for amplifying the event;

(b) performing an amplification reaction sufficient to produce a DNA amplicon using the pair of DNA molecules; and

(c) detecting the presence of the DNA amplicon in the reaction,

wherein the DNA amplicon comprises a nucleotide sequence at least 99% (e.g., at least 99.1, 99.2, 99.3, 99.4, 99.5 99.6, 99.7, 99.8% e.g., 100%) identical to SEQ ID NOs: SEQ ID NO: 1-19 (e.g., 6 or 9), and wherein presence of the amplicon is diagnostic for the recombinant DNA of tobacco event A3-29-305-17-09-18 or progeny thereof in the sample.

According to a specific embodiment the recombinant DNA molecule comprises:

a) a nucleotide sequence at least 99% identical to SEQ ID NO: 1-19 (e.g., 6 or 9); or

b) a nucleotide sequence completely complementary to (a).

According to a specific embodiment, the progeny is listed in any one of Tables 20, 21, 21a and 22.

According to a specific embodiment, the recombinant DNA molecule is derived from a tobacco event or progeny thereof comprising SEQ ID NOs: 1-19 (e.g., 6 or 9). sequences at least 99% identical thereto or complete complements thereof.

According to a specific embodiment, the nucleotide sequence is as set forth in SEQ ID NOs: 6 or 9.

As used herein “recombinant DNA” refers to a synthetic DNA which comprises a sequence of a transgene, a cis-acting regulatory sequence (e.g., promoter, enhancer, terminator) or sequence of the DNA cassette used for the expression (e.g., left border, right border etc.).

According to a specific embodiment, the recombinant DNA is devoid of introne sequences.

According to a specific embodiment, the junction comprises the recombinant sequence as well as the genomic sequence of the tobacco plant in a 5′ to 3′ or 3′ to 5′ orientation dependent on the integration of the recombinant sequence in the sense or anti-sense strand of the genomic DNA.

As used, herein “sample” refers to a composition that is either substantially pure tobacco DNA or a composition that contains tobacco DNA. In either case, the sample is a biological sample, i.e., it contains biological materials (but may also contain non-biological material), including but not limited to DNA obtained or derived from, either directly or indirectly, from the genome of tobacco comprising the event or progeny thereof. “Directly” refers to the ability of the skilled artisan to directly obtain DNA from the tobacco genome by fracturing tobacco cells (or by obtaining samples of tobacco that contain fractured tobacco cells) and exposing the genomic DNA for the purposes of detection.

“Indirectly” refers to the ability of the skilled artisan to obtain the target or specific reference DNA, i.e. a novel and unique junction described herein as being diagnostic for the presence of the event in a particular sample, by means other than by direct via fracturing of tobacco cells or obtaining a sample of tobacco that contains fractured tobacco cells. Such indirect means include, but are not limited to, amplification nucleotide sequence that is comprises in the event using a particular probe(s) or primers designed to bind with specificity to the target sequence, or amplification of a DNA that can be measured and characterized, i.e.

measured by separation from other sequences of DNA through some efficient matrix such as an agarose or acrylamide gel or the like, or characterized by direct sequence analysis of the amplicon or cloning of the amplicon into a vector and direct sequencing of the inserted amplicon present within such vector. Alternatively, a nucleotide sequence of DNA corresponding to the position within the tobacco chromosome at which the transgenic DNA was inserted into the tobacco chromosome and which can be used to define the event, can be cloned by various means and then identified and characterized for its presence in a particular sample or in a particular tobacco genome. Such DNA sequences are referred to as junction sequence or sequences, and can be any length of inserted DNA and adjacent (flanking) tobacco chromosome DNA so long as the point of joining between the inserted DNA and the tobacco genome is included in the sequence. SEQ ID NO: 1-19 (e.g., 6 or 9) (or homologs thereof, at least 99.1, 99.2, 99.3, 99.4, 99.5 99.6, 99.7, 99.8% e.g., 100% identical thereto) and the reverse complement of each of these, are representative of such segments (as well as the at least 99% identity homologs that are defined above).

The specific sequences identified herein are present uniquely in the event or the construct comprised therein, and the identification of these sequences, whether by direct sequence analysis, by detecting probes bound to such sequences, or by observing the size or the composition of particular amplicons described herein, when present in a particular tobacco germplasm or genome and/or present in a particular biological sample containing tobacco DNA, are diagnostic for the presence of the event, or the construct comprised therein, in such sample. It is known that the flanking genomic sequences, i.e., the tobacco genome segments of DNA sequence adjacent to the inserted transgenic DNA) are subject to slight variability and as such, the limitation of at least 99% or greater (e.g., at least 99.1, 99.2, 99.3, 99.4, 99.5 99.6, 99.7, 99.8% e.g., 100%) identity is with reference to such anomalies or polymorphisms from tobacco genome to tobacco genome.

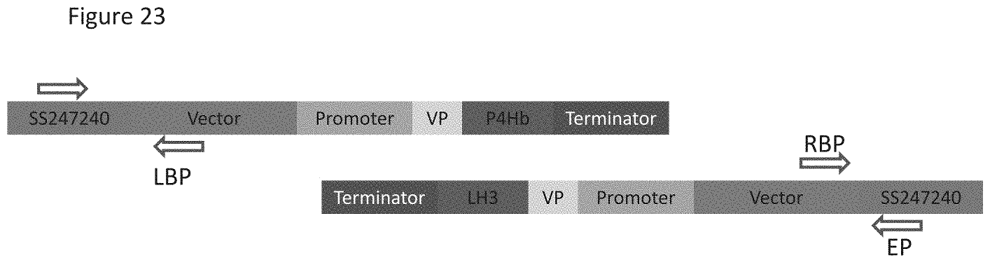

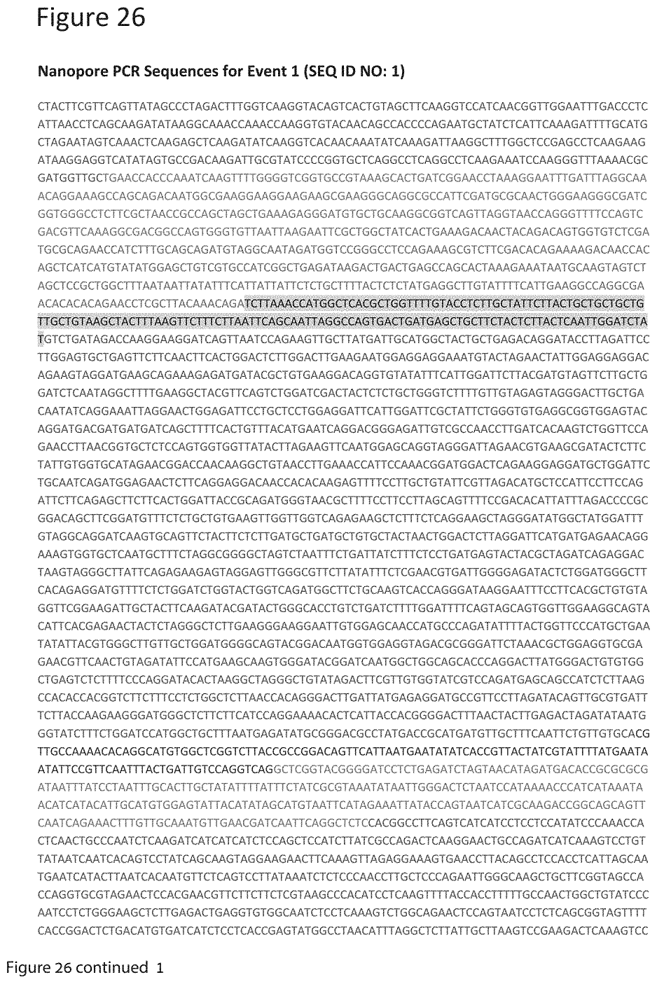

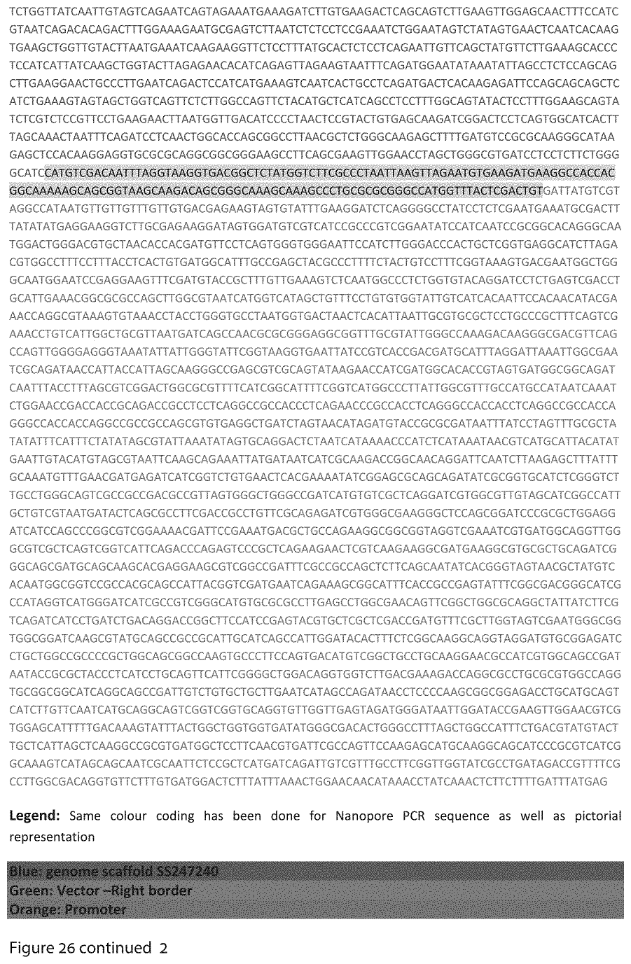

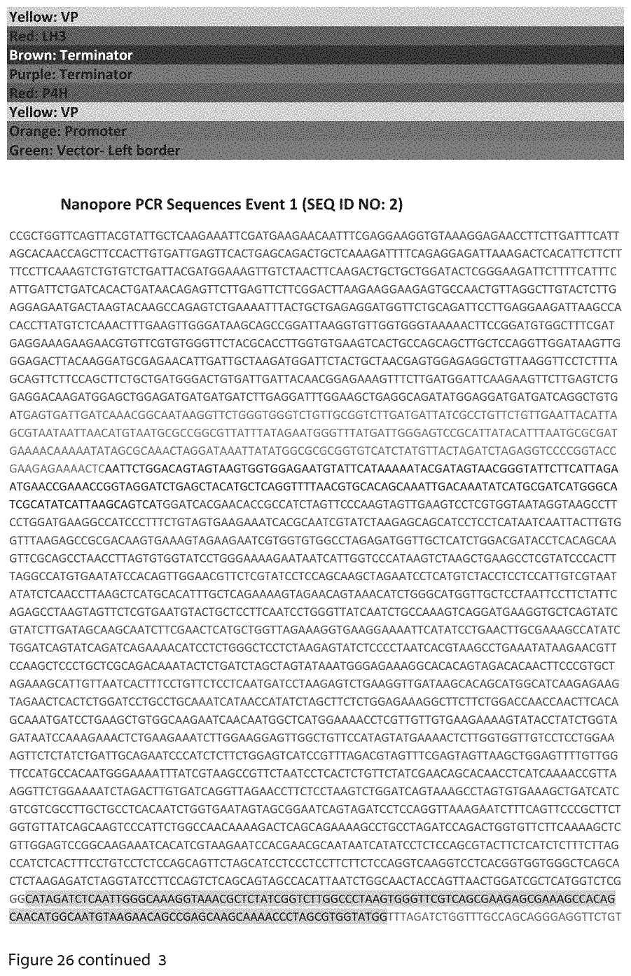

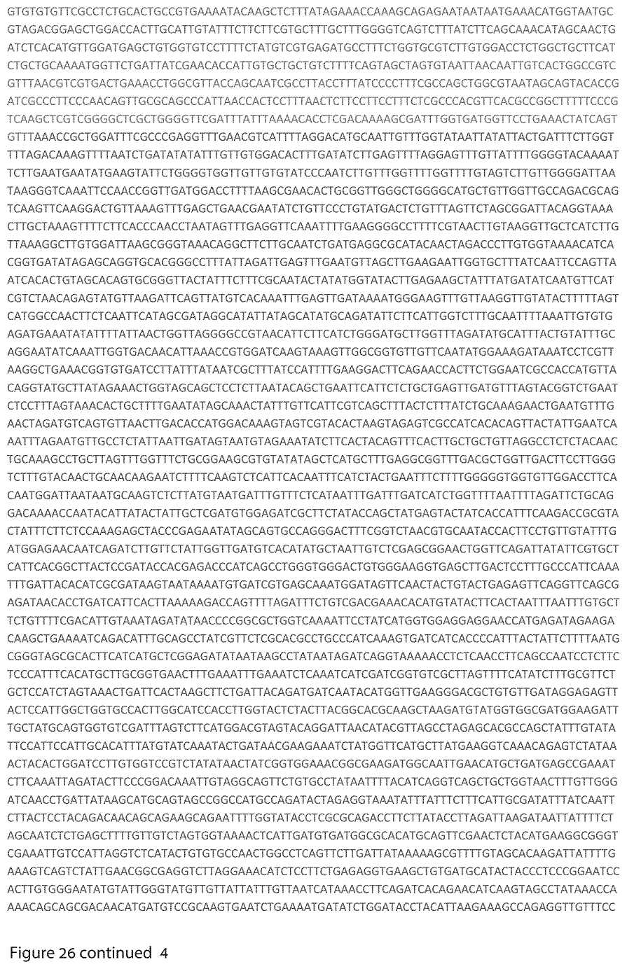

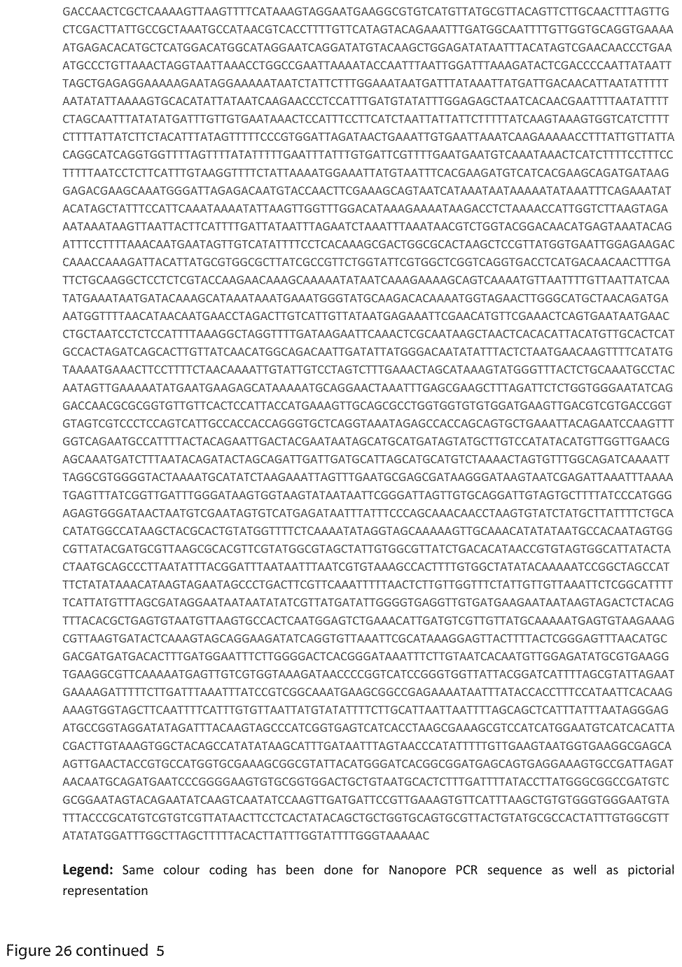

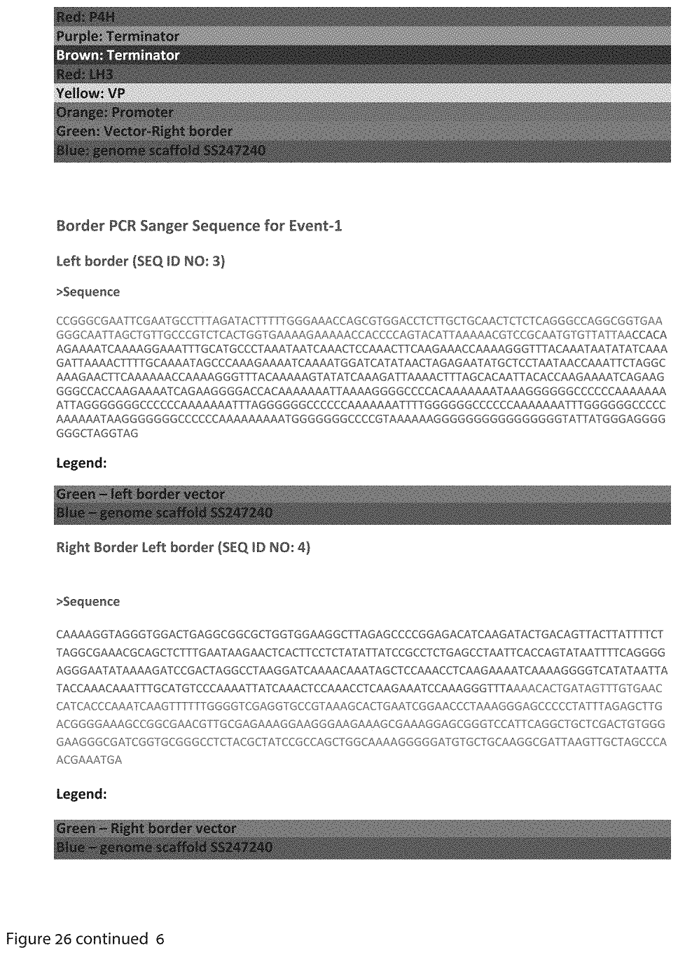

The position/orientation of the nucleotide sequences (transgenes) of the present invention are illustrated in FIGS. 23, 27, 31, 35, and 38. SEQ ID NOs: 1-19 of representative amplicons are illustrated in FIGS. 26, 30, 34, 37 and 40. The presence of one (e.g., SEQ ID NOs: 6 or 9), or two, or more of these nucleotide sequences in a sample, when such a sample contains tobacco cells or portions thereof and thus tobacco DNA (optionally any of SEQ ID NOs: 1-5. 7-8, 10-19 and sequences at least 99% identity thereof ore complete complements thereof), are diagnostic for the presence of the event.

It is intended by use of the word “derived” that a particular DNA molecule is in the tobacco plant genome, or is capable of being detected in tobacco plant DNA. “Capable of being detected” refers to the ability of a particular DNA sequence to be amplified and its size and or sequence characterized or elucidated by DNA sequence analysis, and can also refer to the ability of a probe to bind specifically to the particular DNA sequence, i.e. the target DNA sequence, and the subsequent ability to detect the binding of the probe to the target. The particular DNA segment or target DNA segment of the present invention is present within tobacco that contains the event.

The DNA molecules of the present invention may be unique to the junctions on either end of the inserted transgenic event DNA and the tobacco genome DNA that is adjacent to, i.e. flanking, each end of the inserted DNA, or unique to the tobacco event inserted DNA. These molecules, when present in a particular sample analyzed by the methods described herein using the probes, primers and in some cases using DNA sequence analysis, may be diagnostic for the presence of an amount of event tobacco in that sample. Such DNA molecules unique to the tobacco event DNA can be identified and characterized in a number of ways, including by use of probe nucleic acid molecules designed to bind specifically to the unique DNA molecules followed by detection of the binding of such probes to the unique DNA, and by thermal amplification methods that use at least two different DNA molecules that act as probes but the sequence of such molecules may be somewhat less specific than the probes described above. The skilled artisan understands that contacting a particular target DNA with a probe or primer under appropriate hybridization conditions will result in the binding of the probe or primer to the targeted DNA segment.

The DNA molecules of the present invention may be target segments of DNA that may be capable of amplification and, when detected as one or more amplicons of the represented length obtained by amplification methods of a particular sample, may be diagnostic for the presence of event, or the construct comprised therein, in such sample. Such DNA molecules or polynucleotide segments may have the nucleotide sequences as set forth in each of SEQ ID NO: 6 or 9 and optionally SEQ ID NOs: 1-5, 7-8, 10-18 and/or 19 or sequences at least 99% identical thereto (see above), and are further defined herein and in the examples below. Primer molecules and/or probes may be provided in kit form along with the necessary reagents, including controls, and packaged together with instructions for use.

Probes for use herein may comprise DNA molecules or polynucleotide segments of sufficient length to function under stringent hybridization conditions as defined herein to bind with a particular target DNA segment, i.e., a unique segment of DNA present within and diagnostic for the presence of, event DNA in a sample. Such a probe can be designed to bind only to a single junction or other novel sequence present only in the tobacco event DNA, or to two or more such single junction segments. The detection of the binding of such a probe to a DNA molecule in a particular sample suspected of containing tobacco DNA is diagnostic for the presence of tobacco event in the sample.

Since the present event comprises a plurality of junctions, a multiplex amplification reaction to a plurality of junctions can be performed (i.e., using simultaneously more than a primer pair).

Primers may comprise pairs of different oligonucleotides or polynucleotide segments for use in a thermal amplification reaction which amplifies a particular DNA target segment. Each primer in the pair is designed to bind to a rather specific segment of DNA within or near to a segment of DNA of interest for amplification. The primers bind in such way that these then act as localized regions of nucleic acid sequence polymerization resulting in the production of one or more amplicons (amplified target segments of DNA). In the present invention, use of primers designed to bind to unique segments of tobacco event DNA in a particular biological sample and that amplify particular amplicons containing one or more of the junction segments described herein, and the detection and/or characterization of such amplicons upon completion or termination of the polymerase reaction, is diagnostic for the presence of the tobacco event in the particular sample. The skilled artisan is well familiar with this amplification method and no recitation of the specifics of amplification is necessary here.

As used herein a “probe” refers to an isolated nucleic acid sequence to which may be attached a conventional detectable label or reporter molecule, e.g., a radioactive isotope, ligand, chemiluminescent agent, fluorescent agent or enzyme. Such a probe is complementary to a strand of a target nucleic acid, in the case of the present invention, to a strand of DNA from tobacco whether from an event containing plant or from a sample that includes the event DNA. Probes according to the present invention include not only deoxyribonucleic or ribonucleic acids, but also polyamides and other probe materials that bind specifically to a target DNA sequence and can be used to detect the presence of the event.

DNA primers are isolated polynucleic acids that are annealed to a complementary event DNA strand (target DNA strand) by nucleic acid hybridization to form a hybrid between the primer and the target DNA strand, then extended along the target DNA strand by a polymerase, e.g., a DNA polymerase. A DNA primer pair or a DNA primer set of the present invention refer to two DNA primers useful for amplification of a target nucleic acid sequence, i.e., event A3-29-305-17-09, by the polymerase chain reaction (PCR) or other conventional polynucleic acid amplification methods.

DNA probes and DNA primers may be at least 11 nucleic acids or more in length, or may be at least 18 nucleic acids or more, at least 24 nucleic acids or more, or at least 30 nucleic acids or more (e.g., 11-100, 15-100, 20-100, 30-100, 11-50, 15-50, 20-50, 30-50 nucleotides in length). Such probes and primers are selected to be of sufficient length to hybridize specifically to a target sequence under high stringency hybridization conditions. Preferably, probes and primers according to the present invention have complete sequence similarity with the target sequence, although probes differing from the target sequence that retain the ability to hybridize to target sequences may be designed by conventional methods (e.g., comprising at least 1 mismatch, 2 mismatches, 3 mismatches, 4 mismatches, 5 mismatches or mode, e.g., at least 10 mismatches on a sequence of at least 300 bp).

Primers and probes based on the flanking genomic DNA and insert (recombinant) sequences disclosed herein can be used to confirm (and, if necessary, to correct) the disclosed DNA sequences by conventional methods, e.g., by re-cloning and sequencing such DNA molecules.

According to a specific embodiment, the nucleic acid probes and primers of the present invention hybridize under stringent conditions to a target DNA molecule (i.e., the event). Any conventional or no-conventional nucleic acid hybridization or amplification method can be used to identify the presence of DNA from a transgenic plant in a sample.

Polynucleic acid molecules also referred to as nucleic acid segments or fragments thereof are capable of specifically hybridizing to other nucleic acid molecules under certain circumstances. As used herein, two polynucleic acid molecules are capable of specifically hybridizing to one another if the two molecules are capable of forming an anti-parallel, double- stranded nucleic acid structure. A nucleic acid molecule is the to be the “complement” of another nucleic acid molecule if they exhibit complete complementarity. As used herein, molecules are to exhibit “complete complementarity” when every nucleotide of one of the molecules is complementary to a nucleotide of the other. Two molecules are the to be “minimally complementary” if they can hybridize to one another with sufficient stability to permit them to remain annealed to one another under at least conventional “low-stringency” conditions. Similarly, the molecules are the to be “complementary” if they can hybridize to one another with sufficient stability to permit them to remain annealed to one another under conventional “high-stringency” conditions. Conventional stringency conditions are described by Sambrook et ah, 1989, and by Haymes et ah, In: Nucleic Acid Hybridization, A Practical Approach, IRL Press, Washington, DC (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. Appropriate stringency conditions that promote DNA hybridization, for example, 6.0× sodium chloride/sodium citrate (SSC) at about 45 ° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65 ° C. Both temperature and salt may be varied, or either the temperature or the salt concentration may be held constant while the other variable is changed. In a preferred embodiment, a polynucleic acid of the present invention will specifically hybridize to one or more of the nucleic acid molecules set forth in SEQ ID NO: 6 or 9 and optionally SEQ ID NOs: 1-5, 7-8, 10-18 and/or 19, or complements thereof or fragments of either under stringent conditions, such as described herein and in the art.

Examples of probes and primers that can be used are provided in Example 2 as described below.

The hybridization of the probe to the target DNA molecule can be detected by any number of methods known to those skilled in the art, these can include, but are not limited to, fluorescent tags, radioactive tags, antibody based tags, fluorescent tags and chemiluminescent tags.

Regarding the amplification of a target nucleic acid sequence (e.g., by PCR) using a particular amplification primer pair, “stringent conditions” are conditions that permit the primer pair to hybridize only to the target nucleic acid sequence to which a primer having the corresponding wild-type sequence (or its complement) would bind and preferably to produce a unique amplification product, the amplicon, in a DNA thermal amplification reaction. Examples of such conditions are described in Example 2 of the Examples section which follows.

The term “specific for (a target sequence)” indicates that a probe or primer hybridizes, e.g., under stringent hybridization conditions, only to the target sequence in a sample comprising the event or that unavoidable hybridization to sequences that do not make up the event can be easily distinguishable (e.g., by size, sequence etc.).

As used herein, “amplified DNA” or “amplicon” refers to the product of polynucleic acid amplification method directed to a target polynucleic acid molecule that is part of a polynucleic acid template.

For example, to determine whether a tobacco plant resulting from a sexual cross contains the event of the present invention, DNA that is extracted from a tobacco plant tissue sample may be subjected to a polynucleic acid amplification method using a primer pair that includes a first primer derived from a genomic DNA sequence in the region flanking the heterologous inserted DNA of event and is elongated by polymerase 5′ to 3′ in the direction of the inserted DNA. The second primer is derived from the heterologous inserted DNA molecule is elongated by the polymerase 5′ to 3′ in the direction of the flanking genomic DNA from which the first primer is derived.

Alternatively, a primer pair can be derived from genomic sequence on both sides of the inserted heterologous DNA so as to produce an amplicon that includes the entire insert polynucleotide sequence.

A member of a primer pair derived from the plant genomic sequence adjacent to the inserted transgenic DNA is located a distance from the inserted DNA sequence, this distance can range from one nucleotide base pair up to about twenty thousand nucleotide base pairs.

The use of the term “amplicon” specifically excludes primer dimers that may be formed in the DNA thermal amplification reaction.

For practical purposes, one should design primers, which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TAQMAN®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. In addition, primers should be designed such that they cover (by amplification) a small portion of the tobacco genome. Such a small portion should be sufficient to identify the event even if longer genomic sequences are lost due to crossing with another genetic background.

Examples of specific primers, which can be used in accordance with the teachings of the invention, are provided in Example 2, which follows (e.g., SEQ ID NOs: 27-47).

Polynucleic acid amplification can be accomplished by any of the various polynucleic acid amplification methods known in the art, including the polymerase chain reaction (PCR). Amplification methods are known in the art and are described, inter alia, in U.S. Pat. Nos. 4,683,195 and 4,683,202 and in PCR Protocols: A Guide to Methods and Applications, ed. Innis et ah, Academic Press, San Diego, 1990. PCR amplification methods have been developed to amplify up to 22 kb (kilobase) of genomic DNA and up to 42 kb of bacteriophage DNA (Cheng et al, Proc. Natl. Acad. Sci. USA 91:5695-5699, 1994). These methods as well as other methods known in the art of DNA amplification may be used in the practice of the present invention.

The diagnostic amplicon produced by these methods may be detected by a plurality of techniques.

Sanger sequencing and Nanopore-based sequencing are shown at great details in Example 2 of the Examples section which follows.

Another such method is Genetic Bit Analysis (Nikiforov, et al. Nucleic Acid Res. 22:4167-4175, 1994) where a DNA oligonucleotide is designed that overlaps both the adjacent flanking genomic DNA sequence and the inserted DNA sequence. The oligonucleotide is immobilized in wells of a microtiter plate. Following PCR of the region of interest (using one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic sequence due to successful amplification, hybridization, and single base extension.

Another method is the Pyrosequencing technique as described by Winge (Innov. Pharma. Tech. 00: 18-24, 2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen, et al., (Genome Res. 9:492- 498, 1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Taqman® (PE Applied Biosystems, Foster City, Calif.) is described as a method of detecting and quantifying the presence of a DNA sequence and is fully understood in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular Beacons have been described for use in sequence detection as described in Tyangi, et al. (Nature Biotech.U:303-308, 1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

DNA detection kits that are based on DNA amplification methods contain DNA primer molecules that hybridize specifically to a target DNA and amplify a diagnostic amplicon under the appropriate reaction conditions. The kit may provide an agarose gel based detection method or any number of methods of detecting the diagnostic amplicon that are known in the art. DNA detection kits can be developed using the compositions disclosed herein and are useful for identification of tobacco event DNA in a sample and can be applied to methods for breeding tobacco plants containing event DNA.

The invention provides exemplary DNA molecules that can be used either as primers or probes for detecting the presence of DNA derived from a tobacco plant comprising event DNA in a sample. Such primers or probes are specific for a target nucleic acid sequence and as such are useful for the identification of tobacco event nucleic acid sequence by the methods of the invention described herein.

As mentioned, probes and primers according to the invention may have complete sequence identity with the target sequence, although primers and probes differing from the target sequence that retain the ability to hybridize preferentially to target sequences may be designed by conventional methods. In order for a nucleic acid molecule to serve as a primer or probe it need only be sufficiently complementary in sequence to be able to form a stable double- stranded structure under the particular solvent and salt concentrations employed. Any conventional nucleic acid hybridization or amplification method can be used to identify the presence of transgenic DNA from tobacco event in a sample. Probes and primers are generally at least about 11 nucleotides, at least about 18 nucleotides, at least about 24 nucleotides, or at least about 30 nucleotides or more in length. Such probes and primers hybridize specifically to a target DNA sequence under stringent hybridization conditions.

According to a specific embodiment the primers or probes are at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 nucleotides in length (e.g., 100% complementary to SEQ ID NO: 1-19.

Conventional stringency conditions are described by Sambrook et ah, 1989, and by Haymes et ah, In: Nucleic Acid Hybridization, A Practical Approach, IRL Press, Washington, DC (1985).

Any number of methods well known to those skilled in the art can be used to isolate and manipulate a DNA molecule, or fragment thereof, disclosed in the invention, including thermal amplification methods. DNA molecules, or fragments thereof, can also be obtained by other techniques such as by directly synthesizing the fragment by chemical means, as is commonly practiced by using an automated oligonucleotide synthesizer.

The DNA molecules and corresponding nucleotide sequences provided herein are therefore useful for, among other things, identifying tobacco event, selecting plant varieties or hybrids comprising tobacco event, detecting the presence of DNA derived from the transgenic tobacco event in a sample, and monitoring samples for the presence and/or absence of tobacco event or plant parts derived from tobacco plants comprising event.

Thus, according to an aspect of the invention there is provided a method of producing a plant having an improved agricultural trait, the method comprising:
(a) subjecting the plant comprising the event to a breeding program and/or transgenesis and/or genome editing; and
(b) selecting a plant exhibiting an improved agricultural trait.

Transgenic transformation and genome editing techniques are well known to the skilled artisan.

Regardless of the technique used for the identification, once procollagen-expressing progeny are identified, such plants are further cultivated under conditions which maximize expression thereof. Progeny resulting from transformed plants can also be selected, by verifying presence of exogenous mRNA and/or polypeptides by using nucleic acid or protein probes (e.g. antibodies). The latter approach enables localization of the expressed polypeptide components (by for example, probing fractionated plants extracts) and thus also verifies a potential for correct processing and assembly.

Following cultivation of such plants, the procollagen is typically harvested. Plant tissues/cells are harvested at maturity, and the procollagen molecules are isolated using any biochemical method known in the art.

Thus, according to an aspect of the invention there is provided method of producing procollagen, the method comprising:
(a) growing the plant comprising the event; and
(b) isolating the procollagen from the plant.

Also provided is a procollagen obtainable according to the method as described herein.

It will be appreciated that the plant can be grown according to the demands of the selected cultivar. For instance the present inventors were able to generate hybrids that comprise the event and are capable of high yield procollagen production (as described above, e.g., above 60 mg/plant) at a temperature range of 12-36° C.

Thus, embodiments of the present invention further provide for a method of purifying procollagen.

The method comprising providing a procollagen preparation (a product of procollagen isolation) and purifying the procollagen.

Procollagen may be fully purified or partially purified using any protein purification technique known in the art. These methods are typically based on size, charge or binding affinity purification.

According to one embodiment, the procollagen is comprised in a procollagen-containing composition, in which at least 0.1%, at least 0.25%, at least 0.5%, at least 1%, at least 2.5%, at least 5%, at least 10%, at least 20%, at least 30%, at least 40%, at least 50% , at least 60%, at least 70%, at least 80%, at least 90%, at least 92%, at least 93%, at least 94%, at least 95%, at least 96%, at least 97%, at least 99% or 100% is procollagen.

Other components comprised in the procollagen composition may include but are not limited to collagen, hyaluronic acid, alginate, carboxymethylcellulose, hydroxymethylcellulose, hydroxyethylcellulose, oxidized cellulose, cellulose whiskers, and starch.

As used herein, “purifying” refers to the isolation of the protein from its natural environment or site of accumulation within the recombinant host. Separation from small molecules is typically effected by dialysis such as using cellulose membranes. Gel-filtration chromatorgraphy is typically used as a more discriminative technique. Alternatively or additionally, salting-out is used, such as with ammonium sulfate which is typically used for protein purification e.g., to precipitate fibrinogen. Yet alternatively or additionally, ion exchange chromatography is used to separate procollagen on the basis of net charge. Affinity chromatography is another powerful approach for isolation of proteins of interest. More specifically, antibodies can be used or affinity-binding methods based on the protein's natural attractive forces to certain chemical groups.

Exemplary methods of purifying or semi-purifying procollagen of the present invention are described in detail in the Examples section, which follows.

Procollagen may be further processed to collagen.

As used, herein “collagen” relates to a polypeptide having a triple helix structure and containing a repeating Gly-X-Y triplet, where X and Y can be any amino acid but are frequently the imino acids proline and hydroxyproline. According to the present invention, the collagen is type I collagen devoid of the N and C propeptides.

The collagen may be telocollagen or atelocollagen.

According to one embodiment, the collagen of the present invention comprises a sufficient portion of its telopeptides such that under suitable conditions it is capable of forming fibrils.

Thus, for example, the collagen may be atelocollagen, a telocollagen or digested procollagen.

As used herein, the term “atelocollagen” refers to collagen molecules lacking both the N- and C-terminal propeptides typically comprised in procollagen, but including a sufficient portion of its telopeptides such that under suitable conditions it is capable of forming fibrils.

The term “telocollagen” as used herein, refers to collagen molecules that lack both the N- and C-terminal propeptides typically comprised in procollagen but still contain the telopeptides. The telopeptides of fibrillar collagen are the remnants of the N-and C-terminal propeptides following digestion with native N/C proteinases.

Proteases capable of correctly cleaving recombinant propeptide or telopeptide-comprising collagen are known in the art. These include certain plant derived proteases e.g. ficin (EC 3.4.22.3) and certain bacterial derived proteases e.g. subtilisin (EC 3.4.21.62), neutrase.

The procollagen or telocollagen is contacted with the proteases under conditions such that the proteases are able to cleave the propeptides or telopeptides therefrom. Typically, the conditions are determined according to the particular protease selected. Thus, for example procollagen may be incubated with a protease for up to 15 hours, at a concentration of 1-25 mg/ml and a temperature of about 10-20 ° C.

Following protease digestion, the generated atelocollagen may be further purified e.g. by salt precipitation, as described in WO2009/053985 so that the end product comprises a purified composition of atelocollagen having been processed from plant or plant-cell generated procollagen by a protease selected from the group consisting of neutrase, subtilisin, ficin and recombinant human trypsin and analyzed using methods known in the art (e.g. size analysis via Coomassie staining, Western analysis, etc.).

Following purification, the atelocollagen may be resolubilized by addition of acidic solutions (e.g. 10 mM HC1). Such acidic solutions are useful for storage of the purified atelocollagen.

Following digestion e.g., with ficin, the atelocollagen maintains its ability to form fibrils upon neutralization of the above described acid solutions. According to one embodiment, at least 70% of the purified and resolubilized atelocollagen generated according to the method of the present invention is capable of forming fibrils. According to one embodiment, at least 90% of the purified and resolubilized atelocollagen generated according to the method of the present invention is capable of forming fibrils.

The ability to form fibrils demonstrates that the generated atelocollagen is useful for medical purposes including, but not limited to cosmetic surgery, healing aid for burn patients, reconstruction of bone and a wide variety of dental, orthopedic and surgical purposes.

According to another embodiment, the collagen is a mixture of the types of collagen and/or procollagen described above.

Regardless of the method of production, once the procollagen or collagen is at hand it can be administered to the subject per se or in a pharmaceutical composition or in a medical device.

As used herein, a “pharmaceutical composition” refers to a preparation of the active ingredients described herein with other chemical components such as physiologically suitable carriers and excipients. The purpose of the pharmaceutical composition is to facilitate administration of the active ingredients (e.g., procollagen) to the subject.

As used herein, the term “active ingredient” refers to the procollagen or collagen accountable for the intended biological effect (i.e., promoting wound healing and treating fibrosis).

Hereinafter, the phrases “physiologically acceptable carrier” and “pharmaceutically acceptable carrier”, which may be interchangeably used, refer to a carrier or a diluent that do not cause significant irritation to the subject and do not abrogate the biological activity and properties of the administered active ingredients. An adjuvant is included under these phrases.

Herein, the term “excipient” refers to an inert substance added to the pharmaceutical composition to further facilitate administration of an active ingredient of the present invention.

Techniques for formulation and administration of drugs may be found in “Remington's Pharmaceutical Sciences,” Mack Publishing Co., Easton, PA, latest edition, which is incorporated herein by reference.

The pharmaceutical composition may be formulated as a unit dosage form. In such form, the preparation is subdivided into unit doses containing appropriate quantities of the active ingredients such as for a single administration. The unit dosage form can be a packaged preparation, the package containing discrete quantities of preparation, for example, an adhesive bandage, a non-adhesive bandage, a wipe, a baby wipe, a gauze, a pad and a sanitary pad.

The pharmaceutical compositions of the present invention may be applied in a local manner, for example, via administration of the compositions directly onto a tissue region (e.g. wound) of the subject. Suitable routes of administration of pharmaceutical compositions may, for example, include topical (e.g., to a keratinous tissue, such as the skin, hair, nail, scalp), subcutaneous, mucosal (e.g., oral, vaginal, eye), intramuscular administrations.

The pharmaceutical compositions of the present invention may also be applied via injecting the composition including the active ingredient and a physiologically acceptable carrier. For local administration, the compositions may be injected into the wound, and/or into healthy tissue (e.g., skin) that surrounds the wounded tissue, or both e.g., subcutaneous.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

The active ingredient may also be in a powder form for constitution with a suitable vehicle, e.g., sterile, pyrogen-free water-based solution, before use.

Pharmaceutical compositions for use in accordance with the present invention thus may be formulated in conventional manner using one or more physiologically acceptable carriers comprising excipients and auxiliaries, which facilitate processing of the active ingredients into preparations. Proper formulation is dependent upon the administration approach chosen.

Determination of a therapeutically effective amount is well within the capability of those skilled in the art, especially in light of the detailed disclosure provided herein. Treatment can be effected prior to the formation of massive scar tissue for instance, such as prior to the recruitment of fibroblasts to the affected site. However, the present invention also envisages administering the procollagen or collagen at any other stage of healing.

For any preparation used in the method of the invention, the therapeutically effective amount or dose can be estimated initially from in vitro assays. In addition, a dose can be formulated in tissue culture systems or in animal models to achieve a desired concentration or titer. Animal models may be used in order to establish criteria for administration. For example, a diabetic rat or mouse wound model may be used [Galeano et al., Diabetes. (2004) 53(9):2509-17]. Outcome measures such as perfusion and survival, as well as histological and functional criteria, can be employed to assess the efficacy of the different parameters, in order to approach optimal efficiency.

Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the type of formulation employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p.1.)

Depending on the severity of the condition (e.g., the area, depth and degree of the wound or the scar) and the responsiveness of the skin, dosing can be of a single or a plurality of administrations, with course of treatment ranging from several days to several weeks or until cure is effected or diminution of the condition is achieved. In exemplary embodiments, the pharmaceutical composition of the present invention is administered at least once a day.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

Since the pharmaceutical compositions of the present invention are utilized in vivo, the compositions are preferably of high purity and substantially free of potentially harmful contaminants, e.g., at least National Food (NF) grade, generally at least analytical grade, and preferably at least pharmaceutical grade. To the extent that a given compound must be synthesized prior to use, such synthesis or subsequent purification shall preferably result in a product that is substantially free of any potentially contaminating toxic agents that may have been used during the synthesis or purification procedures.

To improve therapeutic efficacy, additional agents may be incorporated into the pharmaceutical compositions of the present invention. Agents for promoting wound healing, treating fibrosis and/or promoting angiogenesis can be formulated in a single composition together with the procollagen (e.g., single container) or collagen or when desired, packed in separate containers and included in an article of manufacture, which may further comprise instructions for use. Such agents include, but are not limited to, extracellular matrix components (e.g. vitronectin, laminin, collagen, elastin), growth factors (e.g. FGF 1, FGF 2, IGF 1, IGF 2, PDGF, EGF, KGF, HGF, VEGF, SDF-1, GM-CSF, CSF, G-CSF, TGF alpha, TGF beta, NGF, PDWHF and ECGF), hypoxia inducible factors (e.g. HIF-1 alpha and beta and HIF-2), hormones (e.g., insulin, growth hormone (GH), CRH, Leptin, Prolactin, oxandrolone and TSH), angiogenic factors (e.g., angiogenin and angiopoietin), coagulation and anticoagulation factors (e.g., Factor I, Factor XIII, tissue factor, calcium, vWF, protein C, protein S, protein Z, fibronectin, antithrombin, heparin, plasminogen, low molecular weight heparin (Clixan), high molecular weight kininogen (HMWK), prekallikrein, plasminogen activator inhibitor-1 (PAI1), plasminogen activator inhibitor-2 (PAI2), urokinase, thrombomoduline, tissue plasminogen activator (tPA), alpha 2-antiplasmin and Protein Z-related protease inhibitor (ZPI)), cytokines (IL-1 alpha, IL-1 beta, IL-2, IL-3, IL-4, IL-5, IL-6, IL-7, IL-8, IL-9, IL-10, IL-11, IL-12, IL-13 and IFN-alpha, IFN, beta, and IFN-gamma), Bone morphogenetic proteins (BMPs), chemokines (e.g., MCP-1 or CCL2), enzymes (e.g. endoglycosidases, exoglycosidases, endonucleases, exonucleases, peptidases, lipases, oxidases, decarboxylases, hydrases, chondroitinase, chondroitinase ABC, chondroitinase AC, hyaluronidase, keratanase, heparanases, heparanase splice variance, collagenase, trypsin, catalases), neurotransmitters (e.g., acetylcholine and monoamines), neuropeptides (e.g. substance P), vitamins (e.g., D-biotin, Choline Chloride, Folic acid, Myo-inositol, Niacinamide, D-Pantothenic acid, Calcium salts, Pyridoxal.HC1, Pyrodixine.HC1, Riboflavin, Thiamine.HC1, Vitamin B12, vitamin E, vitamin C, vitamin D, vitamin B1-6, vitamin K, vitamin A and vitamin PP), carbohydrates (e.g. Mono/Di/Polysacharides including glucose, mannose, maltose and fructose), ions, chelators (e.g. Fe chelators, Ca chelators), antioxidants (e.g., Vitamin E, Quarcetin, superoxide scavengers, Superoxide dismutase, H₂O₂ scavengers, free radicals scavengers, Fe scavengers), fatty acids (e.g., Triglycerides, Phospholipids, Cholesterols, free fatty acids and non free fatty acids, fatty alcohol, Linoleic acid, oleic acid and lipoic acid), antibiotics (e.g., Penicillins, Cephalosporins and Tetracyclines), analgesics, anesthetics, antibacterial agents, anti-yeast agents, anti-fungal agents, antiviral agents, pro-biotic agents, anti-protozal agents, anti-pruritic agents, anti-dermatitis agents, anti-emetics, anti-inflammatory agents, anti-hyperkeratolyic agents, antiperspirants, anti-psoriatic agents, anti-seborrheic agents, antihistamine agents, amino acids (e.g., essential and nonessential, especially glutamine and arginine), salts sulfates (e.g. Calcium Sulfate), steroids (e.g., androgens, estrogens, progestagens, glucocorticoids and mineralocorticoids), catecholamines (e.g., Epinephrine and Nor-epinephrine), Nucleosides and Nucleotides (e.g., Purins and Pyrimidines), Prostaglandins (e.g. Prostaglandin E2), Leucotriens, Erythropoietins (e.g. Thrombopoietin), Proteoglycans (e.g. Heparan sulfate, keratan sulfate), Hydroxyapatites (e.g. Hydroxyapatite (Ca₁₀(PO₄)₆(OH)₂)), Haptoglobins (Hp1-1, Hp2-2 and Hp1-2), Superoxide dismutases (e.g. SOD 1/2/3), Nitric Oxides, Nitric Oxide donors (e.g. nitroprusside, Sigma Aldrich, St. Louis, Mo., USA, Glutathione peroxidases, Hydrating compounds (e.g. vasopressin), cells (e.g. Platelets), cell medium (e.g. M199, DMEM/F12, RPMI, Iscovs), serum (e.g. human serum, fetal calf serum, fetal bovine serum), buffers (e.g., HEPES, Sodium Bicarbonate), detergents (e.g., Tween), disinfectants, herbs, fruit extracts, vegetable extracts (e.g. cabbage, cucumber), flower extracts, plant extracts, flavinoids (e.g. pomegranate juice), spices, leaves (e.g. Green tea, Chamomile), Polyphenols (e.g. Red Wine), honey, lectins, microparticles, nanoparticles (liposomes), micelles, calcium carbonate (CaCO₃, e.g.

precipitated calcium carbonate, ground/pulverized calcium carbonate, albacar, PCC, GCC), calcite, limestone, crushed marble, ground limestone, lime, and chalk (e.g. whiting chalk, champagne chalk, french chalk).

The present compositions may also contain ingredients, substances, elements and materials containing, hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

The present compositions may be combined with substances such as benzol peroxide, vasoconstrictors, vasodilatators, salicylic acid, retinoic acid, azelaic acid, lactic acid, glycolic acid, pyreuric acid, tannins, benzlidenecamphor and derivatives thereof, alpha hydroxyis, surfactants.

Compositions of some embodiments of the present invention may be bioconjugated to polyethylenglycol (e.g. PEG, SE-PEG) which preserves the stability (e.g., against protease activities) and/or solubility (e.g., within a biological fluid such as blood, digestive fluid) of the active ingredients while preserving their biological activity and prolonging their half-life.

The compositions of the present invention can be formulated as putty, ointment, inhalants, woven/non-woven pads, bandages, sponge, gels or hydrogels, (formulated with for example, gelatin, hyaluronic acid) or on the basis of polyacrylate or an oleogel (e.g. made of water and Eucerin).

Oleogels comprising both an aqueous and a fatty phase are based particularly on Eucerinum anhydricum, a basis of wool wax alcohols and paraffin, wherein the percentage of water and the basis can vary. Furthermore additional lipophilic components for influencing the consistency can be added, e.g. glycerin, polyethylene glycols of different chain lengths, e.g. PEG400, plant oils such as almond oil, liquid paraffin, neutral oil and the like. The hydrogels of the present invention can be produced through the use of gel-forming agents and water, wherein the first are selected especially from natural products such as cellulose derivatives, such as cellulose ester and ether, e.g. hydroxyethyl-hydroxypropyl derivatives, e.g. tylose, or also from synthetic products such as polyacrylic acid derivatives, such as Carbopol or Carbomer, e.g. P934, P940, P941. They can be produced or polymerized based on known regulations, from alcoholic suspensions by adding bases for gel formation.

Exemplary amounts of procollagen in the gel include 0.01-30 g per 100g of gel, 0.01-10 g per 100 g of gel, 0.01-8 g per 100 g of gel, 0.1-5 g per 100 g of gel.

In addition, the pharmaceutical compositions of this aspect of the present invention also include a dermatologically acceptable carrier.

The phrase “dermatologically acceptable carrier”, refers to a carrier, which is suitable for topical application onto the skin, i.e., keratinous tissue, has good aesthetic properties, is compatible with the active agents of the present invention and any other components, and is safe and non-toxic for use in mammals.

In order to enhance the percutaneous absorption of the active, one or more of a number of agents can be added to the pharmaceutical compositions including, but not limited to, dimethylsulfoxide, dimethylacetamide, dimethylformamide, surfactants, azone, alcohol, acetone, propylene glycol and polyethylene glycol.

The carrier utilized in the compositions of the invention can be in a wide variety of forms. These include emulsion carriers, including, but not limited to, oil-in-water, water-in-oil, water-in-oil-in-water, and oil-in-water-in-silicone emulsions, a cream, an ointment, an aqueous solution, a lotion, a soap, a paste, an emulsion, a gel, a spray, a foam or an aerosol. As will be understood by the skilled artisan, a given component will distribute primarily into either the water or oil/silicone phase, depending on the water solubility/dispersibility of the component in the composition.

Emulsions according to the present invention generally contain a pharmaceutically effective amount of the agent disclosed herein and a lipid or oil. Lipids and oils may be derived from animals, plants, or petroleum and may be natural or synthetic (i.e., man-made). Examples of suitable emulsifiers are described in, for example, U.S. Pat. No. 3,755,560, issued to Dickert, et al. Aug. 28, 1973; U.S. Pat. No. 4,421,769, issued to Dixon, et al., Dec. 20, 1983; and McCutcheon's Detergents and Emulsifiers, North American Edition, pages 317-324 (1986), each of which is fully incorporated by reference in its entirety.

The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending on the desired product form.

Examples of suitable carriers comprising oil-in-water emulsions are described in U.S. Pat. No. 5,073,371 to Turner, D. J. et al., issued Dec. 17, 1991, and U.S. Pat. No. 5,073,372, to Turner, D. J. et al., issued Dec. 17, 1991 each of which is fully incorporated by reference in its entirety. An especially preferred oil-in-water emulsion, containing a structuring agent, hydrophilic surfactant and water, is described in detail hereinafter.

A preferred oil-in-water emulsion comprises a structuring agent to assist in the formation of a liquid crystalline gel network structure. Without being limited by theory, it is believed that the structuring agent assists in providing rheological characteristics to the composition which contribute to the stability of the composition. The structuring agent may also function as an emulsifier or surfactant.

A wide variety of anionic surfactants are also useful herein. See, e.g., U.S. Pat. No. 3,929,678, to Laughlin et al., issued Dec. 30, 1975, which is fully incorporated by reference in its entirety. In addition, amphoteric and zwitterionic surfactants are also useful herein.

The pharmaceutical compositions of the present invention can be formulated in any of a variety of forms utilized by the pharmaceutical or cosmetic industry for skin application including solutions, lotions, sprays, creams, ointments, salves, gels, oils, wash, etc., as described below.

The pharmaceutical or cosmetic compositions of the present invention may be formulated to be sufficiently viscous so as to remain on the treated skin area, does not readily evaporate, and/or is not easily removed by rinsing with water, but rather is removable with the aid of soaps, cleansers and/or shampoos.

Methods for preparing compositions having such properties are well known to those skilled in the art, and are described in detail in Remington's Pharmaceutical Sciences, 1990 (supra); and Pharmaceutical Dosage Forms and Drug Delivery Systems, 6th ed., Williams & Wilkins (1995).

The topical compositions of the subject invention, including but not limited to lotions and creams, may comprise a dermatologically acceptable emollient. As used herein, “emollient” refers to a material useful for the prevention or relief of dryness, as well as for the protection of the skin. Wide varieties of suitable emollients are known and may be used herein. See, e.g., Sagarin, Cosmetics, Science and Technology, 2nd Edition, Vol. 1, pp. 3243 (1972), which contains numerous examples of materials suitable as an emollient and is fully incorporated herein by reference. A preferred emollient is glycerin.

Lotions and creams according to the present invention generally comprise a solution carrier system and one or more emollients.

The topically applied pharmaceutical or cosmetic composition of the present invention may also include additional components, which are added, for example, in order to enrich the pharmaceutical or cosmetic compositions with fragrance and skin nutrition factors.

Such components are selected suitable for use on human keratinous tissue without inducing toxicity, incompatibility, instability, allergic response, and the like within the scope of sound medical judgment. In addition, such optional components are useful provided that they do not unacceptably alter the benefits of the active compounds of the invention.

The CTFA Cosmetic Ingredient Handbook, Second Edition (1992) describes a wide variety of non-limiting cosmetic ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin-conditioning agents (e.g., humectants, including miscellaneous and occlusive), skin soothing and/or healing agents (e.g., panthenol and derivatives e.g., ethyl panthenol), aloe vera, pantothenic acid and its derivatives, allantoin, bisabolol, and dipotassium glycyffhizinate, skin treating agents, thickeners, and vitamins and derivatives thereof.

It will be appreciated that the procollagen of the present invention may be incorporated into products already developed or being developed by cosmetic companies, including but not limited to Estee Lauder, Helena Rubinstein and L′Oreal.

The pharmaceutical or cosmetic compositions of the present invention can be applied directly to the skin. Alternatively, it can be delivered via normal skin application by various transdermal drug delivery systems, which are known in the art, such as transdermal patches that release the composition into the skin in a time released manner. Other drug delivery systems known in the art include pressurized aerosol bottles, iontophoresis or sonophoresis. Iontophoresis is employed to increase skin permeability and facilitate transdermal delivery. U.S. Pat. Nos. 5,667,487 and 5,658,247 discloses an ionosonic apparatus suitable for the ultrasonic-iontophoretically-mediated transport of therapeutic agents across the skin. Alternatively, or in addition, liposomes or micelles may also be employed as a delivery vehicle.

Since wounds and ischemia may engage the scalp, the pharmaceutical compositions of the present invention further include emollients, surfactants and/or conditioners, which are suitable for use on the scalp skin and hair.

The emollients include, but are not limited to, hydrocarbon oils and waxes, such as mineral oil, petrolatum, and the like, vegetable and animal oils and fats, such as olive oil, palm oil, castor oil, corn oil, soybean oil, and the like, and lanolin and its derivatives, such as lanolin, lanolin oil, lanolin wax, lanolin alcohols, and the like. Other emollients include esters of fatty acids having 10 to 20 carbon atoms, such as including myristic, stearic, isostearic, palmitic, and the like, such as methyl myristate, propyl myristate, butyl myristate, propyl stearate, propyl isostearate, propyl palmitate, and the like. Other emollients include fatty acids having 10 to 20 carbon atoms, including stearic, myristic, lauric, isostearic, palmitic, and the like. Emollients also include fatty alcohols having 10 to 20 carbon atoms, such as cetyl, myristyl, lauryl, isostearyl, stearyl and the like.

An emulsifier/surfactant is preferably utilized when formulating the pharmaceutical compositions of the present invention for use on hair.

Examples of surfactants include, but are not limited to, spolyoxyalkylene oxide condensation products of hydrophobic alkyl, alkene, or alkyl aromatic functional groups having a free reactive hydrogen available for condensation with hydrophilic alkylene oxide, polyethylene oxide, propylene oxide, butylene oxide, polyethylene oxide or polyethylene glycol. Particularly effective are the condensation products of octylphenol with ˜7 to ˜13 moles of ethylene oxide, sold by the Rohm & Haas Company under their trademark TRITON 100® series products.

Other ingredients such as, fragrances, stabilizing agents, dyes, antimicrobial agents, antibacterial agents, antiagglomerates, ultraviolet radiation absorbers, and the like are also included in the composition of the present invention, which is formulated for use on hair.

A conditioner agent stable to acid hydrolysis, such as a silicone compound having at least one quaternary ammonium moiety along with an ethoxylated monoquat is preferably also utilized in order to stabilize and optionally thicken the composition of the present invention, which is formulated for use on hair.

An optional thickener also can be included to improve composition esthetics and facilitate application of the composition to the hair. Exemplary thickeners are methylcellulose, hydroxybutyl methylcellulose, hydroxypropylcellulose, hydroxypropyl methylcellulose, hydroxyethyl ethylcellulose and hydroxyethylcellulose, di (hydrogenated tallow) phthalic acid amide, crosslinked maleic anhydride-methyl vinyl ether copolymer, guar gum, xanthan gum and gum arabic.

The carrier of the conditioning composition is predominantly water, but organic solvents also can be included in order to facilitate manufacturing of the composition or to provide esthetic properties, such as viscosity control. Suitable solvents include the lower alcohols like ethyl alcohol and isopropyl alcohol; glycol ethers, like 2-butoxyethanol, ethylene glycol monoethyl ether, propylene glycol and diethylene glycol monoethyl ether or monomethyl ether and mixtures thereof. Non-limiting conditioning agents which may be used in opaque conditioners include: stearyltrimethylammonium chloride; behenetrimethylammonium chloride; cetrimonium bromide; soytrimonium chloride; tallowtrimonium chloride; dihyrogenatedtallowdimethylammonium chloride; behentrimethylammonium methosulfate; Peg-2 Oleammonium chloride; dihyrogenatedtallowdimethylammonium bromide; dihyrogenatedtallowdimethylammonium methosulfate; palmityltrimethylammonium chloride; hydrogenated tallowtrimethylammonium chloride; hydrogenated tallowtrimethylammonium bromide; dicetyidimethylammonium chloride; distearyldimethylammonium chloride; dipalmityidimethylammonium chloride; hydrogenated tallowtrimethylammonium methosulfate; cetrimonium tosylate; eicosyltrimethylammonium chloride and ditallowdimethylammonium chloride.

Shampoo formulations are sometimes advantageous for treating scalp skin conditions (e.g. lesions, psoriasis).

The hair shampoo composition of the present invention may be provided in any form selected from liquid, powder, gel and granule as needed. A liquid composition using water or a lower alcohol as a solvent is preferred, with a liquid composition using water being especially preferred. Shampoo compositions which may be used according to the teachings of the present invention are further described in U.S. Pat. No. 6194363 and U.S. Pat. No. 6007802.

It will be appreciated that the procollagen of the present invention may be incorporated into biocompatible and/or biodegradable polymer-based matrices, including sheets, films, membranes sponges and gels, as described in WO2009/128076. Other exemplary applications are described in WO2014/147622.

Also collagen produced as described herein can be included in 3D bioprinting of tissues and organs.

Recently, 3D bioprinting is being gaining momentum in many medicinal applications to address the need for complex scaffolds, tissues and organs suitable for transplantation and tissue modeling. To this end the collagen is chemically modified to adapt the biological molecules for printing, such that the Biolnk maintains controlled fluidity during printing, and cures to form hydrogel when irradiated by light ranging from UV to visible light. The unique viscosity and shear thinning properties of the modified Collagen allow the flexibility to easily formulate Biolnks for different printing technologies including extrusion, ink-jet, Laser Induced Forward Transfer (LIFT) and Stereolithography. The control of chemical modification in combination with illumination energy allows tight control on the physical properties of the resulting scaffolds to match natural tissues properties, from stiff cartilage to soft adipose.

The collagen produced as described herein can be used for aesthetic and plastic surgery indications. For this purpose, the collagen can be used alone or in combination with other components, such as hyaluronic acid, to form a dermal filler composition that can be used for injection under the skin as a filler.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first, indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Selection of high procollagen yielding lines in F5 and F6 pedigrees derived from A3-29 F1 and comparison to Z1

The breeding program is aimed at developing transgenic tobacco plants lines with high yields of human type I procollagen (PC). The breeding program is based on tobacco plants transformed with 5 human genes [see WO2006/035442]. The goal of the breeding program is to increase the copy number of the transgenes in the hemizygous A3-29 line through repeated cycles of self crossing and selection of high yield progenies, which eventually lead to enhanced homozygocity. Homozygous lines are preferred both for its demonstrated higher procollagen yields and the option for propagation via seeds. Seed-based propagation should significantly reduce plantlet costs and shorten the cycle to achieve plantlets for commercial production.

The seed-progenitor lines selected for the study are 23 F5 sibling progeny descendants of the A3-29 F1 line (FIG. 1). Among the 23 F5 siblings, 17 lines were selected as probable-best-candidates for further screening at F6 generation, based on their PC yield.

As the breeding program proceeds to further advanced filial generations, the higher the chances are that the original hemizygous plant becomes homozygotized. This study evaluated the level of PC production in best isolated lines to determine the best candidates for replacing the current hemizygous Z1 line (as described above).

Objectives

The scope of this experiment was to isolate best-pro-collagen yielding plant lines from the breeding program (line A3-29 pedigrees), as compared to the PC production line, Zl. The level of heterogeneity among individual plants was assessed, as well as confirmation of the required genetic makeup by western blotting (WB) for Coll alpha 1, Col1 alpha 2; P4H alpha and P4H beta. F6 seeds from selected F5 plants and F7 seeds from selected F6 plants were collected for further field tests.

This example summarizes two consecutive breeding cycles (F5 to F6 and F6 to F7).

Materials and Methods

Assessment of Procollagen Level in Leaves by ELISA:

A sandwich ELISA was developed to quantify the amount of procollagen in the leaves extract. This ELISA assay is based on the specific capture of procollagen by a layer of Mouse anti human procollagen type I C-terminus (TAKARA cat. No. MO12), followed by Rat monoclonal antibody to procollagen type I-N terminus clone M-58 (Millipore MAB1912). Quantization is achieved by using ALP-conjugated goat anti rat antibody (Chemicon International Cat# AP136A) followed by a colorimetric assay of a suitable substrate.

Assessment of Collagen by Western Blot:

Leaves extract or purified material is first separated using SDS-PAGE and the separated proteins transferred onto nitrocellulose membranes. Collagen related peptides are detected by using anti collagen Typel rabbit polyclonal antibody (Millipore #234167) or Rabbit anti-Human collagen type I polyclonal antibody (Chemicon #AB745). The membranes are later probed with ALP conjugated affinity purified Goat anti Rabbit IgG (Chemicon # AP132A) followed by appropriate substrate (SIGMA FAST BCIP/NBT: Sigma #B5655).

Plants Propagation and Cultivation

F5 Breeding Cycle

Lines Z1 and A3-29 F1 plantlets were propagated via tissue culture and hardened in a greenhouse. Plantlets of the 23 F5 lines which were propagated via seeds were planted in trays. FIG. 1 describes the pedigree crossing. A3-29 is also described in WO2009/128076. The specific sequences were verified by cDNA analysis (SEQ ID NOs: 20-24) and relevant AA sequences (SEQ ID Nos 25-26).

F6 Breeding Cycle

Line Z1 and A3-29 F1 plantlets were propagated via tissue culture and hardened for about 2.5 weeks at the greenhouse.

Plantlets of the 25 seed-based lines were propagated at a nursery.

Pedigrees and Field Planning

F5 Breeding Cycle:

Each of A3-29 F5 lines was planted in a distinct row. Control A3-29 F1 and Z1 plants were set in the middle of each row to assure equal comparison of control plants to tested plant lines.

Below the list of F5 pedigrees in that study (Table 1)

TABLE 1

| Line name | Generation | Propagation method |
|---|---|---|
| A3-29 | F1 | Tissue culture |
| Z1 | F1 | Tissue culture |
| A3-29-353-4-9 | F4 | Seed |
| A3-29-353-4-19 | F4 | Seed |
| A3-29-353-4-22 | F4 | Seed |
| A3-29-353-4-42 | F4 | Seed |
| A3-29-353-4-44 | F4 | Seed |
| A3-29-353-4-72 | F4 | Seed |
| A3-29-305-17-1 | F4 | Seed |
| A3-29-305-17-2 | F4 | Seed |
| A3-29-305-17-9 | F4 | Seed |
| A3-29-305-17-17 | F4 | Seed |
| A3-29-305-17-18 | F4 | Seed |
| A3-29-305-13-18 | F4 | Seed |
| A3-29-305-13-31 | F4 | Seed |
| A3-29-434-19-15 | F4 | Seed |
| A3-29-434-19-11 | F4 | Seed |
| A3-29-434-19-10 | F4 | Seed |
| A3-29-434-19-24 | F4 | Seed |
| A3-29-434-19-17 | F4 | Seed |
| A3-29-434-19-23 | F4 | Seed |
| A3-29-353-17-14 | F4 | Seed |
| A3-29-353-17-20 | F4 | Seed |
| A3-29-353-17-33 | F4 | Seed |
| A3-29-353-17-23 | F4 | Seed |

F6 Breeding Cycle:

In the following cycle, seeds of all 17 “winners” (see above) were grown in trays and were analysed as pools at the seedlings stage. Super-Family 353-04-19 was expanded by 4 extra F6 families. Three different F5 seed lines, pedigrees of A3-29-366-02 and Z1 were analysed as references and control (see FIG. 12).

13 high PC yielding F6 lines were selected and were planted. Each line was planted in a distinct row. Control A3-29 F1 and Z1 plants were set in the middle of each row to assure equal comparison of control plants to tested plant lines.

Table 2 below lists F5-F6 pedigrees in this study.

TABLE 2

| Line name | Generation | Propagation method |
|---|---|---|
| A3-29 | F1 | Tissue culture |
| Z1 | F1 | Tissue culture |
| A3-29-305-17-09-10 | F5 | Seed |
| A3-29-305-17-09-15 | F5 | Seed |
| A3-29-305-17-09-16 | F5 | Seed |
| A3-29-305-17-09-18 | F5 | Seed |
| A3-29-305-17-09-25 | F5 | Seed |
| A3-29-305-17-09-31 | F5 | Seed |
| A3-29-305-17-09-37 | F5 | Seed |
| A3-29-305-17-17-16 | F5 | Seed |
| A3-29-305-17-17-7 | F5 | Seed |
| A3-29-353-04-19-5 | F5 | Seed |
| A3-29-353-04-19-9 | F5 | Seed |
| A3-29-353-04-19-19 | F5 | Seed |
| A3-29-353-04-19-24 | F5 | Seed |
| A3-29-353-04-19-27 | F5 | Seed |
| A3-29-353-04-19-30 | F5 | Seed |
| A3-29-353-04-42-8 | F5 | Seed |
| A3-29-434-19-15-25 | F5 | Seed |
| A3-29-434-19-15-34 | F5 | Seed |
| A3-29-434-19-15-40 | F5 | Seed |
| A3-29-366-02-1 | F4 | Seed |
| A3-29-366-02-8 | F4 | Seed |
| A3-29-366-02-10 | F4 | Seed |

Leaves Sampling and Analysis

F5 Cycle

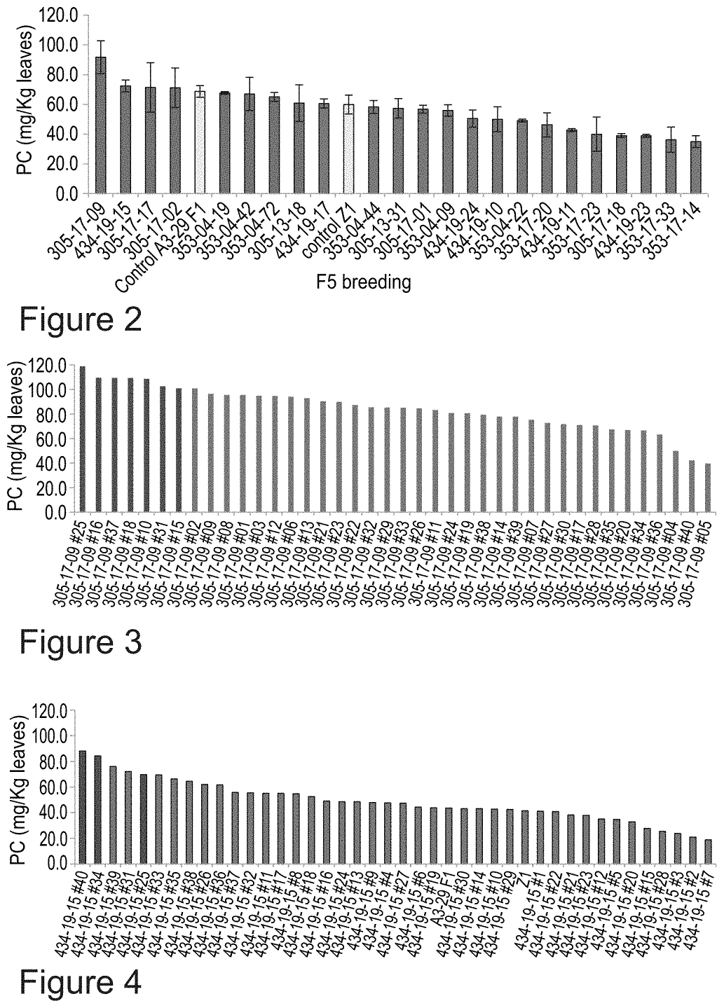

Pooled procollagen samplings were first performed at ˜45 days' post planting in a greenhouse: Each individual plant was sampled into a pooled line bag by collecting 6 leaves located at positions 7 to 12 from bottom. Each of the 23 selected lines was assayed as well as control plants. In each of the control lines, 3 leaves pools were sampled and three 100g samples were processed. In each of the F5 lines, 2 leaves pools were sampled (see FIG. 2).

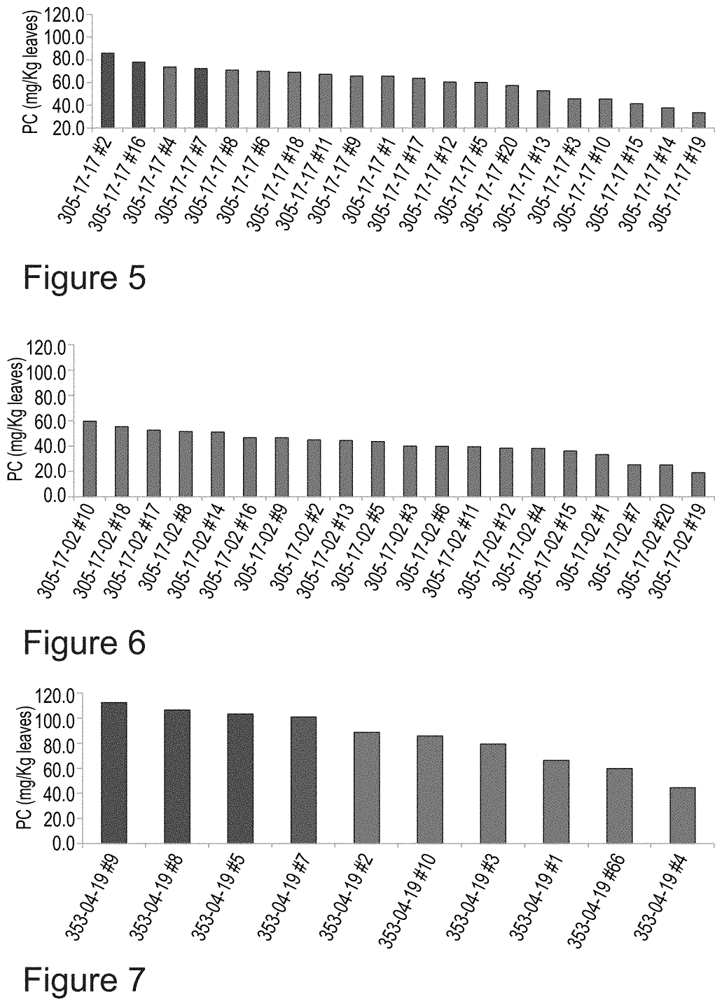

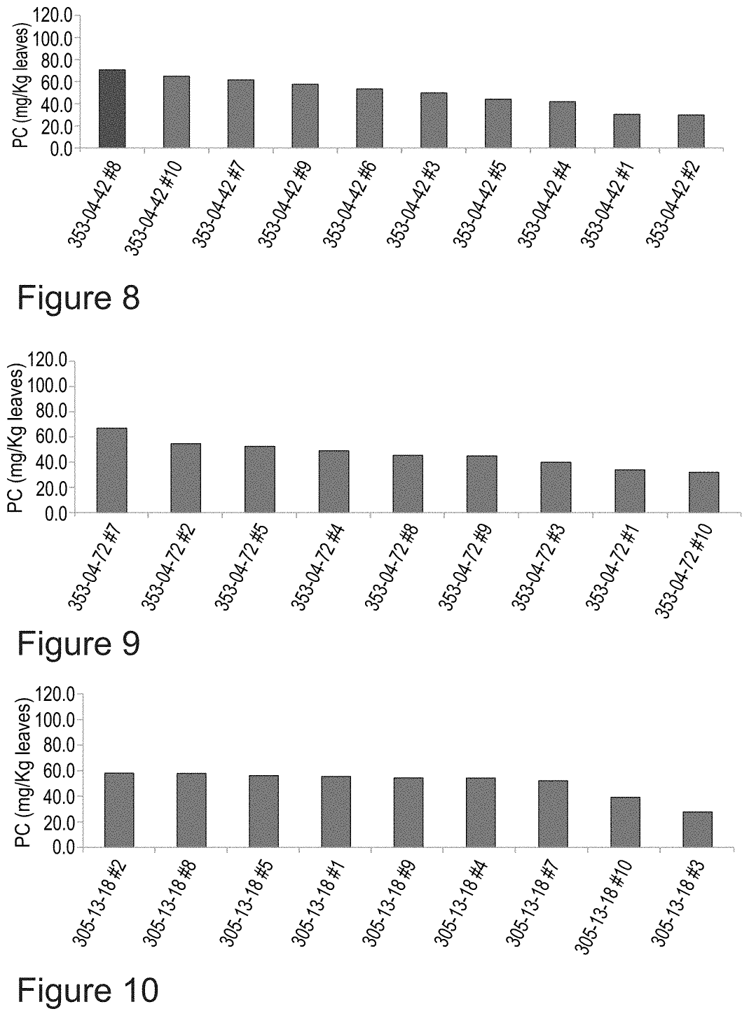

8 best PC-yielding lines were selected for individual plant PC analysis: all individual plants among the 2 top yielding lines and up to 20 randomly individual plants in the 6 next-best lines. A fixed number and position of subsequent leaves at position 10-13 through 16-18 leaves were sampled from each of the described selected plants were analysed (see FIG. 3-10).

Results were analysed relatively to 6 control samples (3 of Z and 3 of A3-29) that were repeatedly analysed in parallel in each ELISA plate, in order to cancel the variance between different tests.

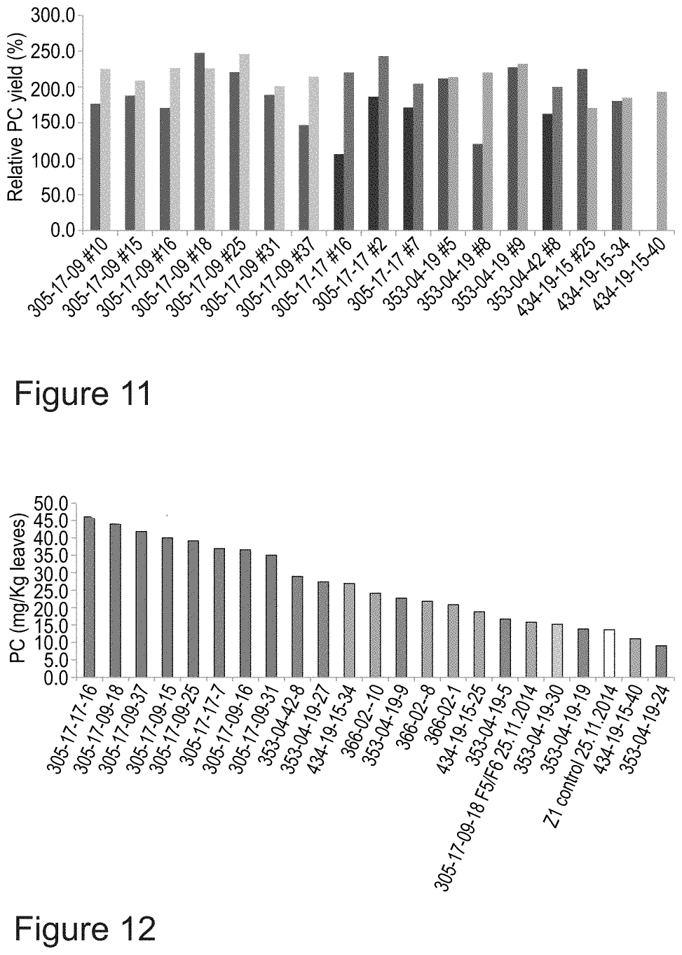

17 best PC yielding individual plants out of 5 different pedigrees were analysed again, in a comparative “almost-winners” ELISA (FIG. 11).

Self-pollinated mature seeds were collected out of each of the selected plants.

F6 Cycle

Forty seedlings, at 3 to 4 weeks old were sampled and pooled into ˜40-60 gr single samples, grounded and were assayed via ELISA. (FIG. 12).

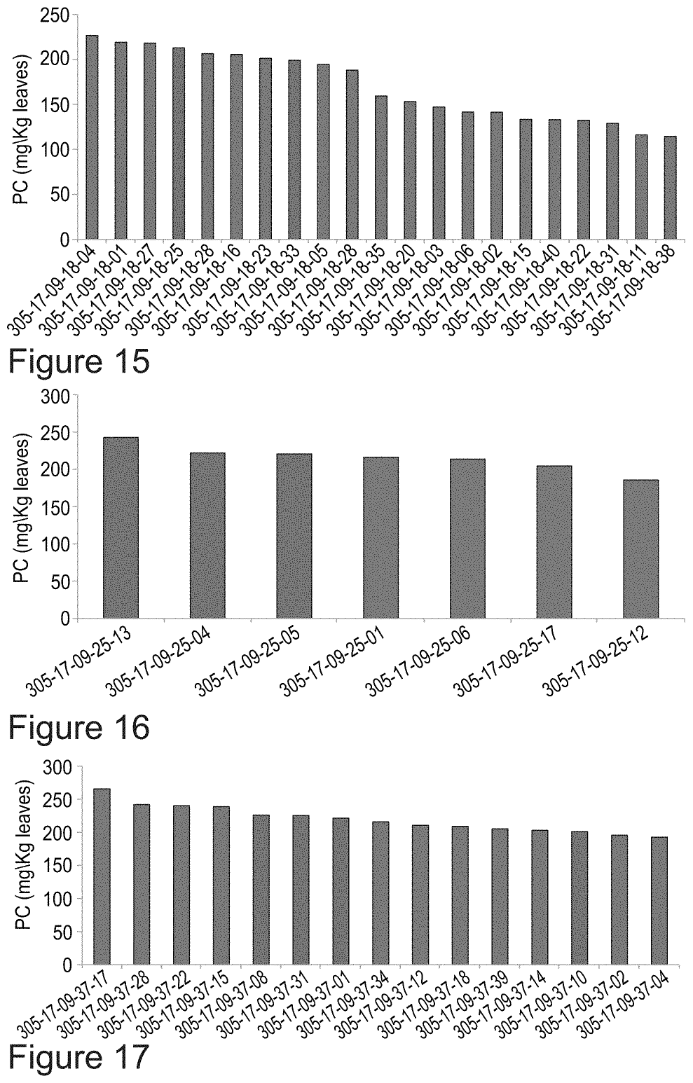

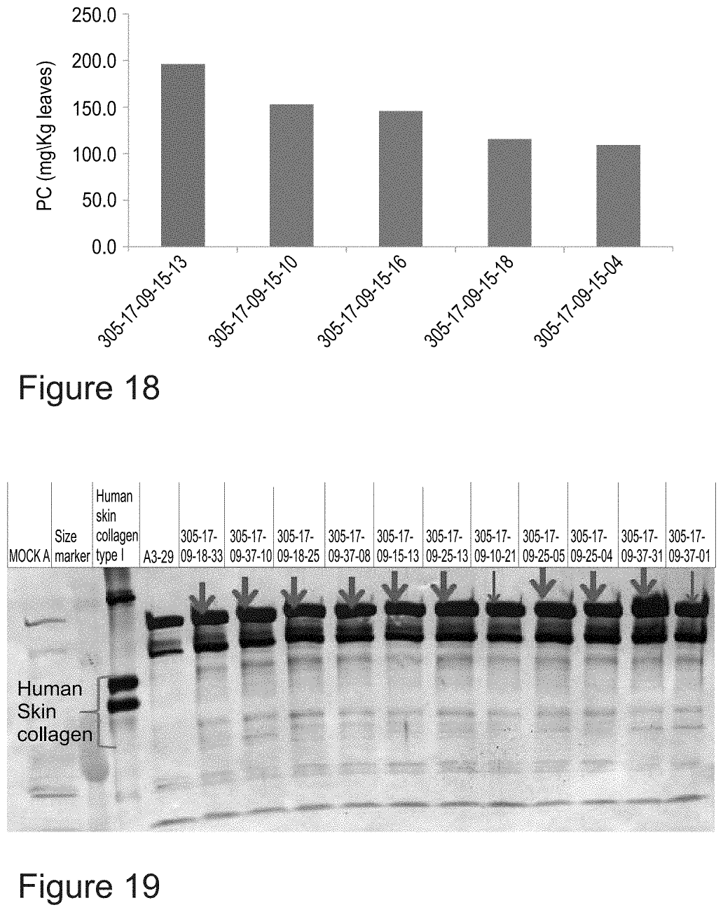

11 high PC yielding lines were selected for individual plant procollagen analysis in both of two best PC yielding families, two extra F6 lines were added to the individual plant analysis, (305-17-09-10 and 305-17-17-02 (FIG. 12). Individual procollagen samplings were performed 61 days after planting at the greenhouse and analysed with the following exceptions: Each individual plant was similarly sampled: A fixed number and position of 3 to 6 subsequent leaves located at positions between 10-13 through 16-18 from bottom (pending plant growth) were sampled. Each of the 13 lines were assayed, as well as 24 control plants of A3-29 F1 line. For control plants, pools were sampled in lower position: positions 5-7 to 9-12 from bottom. 3 samples from each of the pool bags were taken and three 100g samples were grounded to best represent the pool samplings. About 20 tubes of each such sample were prepared for ELISA assay. Two independent ELISA assays were run with the controls and selected plants. Results were analysed compares to control samples (see FIG. 13). 5 lines were selected for individual plants analysis (see FIG. 14-18).

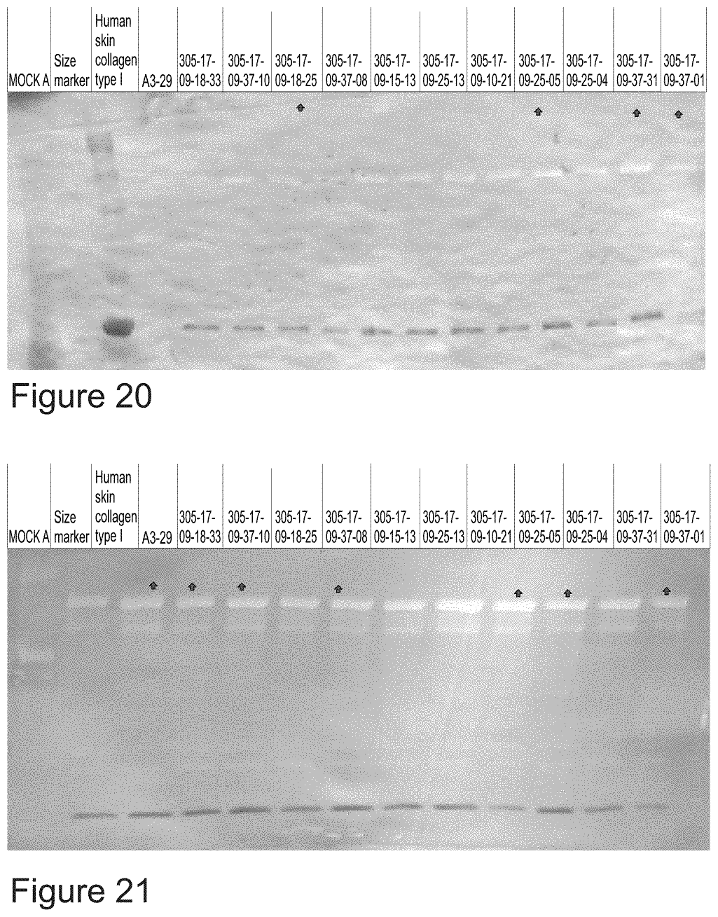

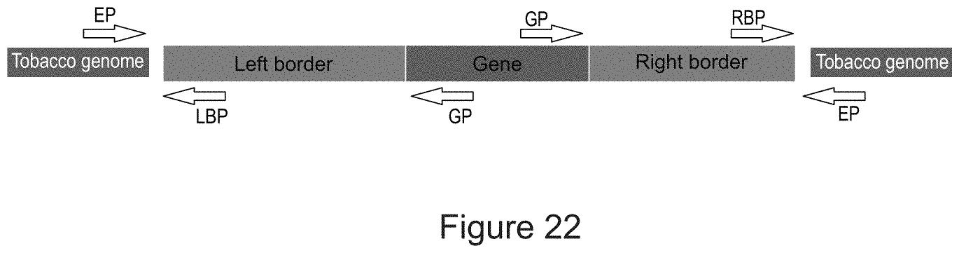

Winner plants were tested by western blot analysis (extract digested to collagen) to assure presence of the relevant proteins in comparison to control plants (FIGS. 19-21).

Selected plants were propagated via twigs and tissue culture for introduction into the master plant bank and used for self-seed production for further breeding selections.

### Example 2

### Event characterization in A3-29-305-17-09-18 F5

Genomic DNA samples of A3-29-305-17-09-18 F5, a genetically modified plant with 4 different recombinant DNA constructs, were analyzed. Illumina short reads, illumina short read mate pair, Nanopore ultra long reads and Nanopore-based sequencing technologies were employed. PCR and Sanger sequencing were used to validate the results. A total of 5 insertion events were identified.

All primers sequences, descriptions and references to the relevant figures are detailed in tables #35-38.

TABLE 3
The 5 insertion events,

| | | Gene | | | Gene | | |
|---|---|---|---|---|---|---|---|
| | Border | PCR by | | | PCR by | Border | |
| Sanger | PCR | Nanopore | | | Nanopore | PCR | Sanger |
| Left Border | | | Event | Gene | Right Border | | |
| ✓ | ✓ | ✓ | Event | P4H beta + | ✓ | ✓ | ✓ |
| | | | 1 | LH3 | | | |
| ✓ | ✓ | ✓ | Event | P4H | ✓ | ✓ | ✓ |
| | | | 2 | alpha | | | |
| ✓ | ✓ | ✓ | Event | Col | — | ✓ | — |
| | | | 3 | aplha2 | | | |
| ✓ | ✓ | ✓ | Event | P4H beta + | — | ✓ | — |
| | | | 4 | LH3 | | | |
| ✓ | ✓ | ✓ | Event | Col | — | ✓ | — |
| | | | 5 | alpha 1 | | | |

Note:
Border PCR: Once the events are identified, primers are designed for left and right borders of the insert (Vector region). These primers are used in combination with genome (event) specific primers.
Gene PCR: PCR was done using gene specific primers in combination with genome (event specific) primers. The amplicons are run using Nanopore sequencing platform. See Table 35.

Event 1 - P4H beta+LH3

Event 1 position is illustrated in FIG. 23. The construct is shown in the upper scheme.

TABLE 4
Event information in a tabular format

| S. | | | | |
|---|---|---|---|---|
| No | Description | Start | End | Strand |
| 1 | Ntab-TN90_AYMY- | 1 | 4706 | + |
| | SS247240 | | | |
| 2 | P4H beta + LH3 | 228 | 5240 | + |
| 3 | P4H beta + LH3 | 5241 | 8118 | − |
| 4 | P4H beta + LH3 | 8119 | 8445 | + |
| 5 | Ntab-TN90_AYMY- | 4707 | 11756 | + |
| | SS247240 | | | |

TABLE 5
Primer set used for Nanopore-based sequencing

| | | | | | Expected |
|---|---|---|---|---|---|
| | | | | Expected | product |
| | | | Reverse | product | size in |
| S. | | Forward | Primer/ | size in | Trans- |
| No | Junction | Primer | SEQ ID NO: | control | formed |
| 1 | Right | Primer: | Primer: | No | −796 bp |
| | Junction | Event1- | Event 1- | Amplifi- | |
| | SEQ ID | F_Rightjunc | R_Rightjunc | cation | |
| | NOs of | GTCTTATCTT | ACACAACA | | |
| | primers: | CAGCCGACG | ACCACCCC | | |
| | 27-28 | C | AGAA | | |
| 2 | Left | Primer: | Primer: | 656 bp | −8873 bp |
| | Junction | Event1- | Event1- | | |
| | SEQ ID | F_Leftjunc | R_Leftjunc | | |
| | NOs of | CCCCTTCTGA | TCCCCTGA | | |
| | primers: | TTTTCTTGGT | AACTTTGG | | |
| | 29-30 | GT | TCCA | | |

right junction = right border

### Note:

In Event1—Rightjunc primer amplification. For a transformed line a ˜800 bp product is expected. However, in control and transformed line a non-specific amplification band of 350 bp is expected.

In Event1—Leftjunc primer amplification (spanning entire insert along with left junction); For transformed line a ˜8000 bp product is expected. A non-specific amplification band of ˜2500 bp is present in the transformed line.

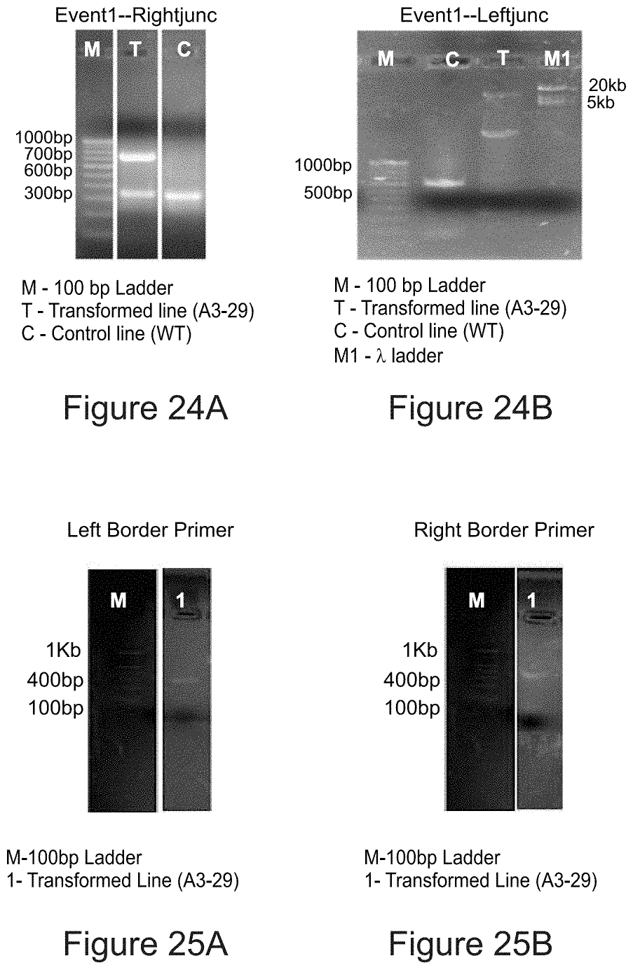

The sequencing of the PCR product from transformed line using primer Event1—Rightjunc (FIGS. 24A, B) is as follows. The sequence captures the junction between the construct and the genome contig.

TABLE 6
Primer set used for border PCR
(Sanger validated)

| P411 beta + | Genome | Border | Product |
|---|---|---|---|
| LH3 | Primer/ | Primer/ | size |
| Left Junction | Primer: | Primer: RP1 | −400 bp |
| Primer | E1 F_LF | TGATTTATAAGG | |
| SEQ ID NOs of | CCCCTTCTGA | GATTTTGCCGAT | |
| primers: 29, 31 | TTTTCTTGGT | | |
| | GT | | |
| Right Junction | Primer: | Primer: FP1 | −500 bp |
| Primer | E1 R_LF | AACCCTGGCGTT | |
| SEQ ID NOs of | TCCCCTGAAA | ACCCAACT | |
| primers: 30, 34 | CTTTGGTCCA | | |

FIGS. 25 and 26 show the results of event 1 amplification by gel electrophoresis and sequencing, respectively.

Conclusion

Construct inserted as Eventl (P4H beta +LH3), was confirmed at left and right borders through Nanopore-based sequencing as well as Sanger sequencing methods.

Event 2-P4H alpha

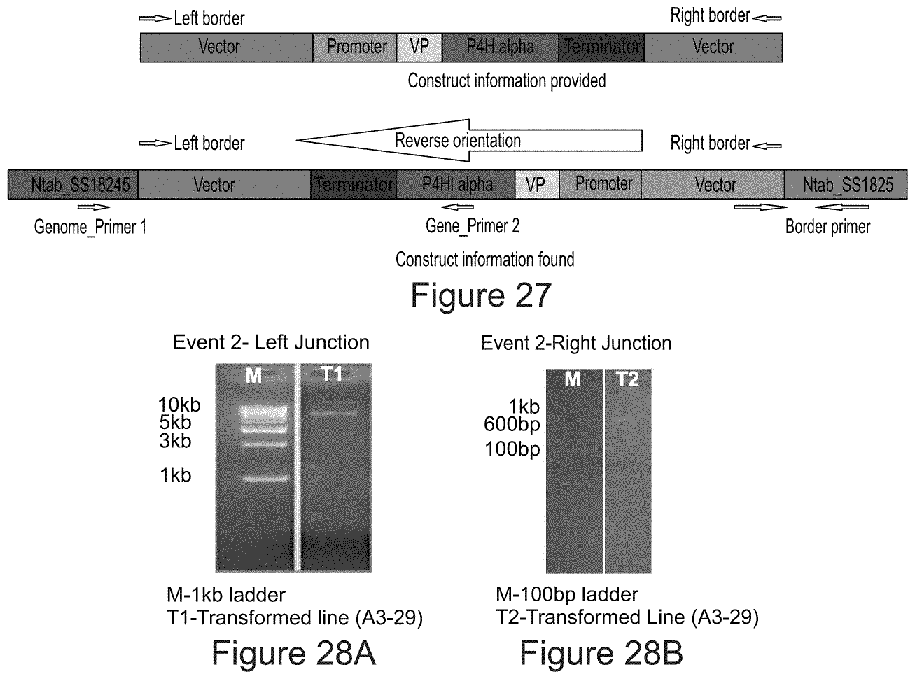

FIG. 27 shows a schematic illustration of event 2 integration in the genome of the plant.

TABLE 7
Event information in a tabular format

| Sl | | | | |
|---|---|---|---|---|
| No | Description | Start | End | Strand |
| 1 | Ntab-TN90_AYMY-SS1825 | 51828 | 52215 | + |
| 2 | P4H alpha | 492 | 3253 | + |
| 3 | P4H alpha | 3801 | 5104 | − |
| 5 | Ntab-TN90_AYMY-SS1825 | 52216 | 52777 | − |

TABLE 8
Primer set used for Nanopore-based sequencing

| P4H | | | Product |
|---|---|---|---|
| Alpha | Genome_Primer | Gene_Primer | size |
| Left | Primer: | Primer: | −5 kb |
| Junction | MP_Col_9R | P4Halpha-F-start | |
| Primer | AATTGTTCTGTGA | CACCCAGGATTCTTCA | |
| SEQ ID | AGGCGGG | CTTCTA | |
| NOs of | | | |
| primers: | | | |
| 37 33 | | | |
| Right | Primer: | Primer: FP1 | 800 bp |
| Junction | 1825R | AACCCTGGCGTTACCC | |
| Primer | TGTGTTTGGGGGT | AACT | |
| SEQ ID | TGAGGAT | | |
| NOs of | | | |
| primers: | | | |
| 35 34 | | | |

Event characterization is shown in FIGS. 28A-B, as obtained by nanopore-based sequencing.

The event was further characterized by Sanger PCR, primers of which are listed below and Table 9.

TABLE 9
Primer set used for border PCR
(Sanger validated)

| P4H | | | Product |
|---|---|---|---|
| Alpha | Genome_Primer | Border_Primer | size |
| Left | Primer: 1825F | Primer: RP1 | −400 bp |
| Junction | GTTTGCATACGCTTG | TGATTTATAAGGG | |
| Primer | GGTGG | ATTTTGCCGAT | |
| SEQ ID | | | |
| NOs of | | | |
| primers: | | | |
| 36 31 | | | |
| SEQ ID | Primer | Primer: RP3 | 500 bp |
| NOs of | MP_Col_: 9R | ATTTTGCCGATTT | |
| primers: | AATTGTTCTGTGAAG | CGGAACC | |
| 37 38 | GCGGG | | |
| Right | Primer: 1825R | Primer: FP1 | −800 bp |
| Junction | TGTGTTTGGGGGTTG | AACCCTGGCGTTA | |
| Primer | AGGAT | CCCAACT | |
| SEQ ID | | | |
| NOs of | | | |
| primers: | | | |
| 35 34 | | | |

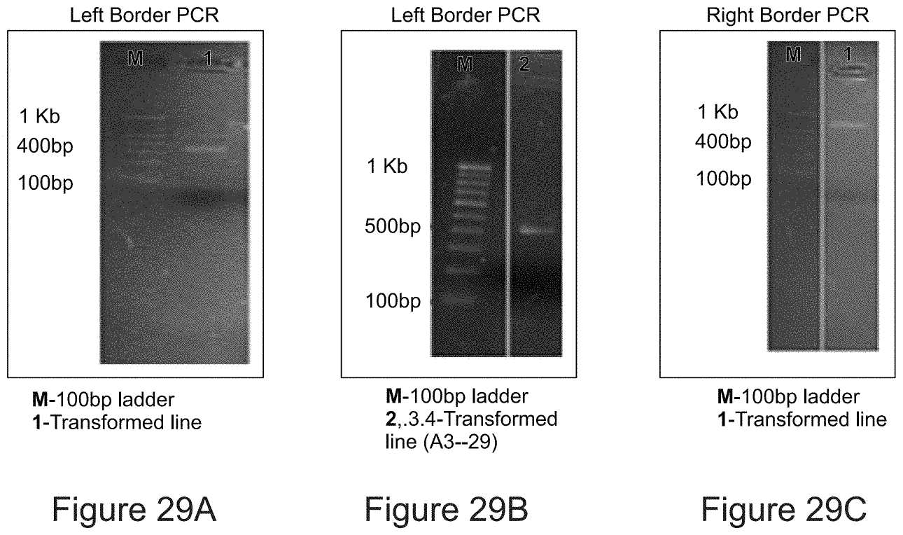

Results are shown in FIGS. 29A-C to 30, by gel electrophoresis and sequencing, respectively.

For the construct inserted as Event2 for P4H alpha, the left and right borders were confirmed through Nanopore-based sequencing as well as Sanger sequencing methods. The insert is in reverse orientation (FIG. 27).

Event 3 -Col alpha 2

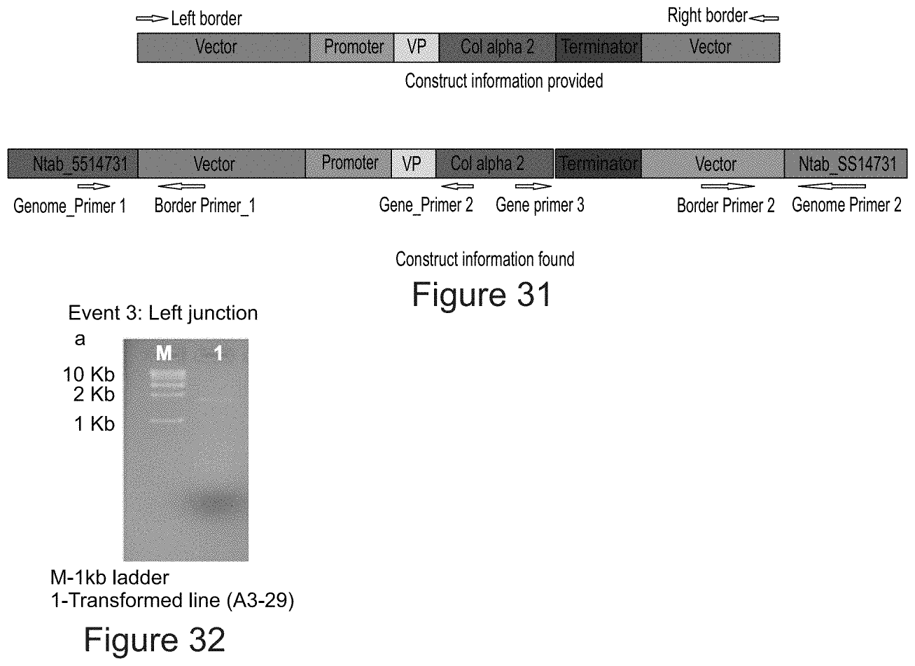

FIG. 31 shows a schematic diagram of event-3 position in the genome.

TABLE 10
Event information in a Tabular format

| Sl | | | | |
|---|---|---|---|---|
| No | Description | Start | End | Strand |
| 1 | Ntab-TN90_AYMY- | 8 | 2602 | − |
| | SS14731 | | | |
| 2 | Col alpha 2 | 227 | 7875 | + |

FIG. 32 shows insert characterization using event-3 left junction primer.

TABLE 11
Primer set used for Nanopore-based sequencing

| Col | Genome | | Product |
|---|---|---|---|
| alpha 2 | Primer 1 | Gene_Primer 2 | size |
| Left | Primer: | Primer: | −6 kb |
| Junction | MP_Col_5R | Colalpha2-R-Start | |
| Primer | TCATCAAGG | AGACTCGCCTTTTGATC | |
| SEQ ID | ACCTGCGTT | CAG | |
| NOs of | CAA | | |
| primers: | | | |
| 39 40 | | | |

TABLE 12
Primer set used for border PCR
(Validated by Sanger)

| Col | | | Product |
|---|---|---|---|
| alpha 2 | Genome_Primer | Border_Primer | size |
| Left | Primer: 14731F | Primer: RP1 | 800 bp |
| Junction | AGGAGTCGTTGTTG | TGATTTATAAGGG | |
| Primer | TTGGTT | ATTTTGCCGAT | |
| SEQ ID | | | |
| NOs of | | | |
| primers: | | | |
| 41 31 | | | |
| SEQ ID | Primer: | Primer: RP2 | −2 kb |
| NOs of | MP_Col_5R | ATAAGGGATTTTG | |
| primers: | TCATCAAGGACCTG | CCGATTT | |
| 39 42 | CATCG | | |
| TCAA | | | |

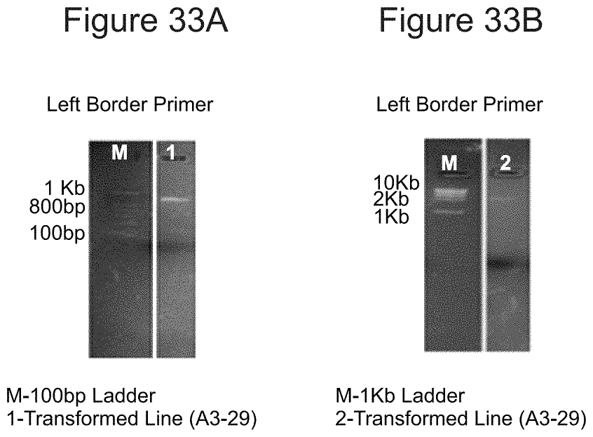

FIGS. 33A-B shows Border junction PCR: FIGS. 33A-B) Left border PCR using genome prime and border primers, amplicon size, 1-800bp, 2-˜2Kb

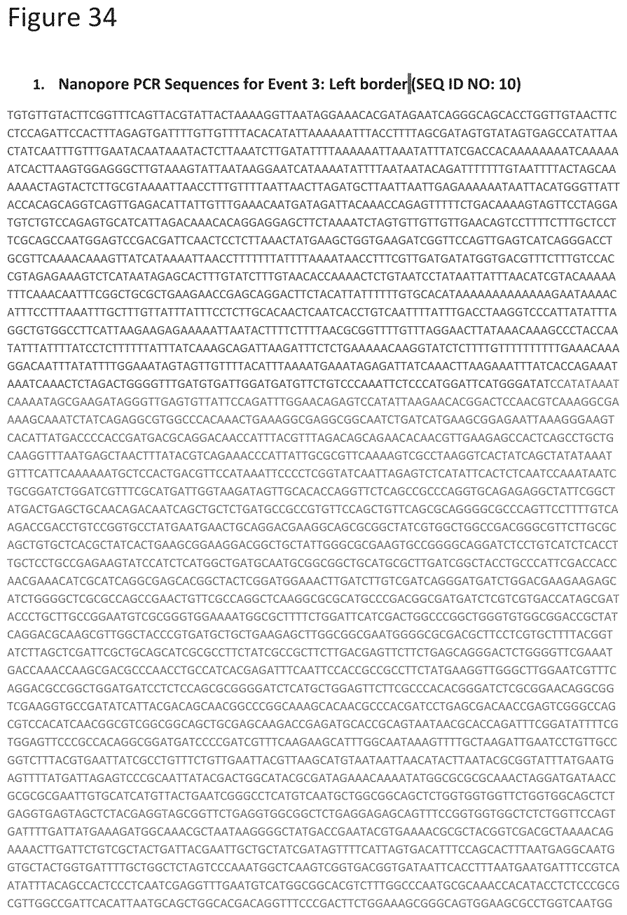

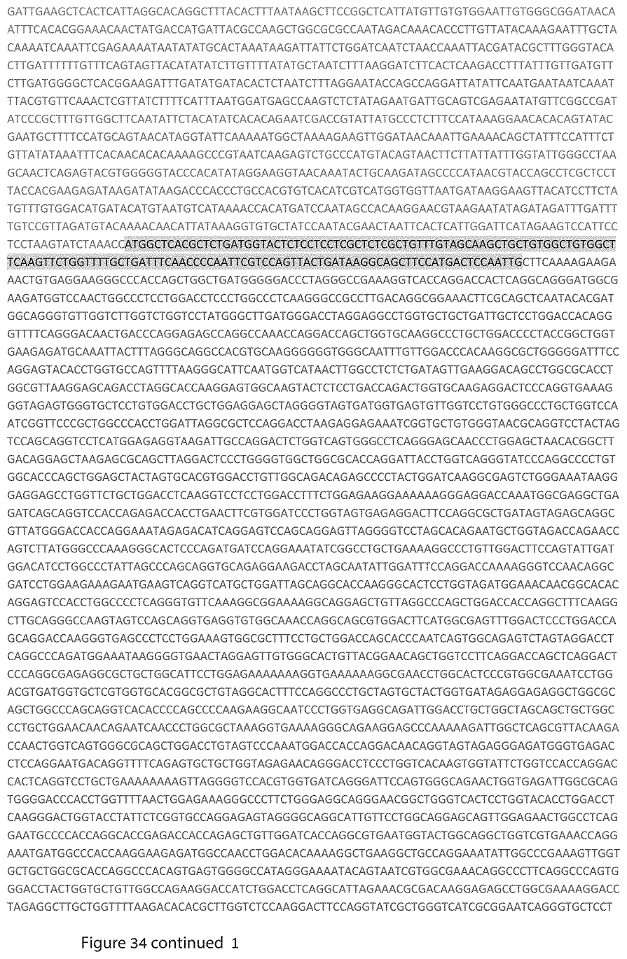

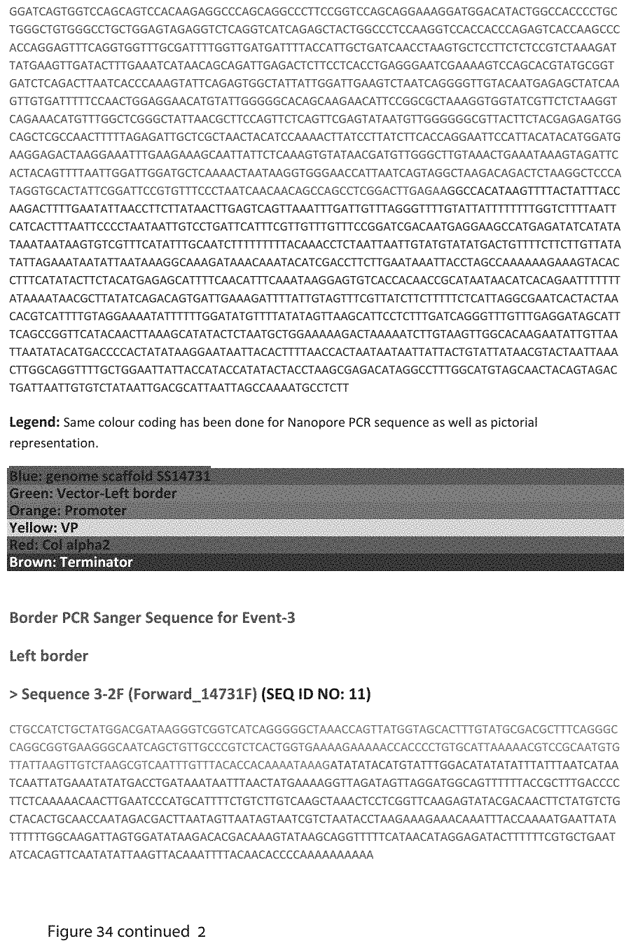

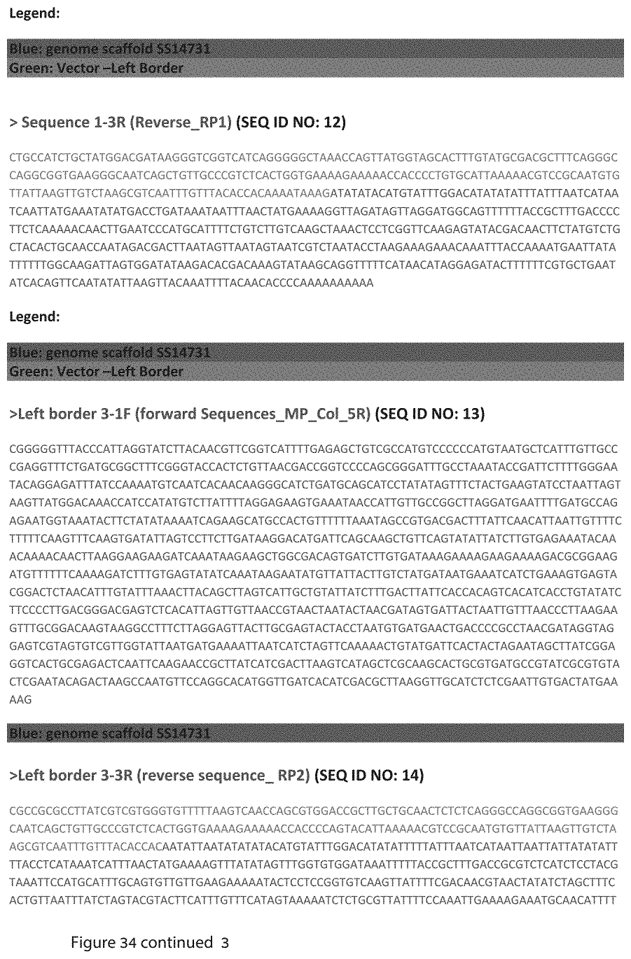

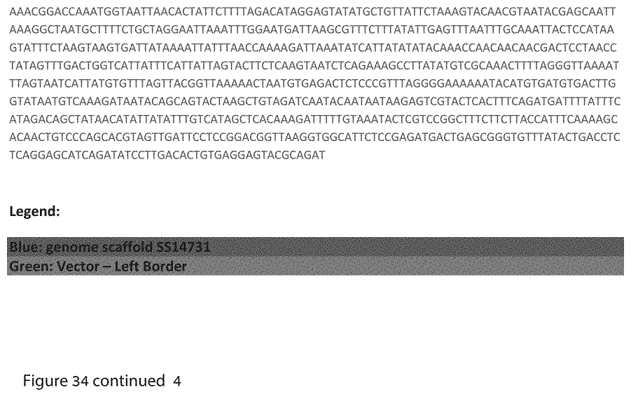

The results of Nanopore-based sequencing and Sanger sequencing are shown in FIG. 34.

Construct inserted as Event3 for Col alpha 2- the left border was confirmed through Nanopore-based sequencing as well as Sanger sequencing methods. Border PCR amplified the right borders but Sanger sequencing failed due to technical reasons. In FIG. 34, the regions (Right border vector and genome scaffold) are marked in lighter colors.

Event 4-P4H beta (LH3)

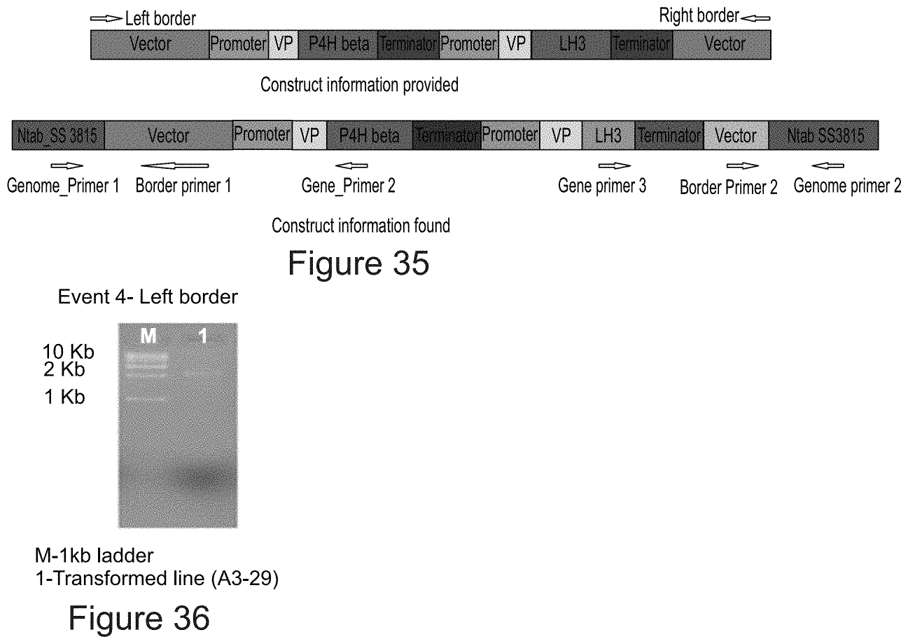

FIG. 35 shows a schematic diagram of event-4 position in the genome.

TABLE 13
Event information in Tabular format

| Sl | | | | |
|---|---|---|---|---|
| No | Description | Start | End | Strand |
| 1 | Ntab-TN90_AYMY-SS3815 | 38180 | 38667 | + |
| 2 | P4H beta + LH3 | 2851 | 4929 | + |

TABLE 14
Primer set used for Nanopore-based sequencing

| | | | Product |
|---|---|---|---|
| P4H beta + LH3 | Genome Primer | Border Primer | size |
| Left Junction | Primer: 3815F | Primer: 3815R | −5.5 Kb |
| Primer | TAAGCAGACAACC | TAAGGTTCGCCGG | |
| SEQ ID NOs of | ACGCGAT | TGCTATG | |
| primers: 43 44 | | | |

FIG. 36 shows insert characterization using event-4 left junction primer.

TABLE 15
Primer set used for Border PCR
(Validated by Sanger sequencing)

| | | | Product |
|---|---|---|---|
| P4H beta + LH3 | Genome_Primer | Border_Primer | size |
| Left Junction | Primer: 3815F | Primer: RP1 | −1.5 Kb |
| Primer | TAAGCAGACAACC | TGATTTATAAGGG | |
| SEQ ID NOs of | ACGCGAT | ATTTTGCCGAT | |
| primers: 43 31 | | | |

Construct inserted as Event 4 for P4H beta (LH3), the left border was confirmed through Nanopore-based sequencing as well as Sanger sequencing methods. Border PCR amplified the right borders but Sanger sequencing failed due to technical reasons.

Event 5 - Col alpha 1

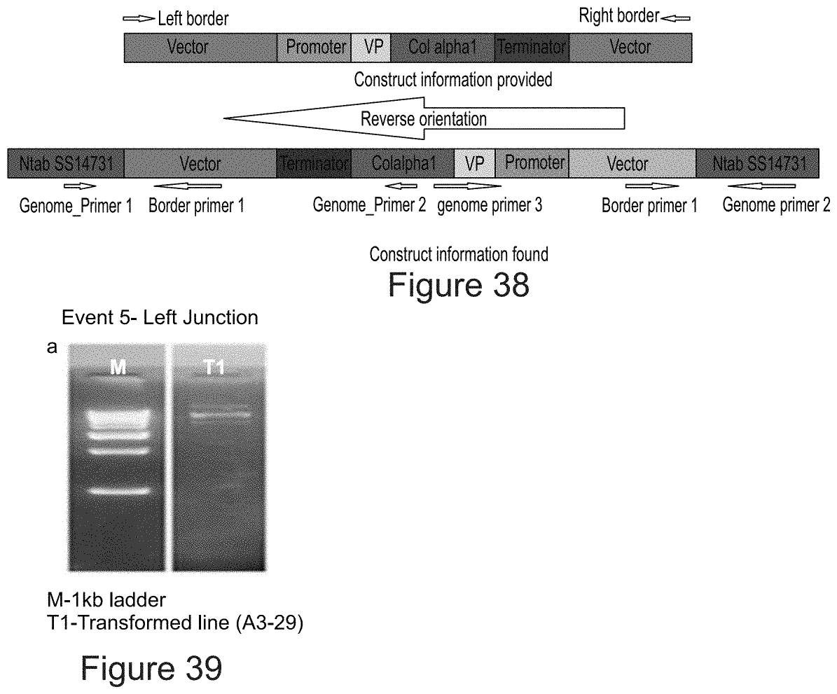

FIG. 38 shows a schematic diagram of event-5 position in the genome.

TABLE 16
Event information in Tabular format

| Sl | | | | |
|---|---|---|---|---|
| No | Description | Start | End | Strand |
| 1 | Ntab-TN90_AYMY-SS14731 | 48 | 1311 | + |
| 2 | Col alpha 1 | 10 | 2832 | + |
| 3 | Col alpha 1 | 7300 | 9304 | − |

TABLE 17
Primer set used for Nanopore-based sequencing

| Col | | | Product |
|---|---|---|---|
| alpha 1 | Genome_Primer | Gene_Primer | size |
| Left | Primer: | Primer: | −5.5 kb |
| Junction | MP_Col_4R | Colalpha1_R-end | |
| Primer | TGGATCAACTTAG | CACATCAAAACCGAA | |
| SEQ ID | CGGGAGT | CTCTTGA | |
| NOs of | | | |
| primers: | | | |
| 45 46 | | | |

FIG. 39 shows Insert characterization using event-5 left junction primer.

TABLE 18
Primer set used for Border PCR
(Validated by Sanger)

| Col | | | Product |
|---|---|---|---|
| alpha 1 | Genome_Primer | Border_Primer | size |
| Left | Primer: | Primer: RP2 | −3 Kb |
| Junction | MP_Col_ 3R | ATAAGGGATTTTG | |
| Primer | ACGGTTTTAAAGT | CCGATTTCG | |
| SEQ ID | CTTGCAACC | | |
| NOs of | | | |
| primers: | | | |
| 47 42 | | | |
| SEQ ID | Primer: | Primer: RP2 | −2 Kb |
| NOs of | MP_Col_4R | ATAAGGGATTTTG | |
| primers: | TGGATCAACTTAG | CCGATTTCG | |
| 45 42 | CGGGAGT | | |

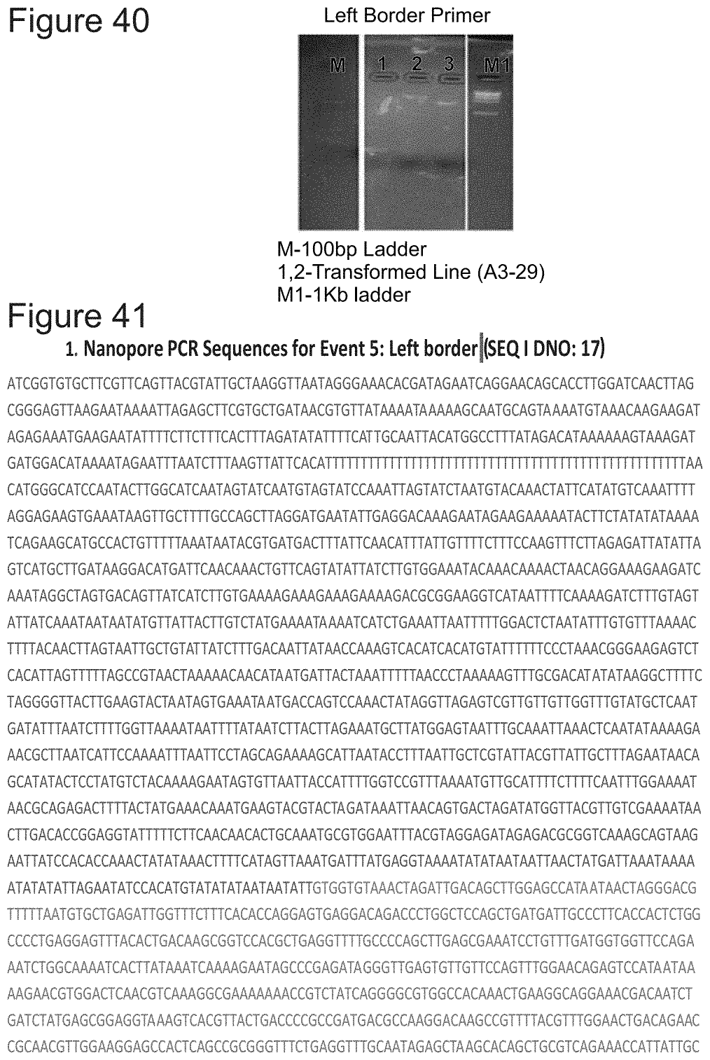

FIG. 40 shows the border junction PCR: Left border PCR using genome primer and border primer, amplicon size 2-˜3Kb, 3-2Kb.

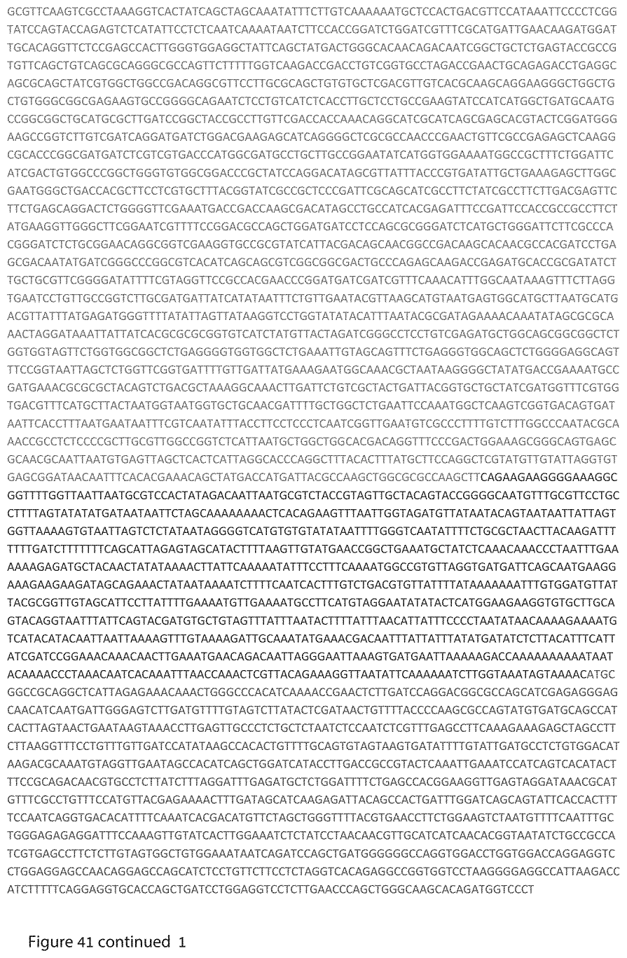

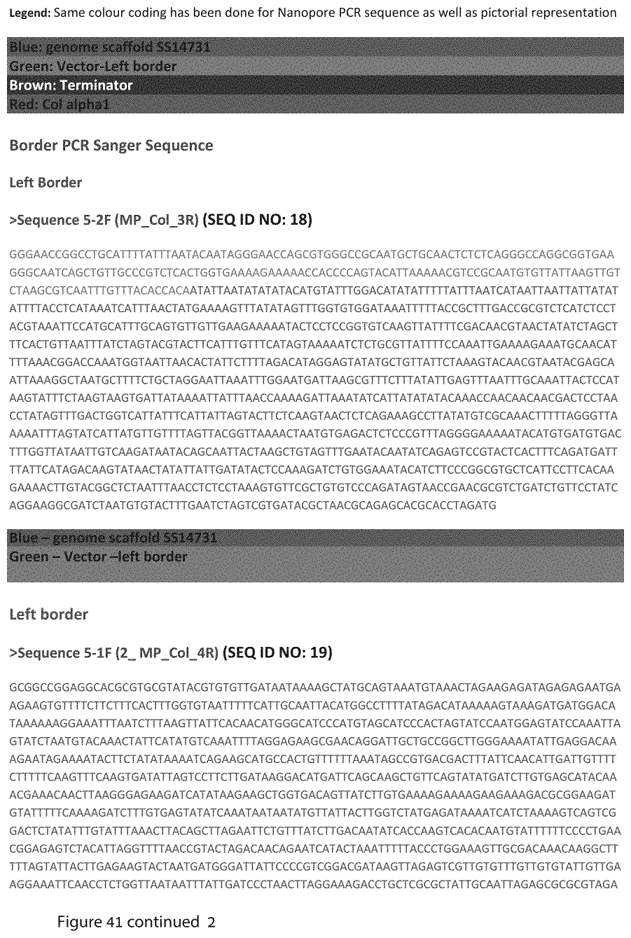

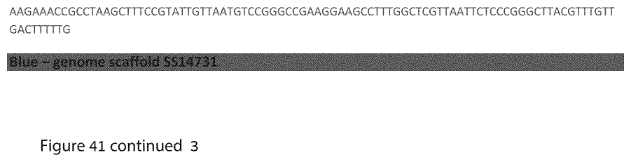

For construct inserted as Event5 for col alpha1- the left border was confirmed through Nanopore-based sequencing as well as Sanger sequencing methods. Col alphal is reverse oriented. Border PCR amplified the right borders but Sanger sequencing failed due to technical reasons. In FIG. 41, the regions (Right border vector and genome scaffold) are marked in lighter colors.

### Example 3

### Generation of Procollagen Producing Plant Varieties Objectives

The objective of this study was to examine possible new varieties high PC yielding and better agriculture performing production lines;

In order to do so, in parallel to the variety screening, a crossing program was conducted, in order to introduce all 5 PC transgenes into these varieties, based on 5 different hemizygous donors: A3-29-305-17-09-18 F6 bulk; A3-29-305 17 09 18 33 2 F7; A3-29-305-17-09-18-33-10 F7; A3-29-305 17 09 25 04 19 F7; A3-29-305 17 09 37 28 31 F7.

The major scope was to select lines with potential for an improved total biomass and PC yields compared to line A3-29-305-17-09-18 F5.

### MATERIALS AND METHODS

### Variety Screening and Crossing Program

Seeds from 28 different varieties were planted at anursery and 40-50 days later transplanted in four production areas; Merom Golan (MG), Ein Yahav (EY), Kalia (K) and in an experimental green house. Plantlets were transplanted at a density of 5 plants/meter row.

F1 screening

F1 seeds were harvested from the crosses and planted at a nursery. Transplantations were done in two waves.

### Field Planning

### Variety Screening and Crossing Program

In each production location, 6-10 plants of each variety were transplanted (Table 19 below).

At a greenhouse, 4 plants of each variety were transplanted, at a density of 2.5/meter row and 4 plants of each of 5 PC transgene donors (see below), in tree flowcharts (two rows each). Plants were monitored during all growing season.

In each plant at the green house, one inflorescence was covered by a paper bag, in order to maintain the line (producing more self-pollinated seeds).

For the crossing program, ten inflorescences in each variant were male sterilized in order to perform crossbreeding: each two inflorescences in each variety were crossbred with a different PC transgenes male donor (total of 10 crosses/variety) (Table 20, below).

The male donors were 4 selected F8 lines (A3-29-305 17 09 18 33 2, A3 29 305-17-09-18-33-10, A3-29-305-17-09-25-04-19, A3-29-305-17-09-37-28-31) and the production line (A3-29-305-17-09-18 F5).

TABLE 19
Varieties that were planted in different production areas, and number of
plants of each variety in each site. Number of repetition determined
according to number of developed plantlets.

| Code | Genotype | EY | Kalia | MG |
|---|---|---|---|---|
| 1 | N. tabacum cv. Cuban habano 2000 | 6 | 6 | 10 |
| 2 | N. tabacum cv. Burley Original | 6 | 6 | 6 |
| 3 | N. glauca Blue tree | | | 10 |
| 4 | N. tabacum cv. Virginia | | | 10 |
| 5 | N. tabacum cv.KY160 | 6 | 6 | 1 |
| 6 | N. tabacum cv. Virginia K326 | 6 | 6 | 1 |
| 7 | N. tabacum cv. Virginia K358 | 6 | 6 | 2 |
| 8 | N. tabacum cv. Burley TN86 | 3 | 3 | 6 |
| 9 | N. tabacum cv. Burley TN90 | 6 | 6 | 7 |
| 10 | N. tabacum cv. PG04 | 5 | 4 | 7 |
| 11 | N. tabacum cv. KY171LC | 6 | 5 | 10 |
| 12 | N. tabacum cv. Maryland | 6 | 6 | 9 |
| 13 | N. tabacum cv. Samsun NN | 6 | 5 | 6 |
| 14 | N. tabacum cv. MD 609 | | | 6 |
| 15 | N. tabacum cv. Tukish izmir | | | 6 |
| 16 | N. tabacum cv. Virginia gold 1 | 6 | 6 | 6 |
| 17 | N. tabacum cv. Narrow leat Madole | | | 6 |
| 18 | N. tabacum cv. Banket AA | | | 6 |
| 19 | N. tabacum cv. Lizard tail orinoco | 6 | 6 | 1 |
| 20 | N. tabacum cv. Virginia k346 | | | 6 |
| 21 | N. tabacum cv. Black mammoth | 6 | 6 | 6 |
| 22 | N. tabacum cv. Cuban criollo 98 | | | 6 |
| 37 | N. rustica | | | 6 |
| 38 | N. tabacum improved madole | 3 | 6 | 6 |
| 39 | N. tabacum perique | 6 | 6 | 6 |
| 40 | N. tabacum little wood | 6 | 6 | 6 |
| 41 | N. tabacum cv.KY160 | | | 6 |
| 42 | N. tabacum cv. Burley hampton | 6 | 6 | |

TABLE 20
Crossbreeding that were conducted at a greenhouse. Each variety
was introduced to the 5 PC producing transgenes by 5 different donors.

| Code | |
|---|---|
| Name | Cross |
| Vs-1 | [Cuban habano 2000NN (1) × A3-29-305-17-09-18 F5 (43)] F1 |
| Vs-2 | [Cuban habano 2000NN (1) × A3-29-305-17-09-18-33-2 F7 (44)] F1 |
| Vs-3 | [Cuban habano 2000NN (1) × A3-29-305-17-09-18-33-10F7 (45) F1 |
| Vs-4 | [Cuban habano 2000NN (1) × A3-29-305-17-09-25-04-19 F7 (46)] F1 |
| Vs-5 | [Cuban habano 2000NN (1) × A3-29-305-17-09-37-28-31 F7 (47)] F1 |
| Vs-6 | [Burley OriginalNN (2) × A3-29-305-17-09-18F5 (43)] F1 |
| Vs-7 | [Burley OriginalNN (2) × A3-29-305-17-09-18-33-2 F7 (44)] F1 |
| Vs-8 | [Burley OriginalNN (2) × A3-29-305-17-09-18-33-10F7 (45)] F1 |
| Vs-9 | [Burley OriginalNN (2) × A3-29-305-17-09-25-04-19 F7 (46)] F1 |
| Vs-10 | [Burley OriginalNN (2) × A3-29-305-17-09-37-28-31 F7 (47)] F1 |
| Vs-11 | [KY160NN (5) × A3-29-305-17-09-18F5 (43)] F1 |
| Vs-12 | [KY160NN (5) × A3-29-305-17-09-18-33-2 F7 (44)] F1 |
| Vs-13 | [KY160NN (5) × A3-29-305-17-09-18-33-10F7 (45)] F1 |
| Vs-14 | [KY160NN (5) × A3-29-305-17-09-25-04-19 F7 (46)] F1 |
| Vs-15 | [KY160NN (5) × A3-29-305-17-09-37-28-31 F7 (47)] F1 |
| Vs-16 | [Virginia K326NN (6) × A3-29-305-17-09-18F5 (43)] F1 |
| Vs-17 | [Virginia K326NN (6) × A3-29-305-17-09-18-33-2 F7 (44)] F1 |
| Vs-18 | [Virginia K326NN (6) × A3-29-305-17-09-18-33-10F7 (45)] F1 |
| Vs-19 | [Virginia K326NN (6) × A3-29-305-17-09-25-04-19 F7 (46)] F1 |
| Vs-20 | [Virginia K326NN (6) × A3-29-305-17-09-37-28-31 F7 (47)] F1 |
| Vs-21 | [Virginia K358NN (7) × A3-29-305-17-09-18F5 (43)] F1 |
| Vs-22 | [Virginia K358NN (7) × A3-29-305-17-09-18-33-2 F7 (44)] F1 |
| Vs-23 | [Virginia K358NN (7) × A3-29-305-17-09-18-33-10F7 (45)] F1 |
| Vs-24 | [Virginia K358NN (7) × A3-29-305-17-09-25-04-19 F7 (46)] F1 |
| Vs-25 | [Virginia K358NN (7) × A3-29-305-17-09-37-28-31 F7 (47)] F1 |
| Vs-26 | [Burley TN86NN (8) × A3-29-305-17-09-18F5 (43)] F1 |
| Vs-27 | [Burley TN86NN (8) × A3-29-305-17-09-18-33-2 F7 (44)] F1 |
| Vs-28 | [Burley TN86NN (8) × A3-29-305-17-09-18-33-10F7 (45)] F1 |
| Vs-29 | [Burley TN86NN (8) × A3-29-305-17-09-25-04-19 F7 (46)] F1 |
| Vs-30 | [Burley TN86NN (8) × A3-29-305-17-09-37-28-31 F7 (47)] F1 |
| Vs-31 | [Burley TN90NN (9) × A3-29-305-17-09-18F5 (43)] F1 |
| Vs-32 | [Burley TN90NN (9) × A3-29-305-17-09-18-33-2 F7 (44)] F1 |
| Vs-33 | [Burley TN90NN (9) × A3-29-305-17-09-18-33-10F7 (45)] F1 |
| Vs-34 | [Burley TN90NN (9) × A3-29-305-17-09-25-04-19 F7 (46)] F1 |
| Vs-35 | [Burley TN90NN (9) × A3-29-305-17-09-37-28-31 F7 (47)] F1 |
| Vs-36 | [PG04NN (10) × A3-29-305-17-09-18F5 (43)] F1 |
| Vs-37 | [PG04NN (10) × A3-29-305-17-09-18-33-2 F7 (44)] F1 |
| Vs-38 | [PG04NN (10) × A3-29-305-17-09-18-33-10F7 (45)] F1 |
| Vs-39 | [PG04NN (10) × A3-29-305-17-09-25-04-19 F7 (46)] F1 |
| Vs-40 | [PG04NN (10) × A3-29-305-17-09-37-28-31 F7 (47)] F1 |
| Vs-41 | [KY171LCNN (11) × A3-29-305-17-09-18F5 (43)] F1 |
| Vs-42 | [KY171LCNN (11) × A3-29-305-17-09-18-33-2 F7 (44)] F1 |
| Vs-43 | [KY171LCNN (11) × A3-29-305-17-09-18-33-10F7 (45)] F1 |
| Vs-44 | [KY171LCNN (11) × A3-29-305-17-09-25-04-19 F7 (46)] F1 |
| Vs-45 | [KY171LCNN (11) × A3-29-305-17-09-37-28-31 F7 (47)] F1 |
| Vs-46 | [N tabacum cv. MarylandNN (12) × A3-29-305-17-09-18F5 (43)] F1 |
| Vs-47 | [N tabacum cv. MarylandNN (12) × A3-29-305-17-09-18-33-2 F7 (44)] F1 |
| Vs-48 | [N tabacum cv. MarylandNN (12) × A3-29-305-17-09-18-33-10F7 (45)] F1 |
| Vs-49 | [N tabacum cv. MarylandNN (12) × A3-29-305-17-09-25-04-19 F7 (46)] F1 |
| Vs-50 | [N tabacum cv. MarylandNN (12) × A3-29-305-17-09-37-28-31 F7 (47)] F1 |
| Vs-56 | [N tabacum cv. MD 609NN (14) × A3-29-305-17-09-18F5 (43)] F1 |
| Vs-57 | [N tabacum cv. MD 609NN (14) × A3-29-305-17-09-18-33-2 F7 (44)] F1 |
| Vs-58 | [N tabacum cv. MD 609NN (14) × A3-29-305-17-09-18-33-10F7 (45)] F1 |
| Vs-59 | [N tabacum cv. MD 609NN (14) × A3-29-305-17-09-25-04-19 F7 (46)] F1 |
| Vs-60 | [N tabacum cv. MD 609NN (14) × A3-29-305-17-09-37-28-31 F7 (47)] F1 |
| Vs-61 | [N tabacum cv. Narrow leat MadoleNN (17) × A3-29-305-17-09-18F5 (43)] F1 |
| Vs-62 | [N tabacum cv. Narrow leat MadoleNN (17) × A3-29-305-17-09-18-33-2 F7 (44)] F1 |
| Vs-63 | [N tabacum cv. Narrow leat MadoleNN (17) × A3-29-305-17-09-18-33-10F7 (45)] F1 |
| Vs-64 | [N tabacum cv. Narrow leat MadoleNN (17) × A3-29-305-17-09-25-04-19 F7 (46)] F1 |
| Vs-65 | [N tabacum cv. Narrow leat MadoleNN (17) × A3-29-305-17-09-37-28-31 F7 (47)] F1 |
| Vs-66 | [N tabacum cv. Banket AANN (18) × A3-29-305-17-09-18F5 (43)] F1 |
| Vs-67 | [N tabacum cv. Banket AANN (18) × A3-29-305-17-09-18-33-2 F7 (44)] F1 |
| Vs-68 | [N tabacum cv. Banket AANN (18) × A3-29-305-17-09-18-33-10F7 (45)] F1 |
| Vs-69 | [N tabacum cv. Banket AANN (18) × A3-29-305-17-09-25-04-19 F7 (46)] F1 |
| Vs-70 | [N tabacum cv. Banket AANN (18) × A3-29-305-17-09-37-28-31 F7 (47)] F1 |
| Vs-71 | [N tabacum cv. Virginia k346NN (20) × A3-29-305-17-09-18F5 (43)] F1 |
| Vs-72 | [N tabacum cv. Virginia k346NN (20) × A3-29-305-17-09-18-33-2 F7 (44)] F1 |
| Vs-73 | [N tabacum cv. Virginia k346NN (20) × A3-29-305-17-09-18-33-10F7 (45)] F1 |
| Vs-74 | [N tabacum cv. Virginia k346NN (20) × A3-29-305-17-09-25-04-19 F7 (46)] F1 |
| Vs-75 | [N tabacum cv. Virginia k346NN (20) × A3-29-305-17-09-37-28-31 F7 (47)] F1 |
| Vs-76 | [N tabacum cv. Black mammoth NN (21) × A3-29-305-17-09-18F5 (43)] F1 |
| Vs-77 | [N tabacum cv. Black mammoth NN (21) × A3-29-305-17-09-18-33-2 F7 (44)] F1 |
| Vs-78 | [N tabacum cv. Black mammoth NN (21) × A3-29-305-17-09-18-33-10F7 (45)] F1 |
| Vs-79 | [N tabacum cv. Black mammoth NN (21) × A3-29-305-17-09-25-04-19 F7 (46)] F1 |
| Vs-80 | [N tabacum cv. Black mammoth NN (21) × A3-29-305-17-09-37-28-31 F7 (47)] F1 |
| Vs-81 | [N tabacum cv. Cuban criollo 98NN (22) × A3-29-305-17-09-18F5 (43)] F1 |
| Vs-82 | [N tabacum cv. Cuban criollo 98NN (22) × A3-29-305-17-09-18-33-2 F7 (44)] F1 |
| Vs-83 | [N tabacum cv. Cuban criollo 98NN (22) × A3-29-305-17-09-18-33-10F7 (45)] F1 |
| Vs-84 | [N tabacum cv. Cuban criollo 98NN (22) × A3-29-305-17-09-25-04-19 F7 (46)] F1 |
| Vs-85 | [N tabacum cv. Cuban criollo 98NN (22) × A3-29-305-17-09-37-28-31 F7 (47)] F1 |
| Vs-86 | [Nicotiana rusticaNN (37) × A3-29-305-17-09-18F5 (43)] F1 |
| Vs-87 | [Nicotiana rusticaNN (37) × A3-29-305-17-09-18-33-2 F7 (44)] F1 |
| Vs-88 | [Nicotiana rusticaNN (37) × A3-29-305-17-09-18-33-10F7 (45)] F1 |
| Vs-89 | [Nicotiana rusticaNN (37) × A3-29-305-17-09-25-04-19 F7 (46)] F1 |
| Vs-90 | [Nicotiana rusticaNN (37) × A3-29-305-17-09-37-28-31 F7 (47)] F1 |
| Vs-91 | [Nicotiana tabacum improved madole NN (38) × A3-29-305-17-09-18F5 (43)] F1 |
| Vs-92 | [Nicotiana tabacum improved madole NN (38) × A3-29-305-17-09-18-33-2 F7 (44)] F1 |
| Vs-93 | [Nicotiana tabacum improved madole NN (38) × A3-29-305-17-09-18-33-10F7 (45)] F1 |
| Vs-94 | [Nicotiana tabacum improved madole NN (38) × A3-29-305-17-09-25-04-19 F7 (46)] F1 |
| Vs-95 | [Nicotiana tabacum improved madole NN (38) × A3-29-305-17-09-37-28-31 F7 (47)] F1 |
| Vs-96 | [Nicotiana tabacum perique NN (39) × A3-29-305-17-09-18F5 (43)] F1 |
| Vs-97 | [Nicotiana tabacum perique NN (39) × A3-29-305-17-09-18-33-2 F7 (44)] F1 |
| Vs-98 | [Nicotiana tabacum perique NN (39) × A3-29-305-17-09-18-33-10F7 (45)] F1 |
| Vs-99 | [Nicotiana tabacum perique NN (39) × A3-29-305-17-09-25-04-19 F7 (46)] F1 |
| Vs-100 | [Nicotiana tabacum perique NN (39) × A3-29-305-17-09-37-28-31 F7 (47)] F1 |
| Vs-101 | [Nicotiana tabacum little wood NN (40) × A3-29-305-17-09-18F5 (43)] F1 |
| Vs-102 | [Nicotiana tabacum little wood NN (40) × A3-29-305-17-09-18-33-2 F7 (44)] F1 |
| Vs-103 | [Nicotiana tabacum little wood NN (40) × A3-29-305-17-09-18-33-10F7 (45)] F1 |
| Vs-104 | [Nicotiana tabacum little wood NN (40) × A3-29-305-17-09-25-04-19 F7 (46)] F1 |
| Vs-105 | [Nicotiana tabacum little wood NN (40) × A3-29-305-17-09-37-28-31 F7 (47)] F1 |
| Vs-106 | [Burley hamptonNN (42) × A3-29-305-17-09-18F5 (43)] F1 |
| Vs-107 | [Burley hamptonNN (42) × A3-29-305-17-09-18-33-2 F7 (44)] F1 |
| Vs-108 | [Burley hamptonNN (42) × A3-29-305-17-09-18-33-10F7 (45)] F1 |
| Vs-109 | [Burley hamptonNN (42) × A3-29-305-17-09-25-04-19 F7 (46) F1 |
| Vs-110 | [Burley hamptonNN (42) × A3-29-305-17-09-37-28-31 F7 (47) F1 |
| Vs-111 | [VirginiaNN (4) × A3-29-305-17-09-18F5 (43)] F1 |
| Vs-112 | [VirginiaNN (4) × A3-29-305-17-09-18-33-2 F7 (44)] F1 |
| Vs-113 | [VirginiaNN (4) × A3-29-305-17-09-18-33-10F7 (45)] F1 |
| Vs-114 | [VirginiaNN (4) × A3-29-305-17-09-25-04-19 F7 (46)] F1 |
| Vs-115 | [VirginiaNN (4) × A3-29-305-17-09-37-28-31 F7 (47)] F1 |
| Vs-116 | [Virginia GoldNN (16) × A3-29-305-17-09-18F5 (43)] F1 |
| Vs-117 | [Virginia GoldNN (16) × A3-29-305-17-09-18-33-2 F7 (44)] F1 |
| Vs-118 | [Virginia GoldNN (16) × A3-29-305-17-09-18-33-10F7 (45)] F1 |
| Vs-119 | [Virginia GoldNN (16) × A3-29-305-17-09-25-04-19 F7 (46)] F1 |
| Vs-120 | [Virginia GoldNN (16) × A3-29-305-17-09-37-28-31 F7 (47)] F1 |

### F1Screening

10 plants of each Fl cross were planted at the greenhouse, at 2.5 plant/meter row density, at a block design of 2 rows of 5 plants/cross. Crosses were arranged by groups of the female line, in order to get better impression of the heterotic potential (Table 21, below).

TABLE 21
F1 family's nursery list. Each new variety (maternal line) was crossed with 5
different PC production transgenes donors (paternal line). F1's were transplanted in
two waves, as described above.

| Code | | Planting | Transplanting |
|---|---|---|---|
| Name | Cross | date | date |
| Vs-1 | [Cuban habano 2000NN (1) × A3-29-305-17-09-18F5 (43)] F1 | Nov. 16, 2016 | Jan. 4, 2017 |
| Vs-2 | [Cuban habano 2000NN (1) × A3-29-305-17-09-18-33-2 F7 (44)] F1 | Nov. 16, 2016 | Jan. 4, 2017 |
| Vs-3 | [Cuban habano 2000NN (1) × A3-29-305-17-09-18-33-10F7 (45)] F1 | Nov. 16, 2016 | Jan. 4, 2017 |
| Vs-4 | [Cuban habano 2000NN (1) × A3-29-305-17-09-25-04-19 F7 (46)] F1 | Nov. 16, 2016 | Jan. 4, 2017 |
| Vs-5 | [Cuban habano 2000NN (1) × A3-29-305-17-09-37-28-31 F7 (47)] F1 | Nov. 16, 2016 | Jan. 4, 2017 |
| Vs-6 | [Burley OriginalNN (2) × A3-29-305-17-09-18F5 (43)] F1 | Nov. 16, 2016 | Jan. 4, 2017 |
| Vs-7 | [Burley OriginalNN (2) × A3-29-305-17-09-18-33-2 F7 (44)] F1 | Nov. 16, 2016 | Jan. 4, 2017 |
| Vs-8 | [Burley OriginalNN (2) × A3-29-305-17-09-18-33-10F7 (45)] F1 | Nov. 16, 2016 | Jan. 4, 2017 |
| Vs-9 | [Burley OriginalNN (2) × A3-29-305-17-09-25-04-19 F7 (46)] F1 | Nov. 16, 2016 | Jan. 4, 2017 |
| Vs-10 | [Burley OriginalNN (2) × A3-29-305-17-09-37-28-31 F7 (47)] F1 | Nov. 16, 2016 | Jan. 4, 2017 |
| Vs-12 | [KY160NN (5) × A3-29-305-17-09-18-33-2 F7 (44)] F1 | Nov. 16, 2016 | Jan. 4, 2017 |
| Vs-14 | [KY160NN (5) × A3-29-305-17-09-25-04-19 F7 (46)] F1 | Nov. 16, 2016 | Jan. 4, 2017 |
| Vs-15 | [KY160NN (5) × A3-29-305-17-09-37-28-31 F7 (47)] F1 | Nov. 16, 2016 | Jan. 4, 2017 |
| Vs-17 | [Virginia K326NN (6) × A3-29-305-17-09-18-33-2 F7 (44)] F1 | Nov. 16, 2016 | Jan. 4, 2017 |
| Vs-21 | [Virginia K358NN (7) × A3-29-305-17-09-18F5 (43)] F1 | Mar. 27, 2017 | May 15, 2017 |
| Vs-22 | [Virginia K358NN (7) × A3-29-305-17-09-18-33-2 F7 (44)] F1 | Mar. 27, 2017 | May 15, 2017 |
| Vs-23 | [Virginia K358NN (7) × A3-29-305-17-09-18-33-10F7 (45)] F1 | Mar. 27, 2017 | May 15, 2017 |
| Vs-24 | [Virginia K358NN (7) × A3-29-305-17-09-25-04-19 F7 (46)] F1 | Mar. 27, 2017 | May 15, 2017 |
| Vs-25 | [Virginia K358NN (7) × A3-29-305-17-09-37-28-31 F7 (47)] F1 | Mar. 27, 2017 | May 15, 2017 |
| Vs-26 | [Burley TN86NN (8) × A3-29-305-17-09-18F5 (43)] F1 | Mar. 27, 2017 | May 15, 2017 |
| Vs-27 | [Burley TN86NN (8) × A3-29-305-17-09-18-33-2 F7 (44)] F1 | Nov. 16, 2016 | Jan. 4, 2017 |
| Vs-29 | [Burley TN86NN (8) × A3-29-305-17-09-25-04-19 F7 (46)] F1 | Mar. 27, 2017 | May 15, 2017 |
| Vs-30 | [Burley TN86NN (8) × A3-29-305-17-09-37-28-31 F7 (47)] F1 | Mar. 27, 2017 | May 15, 2017 |
| Vs-31 | [Burley TN90NN (9) × A3-29-305-17-09-18F5 (43)] F1 | Nov. 16, 2016 | Jan. 4, 2017 |
| Vs-32 | [Burley TN90NN (9) × A3-29-305-17-09-18-33-2 F7 (44)] F1 | Nov. 16, 2016 | Jan. 4, 2017 |
| Vs-33 | [Burley TN90NN (9) × A3-29-305-17-09-18-33-10F7 (45)] F1 | Nov. 16, 2016 | Jan. 4, 2017 |
| Vs-34 | [Burley TN90NN (9) × A3-29-305-17-09-25-04-19 F7 (46)] F1 | Mar. 27, 2017 | May 15, 2017 |
| Vs-35 | [Burley TN90NN (9) × A3-29-305-17-09-37-28-31 F7 (47)] F1 | Nov. 16, 2016 | Jan. 4, 2017 |
| Vs-36 | [PG04NN (10) × A3-29-305-17-09-18F5 (43)] F1 | Mar. 27, 2017 | May 15, 2017 |
| Vs-37 | [PG04NN (10) × A3-29-305-17-09-18-33-2 F7 (44)] F1 | Nov. 16, 2016 | Jan. 4, 2017 |
| Vs-38 | [PG04NN (10) × A3-29-305-17-09-18-33-10F7 (45)] F1 | Nov. 16, 2016 | Jan. 4, 2017 |
| Vs-39 | [PG04NN (10) × A3-29-305-17-09-25-04-19 F7 (46)] F1 | Nov. 16, 2016 | Jan. 4, 2017 |
| Vs-42 | [KY171LCNN (11) × A3-29-305-17-09-18-33-2 F7 (44)] F1 | Nov. 16, 2016 | Jan. 4, 2017 |
| Vs-43 | [KY171LCNN (11) × A3-29-305-17-09-18-33-10F7 (45)] F1 | Nov. 16, 2016 | Jan. 4, 2017 |
| Vs-44 | [KY171LCNN (11) × A3-29-305-17-09-25-04-19 F7 (46)] F1 | Nov. 16, 2016 | Jan. 4, 2017 |
| Vs-45 | [KY171LCNN (11) × A3-29-305-17-09-37-28-31 F7 (47)] F1 | Nov. 16, 2016 | Jan. 4, 2017 |
| Vs-46 | [N tabacum cv. MarylandNN (12) × A3-29-305-17-09-18F5 (43)] F1 | Nov. 16, 2016 | Jan. 4, 2017 |
| Vs-47 | [N tabacum cv. MarylandNN (12) × A3-29-305-17-09-18-33-2 F7 (44)] F1 | Nov. 16, 2016 | Jan. 4, 2017 |
| Vs-48 | [N tabacum cv. MarylandNN (12) × A3-29-305-17-09-18-33-10F7 (45)] F1 | Nov. 16, 2016 | Jan. 4, 2017 |
| Vs-49 | [N tabacum cv. MarylandNN (12) × A3-29-305-17-09-25-04-19 F7 (46)] F1 | Mar. 27, 2017 | May 15, 2017 |
| Vs-50 | [N tabacum cv. MarylandNN (12) × A3-29-305-17-09-37-28-31 F7 (47)] F1 | Nov. 16, 2016 | Jan. 4, 2017 |
| Vs-56 | [N tabacum cv. MD 609NN (14) × A3-29-305-17-09-18F5 (43)] F1 | Mar. 27, 2017 | May 15, 2017 |
| Vs-57 | [N tabacum cv. MD 609NN (14) × A3-29-305-17-09-18-33-2 F7 (44)] F1 | Nov. 16, 2016 | Jan. 4, 2017 |
| Vs-58 | [N tabacum cv. MD 609NN (14) × A3-29-305-17-09-18-33-10F7 (45)] F1 | Nov. 16, 2016 | Jan. 4, 2017 |
| Vs-59 | [N tabacum cv. MD 609NN (14) × A3-29-305-17-09-25-04-19 F7 (46)] F1 | Mar. 27, 2017 | May 15, 2017 |
| Vs-60 | [N tabacum cv. MD 609NN (14) × A3-29-305-17-09-37-28-31 F7 (47)] F1 | Mar. 27, 2017 | May 15, 2017 |
| Vs-66 | [N tabacum cv. Banket AANN (18) × A3-29-305-17-09-18F5 (43)] F1 | Mar. 27, 2017 | May 15, 2017 |
| Vs-67 | [N tabacum cv. Banket AANN (18) × A3-29-305-17-09-18-33-2 F7 (44)] F1 | Mar. 27, 2017 | May 15, 2017 |
| Vs-68 | [N tabacum cv. Banket AANN (18) × A3-29-305-17-09-18-33-10F7 (45)] F1 | Mar. 27, 2017 | May 15, 2017 |
| Vs-69 | [N tabacum cv. Banket AANN (18) × A3-29-305-17-09-25-04-19 F7 (46)] F1 | Mar. 27, 2017 | May 15, 2017 |
| Vs-70 | [N tabacum cv. Banket AANN (18) × A3-29-305-17-09-37-28-31 F7 (47)] F1 | Mar. 27, 2017 | May 15, 2017 |
| Vs-77 | [N tabacum cv. Black mammoth NN (21) × A3-29-305-17-09-18-33-2 F7 (44)] F1 | Nov. 16, 2016 | Jan. 4, 2017 |
| Vs-78 | [N tabacum cv. Black mammoth NN (21) × A3-29-305-17-09-18-33-10F7 (45)] F1 | Nov. 16, 2016 | Jan. 4, 2017 |
| Vs-81 | [N tabacum cv. Cuban criollo 98NN (22) × A3-29-305-17-09-18F5 (43)] F1 | Nov. 16, 2016 | Jan. 4, 2017 |
| Vs-82 | [N tabacum cv. Cuban criollo 98NN (22) × A3-29-305-17-09-18-33-2 F7 (44)] F1 | Nov. 16, 2016 | Jan. 4, 2017 |
| Vs-83 | [N tabacum cv. Cuban criollo 98NN (22) × A3-29-305-17-09-18-33-10F7 (45)] F1 | Nov. 16, 2016 | Jan. 4, 2017 |
| Vs-84 | [N tabacum cv. Cuban criollo 98NN (22) × A3-29-305-17-09-25-04-19 F7 (46)] F1 | Nov. 16, 2016 | Jan. 4, 2017 |
| Vs-85 | [N tabacum cv. Cuban criollo 98NN (22) × A3-29-305-17-09-37-28-31 F7 (47)] F1 | Nov. 16, 2016 | Jan. 4, 2017 |
| Vs-88 | [Nicotiana rusticaNN (37) × A3-29-305-17-09-18-33-10F7 (45)] F1 | Nov. 16, 2016 | Jan. 4, 2017 |
| Vs-90 | [Nicotiana rusticaNN (37) × A3-29-305-17-09-37-28-31 F7 (47)] F1 | Nov. 16, 2016 | Jan. 4, 2017 |
| Vs-91 | [Nicotiana tabacum improved madole NN (38) x A3-29-305-17-09-18F5 (43)] F1 | Mar. 27, 2017 | May 15, 2017 |
| Vs-92 | [Nicotiana tabacum improved madole NN (38) × A3-29-305-17-09-18-33-2F7 (44)] F1 | Nov. 16, 2016 | Jan. 4, 2017 |
| Vs-93 | [Nicotiana tabacum improved madole NN (38) × A3-29-305-17-09-18-33-10F7 (45)] F1 | Mar. 27, 2017 | May 15, 2017 |
| Vs-95 | [Nicotiana tabacum improved madole NN (38) × A3-29-305-17-09-37-28-31F7 (47)] F1 | Mar. 27, 2017 | May 15, 2017 |

### Plant Growing and Monitoring

### Variety Screening

Plants were scored visually according to overall agronomic performances, with focus on structure and biomass yield potential (no specific characteristics), in each site separately (Table 22, below). The major scope was to find varieties that are well adapted to the production locations and efficient biomass production potential.

Simultaneously, crossbreeding was conducted at a greenhouse with five male donors.

Plants were grown at the production areas until the observation completed. Crossbreeding Plants were grown at the green house until self-pollinated seeds and cross-pollinated seeds matured to harvest.

### Crossbreeding

The cross-breeding plan was conducted in two waves, due to stem diseases caused by drainage problems.

Priorities for the crossbreeding plan were determined, as well as dropping some of the planned crosses based on continuous screening and monitoring of the varieties at the production location. 17 varieties were identified as potential parents, by visual selection (Table 21a).

### F1 Screening

Plants were grown at a green house. In each plant, one inflorescence was covered by a paper bag, in order to produce self-pollinated seeds (F2 seeds). Plants were grown at the green house until self-pollinated seeds were mature and seeds harvested.

F1 Plants were monitored visually throughout the growing season for agronomic traits, especially for structure and biomass yield potential.

TABLE 21a

| | Female/Male | | | |
|---|---|---|---|---|
| | M.G | E.Y | K | Over all |
| Cuban habano 2000 | + | + | + | + |
| Burley Original | V | ++ | − | V |
| KY160 | V | − | − | + |
| Virginia K326 | V | − | − | + |
| Virginia K358 | VV | + | + | V |
| Burley TN86 | VV | VV | V | VV |
| Burley TN90 | VV | VV | V | VV |
| PG04 | VV | V | +/V | V |
| KY171LC | V | − | +/− | + |
| N tabacum cv. Maryland | V | VV | VV | VV |
| N tabacum cv. MD 609 | VV | | | VV |
| N tabacum cv. Virginia gold 1 | | V | + | + |
| N tabacum cv. Narrow leat | + | | | + |
| Madole | | | | |
| N tabacum cv. Banket AA | VV | | | V |
| N tabacum cv. Black mammoth | V | − | +/− | + |
| Nicotiana tabacum improved | VVV | − | | V |
| madole | | | | |
| Nicotiana tabacum perique | V | − | + | + |

### Analysis

### Molecular Analysis

Out of each F1 family, 5 plants were sampled and analyzed for the presence of each of the 5 PC (procollagen) production genes, using RT-PCR. Plants that showed the presence of at least 3 of the 5 transgenes by the RT-PCR analysis were sampled for further analysis of PC content, using ELISA. For ELISA analysis, all leaves were picked in each plant, 64 days from transplanting, Processed and analysed by ELISA. Additionally, leaves were weighed in each plant (FIG. 42).

### Results

Variety Screening—Scoring and Priorities

TABLE 22
Scores of overall agronomic performances of the varieties that determined
as potential parents, in each site separately and overall score of priority.

| | | | | | | | PC | | Total |
|---|---|---|---|---|---|---|---|---|---|
| | | | | | | | concen- | Leaves | PC |
| | Selected | | | | | | tration | weight | yield |
| | for | | | | PH4- | PH4- | (mg/kg | (gr/ | (mg/ |
| Full Name | ELISA | col1 | Col2 | LH3 | alpha | beta | Leaves) | plant) | plant) |
| [[Cuban habano 2000NN (1) × A3-29-305-17-09- | Yes | Y | N | Y | Y | Y | 94.8 | 1183 | 112.15 |
| 18F5 (43)] ] (Vs-1) (1)″F1 # 1 | | | | | | | | | |
| [[Cuban habano 2000NN (1) × A3-29-305-17-09-18F5 (43)] ] | | N | N | Y | Y | Y | | | |
| (Vs-1) F1 # 8 | | | | | | | | | |
| [[Cuban habano 2000NN (1) × A3-29-305-17-09-18F5 (43)] ] | | N | N | N | Y | Y | | | |
| (Vs-1) F1 # 10 | | | | | | | | | |
| [[Cuban habano 2000NN (1) × A3-29-305-17-09- | Yes | Y | N | Y | Y | Y | 83.5 | 981 | 81.91 |
| 18F5 (43)] ] (Vs-1) (8)″F1 # 8 | | | | | | | | | |
| [[Cuban habano 2000NN (1) × A3-29-305-17-09-18F5 (43)] ] | | N | N | N | Y | Y | | | |
| (Vs-1) F1 # 3 | | | | | | | | | |
| [[Cuban habano 2000NN (1) × A3-29-305-17-09-18-33-2 F7 | | N | N | Y | Y | Y | | | |
| (44)] ] (Vs-2) F1 # 7 | | | | | | | | | |
| [[Cuban habano 2000NN (1) × A3-29-305-17-09-18-33-2 F7 | | N/A | N/A | N/A | N/A | N/A | | | |
| (44)] ] (Vs-2) F1 # 2 | | | | | | | | | |
| [[Cuban habano 2000NN (1) × A3-29-305-17-09-18-33-2 F7 | | N/A | N/A | N/A | N/A | N/A | | | |
| (44)] ] (Vs-2) F1 # 3 | | | | | | | | | |
| [[Cuban habano 2000NN (1) × A3-29-305-17-09- | Yes | Y | Y | Y | Y | Y | 66.5 | 713 | 47.41 |
| 18-33-2 F7 (44)] ] (Vs-2) (10)″F1 # 10 | | | | | | | | | |
| [[Cuban habano 2000NN (1) × A3-29-305-17-09- | Yes | Y | N | Y | Y | Y | 72.1 | 999 | 72.03 |
| 18-33-2 F7 (44)] ] (Vs-2) (5)″F1 # 5 | | | | | | | | | |
| [[Cuban habano 2000NN (1) × A3-29-305-17-09-18-33-10F7 | | Y | Y | Y | Y | Y | | | |
| (45)] ] (Vs-3) F1 # 3 | | | | | | | | | |
| [[Cuban habano 2000NN (1) × A3-29-305-17-09-18-33-10F7 | | N | N | N | N | N | | | |
| (45)] ] (Vs-3) F1 # 2 | | | | | | | | | |
| [[Cuban habano 2000NN (1) × A3-29-305-17-09- | Yes | Y | Y | Y | Y | Y | 83.1 | 647 | 53.77 |
| 18-33-10F7 (45)] ] (Vs-3) (3)″F1 # 3 | | | | | | | | | |
| [[Cuban habano 2000NN (1) × A3-29-305-17-09- | Yes | Y | Y | Y | Y | Y | 65.2 | 661 | 43.10 |
| 18-33-10F7 (45)] ] (Vs-3) (7)″F1 # 7 | | | | | | | | | |
| [[Cuban habano 2000NN (1) × A3-29-305-17-09-18-33-10F7 | | N | N | N | N | N | | | |
| (45)] ] (Vs-3) F1 # 3 | | | | | | | | | |
| [[Cuban habano 2000NN (1) × A3-29-305-17-09-25-04-19 F7 | | N | N | N | N | Y | | | |
| (46)] ] (Vs-4) F1 # 4 | | | | | | | | | |
| [[Cuban habano 2000NN (1) × A3-29-305-17-09- | Yes | Y | Y | Y | Y | Y | 83 | 871 | 72.29 |
| 25-04-19 F7 (46)] ] (Vs-4) (2)″F1 # 2 | | | | | | | | | |
| [[Cuban habano 2000NN (1) × A3-29-305-17-09- | Yes | Y | Y | Y | Y | Y | 70.1 | 862 | 60.43 |
| 25-04-19 F7 (46)] ] (Vs-4) (3)″F1 # 3 | | | | | | | | | |
| [[Cuban habano 2000NN (1) × A3-29-305-17-09-25-04-19 F7 | | N | N | Y | Y | Y | | | |
| (46)] ] (Vs-4) F1 # 1 | | | | | | | | | |
| [[Cuban habano 2000NN (1) × A3-29-305-17-09-25-04-19 F7 | | Y | Y | Y | Y | Y | | | |
| (46)] ] (Vs-4) F1 # 3 | | | | | | | | | |
| [[Cuban habano 2000NN (1) × A3-29-305-17-09- | Yes | Y | Y | Y | Y | Y | 54.2 | 591 | 32.03 |
| 37-28-31 F7 (47)] ] (Vs-5) (1)″F1 # 1 | | | | | | | | | |
| [[Cuban habano 2000NN (1) × A3-29-305-17-09- | Yes | Y | Y | Y | Y | Y | 93.7 | 1144 | 107.19 |
| 37-28-31 F7 (47)] ] (Vs-5) (2)″F1 # 2 | | | | | | | | | |
| [[Cuban habano 2000NN (1) × A3-29-305-17-09-37-28-31 F7 | | N | N | Y | Y | Y | | | |
| (47)] ] (Vs-5) F1 # 3 | | | | | | | | | |
| [[Cuban habano 2000NN (1) × A3-29-305-17-09-37-28-31 F7 | | Y | Y | Y | Y | Y | | | |
| (47)] ] (Vs-5) F1 # 4 | | | | | | | | | |
| [[Cuban habano 2000NN (1) × A3-29-305-17-09-37-28-31 F7 | | N | N | N | Y | Y | | | |
| (47)] ] (Vs-5) F1 # 1 | | | | | | | | | |
| [[Burley OriginaINN (2) × A3-29-305-17-09-18F5 | | N | N | N | Y | Y | | | |
| (43)] ] (Vs-6) F1 # 8 | | | | | | | | | |
| [[Burley OriginaINN (2) × A3-29-305-17-09-18F5 | | Y | N | N | Y | Y | | | |
| (43)] ] (Vs-6) F1 # 6 | | | | | | | | | |
| [[Burley OriginaINN (2) × A3-29-305-17-09-18F5 | Yes | Y | Y | Y | Y | Y | 56.9 | 962 | 54.74 |
| (43)] ] (Vs-6) (3)″F1 # 3 | | | | | | | | | |
| [[Burley OriginaINN (2) × A3-29-305-17-09-18F5 | | N/A | N/A | N/A | N/A | N/A | | | |
| (43)] ] (Vs-6) F1 # 1 | | | | | | | | | |
| [[Burley OriginaINN (2) × A3-29-305-17-09-18F5 | | Y | N | Y | Y | Y | | | |
| (43)] ] (Vs-6) F1 # 2 | | | | | | | | | |
| [[Burley OriginaINN (2) × A3-29-305-17-09-18-33-2 F7 (44)] ] | | Y | N | Y | Y | Y | | | |
| (Vs-7) F1 # 1 | | | | | | | | | |
| [[Burley OriginaINN (2) × A3-29-305-17-09-18-33- | Yes | Y | Y | Y | Y | Y | 40 | 828 | 33.12 |
| 2 F7 (44)] ] (Vs-7) (2)″F1 # 2 | | | | | | | | | |
| [[Burley OriginaINN (2) × A3-29-305-17-09-18-33-2 F7 (44)] ] | | N | N | N | N | Y | | | |
| (Vs-7) F1 # 1 | | | | | | | | | |
| [[Burley OriginaINN (2) × A3-29-305-17-09-18-33-2 F7 (44)] ] | | Y | N | Y | Y | Y | | | |
| (Vs-7) F1 # 2 | | | | | | | | | |
| [[Burley OriginaINN (2) × A3-29-305-17-09-18-33-2 F7 (44)] ] | | Y | N | Y | Y | Y | | | |
| (Vs-7) F1 # 1 | | | | | | | | | |
| [[Burley OriginaINN (2) × A3-29-305-17-09-18-33- | Yes | Y | N | Y | Y | Y | 44 | 1575 | 69.30 |
| 10F7 (45)] ] (Vs-8) (1)″F1 # 1 | | | | | | | | | |
| [[Burley OriginaINN (2) × A3-29-305-17-09-18-33-10F7 (45)] ] | | N | N | N | Y | Y | | | |
| (Vs-8) F1 # 1 | | | | | | | | | |
| [[Burley OriginaINN (2) × A3-29-305-17-09-18-33- | Yes | Y | N | Y | Y | Y | 32.4 | 1081 | 35.02 |
| 10F7 (45)] ] (Vs-8) (3)″F1 # 3 | | | | | | | | | |
| [[Burley OriginaINN (2) × A3-29-305-17-09-18-33-10F7 (45)] ] | | N | N | N | Y | Y | | | |
| (Vs-8) F1 # 1 | | | | | | | | | |
| [[Burley OriginaINN (2) × A3-29-305-17-09-18-33-10F7 (45)] ] | | Y | N | Y | Y | Y | | | |
| (Vs-8) F1 # 2 | | | | | | | | | |
| [[Burley OriginaINN (2) × A3-29-305-17-09-25-04-19 F7 (46)] ] | | N | Y | Y | N | Y | | | |
| (Vs-9) F1 # | | | | | | | | | |
| [[Burley OriginaINN (2) × A3-29-305-17-09-25-04-19 F7 (46)] ] | | N | N | N | N | N | | | |
| (Vs-9) F1 # 1 | | | | | | | | | |
| [[Burley OriginaINN (2) × A3-29-305-17-09-25-04-19 F7 (46)] ] | | N/A | N/A | N/A | N/A | N/A | | | |
| (Vs-9) F1 # 2 | | | | | | | | | |
| [[Burley OriginaINN (2) × A3-29-305-17-09-25-04-19 F7 (46)] ] | | Y | N | N | N | N | | | |
| (Vs-9) F1 # 4 | | | | | | | | | |
| [[Burley OriginaINN (2) × A3-29-305-17-09-25-04-19 F7 (46)] ] | | N/A | N/A | N/A | N/A | N/A | | | |
| (Vs-9) F1 # 6 | | | | | | | | | |
| [[Burley OriginaINN (2) × A3-29-305-17-09-37-28-31 F7 (47)] ] | | N/A | N/A | N/A | N/A | N/A | | | |
| (Vs-10) F1 # 1 | | | | | | | | | |
| [[Burley OriginaINN (2) × A3-29-305-17-09-37-28-31 F7 (47)] ] | | Y | N | N | Y | Y | | | |
| (Vs-10) F1 # 2 | | | | | | | | | |
| [[Burley OriginaINN (2) × A3-29-305-17-09-37-28- | Yes | Y | Y | Y | Y | Y | 44.5 | 762 | 33.91 |
| 31 F7 (47)] ] (Vs-10) (3)″F1 # 3 | | | | | | | | | |
| [[Burley OriginaINN (2) × A3-29-305-17-09-37-28- | Yes | Y | Y | Y | Y | Y | 31.4 | 582 | 18.27 |
| 31 F7 (47)] ] (Vs-10) (4)″F1 # 4 | | | | | | | | | |
| [[Burley OriginaINN (2) × A3-29-305-17-09-37-28-31 F7 (47)] ] | | Y | Y | Y | Y | Y | | | |
| (Vs-10) F1 # 1 | | | | | | | | | |
| [[KY160NN (5) × A3-29-305-17-09-18-33-2 F7 (44)] ] | | N/A | N/A | N/A | N/A | N/A | | | |
| (Vs-12) F1 # 4 | | | | | | | | | |
| [[KY160NN (5) × A3-29-305-17-09-18-33-2 F7 (44)] ] | | N | N | N/ | N | N | | | |
| (Vs-12) F1 # 3 | | | | | | | | | |
| [[KY160NN (5) × A3-29-305-17-09-18-33-2 F7 (44) ] ] | Yes | Y | Y | Y | Y | Y | 44.1 | 811 | 35.77 |
| (Vs-12) (3)″F1 # 3 | | | | | | | | | |
| [[KY160NN (5) × A3-29-305-17-09-18-33-2 F7 (44) ] ] | | Y | N | Y | Y | Y | | | |
| (Vs-12) F1 # 2 | | | | | | | | | |
| [[KY160NN (5) × A3-29-305-17-09-18-33-2 F7 (44) | | N/A | N/A | N/A | N/A | N/A | | | |
| (Vs-12) F1 # 3 | | | | | | | | | |
| [[KY160NN (5) × A3-29-305-17-09-25-04-19 F7 | Yes | Y | N | Y | Y | Y | 34.8 | 820 | 28.54 |
| (46)] ] (Vs-14) (1)″F1 # 1 | | | | | | | | | |
| [[KY160NN (5) × A3-29-305-17-09-25-04-19 F7 | | N | N | N | Y | Y | | | |
| (46)] ] (Vs-14) F1 # 2 | | | | | | | | | |
| [[KY160NN (5) × A3-29-305-17-09-25-04-19 F7 | | Y | N | N | Y | Y | | | |
| (46)] ] (Vs-14) F1 # 3 | | | | | | | | | |
| [[KY160NN (5) × A3-29-305-17-09-25-04-19 F7 | | N/A | N/A | N/A | N/A | N/A | | | |
| (46)] ] (Vs-14) F1 # 4 | | | | | | | | | |
| [[KY160NN (5) × A3-29-305-17-09-25-04-19 F7 | | N | N | N | N | N | | | |
| (46)] ] (Vs-14) F1 # 5 | | | | | | | | | |
| [[KY160NN (5) × A3-29-305-17-09-37-28-31 F7 | Yes | Y | Y | Y | Y | Y | 50.5 | 836 | 42.22 |
| (47)] ] (Vs-15) (1)″F1 # 1 | | | | | | | | | |
| [[KY160NN (5) × A3-29-305-17-09-37-28-31 F7 | | Y | N | Y | Y | Y | | | |
| (47)] ] (Vs-15) F1 # 5 | | | | | | | | | |
| [[KY160NN (5) × A3-29-305-17-09-37-28-31 F7 | Yes | Y | Y | Y | Y | Y | 35.5 | 1507 | 53.50 |
| (47)] ] (Vs-15) (3)″F1 # 3 | | | | | | | | | |
| [[KY160NN (5) × A3-29-305-17-09-37-28-31 F7 | | Y | N | Y | Y | Y | | | |
| (47)] ] (Vs-15) F1 # 4 | | | | | | | | | |
| [[KY160NN (5) × A3-29-305-17-09-37-28-31 F7 | | N | N | Y | Y | Y | | | |
| (47)] ] (Vs-15) F1 # 3 | | | | | | | | | |
| [[Virginia K326NN (6) × A3-29-305-17-09-18-33-2 | Yes | Y | Y | Y | Y | Y | 71.2 | 898 | 63.94 |
| F7 (44)] ] (Vs-17) (1)″F1 # 1 | | | | | | | | | |
| [[Virginia K326NN (6) × A3-29-305-17-09-18-33-2 F7 (44)] ] | | Y | N | Y | Y | Y | | | |
| (Vs-17) F1 # 2 | | | | | | | | | |
| [[Virginia K326NN (6) × A3-29-305-17-09-18-33-2 F7 (44)] ] | | N | N | N | Y | Y | | | |
| (Vs-17) F1 # 4 | | | | | | | | | |
| [[Virginia K326NN (6) × A3-29-305-17-09-18-33-2 F7 (44)] ] | | Y | N | Y | Y | Y | | | |
| (Vs-17) F1 # 6 | | | | | | | | | |
| [[Virginia K326NN (6) × A3-29-305-17-09-18-33-2 | Yes | Y | Y | Y | Y | Y | 81.2 | 985 | 79.98 |
| F7 (44)] ] (Vs-17) (5)″F1 # 5 | | | | | | | | | |
| [[Burley TN86NN (8) × A3-29-305-17-09-18-33-2 | | Y | N | Y | Y | Y | | | |
| F7 (44)] ] (Vs-27) F1 # | | | | | | | | | |
| [[Burley TN86NN (8) × A3-29-305-17-09-18-33-2 | | N | N | N | N | N | | | |
| F7 (44)] ] (Vs-27) F1 # | | | | | | | | | |
| [[Burley TN86NN (8) × A3-29-305-17-09-18-33-2 | Yes | Y | Y | Y | Y | Y | 33.9 | 980 | 33.22 |
| F7 (44)] ] (Vs-27) (3)″F1 # 3 | | | | | | | | | |
| [[Burley TN86NN (8) × A3-29-305-17-09-18-33-2 | Yes | Y | Y | Y | Y | Y | 46.7 | 713 | 33.30 |
| F7 (44)] ] (Vs-27) (4)″F1 # 4 | | | | | | | | | |
| [[Burley TN86NN (8) × A3-29-305-17-09-18-33-2 | | Y | N | N | Y | Y | | | |
| F7 (44)] ] (Vs-27) F1 # | | | | | | | | | |
| [[Burley TN90NN (9) × A3-29-305-17-09-18F5 | Yes | Y | Y | Y | Y | Y | 54.5 | 925 | 50.41 |
| (43)] ] (Vs-31) (1)″F1 # 1 | | | | | | | | | |
| [[Burley TN90NN (9) × A3-29-305-17-09-18F5 | | Y | N | Y | Y | Y | | | |
| (43)] ] (Vs-31) F1 # | | | | | | | | | |
| [[Burley TN90NN (9) × A3-29-305-17-09-18F5 | | Y | N | Y | Y | Y | | | |
| (43)] ] (Vs-31) F1 # | | | | | | | | | |
| [[Burley TN90NN (9) × A3-29-305-17-09-18F5 | Yes | Y | Y | Y | Y | Y | 77.1 | 1058 | 81.57 |
| (43)] ] (Vs-31) (8)″F1 # 8 | | | | | | | | | |
| [[Burley TN90NN (9) × A3-29-305-17-09-18F5 | | Y | N | Y | Y | Y | | | |
| (43)] ] (Vs-31) F1 # | | | | | | | | | |
| [[Burley TN90NN (9) × A3-29-305-17-09-18-33-2 | Yes | Y | Y | Y | Y | Y | 59.9 | 1033 | 61.88 |
| F7 (44)] ] (Vs-32) (6)″F1 # 6 | | | | | | | | | |
| [[Burley TN90NN (9) × A3-29-305-17-09-18-33-2 | Yes | Y | Y | Y | Y | Y | 43.3 | 978 | 42.35 |
| F7 (44)] ] (Vs-32) (2)″F1 # 2 | | | | | | | | | |
| [[Burley TN90NN (9) × A3-29-305-17-09-18-33-2 | | Y | Y | Y | Y | Y | | | |
| F7 (44)] ] (Vs-32) F1 # | | | | | | | | | |
| [[Burley TN90NN (9) × A3-29-305-17-09-18-33-2 | | Y | Y | Y | Y | Y | | | |
| F7 (44)] ] (Vs-32) F1 # | | | | | | | | | |
| [[Burley TN90NN (9) × A3-29-305-17-09-18-33-2 | | Y | Y | Y | Y | Y | | | |
| F7 (44)] ] (Vs-32) F1 # | | | | | | | | | |
| [[Burley TN90NN (9) × A3-29-305-17-09-18-33- | Yes | Y | Y | Y | Y | Y | 46 | 895 | 41.17 |
| 10F7 (45)] ] (Vs-33) (1)″F1 # 1 | | | | | | | | | |
| [[Burley TN90NN (9) × A3-29-305-17-09-18-33- | Yes | Y | Y | Y | Y | Y | 59 | 768 | 45.31 |
| 10F7 (45)] ] (Vs-33) (2)″F1 # 2 | | | | | | | | | |
| [[Burley TN90NN (9) × A3-29-305-17-09-18-33-10F7 (45)] ] | | N | N | N | Y | Y | | | |
| (Vs-33) F1 # | | | | | | | | | |
| [[Burley TN90NN (9) × A3-29-305-17-09-18-33-10F7 (45)] ] | | N | N | N | N | N | | | |
| (Vs-33) F1 # | | | | | | | | | |
| [[Burley TN90NN (9) × A3-29-305-17-09-18-33-10F7 (45)] ] | | Y | Y | Y | Y | Y | | | |
| (Vs-33) F1 # | | | | | | | | | |
| [[Burley TN90NN (9) × A3-29-305-17-09-37-28-31 | Yes | Y | Y | Y | Y | Y | 71.9 | 671 | 48.24 |
| F7 (47)] ] (Vs-35) (1)″F1 # 1 | | | | | | | | | |
| [[Burley TN90NN (9) × A3-29-305-17-09-37-28-31 F7 (47)] ] | | N | N | N | Y | Y | | | |
| (Vs-35) F1 # | | | | | | | | | |
| [[Burley TN90NN (9) × A3-29-305-17-09-37-28-31 | Yes | Y | N | Y | Y | Y | 65.7 | 544 | 35.74 |
| F7 (47)] ] (Vs-35) (6)″F1 # 6 | | | | | | | | | |
| [[Burley TN90NN (9) × A3-29-305-17-09-37-28-31 F7 (47)] ] | | Y | N | Y | Y | Y | | | |
| (Vs-35) F1 # | | | | | | | | | |
| [[Burley TN90NN (9) × A3-29-305-17-09-37-28-31 F7 (47)] ] | | N | N | N | Y | Y | | | |
| (Vs-35) F1 # | | | | | | | | | |
| [[PG04NN (10) × A3-29-305-17-09-18-33-2 F7 | Yes | Y | N | Y | Y | Y | 55.3 | 745 | 41.20 |
| (44)] ] (Vs-37) (1)″F1 # 1 | | | | | | | | | |
| [[PG04NN (10) × A3-29-305-17-09-18-33-2 F7 | Yes | Y | N | Y | Y | Y | 43.1 | 958 | 41.29 |
| (44)] ] (Vs-37) (2)″F1 # 2 | | | | | | | | | |
| [[PG04NN (10) × A3-29-305-17-09-18-33-2 F7 | | N | N | N | Y | Y | | | |
| (44)] ] (Vs-37) F1 # | | | | | | | | | |
| [[PG04NN (10) × A3-29-305-17-09-18-33-2 F7 | | N | N | N | N | N | | | |
| (44)] ] (Vs-37) F1 # | | | | | | | | | |
| [[PG04NN (10) × A3-29-305-17-09-18-33-2 F7 | | Y | N | N | Y | Y | | | |
| (44)] ] (Vs-37) F1 # | | | | | | | | | |
| [[PG04NN (10) × A3-29-305-17-09-18-33-10F7 | Yes | Y | Y | Y | Y | Y | 42.2 | 1028 | 43.38 |
| (45)] ] (Vs-38) (1)″F1 # 1 | | | | | | | | | |
| [[PG04NN (10) × A3-29-305-17-09-18-33-10F7 | | Y | N | Y | Y | Y | | | |
| (45)] ] (Vs-38) F1 # | | | | | | | | | |
| [[PG04NN (10) × A3-29-305-17-09-18-33-10F7 | Yes | Y | Y | Y | Y | Y | 48.8 | 1103 | 53.83 |
| (45)] ] (Vs-38) (3)″F1 # 3 | | | | | | | | | |
| [[PG04NN (10) × A3-29-305-17-09-18-33-10F7 | | Y | N | Y | Y | Y | | | |
| (45)] ] (Vs-38) F1 # | | | | | | | | | |
| [[PG04NN (10) × A3-29-305-17-09-18-33-10F7 | | Y | N | Y | Y | Y | | | |
| (45)] ] (Vs-38) F1 # | | | | | | | | | |
| [[PG04NN (10) × A3-29-305-17-09-25-04-19 F7 | Yes | Y | Y | Y | Y | Y | 61.1 | 1259 | 76.92 |
| (46)] ] (Vs-39) (1)″F1 # 1 | | | | | | | | | |
| [[PG04NN (10) × A3-29-305-17-09-25-04-19 F7 | | N | N | N | N | N | | | |
| (46)] ] (Vs-39) F1 # | | | | | | | | | |
| [[PG04NN (10) × A3-29-305-17-09-25-04-19 F7 | Yes | Y | N | Y | Y | Y | 66 | 1048 | 69.17 |
| (46)] ] (Vs-39) (3)″F1 # 3 | | | | | | | | | |
| [[PG04NN (10) × A3-29-305-17-09-25-04-19 F7 | | N/A | N/A | N/A | N/A | N/A | | | |
| (46)] ] (Vs-39) F1 # | | | | | | | | | |
| [[PG04NN (10) × A3-29-305-17-09-25-04-19 F7 | | Y | N | Y | Y | Y | | | |
| (46)] ] (Vs-39) F1 # | | | | | | | | | |
| [[KY171LCNN (11) × A3-29-305-17-09-18-33-2 F7 | Yes | Y | Y | Y | Y | Y | 73.6 | 1283 | 94.43 |
| (44)] ] (Vs-42) (1)″F1 # 1 | | | | | | | | | |
| [[KY171LCNN (11) × A3-29-305-17-09-18-33-2 F7 | Yes | Y | Y | Y | Y | Y | 67.2 | 532 | 35.75 |
| (44)] ] (Vs-42) (2)″F1 # 2 | | | | | | | | | |
| [[KY171LCNN (11) × A3-29-305-17-09-18-33-2 F7 | | Y | Y | Y | Y | Y | | | |
| (44)] ] (Vs-42) F1 # | | | | | | | | | |
| [[KY171LCNN (11) × A3-29-305-17-09-18-33-2 F7 | | Y | Y | Y | Y | Y | | | |
| (44)] ] (Vs-42) F1 # | | | | | | | | | |
| [[KY171LCNN (11) × A3-29-305-17-09-18-33-2 F7 | | Y | N | Y | Y | Y | | | |
| (44)] ] (Vs-42) F1 # | | | | | | | | | |
| [[KY171LCNN (11) × A3-29-305-17-09-18-33-10F7 | Yes | Y | Y | Y | Y | Y | 47.4 | 1169 | 55.41 |
| (45)] ] (Vs-43) (1)″F1 # 1 | | | | | | | | | |
| [[KY171LCNN (11) × A3-29-305-17-09-18-33-10F7 | Yes | Y | Y | Y | Y | Y | 42.3 | 907 | 38.37 |
| (45)] ] (Vs-43) (2)″F1 # 2 | | | | | | | | | |
| [[KY171LCNN (11) × A3-29-305-17-09-18-33-10F7 | | Y | Y | Y | Y | Y | | | |
| (45)] ] (Vs-43) F1 # | | | | | | | | | |
| [[KY171LCNN (11) × A3-29-305-17-09-18-33-10F7 | | Y | N | Y | Y | Y | | | |
| (45)] ] (Vs-43) F1 # | | | | | | | | | |
| [[KY171LCNN (11) × A3-29-305-17-09-18-33-10F7 | | Y | N | Y | Y | Y | | | |
| (45)] ] (Vs-43) F1 # | | | | | | | | | |
| [[KY171LCNN (11) × A3-29-305-17-09-25-04-19 F7 | | N | N | N | Y | Y | | | |
| (46)] ] (Vs-44) F1 # | | | | | | | | | |
| [[KY171LCNN (11) × A3-29-305-17-09-25-04-19 F7 | | Y | N | Y | Y | Y | | | |
| (46)] ] (Vs-44) F1 # | | | | | | | | | |
| [[KY171LCNN (11) × A3-29-305-17-09-25-04-19 F7 | | Y | N | Y | Y | Y | | | |
| (46)] ] (Vs-44) F1 # | | | | | | | | | |
| [[KY171LCNN (11) × A3-29-305-17-09-25-04-19 F7 | Yes | Y | Y | Y | Y | Y | 76.4 | 945 | 72.20 |
| (46)] ] (Vs-44) (4)″F1 # 4 | | | | | | | | | |
| [[KY171LCNN (11) × A3-29-305-17-09-25-04-19 F7 | | Y | N | Y | Y | Y | | | |
| (46)] ] (Vs-44) F1 # | | | | | | | | | |
| [[KY171LCNN (11) × A3-29-305-17-09-37-28-31 F7 | | N/A | N/A | N/A | N/A | N/A | | | |
| (47)] ] (Vs-45) F1 # | | | | | | | | | |
| [[KY171LCNN (11) × A3-29-305-17-09-37-28-31 F7 | | Y | N | Y | Y | Y | | | |
| (47)] ] (Vs-45) F1 # | | | | | | | | | |
| [[KY171LCNN (11) × A3-29-305-17-09-37-28-31 F7 | | N | N | N | N | N | | | |
| (47)] ] (Vs-45) F1 # | | | | | | | | | |
| [[KY171LCNN (11) × A3-29-305-17-09-37-28-31 F7 | | Y | N | Y | Y | Y | | | |
| (47)] ] (Vs-45) F1 # | | | | | | | | | |
| [[KY171LCNN (11) × A3-29-305-17-09-37-28-31 F7 | Yes | Y | Y | Y | Y | Y | 58 | 696 | 40.37 |
| (47)] ] (Vs-45) (6)″F1 # 6 | | | | | | | | | |
| [[N tabacum cv. MarylandNN (12) × A3-29-305- | Yes | Y | Y | Y | Y | Y | 69.7 | 940 | 65.52 |
| 17-09-18F5 (43)] ] (Vs-46) (1)″F1 # 1 | | | | | | | | | |
| [[N tabacum cv. MarylandNN (12) × A3-29-305- | Yes | Y | Y | Y | Y | Y | 59.9 | 456 | 27.31 |
| 17-09-18F5 (43)] ] (Vs-46) (2)″F1 # 2 | | | | | | | | | |
| [[N tabacum cv. MarylandNN (12) × A3-29-305-17-09-18F5 | | Y | Y | Y | Y | Y | | | |
| (43)] ] (Vs-46) F1 # | | | | | | | | | |
| [[N tabacum cv. MarylandNN (12) × A3-29-305-17-09-18F5 | | Y | N | Y | Y | Y | | | |
| (43)] ] (Vs-46) F1 # | | | | | | | | | |
| [[N tabacum cv. MarylandNN (12) × A3-29-305-17-09-18F5 | | N/A | N/A | N/A | N/A | N/A | | | |
| (43)] ] (Vs-46) F1 # | | | | | | | | | |
| [[N tabacum cv. MarylandNN (12) × A3-29-305- | Yes | Y | Y | Y | Y | Y | 63.6 | 599 | 38.10 |
| 17-09-18-33-2 F7 (44)] ] (Vs-47) (1)″F1 # 1 | | | | | | | | | |
| [[N tabacum cv. MarylandNN (12) × A3-29-305-17-09-18-33-2 | | N | N | N | Y | Y | | | |
| F7 (44)] ] (Vs-47) F1 # | | | | | | | | | |
| [[N tabacum cv. MarylandNN (12) × A3-29-305-17-09-18-33-2 | | Y | N | Y | Y | Y | | | |
| F7 (44)] ] (Vs-47) F1 # | | | | | | | | | |
| [[N tabacum cv. MarylandNN (12) × A3-29-305- | Yes | Y | Y | Y | Y | Y | 54.4 | 1015 | 55.22 |
| 17-09-18-33-2 F7 (44)] ] (Vs-47) (4)″F1 # 4 | | | | | | | | | |
| [[N tabacum cv. MarylandNN (12) × A3-29-305-17-09-18-33-2 | | Y | Y | Y | Y | Y | | | |
| F7 (44)] ] (Vs-47) F1 # | | | | | | | | | |
| [[N tabacum cv. MarylandNN (12) × A3-29-305- | Yes | Y | Y | Y | Y | Y | 46.5 | 1366 | 63.52 |
| 17-09-18-33-10F7 (45)] ] (Vs-48) (1)″F1 # 1 | | | | | | | | | |
| [[N tabacum cv. MarylandNN (12) × A3-29-305-17-09-18-33- | | N | N | N | Y | Y | | | |
| 10F7 (45)] ] (Vs-48) F1 # | | | | | | | | | |
| [[N tabacum cv. MarylandNN (12) × A3-29-305-17-09-18-33- | | N/A | N/A | N/A | N/A | N/A | | | |
| 10F7 (45)] ] (Vs-48) F1 # | | | | | | | | | |
| [[N tabacum cv. MarylandNN (12) × A3-29-305-17-09-18-33- | | N | N | N | Y | Y | | | |
| 10F7 (45)] ] (Vs-48) F1 # | | | | | | | | | |
| [[N tabacum cv. MarylandNN (12) × A3-29-305-17-09-18-33- | | N | N | N | N | Y | | | |
| 10F7 (45)] ] (Vs-48) F1 # | | | | | | | | | |
| [[N tabacum cv. MarylandNN (12) × A3-29-305-17-09-37-28-31 | | N | N | Y | Y | Y | | | |
| F7 (47)] ] (Vs-50) F1 # | | | | | | | | | |
| [[N tabacum cv. MarylandNN (12) × A3-29-305-17-09-37-28-31 | | Y | N | Y | Y | Y | | | |
| F7 (47)] ] (Vs-50) F1 # | | | | | | | | | |
| [[N tabacum cv. MarylandNN (12) × A3-29-305-17-09-37-28-31 | | Y | N | Y | Y | Y | | | |
| F7 (47)] ] (Vs-50) F1 # | | | | | | | | | |
| [[N tabacum cv. MarylandNN (12) × A3-29-305- | Yes | Y | Y | Y | Y | Y | 39.1 | 1044 | 40.82 |
| 17-09-37-28-31 F7 (47)] ] (Vs-50) (4)″F1 # 4 | | | | | | | | | |
| [[N tabacum cv. MarylandNN (12) × A3-29-305-17-09-37-28-31 | | Y | N | Y | Y | Y | | | |
| F7 (47)] ] (Vs-50) F1 # | | | | | | | | | |
| [[N tabacum cv. MD 609NN (14) × A3-29-305-17-09-18-33-2 F7 | | N | N | N | Y | Y | | | |
| (44)] ] (Vs-57) F1 # | | | | | | | | | |
| [[N tabacum cv. MD 609NN (14) × A3-29-305-17-09-18-33-2 F7 | | N | N | N | N | Y | | | |
| (44)] ] (Vs-57) F1 # | | | | | | | | | |
| [[N tabacum cv. MD 609NN (14) × A3-29-305-17- | Yes | Y | N | Y | Y | Y | 39.4 | 851 | 33.53 |
| 09-18-33-2 F7 (44)] ] (Vs-57) (3)″F1 # 3 | | | | | | | | | |
| [[N tabacum cv. MD 609NN (14) × A3-29-305-17-09-18-33-2 F7 | | N | N | N | N | Y | | | |
| (44)] ] (Vs-57) F1 # | | | | | | | | | |
| [[N tabacum cv. MD 609NN (14) × A3-29-305-17- | Yes | Y | N | Y | Y | Y | 29.8 | 648 | 19.31 |
| 09-18-33-2 F7 (44)] ] (Vs-57) (5)″F1 # 5 | | | | | | | | | |
| [[N tabacum cv. MD 609NN (14) × A3-29-305-17-09-18-33- | | N/A | N/A | N/A | N/A | N/A | | | |
| 10F7 (45)] ] (Vs-58) F1 # | | | | | | | | | |
| [[N tabacum cv. MD 609NN (14) × A3-29-305-17- | Yes | Y | Y | Y | Y | Y | 42 | 363 | 15.25 |
| 09-18-33-10F7 (45)] ] (Vs-58) (2)″F1 # 2 | | | | | | | | | |
| [[N tabacum cv. MD 609NN (14) × A3-29-305-17- | Yes | Y | N | Y | Y | Y | 41.7 | 1045 | 43.58 |
| 09-18-33-10F7 (45)] ] (Vs-58) (3)″F1 # 3 | | | | | | | | | |
| [[N tabacum cv. MD 609NN (14) × A3-29-305-17-09-18-33- | | Y | N | Y | Y | Y | | | |
| 10F7 (45)] ] (Vs-58) F1 # | | | | | | | | | |
| [[N tabacum cv. MD 609NN (14) × A3-29-305-17-09-18-33- | | N | N | Y | Y | Y | | | |
| 10F7 (45)] ] (Vs-58) F1 # | | | | | | | | | |
| [[N tabacum cv. Black mammoth NN (21) × A3-29-305-17-09- | | Y | N | Y | Y | Y | | | |
| 18-33-2 F7 (44)] ] (Vs-77) F1 # | | | | | | | | | |
| [[N tabacum cv. Black mammoth NN (21) × A3- | Yes | Y | Y | Y | Y | Y | 56.7 | 868 | 49.22 |
| 29-305-17-09-18-33-2 F7 (44)] ] (Vs-77) (2)″F1 # 2 | | | | | | | | | |
| [[N tabacum cv. Black mammoth NN (21) × A3-29-305-17-09- | | N | N | Y | Y | Y | | | |
| 18-33-2 F7 (44)] ] (Vs-77) F1 # | | | | | | | | | |
| [[N tabacum cv. Black mammoth NN (21) × A3-29-305-17-09- | | Y | N | Y | Y | Y | | | |
| 18-33-2 F7 (44)] ] (Vs-77) F1 # | | | | | | | | | |
| [[N tabacum cv. Black mammoth NN (21) × A3-29-305-17-09- | | Y | N | Y | Y | Y | | | |
| 18-33-2 F7 (44)] ] (Vs-77) F1 # | | | | | | | | | |
| [[N tabacum cv. Black mammoth NN (21) × A3-29-305-17-09- | | Y | N | Y | Y | Y | | | |
| 18-33-10F7 (45)] ] (Vs-78) F1 # | | | | | | | | | |
| [[N tabacum cv. Black mammoth NN (21) × A3- | Yes | Y | Y | Y | Y | Y | 48.5 | 623 | 30.22 |
| 29-305-17-09-18-33-10F7 (45)] ] (Vs-78) (2)″F1 # 2 | | | | | | | | | |
| [[N tabacum cv. Black mammoth NN (21) × A3- | Yes | Y | Y | Y | Y | Y | 32 | 546 | 17.47 |
| 29-305-17-09-18-33-10F7 (45)] ] (Vs-78) (3)″F1 # 3 | | | | | | | | | |
| [[N tabacum cv. Black mammoth NN (21) × A3-29-305-17-09- | | Y | N | Y | Y | Y | | | |
| 18-33-10F7 (45)] ] (Vs-78) F1 # | | | | | | | | | |
| [[N tabacum cv. Black mammoth NN (21) × A3-29-305-17-09- | | Y | Y | Y | Y | Y | | | |
| 18-33-10F7 (45)] ] (Vs-78) F1 # | | | | | | | | | |
| [[N tabacum cv. Cuban criollo 98NN (22) × A3-29-305-17-09- | | Y | N | Y | Y | Y | | | |
| 18F5 (43)] ] (Vs-81) F1 # | | | | | | | | | |
| [[N tabacum cv. Cuban criollo 98NN (22) × A3-29-305-17-09- | | Y | N | Y | Y | Y | | | |
| 18F5 (43)] ] (Vs-81) F1 # | | | | | | | | | |
| [[N tabacum cv. Cuban criollo 98NN (22) × A3-29-305-17-09- | | N | N | N | Y | Y | | | |
| 18F5 (43)] ] (Vs-81) F1 # | | | | | | | | | |
| [[N tabacum cv. Cuban criollo 98NN (22) × A3-29- | Yes | Y | Y | Y | Y | Y | 62.4 | 768 | 47.92 |
| 305-17-09-18F5 (43)] ] (Vs-81) (4)″F1 # 4 | | | | | | | | | |
| [[N tabacum cv. Cuban criollo 98NN (22) × A3-29- | Yes | Y | Y | Y | Y | Y | 75.7 | 1339 | 101.36 |
| 305-17-09-18F5 (43)] ] (Vs-81) (5)″F1 # 5 | | | | | | | | | |
| [[N tabacum cv. Cuban criollo 98NN (22) × A3-29-305-17-09- | | Y | N | Y | Y | Y | | | |
| 18-33-2 F7 (44)] ] (Vs-82) F1 # | | | | | | | | | |
| [[N tabacum cv. Cuban criollo 98NN (22) × A3-29- | Yes | Y | Y | Y | Y | Y | 77.9 | 1349 | 105.09 |
| 305-17-09-18-33-2 F7 (44)] ] (Vs-82) (2)″F1 # 2 | | | | | | | | | |
| [[N tabacum cv. Cuban criollo 98NN (22) × A3-29-305-17-09- | | Y | N | Y | Y | Y | | | |
| 18-33-2 F7 (44)] ] (Vs-82) F1 # | | | | | | | | | |
| [[N tabacum cv. Cuban criollo 98NN (22) × A3-29-305-17-09- | | Y | N | Y | Y | Y | | | |
| 18-33-2 F7 (44)] ] (Vs-82) F1 # | | | | | | | | | |
| [[N tabacum cv. Cuban criollo 98NN (22) × A3-29-305-17-09- | | N | N | Y | Y | Y | | | |
| 18-33-2 F7 (44)] ] (Vs-82) F1 # | | | | | | | | | |
| [[N tabacum cv. Cuban criollo 98NN (22) × A3-29-305-17-09- | | N/A | N/A | N/A | N/A | N/A | | | |
| 18-33-10F7 (45)] ] (Vs-83) F1 # | | | | | | | | | |
| [[N tabacum cv. Cuban criollo 98NN (22) × A3-29-305-17-09- | | Y | N | Y | Y | Y | | | |
| 18-33-10F7 (45)] ] (Vs-83) F1 # | | | | | | | | | |
| [[N tabacum cv. Cuban criollo 98NN (22) × A3-29-305-17-09- | | Y | N | Y | Y | Y | | | |
| 18-33-10F7 (45)] ] (Vs-83) F1 # | | | | | | | | | |
| [[N tabacum cv. Cuban criollo 98NN (22) × A3-29-305-17-09- | | N | N | N | N | Y | | | |
| 18-33-10F7 (45)] ] (Vs-83) F1 # | | | | | | | | | |
| [[N tabacum cv. Cuban criollo 98NN (22) × A3-29- | Yes | Y | Y | Y | Y | Y | 46.9 | 809 | 37.94 |
| 305-17-09-18-33-10F7 (45)] ] (Vs-83) (5)″F1 # 5 | | | | | | | | | |
| [[N tabacum cv. Cuban criollo 98NN (22) × A3-29-305-17-09- | | Y | N | Y | Y | Y | | | |
| 25-04-19 F7 (46)] ] (Vs-84) F1 # | | | | | | | | | |
| [[N tabacum cv. Cuban criollo 98NN (22) × A3-29- | Yes | Y | Y | Y | Y | Y | 69 | 738 | 50.92 |
| 305-17-09-25-04-19 F7 (46)] ] (Vs-84) (2)″F1 # 2 | | | | | | | | | |
| [[N tabacum cv. Cuban criollo 98NN (22) × A3-29-305-17-09- | | N | N | N | N | Y | | | |
| 25-04-19 F7 (46)] ] (Vs-84) F1 # | | | | | | | | | |
| [[N tabacum cv. Cuban criollo 98NN (22) × A3-29- | Yes | Y | Y | Y | Y | Y | 71.7 | 748 | 53.63 |
| 305-17-09-25-04-19 F7 (46)] ] (Vs-84) (4)″F1 # 4 | | | | | | | | | |
| [[N tabacum cv. Cuban criollo 98NN (22) × A3-29-305-17-09- | | Y | Y | Y | Y | Y | | | |
| 25-04-19 F7 (46)] ] (Vs-84) F1 # | | | | | | | | | |
| [[N tabacum cv. Cuban criollo 98NN (22) × A3-29-305-17-09- | | N | N | N | N | N | | | |
| 37-28-31 F7 (47)] ] (Vs-85) F1 # | | | | | | | | | |
| [[N tabacum cv. Cuban criollo 98NN (22) × A3-29-305-17-09- | | N | N | N | N | N | | | |
| 37-28-31 F7 (47)] ] (Vs-85) F1 # | | | | | | | | | |
| [[N tabacum cv. Cuban criollo 98NN (22) × A3-29-305-17-09- | | N | N | N | N | N | | | |
| 37-28-31 F7 (47)] ] (Vs-85) F1 # | | | | | | | | | |
| [[N tabacum cv. Cuban criollo 98NN (22) × A3-29-305-17-09- | | N/A | N/A | N/A | N/A | N/A | | | |
| 37-28-31 F7 (47)] ] (Vs-85) F1 # | | | | | | | | | |
| [[N tabacum cv. Cuban criollo 98NN (22) × A3-29-305-17-09- | | N | N | N | N | N | | | |
| 37-28-31 F7 (47)] ] (Vs-85) F1 # | | | | | | | | | |
| [[Nicotiana rusticaNN (37) × A3-29-305-17-09-18-33-10F7 | | N | N | N | Y | Y | | | |
| (45)] ] (Vs-88) F1 # | | | | | | | | | |
| [[Nicotiana rusticaNN (37) × A3-29-305-17-09-18-33-10F7 | | N | N | N | N | N | | | |
| (45)] ] (Vs-88) F1 # | | | | | | | | | |
| [[Nicotiana rusticaNN (37) × A3-29-305-17-09-18- | Yes | Y | Y | Y | Y | Y | 70.2 | 799 | 56.09 |
| 33-10F7 (45)] ] (Vs-88) (3)″F1 # 3 | | | | | | | | | |
| [[Nicotiana rusticaNN (37) × A3-29-305-17-09-18- | Yes | Y | Y | Y | Y | Y | 60.6 | 739 | 44.78 |
| 33-10F7 (45)] ] (Vs-88) (4)″F1 # 4 | | | | | | | | | |
| [[Nicotiana rusticaNN (37) × A3-29-305-17-09-18-33-10F7 | | Y | Y | Y | Y | Y | | | |
| (45)] ] (Vs-88) F1 # | | | | | | | | | |
| [[Nicotiana rusticaNN (37) × A3-29-305-17-09-37-28-31 F7 | | Y | N | Y | Y | Y | | | |
| (47)] ] (Vs-90) F1 # | | | | | | | | | |
| [[Nicotiana rusticaNN (37) × A3-29-305-17-09-37- | Yes | Y | Y | Y | Y | Y | 63.1 | 944 | 59.57 |
| 28-31 F7 (47)] ] (Vs-90) (2)″F1 # 2 | | | | | | | | | |
| [[Nicotiana rusticaNN (37) × A3-29-305-17-09-37-28-31 F7 | | Y | N | Y | Y | Y | | | |
| (47)] ] (Vs-90) F1 # | | | | | | | | | |
| [[Nicotiana rusticaNN (37) × A3-29-305-17-09-37- | Yes | Y | Y | Y | Y | Y | 54.8 | 820 | 44.94 |
| 28-31 F7 (47)] ] (Vs-90) (4)″F1 #4 | | | | | | | | | |
| [[Nicotiana rusticaNN (37) × A3-29-305-17-09-37-28-31 F7 | | Y | Y | Y | Y | Y | | | |
| (47)] ] (Vs-90) F1 # | | | | | | | | | |
| [[Nicotiana tabacum improved madole NN (38) x A3- | Yes | Y | Y | Y | Y | Y | 75 | 1096 | 82.20 |
| 29-305-17-09-18-33-2 F7 (44)] ] (Vs-92) (6)″F1 # 6 | | | | | | | | | |
| [[Nicotiana tabacum improved madole NN (38) × A3-29-305- | | Y | N | Y | Y | Y | | | |
| 17-09-18-33-2 F7 (44)] ] (Vs-92) F1 # | | | | | | | | | |
| [[Nicotiana tabacum improved madole NN (38) × A3-29-305- | | N | N | N | N | Y | | | |
| 17-09-18-33-2 F7 (44)] ] (Vs-92) F1 # | | | | | | | | | |
| [[Nicotiana tabacum improved madole NN (38) x A3- | Yes | Y | N | Y | Y | Y | 71 | 1208 | 85.77 |
| 29-305-17-09-18-33-2 F7 (44)] ] (Vs-92) (4)″F1 # 4 | | | | | | | | | |

| | | | | | |
|---|---|---|---|---|---|
| [[Nicotiana tabacum improved madole NN (38) × A3-29-305- | Y | N | Y | Y | Y |
| 17-09-18-33-2 F7 (44)] ] (Vs-92) F1 # | | | | | |

### F1's—Transgenes, Biomass and PC

Most of the selected plants showed the presence of all 5 transgenes via RT-PCR analysis. Several plants that showed negative results for one of the genes were selected as controls in order to evaluate the accuracy and sensitivity of the analysis method during the selection cycles of the next generations.

PC production in the plants where Col2 is missing can be explained by that a homotrimer was produced. PC production in plants where other transgenes are missing can be explained by inaccuracy of RT-PCR analysis.

### Example 4

### Breeding Plan of Hemizygous Plants from Nearly Homozygous Line A3-29 F4 with N. tabacum vr. Virginia K358 Variety

In an effort to increase pro-collagen (PC) yield at the manufacturing tobacco lines, the breeding program has investigated the possibility of introducing PC production genes into new tobacco varieties. That was done in order to replace the current production line, A3-29-305-17-09-18, which is based on Samsun NN.

For this purpose, a large screening of different tobacco varieties was performed from which four cultivars were selected as potential substitutes to Samsun NN as a genetic background for PC production. All four lines were crossed with A3-29-305-17-09 F4 plant in order to introduce the collagen production system into these new cultivars: Varieties number 1 (N. tabacum vr. Sylvestris), 3 (N. tabacum vr. Cuban Habano 2000), 11 (N. tabacum vr. Black mammoth) and 15 (N. tabacum vr. Virginia K358). Based on the results of this study, it was decided to focus on the cross A3-29-305-17-09 F4 x N. tabacum Virginia K358.

The best Performing cross (A3-29-305-17-09 F4 x N. tabacum vr. Virginia K358) was selected for further breeding.

The grand objective of this study is to examine possible hybrid-vigor combinations based on the nearly pure homozygous seed-based A3-29 F6 line (at the Samsun background; PY14/006) combined with new Nicotiana genetic backgrounds to thereby, improve total biomass and PC yields, to generate new, cultivar-mixed, future production lines. An initial line will presumably be 50% made of the seed-based line A3-29 (F4) which is donating a full set of PC producing transgenes in the N. tabacum cv. Samsun NN background. It will be combined with a (50%) new genetic background donating superior agronomic traits, to allow elevated total product of PC*biomass yields when compared to the seed-based line A3-29 (F6) alone. The genetic background selected is best yield of biomass and possibly additional desired agronomic traits.

The specific objective of this study is to develop a new breeding line, based on the selected cross (A3-29-305-17-09 F4 x N. tabacum vr. Virginia K358) in order to replace the current production line.

### Materials and Methods

### Plants Propagation, Cultivation and Screening

F1 plants were generated by crossing [A3-29-305-17-09 F4 (famale) x N.tabacum vr. Virginia K358 (male)].

F2-F4 seeds were generated by covering one inflorescence in each plant with a paper bag in order to secure self-pollinated seeds. The mature capsules containing seeds were harvested, after which seeds separated and stored in isolated boxes

Seeds were seeded at a nursery and grown for ˜45 days before transferred to a greenhouse, planting at a density of 2.5 plants/ meter row.

### Vigor and Structure

Plants in each generation were selected visually, according to general agronomical performances. At F4 generation, leaves biomass was measured.

### Genetic Screening

The presence of the 5 transgenes was confirmed by using RT-PCR. RT PCR was designed for each gene based on the confirmed CDNA sequences (SEQ ID Nos. 20-24) with DNA extracted from fresh leaves.

### Procollagen Level

The level of procollagen in the plants was determined by ELISA using standard extraction and ELISA protocols as described above.

F2 generation

35 plants originating from Fl seeds were transplanted at Yessod experimental greenhouse (see Table 25).

In each plant, one inflorescence was covered by a paper bag, in order to produce self-pollinated seeds. At that stage, plants were screened only via the genetic tests but not for PC content. F2 plants were screened twice for the presence of all 5 transgenes, using RT-PCR. At the first screening, 25 out of 35 plants showed the presence of all 5 genes (Table 28). In order to test the efficiency of genetic screening method, 32 plants were selected for self-crossing and further breeding.

F3 generation

About 35 plants, of each 32 F2 families (a total of 1050 plants) were transplanted Yessod experimental greenhouse (Table 26), at a density of 5 plants/meter row. In each plant, one inflorescence was covered by a paper bag, in order to produce self-pollinated seeds.

Plants and plants' families were documented for vigor and structure during all the growing season. Plants of the best agronomic-traits-characterized families were screened by RT-PCR. Based on a series of positive controls, RT-PCR result that was higher than 0.1 was counted as positive (the transgene is present). Best resulting RT-PCR plants were sampled for further analysis of its PC content, using ELISA. For ELISA analysis, 3-4 leaves at a height of 1 meter were picked in each plant, 77 days post transplanting, processed and analysed by ELISA protocol (Table 29).

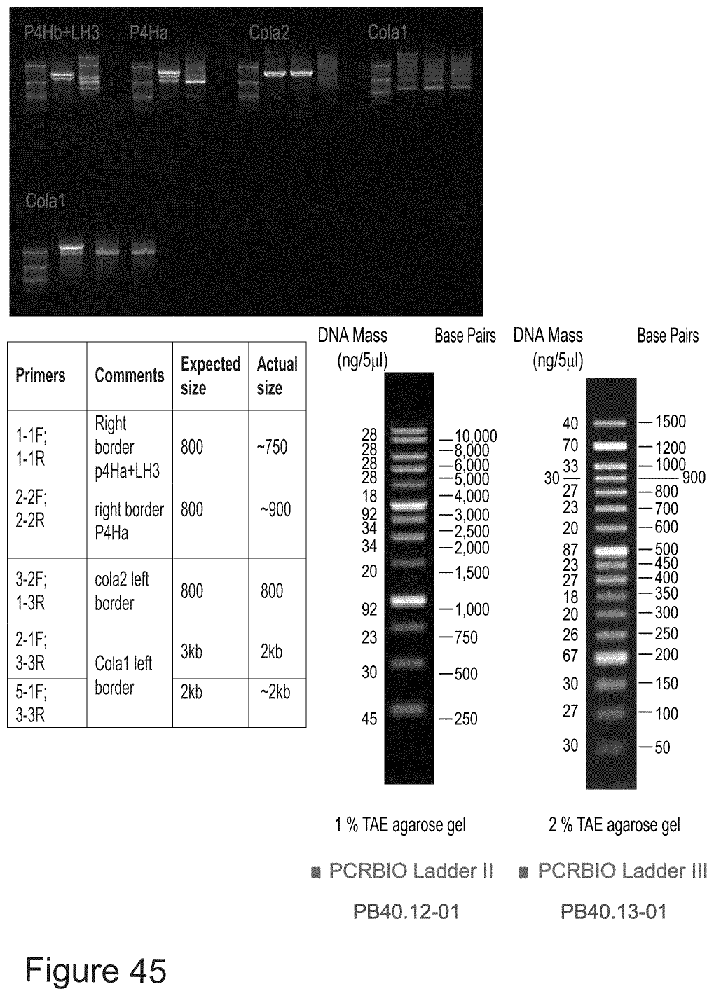

30 plans were selected for further breeding, based on its PC concentration and RT-PCR value of all transgenes (FIG. 45). The selected plants were harvested after self-pollinating seeds were mature, for next generation and further breeding.

F4 generation

About 40 plants, of each 30 F3 selected plant (a total of 1250 plants) were transplanted at Yessod experimental greenhouse (see Table 27), at a density of 5 plants/meter row. In each plant, one inflorescence was covered by a paper bag, in order to produce self-pollinated seeds.

Plants and plants' families were documented for vigor and structure during all the growing season. Plants of the best agronomic-traits-characterized families were screened by RT-PCR. RT-PCR results were compared to a steady N. tabacum gene (scfld8). A value equal or higher than scfld8 gene counted as positive (the transgene is present). Best resulting RT-PCR plants were sampled for further analysis of its PC content, using ELISA. For ELISA analysis, 3-4 leaves at a height of 1 meter were picked in each plant, 77 days post transplanting, Processed and analysed by ELISA protocol (Table 30).

Additionally, in each plant that was sent for ELISA analysis, the leaves were harvested and weighed (Table 30).

30 plans were selected for further breeding, based on leaves weight, PC concentration and RT-PCR results (FIG. 44). Selected plants were harvested after self- pollinating seeds were matured for next generation and further breeding.

TABLE 25
F2 generation plants from F1 self-pollinated seeds.

| Code | F2 name |
|---|---|
| 1 | [K358 × A3-29-305-17-09]-1 F2 |
| 2 | [K358 × A3-29-305-17-09]-2 F2 |
| 3 | [K358 × A3-29-305-17-09]-3 F2 |
| 4 | [K358 × A3-29-305-17-09]-4 F2 |
| 5 | [K358 × A3-29-305-17-09]-5 F2 |
| 6 | [K358 × A3-29-305-17-09]-6 F2 |
| 7 | [K358 × A3-29-305-17-09]-7 F2 |
| 8 | [K358 × A3-29-305-17-09]-8 F2 |
| 9 | [K358 × A3-29-305-17-09]-9 F2 |
| 10 | [K358 × A3-29-305-17-09]-10 F2 |
| 11 | [K358 × A3-29-305-17-09]-11 F2 |
| 12 | [K358 × A3-29-305-17-09]-12 F2 |
| 13 | [K358 × A3-29-305-17-09]-13 F2 |
| 14 | [K358 × A3-29-305-17-09]-14 F2 |
| 15 | [K358 × A3-29-305-17-09]-15 F2 |
| 16 | [K358 × A3-29-305-17-09]-16 F2 |
| 17 | [K358 × A3-29-305-17-09]-17 F2 |
| 18 | [K358 × A3-29-305-17-09]-18 F2 |
| 19 | [K358 × A3-29-305-17-09]-19 F2 |
| 20 | [K358 × A3-29-305-17-09]-20 F2 |
| 21 | [K358 × A3-29-305-17-09]-21 F2 |
| 22 | [K358 × A3-29-305-17-09]-22 F2 |
| 23 | [K358 × A3-29-305-17-09]-23 F2 |
| 24 | [K358 × A3-29-305-17-09]-24 F2 |
| 25 | [K358 × A3-29-305-17-09]-25 F2 |
| 26 | [K358 × A3-29-305-17-09]-26 F2 |
| 27 | [K358 × A3-29-305-17-09]-27 F2 |
| 28 | [K358 × A3-29-305-17-09]-28 F2 |
| 29 | [K358 × A3-29-305-17-09]-29 F2 |
| 30 | [K358 × A3-29-305-17-09]-30 F2 |
| 31 | [K358 × A3-29-305-17-09]-31 F2 |
| 32 | [K358 × A3-29-305-17-09]-32 F2 |
| 33 | [K358 × A3-29-305-17-09]-33 F2 |
| 34 | [K358 × A3-29-305-17-09]-34 F2 |
| 35 | [K358 × A3-29-305-17-09]-35 F2 |

TABLE 26
F3 generation plants from F2 seed bulks. Number of plants
in each family and location in green house (flowchart num)

| | F2 Plant | | No. of |
|---|---|---|---|
| Code | number | F3 name | plants |
| 1 | 1 | [K358 × | 33 |
| | | A3-29-305- | |
| | | 17-09]- | |
| | | 1-bulk F3 | |
| 2 | 2 | [K358 × | 34 |
| | | A3-29-305- | |
| | | 17-09]- | |
| | | 2-bulk F3 | |
| 4 | 4 | [K358 × | 34 |
| | | A3-29-305- | |
| | | 17-09]- | |
| | | 4-bulk F3 | |
| 5 | 5 | [K358 × | 31 |
| | | A3-29-305- | |
| | | 17-09- | |
| | | 5-bulk F3 | |
| 6 | 6 | [K358 × | 32 |
| | | A3-29-305- | |
| | | 17-09]- | |
| | | 6-bulk F3 | |
| 7 | 7 | [K358 × | 32 |
| | | A3-29-305- | |
| | | 17-09]- | |
| | | 7-bulk F3 | |
| 11 | 11 | [K358 × | 31 |
| | | A3-29-305- | |
| | | 17-09]- | |
| | | 11-bulk F3 | |
| 12 | 12 | [K358 × | 32 |
| | | A3-29-305- | |
| | | 17-09]- | |
| | | 12-bulk F3 | |
| 13 | 13 | [K358 × | 32 |
| | | A3-29-305- | |
| | | 17-09]- | |
| | | 13-bulk F3 | |
| 14 | 14 | [K358 × | 32 |
| | | A3-29-305- | |
| | | 17-09]- | |
| | | 14-bulk F3 | |
| 15 | 15 | [K358 × | 32 |
| | | A3-29-305- | |
| | | 17-09]- | |
| | | 15-bulk F3 | |
| 16 | 16 | [K358 × | 32 |
| | | A3-29-305- | |
| | | 17-09]- | |
| | | 16-bulk F3 | |
| 17 | 17 | [K358 × | 34 |
| | | A3-29-305- | |
| | | 17-09]- | |
| | | 17-bulk F3 | |
| 18 | 18 | [K358 × | 35 |
| | | A3-29-305- | |
| | | 17-09]- | |
| | | 18-bulk F3 | |
| 19 | 19 | [K358 × | 32 |
| | | A3-29-305- | |
| | | 17-09]- | |
| | | 19-bulk F3 | |
| 20 | 20 | [K358 × | 33 |
| | | A3-29-305- | |
| | | 17-09]- | |
| | | 20-bulk F3 | |
| 21 | 21 | [K358 × | 33 |
| | | A3-29-305- | |
| | | 17-09]- | |
| | | 21-bulk F3 | |
| 22 | 22 | [K358 × | 34 |
| | | A3-29-305- | |
| | | 17-09]- | |
| | | 22-bulk F3 | |
| 23 | 23 | [K358 × | 30 |
| | | A3-29-305- | |
| | | 17-09]- | |
| | | 23-bulk F3 | |
| 24 | 24 | [K358 × | 31 |
| | | A3-29-305- | |
| | | 17-09]- | |
| | | 24-bulk F3 | |
| 25 | 25 | [K358 × | 32 |
| | | A3-29-305- | |
| | | 17-09]- | |
| | | 25-bulk F3 | |
| 27 | 27 | [K358 × | 31 |
| | | A3-29-305- | |
| | | 17-09]- | |
| | | 27-bulk F3 | |
| 28 | 28 | [K358 × | 34 |
| | | A3-29-305- | |
| | | 17-09]- | |
| | | 28-bulk F3 | |
| 29 | 29 | [K358 × | 30 |
| | | A3-29-305- | |
| | | 17-09]- | |
| | | 29-bulk F3 | |
| 30 | 30 | [K358 × | 36 |
| | | A3-29-305- | |
| | | 17-09]- | |
| | | 30-bulk F3 | |
| 31 | 31 | [K358 × | 32 |
| | | A3-29-305- | |
| | | 17-09]- | |
| | | 31-bulk F3 | |
| 32 | 32 | [K358 × | 31 |
| | | A3-29-305- | |
| | | 17-09]- | |
| | | 32-bulk F3 | |
| 33 | 33 | [K358 × | 32 |
| | | A3-29-305- | |
| | | 17-09]- | |
| | | 33-bulk F3 | |
| 34 | 34 | [K358 × | 35 |
| | | A3-29-305- | |
| | | 17-09]- | |
| | | 34-bulk F3 | |
| 35 | 35 | [K358 × | 34 |
| | | A3-29-305- | |
| | | 17-09]- | |
| | | 35-bulk F3 | |
| 36 | ? | [K358 × | 35 |
| | | A3-29-305- | |
| | | 17-09]- | |
| | | 36-bulk F3 | |
| 37 | ? | [K358 × | 37 |
| | | A3-29-305- | |
| | | 17-09]- | |
| | | 37-bulk F3 | |

TABLE 27
F4 generation plants from F3 seed bulks. Number of plants in each
family (repetition) and location in green house (flowchart num)

| | F2-plant | F3-Plant | Sample | No. of |
|---|---|---|---|---|
| Code | No. | No. | name | plants |
| 1 | 14 | 10 | [K358 × | 41 |
| | | | A3-29- | |
| | | | 305-17- | |
| | | | 09]-14- | |
| | | | 10 bulk | |
| | | | F4 | |
| 2 | 17 | 3 | [K358 × | 43 |
| | | | A3-29- | |
| | | | 305-17- | |
| | | | 09]-17- | |
| | | | 3 bulk | |
| | | | F4 | |
| 3 | 17 | 7 | [K358 × | 43 |
| | | | A3-29- | |
| | | | 305-17- | |
| | | | 09]-17- | |
| | | | 7 bulk | |
| | | | F4 | |
| 4 | 17 | 15 | [K358 × | 42 |
| | | | A3-29- | |
| | | | 305-17- | |
| | | | 09]-17- | |
| | | | 15 bulk | |
| | | | F4 | |
| 5 | 17 | 19 | [K358 × | 42 |
| | | | A3-29- | |
| | | | 305-17- | |
| | | | 09]-17- | |
| | | | 19 bulk | |
| | | | F4 | |
| 6 | 20 | 1 | [K358 × | 43 |
| | | | A3-29- | |
| | | | 305-17- | |
| | | | 09]-20- | |
| | | | 1 bulk | |
| | | | F4 | |
| 7 | 20 | 2 | [K358 × | 42 |
| | | | A3-29- | |
| | | | 305-17- | |
| | | | 09]-20- | |
| | | | 2 bulk | |
| | | | F4 | |
| 8 | 20 | 4 | [K358 × | 44 |
| | | | A3-29- | |
| | | | 305-17- | |
| | | | 09]-20- | |
| | | | 4 bulk | |
| | | | F4 | |
| 9 | 20 | 6 | [K358 × | 40 |
| | | | A3-29- | |
| | | | 305-17- | |
| | | | 09]-20- | |
| | | | 6 bulk | |
| | | | F4 | |
| 10 | 20 | 9 | [K358 × | 44 |
| | | | A3-29- | |
| | | | 305-17- | |
| | | | 09]-20- | |
| | | | 9 bulk | |
| | | | F4 | |
| 11 | 20 | 10 | [K358 × | 42 |
| | | | A3-29- | |
| | | | 305-17- | |
| | | | 09]-20- | |
| | | | 10 bulk | |
| | | | F4 | |
| 12 | 20 | 11 | [K358 × | 42 |
| | | | A3-29- | |
| | | | 305-17- | |
| | | | 09]-20- | |
| | | | 11 bulk | |
| | | | F4 | |
| 13 | 21 | 7 | [K358 × | 43 |
| | | | A3-29- | |
| | | | 305-17- | |
| | | | 09]-21- | |
| | | | 7 bulk | |
| | | | F4 | |
| 14 | 21 | 8 | [K358 × | 42 |
| | | | A3-29- | |
| | | | 305-17- | |
| | | | 09]-21- | |
| | | | 8 bulk | |
| | | | F4 | |
| 15 | 21 | 13 | [K358 × | 43 |
| | | | A3-29- | |
| | | | 305-17- | |
| | | | 09]-21- | |
| | | | 13 bulk | |
| | | | F4 | |
| 16 | 21 | 19 | [K358 × | 44 |
| | | | A3-29- | |
| | | | 305-17- | |
| | | | 09]-21- | |
| | | | 19 bulk | |
| | | | F4 | |
| 17 | 22 | 14 | [K358 × | 42 |
| | | | A3-29- | |
| | | | 305-17- | |
| | | | 09]-22- | |
| | | | 14 bulk | |
| | | | F4 | |
| 18 | 22 | 17 | [K358 × | 41 |
| | | | A3-29- | |
| | | | 305-17- | |
| | | | 09]-22- | |
| | | | 17 bulk | |
| | | | F4 | |
| 19 | 24 | 6 | [K358 × | 42 |
| | | | A3-29- | |
| | | | 305-17- | |
| | | | 09]-24- | |
| | | | 6 bulk | |
| | | | F4 | |
| 20 | 25 | 10 | [K358 × | 40 |
| | | | A3-29- | |
| | | | 305-17- | |
| | | | 09]-25- | |
| | | | 10 bulk | |
| | | | F4 | |
| 21 | 30 | 6 | [K358 × | 44 |
| | | | A3-29- | |
| | | | 305-17- | |
| | | | 09]-30- | |
| | | | 6 bulk | |
| | | | F4 | |
| 22 | 30 | 15 | [K358 × | 40 |
| | | | A3-29- | |
| | | | 305-17- | |
| | | | 09]-30- | |
| | | | 15 bulk | |
| | | | F4 | |
| 23 | 30 | 16 | [K358 × | 12 |
| | | | A3-29- | |
| | | | 305-17- | |
| | | | 09]-30- | |
| | | | 16 bulk | |
| | | | F4 | |
| 24 | 35 | 9 | [K358 × | 45 |
| | | | A3-29- | |
| | | | 305-17- | |
| | | | 09]-35- | |
| | | | 9 bulk | |
| | | | F4 | |
| 25 | 35 | 10 | [K358 × | 43 |
| | | | A3-29- | |
| | | | 305-17- | |
| | | | 09]-35- | |
| | | | 10 bulk | |
| | | | F4 | |
| 26 | 35 | 16 | [K358 × | 41 |
| | | | A3-29- | |
| | | | 305-17- | |
| | | | 09]-35- | |
| | | | 16 bulk | |
| | | | F4 | |
| 27 | 35 | 18 | [K358 × | 43 |
| | | | A3-29- | |
| | | | 305-17- | |
| | | | 09]-35- | |
| | | | 18 bulk | |
| | | | F4 | |
| 28 | 35 | 19 | [K358 × | 42 |
| | | | A3-29- | |
| | | | 305-17- | |
| | | | 09]-35- | |
| | | | 19 bulk | |
| | | | F4 | |

### Results

F2 Generation

TABLE 28
Summary of PCR results for the presence
of all 5 transgenes in F2 generation plants

| Plant | | | | | |
|---|---|---|---|---|---|
| # | col2 | col-1 | P4Hα | P4Hβ | LH3 |
| 1 | Yes | Yes | Yes | Yes | Yes |
| 2 | Yes | Yes | NO | Yes | Yes |
| 3 | Yes | Yes | Yes | Yes | Yes |
| 4 | No | no | NO | no | Yes |
| 5 | Yes | Yes | Yes | Yes | No |
| 6 | Yes | Yes | Yes | Yes | No |
| 7 | Yes | Yes | Yes | Yes | Yes |
| 8 | Yes | Yes | Yes | Yes | Yes |
| 9 | Yes | Yes | Yes | Yes | Yes |
| 10 | Yes | Yes | Yes | Yes | Yes |
| 11 | Yes | Yes | Yes | Yes | Yes |
| 12 | Yes | Yes | Yes | Yes | Yes |
| 13 | Yes | Yes | Yes | Yes | No |
| 14 | Yes | Yes | Yes | Yes | Yes |
| 15 | Yes | Yes | Yes | Yes | Yes |
| 16 | Yes | Yes | Yes | Yes | Yes |
| 17 | Yes | Yes | No | yes | Yes |
| 18 | Yes | Yes | Yes | yes | Yes |
| 19 | Yes | Yes | No | yes | Yes |
| 20 | Yes | Yes | yes | yes | Yes |
| 21 | Yes | Yes | No | yes | Yes |
| 22 | No | Yes | No | No | Yes |
| 23 | yes | Yes | yes | yes | Yes |
| 24 | Yes | Yes | Yes | Yes | Yes |
| 25 | Yes | Yes | Yes | Yes | Yes |
| 26 | Yes | Yes | Yes | Yes | Yes |
| 27 | Yes | Yes | Yes | Yes | No |
| 28 | Yes | Yes | No | ? | Yes |
| 29 | Yes | Yes | yes | ? | Yes |
| 30 | Yes | Yes | yes | ? | Yes |
| 31 | Yes | No | no | ? | Yes |
| 32 | Yes | yes | No | ? | Yes |
| 33 | Yes | yes | yes | ? | No |
| 34 | ? | ? | ? | ? | ? |
| 35 | Yes | Yes | Yes | | Yes |

F3 Generation

TABLE 29
Summary of PC concentration and RT-PCR results in F3 individual plants
that were analyzed in both methods, in comparison to production line “A3-29-305-17-
09-18 F6 Bulk”. 30 best PC yielding plants were selected for further breeding (bold).
Any RT-PCR value which is higher than 0.1 counted to be positive.

| | | Mg | | | | | |
|---|---|---|---|---|---|---|---|
| | | PC/ | | | | | |
| F3 | | Kg | | | | | |
| Family | Plant name | leaves | Col-1 | Col2 | P4Hα | P4Hβ | LH3 |
| 14 | [K358 × A3-29-305-17- | 35 | 0.418 | 0.25 | 0.322 | 0.327 | 0.29 |
| | 09]-14 F3 # 10 | | | | | | |
| 14 | [K358 × A3-29-305-17-09]- | 28.2 | 2.084 | 0.182 | 0.553 | 0.416 | 0.239 |
| | 14 F3 # 7 | | | | | | |
| 14 | [K358 × A3-29-305-17-09]- | 23.8 | 0.335 | 0.343 | 0.12 | 0.767 | 0.434 |
| | 14 F3 # 19 | | | | | | |
| 14 | [K358 × A3-29-305-17-09]- | 21.6 | 1.224 | 0.291 | 0.356 | 0.562 | 0.477 |
| | 14 F3 # 16 | | | | | | |
| 14 | [K358 × A3-29-305-17-09]- | 10.3 | 0.553 | 0.179 | 0.245 | 0.184 | 0.218 |
| | 14 F3 # 5 | | | | | | |
| 15 | [K358 × A3-29-305-17-09]- | 45.1 | 0.676 | 0.222 | 0.644 | 0.48 | 0.451 |
| | 15 F3 # 14 | | | | | | |
| 15 | [K358 × A3-29-305-17-09]- | 21.3 | 0.71 | 0.257 | 0.154 | 0.485 | 0.309 |
| | 15 F3 # 13 | | | | | | |
| 15 | [K358 × A3-29-305-17-09]- | 17.2 | 1.412 | 0.231 | 0.148 | 0.635 | 0.462 |
| | 15 F3 # 2 | | | | | | |
| 17 | [K358 × A3-29-305-17- | 68.6 | 1.196 | 0.281 | 0.238 | 0.021 | 0.218 |
| | 09]-17 F3 # 19 | | | | | | |
| 17 | [K358 × A3-29-305-17- | 64.5 | 6.182 | 0.239 | 0.281 | 0.008 | 0.205 |
| | 09]-17 F3 # 3 | | | | | | |
| 17 | [K358 × A3-29-305-17- | 36.2 | 0.228 | 0.325 | 0.214 | 0.004 | 0.095 |
| | 09]-17 F3 # 7 | | | | | | |
| 17 | [K358 × A3-29-305-17-09]- | 32.2 | 3.112 | 0.248 | 0.205 | 0.007 | 0.176 |
| | 17 F3 # 17 | | | | | | |
| 17 | [K358 × A3-29-305-17- | 31.7 | 0.213 | 0.153 | 0.518 | 0.036 | 0.479 |
| | 09]-17 F3 # 15 | | | | | | |
| 17 | [K358 × A3-29-305-17-09]- | 24.9 | 1.225 | 0.115 | 0.262 | 0.006 | 0.1 |
| | 17 F3 # 10 | | | | | | |
| 17 | [K358 × A3-29-305-17-09]- | 21.8 | 2.417 | 0.235 | 0.044 | 0.015 | 0.214 |
| | 17 F3 # 2 | | | | | | |
| 20 | [K358 × A3-29-305-17- | 74.6 | 5.087 | 0.179 | 0.611 | 0.28 | 0.657 |
| | 09]-20 F3 # 4 | | | | | | |
| 20 | [K358 × A3-29-305-17- | 50.9 | 2.983 | 0.35 | 0.459 | 0.435 | 1.022 |
| | 09]-20 F3 # 10 | | | | | | |
| 20 | [K358 × A3-29-305-17- | 43 | 6.461 | 0.064 | 0.249 | 0.159 | 0.397 |
| | 09]-20 F3 # 9 | | | | | | |
| 20 | [K358 × A3-29-305-17- | 42.2 | 5.915 | 0.198 | 0.293 | 0.07 | 0.209 |
| | 09]-20 F3 # 11 | | | | | | |
| 20 | [K358 × A3-29-305-17- | 36.9 | 1.323 | 0.274 | 0.465 | 0.335 | 0.72 |
| | 09]-20 F3 # 6 | | | | | | |
| 20 | [K358 × A3-29-305-17- | 32.7 | 57.718 | 0.311 | 0.128 | 0.081 | 0.262 |
| | 09]-20 F3 # 1 | | | | | | |
| 20 | [K358 × A3-29-305-17- | 30.8 | 4.256 | 0.068 | 0.337 | 0.218 | 0.667 |
| | 09]-20 F3 # 2 | | | | | | |
| 20 | [K358 × A3-29-305-17-09]- | 18.6 | 7.87 | 0.162 | 0.729 | 0.112 | 0.807 |
| | 20 F3 # 3 | | | | | | |
| 20 | [K358 × A3-29-305-17- | 13.4 | 23.139 | 0.13 | 0.095 | 0.025 | 0.152 |
| | 09]-20 F3 # 19 | | | | | | |
| 21 | [K358 × A3-29-305-17- | 56.4 | 17.001 | 0.253 | 0.492 | 0.202 | 0.395 |
| | 09]-21 F3 # 13 | | | | | | |
| 21 | [K358 × A3-29-305-17- | 40.3 | 4.27 | 0.49 | 0.681 | 0.319 | 0.732 |
| | 09]-21 F3 # 7 | | | | | | |
| 21 | [K358 × A3-29-305-17-09]- | 35.7 | 1.628 | 0.327 | 0.297 | 0.213 | 0.596 |
| | 21 F3 # 19 | | | | | | |
| 21 | [K358 × A3-29-305-17- | 31.5 | 1.595 | 0.237 | 0.156 | 0.104 | 0.179 |
| | 09]-21 F3 # 8 | | | | | | |
| 21 | [K358 × A3-29-305-17-09]- | 22.1 | 11.209 | 0.154 | 0.271 | 0.024 | 0.323 |
| | 21 F3 # 15 | | | | | | |
| 21 | [K358 × A3-29-305-17-09]- | 19.8 | 1.213 | 0.395 | 2.199 | 0.388 | 1.368 |
| | 21 F3 # 2 | | | | | | |
| 21 | [K358 × A3-29-305-17-09]- | 19.2 | 0.288 | 0.247 | 0.217 | 0.217 | 0.6 |
| | 21 F3 # 10 | | | | | | |
| 21 | [K358 × A3-29-305-17-09]- | 7.6 | 0.26 | 0.216 | 1.273 | 0.773 | 2.118 |
| | 21 F3 # 18 | | | | | | |
| 22 | [K358 × A3-29-305-17- | 59.1 | 0.214 | 0.225 | 0.539 | 0.39 | 0.608 |
| | 09]-22 F3 # 14 | | | | | | |
| 22 | [K358 × A3-29-305-17- | 54.3 | 0.755 | 0.084 | 0.41 | 0.282 | 0.491 |
| | 09]-22 F3 # 17 | | | | | | |
| 24 | [K358 × A3-29-305-17-09]- | 67.2 | 0.857 | 0.097 | 0.445 | 0.13 | 0.652 |
| | 24 F3 # 8 | | | | | | |
| 24 | [K358 × A3-29-305-17-09]- | 65 | 1.155 | 0.227 | 0.3 | 0.502 | 0.723 |
| | 24 F3 # 4 | | | | | | |
| 24 | [K358 × A3-29-305-17- | 37.1 | 1.609 | 0.628 | 1.06 | 0.284 | 1.748 |
| | 09]-24 F3 # 6 | | | | | | |
| 24 | [K358 × A3-29-305-17-09]- | 24.1 | 26.019 | 3.446 | 14.629 | 2.684 | 13.657 |
| | 24 F3 # 14 | | | | | | |
| 24 | [K358 × A3-29-305-17-09]- | 21.5 | 10.148 | 2.258 | 2.774 | 1.793 | 7.964 |
| | 24 F3 # 7 | | | | | | |
| 24 | [K358 × A3-29-305-17-09]- | 20 | 1.316 | 0.095 | 0.059 | 0.066 | 0.234 |
| | 24 F3 # 9 | | | | | | |
| 25 | [K358 × A3-29-305-17- | 57.4 | 3.778 | 1.36 | 3.391 | 0.746 | 3.262 |
| | 09]-25 F3 # 10 | | | | | | |
| 25 | [K358 × A3-29-305-17-09]- | 29.9 | 77.068 | 4.853 | 14.501 | 8.136 | 50.585 |
| | 25 F3 # 18 | | | | | | |
| 25 | [K358 × A3-29-305-17-09]- | 23.2 | 74.838 | 3.096 | 6.278 | 0.89 | 4.654 |
| | 25 F3 # 6 | | | | | | |
| 25 | [K358 × A3-29-305-17-09]- | 21.2 | 10.406 | 0.242 | 0.25 | 0.24 | 1.675 |
| | 25 F3 # 8 | | | | | | |
| 25 | [K358 × A3-29-305-17-09]- | 18 | 1.143 | 0.34 | 0.839 | 0.088 | 0.732 |
| | 25 F3 # 2 | | | | | | |
| 30 | [K358 × A3-29-305-17- | 33.8 | 2.613 | 0.274 | 0.554 | 0.535 | 0.605 |
| | 09]-30 F3 # 15 | | | | | | |
| 30 | [K358 × A3-29-305-17- | 30.6 | 14.673 | 0.451 | 1.282 | 0.951 | 0.795 |
| | 09]-30 F3 # 16 | | | | | | |
| 30 | [K358 × A3-29-305-17- | 30.3 | 2.937 | 0.223 | 0.322 | 0.501 | 0.512 |
| | 09]-30 F3 # 6 | | | | | | |
| 30 | [K358 × A3-29-305-17-09]- | 12 | 0.355 | 0.261 | 0.987 | 1.121 | 1.303 |
| | 30 F3 # 17 | | | | | | |
| 35 | [K358 × A3-29-305-17- | 65.1 | 1.165 | 0.438 | 0.44 | 0.688 | 0.592 |
| | 09]-35 F3 # 19 | | | | | | |
| 35 | [K358 × A3-29-305-17- | 54.1 | 0.752 | 0.625 | 0.34 | 0.343 | 0.275 |
| | 09]-35 F3 # 16 | | | | | | |
| 35 | [K358 × A3-29-305-17- | 52.8 | 1.826 | 0.299 | 0.223 | 0.424 | 0.444 |
| | 09]-35 F3 # 18 | | | | | | |
| 35 | [K358 × A3-29-305-17- | 50.4 | 2.064 | 0.62 | 0.782 | 0.808 | 0.824 |
| | 09]-35 F3 # 10 | | | | | | |
| 35 | [K358 × A3-29-305-17- | 42.9 | 1.287 | 0.554 | 0.44 | 0.699 | 0.61 |
| | 09]-35 F3 # 9 | | | | | | |
| 35 | [K358 × A3-29-305-17-09]- | 24.5 | 0.284 | 0.263 | 0.577 | 0.613 | 0.542 |
| | 35 F3 # 7 | | | | | | |
| 35 | [K358 × A3-29-305-17-09]- | 21.6 | 0.581 | 0.237 | 0.403 | 0.303 | 0.313 |
| | 35 F3 # 6 | | | | | | |
| 35 | [K358 × A3-29-305-17-09]- | 13.7 | 0.345 | 0.112 | 0.205 | 0.262 | 0.183 |
| | 35 F3 # 2 | | | | | | |
| 35 | [K358 × A3-29-305-17-09]- | 9.3 | 0.414 | 0.314 | 0.407 | 0.969 | 0.978 |
| | 35 F3 # 8 | | | | | | |
| 35 | [K358 × A3-29-305-17-09]- | 0 | 0.008 | 0.013 | 0.028 | 0.009 | 0.027 |
| | 35 F3 # 3 | | | | | | |
| 37 | [K358 × A3-29-305-17- | 42.6 | 1.655 | 0.36 | 0.303 | 0.218 | 0.201 |
| | 09]-37 F3 # 1 | | | | | | |
| 37 | [K358 × A3-29-305-17- | 36.1 | 0.688 | 0.451 | 0.636 | 0.475 | 0.817 |
| | 09]-37 F3 # 7 | | | | | | |
| 37 | [K358 × A3-29-305-17-09]- | 25.3 | 1.553 | 0.456 | 1.177 | ignore | 0.794 |
| | 37 F3 # 3 | | | | | | |
| 37 | [K358 × A3-29-305-17-09]- | 24.6 | 0.611 | 0.23 | 0.453 | 0.882 | 0.945 |
| | 37 F3 # 9 | | | | | | |
| 37 | [K358 × A3-29-305-17-09]- | 23.6 | 0.355 | 0.526 | 0.525 | 3.298 | 2.055 |
| | 37 F3 # 6 | | | | | | |
| 37 | [K358 × A3-29-305-17-09]- | 21.3 | 2.09 | 0 | 0.487 | 0.853 | 0.965 |
| | 37 F3 # 14 | | | | | | |
| “18” | A3-29-305-17-09-18 F6 | 51.8 | 0.947 | 0.287 | 0.776 | 0.982 | 1.134 |
| | Bulk # 3 | | | | | | |
| “18” | A3-29-305-17-09-18 F6 | 43.8 | 1.676 | 0.592 | 1.266 | 2.558 | 1.732 |
| | Bulk # 1 | | | | | | |
| “18” | A3-29-305-17-09-18 F6 | 40.6 | 1.792 | 0.495 | 1.147 | 1.875 | 1.726 |
| | Bulk # 2 | | | | | | |
| WT | | — | 0 | 0 | 0 | 0 | 0 |
| WT | | — | 0 | 0 | 0 | 0 | 0 |

F4 Generation

TABLE 30
Summary of PC concentration and RT-PCR results in F4 individual plants that
were analyzed in both methods and plants PC yield: leaves biomass, total PC (mg).
28 best performing plants were selected for further breeding (Plant name is bold).
RT-PCR analysis values were compared to the result of another N. tabacum gene:
scfld8. A value in a level of scfld8 and above counted to be positive.

| | | | | | | | | Normal- | |
|---|---|---|---|---|---|---|---|---|---|
| | | | | | | | | ized | Total |
| | | | | | | | | ELISA | PC |
| | | | | | | | Leaves | results | yield/ |
| F4 | | | | | PH4- | PH4- | weight | (mg/kg | plant |
| family | Plant name | col1 | Col2 | LH3 | alpha | beta | (gr) | leaves) | (mg) |
| 4 | [K358 × A3-29-305- | Y | Y | Y | Y | Y | 258 | 46.6 | 12.0 |
| | 17-09]-17-15 F4#11 | | | | | | | | |
| 4 | [K358 × A3-29-305- | Y | Y | Y | Y | Y | 214 | 60.7 | 13.0 |
| | 17-09]-17-15 F4#3 | | | | | | | | |
| 8 | [K358 × A3-29-305- | Y | Y | Y | Y | Y | 1010 | 128.0 | 129.3 |
| | 17-09]-20-4 F4#1 | | | | | | | | |
| 8 | [K358 × A3-29-305- | Y | N | Y | Y | Y | 282 | 47.5 | 13.4 |
| | 17-09]-20-4 F4#10 | | | | | | | | |
| 8 | [K358 × A3-29-305- | Y | Y | Y | Y | Y | 939 | 44.8 | 42.1 |
| | 17-09]-20-4 F4#12 | | | | | | | | |
| 8 | [K358 × A3-29-305- | Y | N | Y | Y | Y | 215 | 68.5 | 14.7 |
| | 17-09]-20-4 F4#14 | | | | | | | | |
| 8 | [K358 × A3-29-305- | Y | Y | Y | Y | Y | 1063 | 112.4 | 119.5 |
| | 17-09]-20-4 F4#15 | | | | | | | | |
| 8 | [K358 × A3-29-305- | Y | Y | Y | Y | Y | 420 | 46.4 | 19.5 |
| | 17-09]-20-4 F4#18 | | | | | | | | |
| 8 | [K358 × A3-29-305- | Y | Y | Y | Y | Y | 807 | 66.0 | 53.3 |
| | 17-09]-20-4 F4#21 | | | | | | | | |
| 8 | [K358 × A3-29-305- | Y | Y | Y | Y | Y | 213 | 118.5 | 25.2 |
| | 17-09]-20-4 F4#23 | | | | | | | | |
| 8 | [K358 × A3-29-305- | Y | Y | Y | Y | Y | 1030 | 139.1 | 143.3 |
| | 17-09]-20-4 F4#24 | | | | | | | | |
| 8 | [K358 × A3-29-305- | Y | Y | Y | Y | Y | 493 | 51.3 | 25.3 |
| | 17-09]-20-4 F4#25 | | | | | | | | |
| 8 | [K358 × A3-29-305- | Y | Y | Y | Y | Y | 223 | 13.1 | 2.9 |
| | 17-09]-20-4 F4#3 | | | | | | | | |
| 8 | [K358 × A3-29-305- | Y | Y | Y | Y | Y | 780 | 116.5 | 90.8 |
| | 17-09]-20-4 F4#5 | | | | | | | | |
| 8 | [K358 × A3-29-305- | Y | Y | Y | Y | Y | 211 | 13.4 | 2.8 |
| | 17-09]-20-4 F4#7 | | | | | | | | |
| 8 | [K358 × A3-29-305- | Y | Y | Y | Y | Y | 475 | 69.3 | 32.9 |
| | 17-09]-20-4 F4#9 | | | | | | | | |
| 9 | [K358 × A3-29-305- | Y | Y | Y | Y | Y | 272 | 5.8 | 1.6 |
| | 17-09]-20-6 F4#11 | | | | | | | | |
| 9 | [K358 × A3-29-305- | Y | Y | Y | Y | Y | 278 | 9.4 | 2.6 |
| | 17-09]-20-6 F4#14 | | | | | | | | |
| 9 | [K358 × A3-29-305- | Y | Y | Y | Y | Y | 1045 | 19.2 | 20.1 |
| | 17-09]-20-6 F4#16 | | | | | | | | |
| 9 | [K358 × A3-29-305- | Y | Y | Y | Y | Y | 295 | 4.3 | 1.3 |
| | 17-09]-20-6 F4#17 | | | | | | | | |
| 9 | [K358 × A3-29-305- | Y | Y | Y | Y | Y | 983 | 129.4 | 127.2 |
| | 17-09]-20-6 F4#19 | | | | | | | | |
| 9 | [K358 × A3-29-305- | Y | Y | Y | Y | Y | 312 | 11.5 | 3.6 |
| | 17-09]-20-6 F4#24 | | | | | | | | |
| 9 | [K358 × A3-29-305- | Y | Y | Y | Y | Y | 269 | 54.5 | 14.7 |
| | 17-09]-20-6 F4#25 | | | | | | | | |
| 9 | [K358 × A3-29-305- | Y | Y | Y | Y | Y | 420 | 10.2 | 4.3 |
| | 17-09]-20-6 F4#4 | | | | | | | | |
| 9 | [K358 × A3-29-305- | Y | Y | Y | Y | Y | 711 | 41.7 | 29.6 |
| | 17-09]-20-6 F4#9 | | | | | | | | |
| 13 | [K358 × A3-29-305- | Y | Y | Y | Y | Y | 881 | 121.7 | 107.2 |
| | 17-09]-21-7F4#10 | | | | | | | | |
| 13 | [K358 × A3-29-305- | Y | Y | Y | Y | Y | 970 | 43.2 | 41.9 |
| | 17-09]-21-7 F4#12 | | | | | | | | |
| 13 | [K358 × A3-29-305- | Y | Y | Y | Y | Y | 825 | | 0.0 |
| | 17-09]-21-7 F4#14 | | | | | | | | |
| 13 | [K358 × A3-29-305- | Y | Y | Y | Y | Y | 365 | 1174 | 42.9 |
| | 17-09]-21-7 F4#18 | | | | | | | | |
| 13 | [K358 × A3-29-305- | Y | Y | Y | Y | Y | 424 | 39.0 | 16.5 |
| | 17-09]-21-7 F4#20 | | | | | | | | |
| 13 | [K358 × A3-29-305- | Y | Y | Y | Y | N | 326 | −12.3 | −4.0 |
| | 17-09]-21-7 F4#21 | | | | | | | | |
| 13 | [K358 × A3-29-305- | Y | N | Y | Y | Y | 547 | 88.9 | 48.6 |
| | 17-09]-21-7 F4#22 | | | | | | | | |
| 13 | [K358 × A3-29-305- | Y | Y | Y | Y | Y | 824 | 1153 | 95.0 |
| | 17-09]-21-7 F4#23 | | | | | | | | |
| 13 | [K358 × A3-29-305- | Y | Y | Y | Y | Y | 984 | | 0.0 |
| | 17-09]-21-7 F4#24 | | | | | | | | |
| 13 | [K358 × A3-29-305- | Y | Y | Y | Y | Y | 955 | | 0.0 |
| | 17-09]-21-7 F4#25 | | | | | | | | |
| 13 | [K358 × A3-29-305- | Y | Y | Y | Y | Y | 327 | 10.7 | 3.5 |
| | 17-09]-21-7 F4#4 | | | | | | | | |
| 13 | [K358 × A3-29-305- | Y | Y | Y | Y | Y | 666 | 98.3 | 65.5 |
| | 17-09]-21-7 F4#6 | | | | | | | | |
| 17 | [K358 × A3-29-305- | Y | Y | Y | Y | Y | 471 | 1422 | 67.0 |
| | 17-09]-22-14 F4#1 | | | | | | | | |
| 17 | [K358 × A3-29-305- | Y | Y | Y | Y | Y | 342 | | 0.0 |
| | 17-09]-22-14 F4#19 | | | | | | | | |
| 17 | [K358 × A3-29-305- | Y | Y | Y | Y | Y | 720 | 64.3 | 46.3 |
| | 17-09]-22-14 F4#21 | | | | | | | | |
| 17 | [K358 × A3-29-305- | Y | Y | Y | Y | Y | 801 | 65.5 | 52.5 |
| | 17-09]-22-14 F4#23 | | | | | | | | |
| 17 | [K358 × A3-29-305- | Y | Y | Y | Y | Y | 621 | 57.9 | 36.0 |
| | 17-09]-22-14 F4#27 | | | | | | | | |
| 17 | [K358 × A3-29-305- | Y | Y | Y | Y | Y | 382 | 48.1 | 18.4 |
| | 17-09]-22-14 F4#3 | | | | | | | | |
| 17 | [K358 × A3-29-305- | Y | Y | Y | Y | Y | 166 | 1.7 | 0.3 |
| | 17-09]-22-14 F4#7 | | | | | | | | |
| 17 | [K358 × A3-29-305- | Y | Y | y | y | Y | 823 | 85.0 | 70.0 |
| | 17-09]-22-14 F4#9 | | | | | | | | |
| 19 | [K358 × A3-29-305- | Y | Y | Y | Y | Y | 476 | 43.0 | 20.5 |
| | 17-09]-24-6 F4#10 | | | | | | | | |
| 19 | [K358 × A3-29-305- | Y | Y | Y | Y | Y | 820 | 50.4 | 41.3 |
| | 17-09]-24-6 F4#16 | | | | | | | | |
| 19 | [K358 × A3-29-305- | Y | Y | N | Y | Y | 289 | 26.3 | 7.6 |
| | 17-09]-24-6 F4#17 | | | | | | | | |
| 19 | [K358 × A3-29-305- | Y | Y | Y | Y | Y | 508 | 51.5 | 26.2 |
| | 17-09]-24-6 F4#2 | | | | | | | | |
| 19 | [K358 × A3-29-305- | Y | Y | Y | Y | Y | 793 | 102.9 | 81.6 |
| | 17-09]-24-6 F4#20 | | | | | | | | |
| 19 | [K358 × A3-29-305- | Y | Y | N | Y | Y | 921 | 52.6 | 48.4 |
| | 17-09]-24-6 F4#21 | | | | | | | | |
| 19 | [K358 × A3-29-305- | Y | Y | Y | Y | Y | 830 | 124.0 | 102.9 |
| | 17-09]-24-6 F4#25 | | | | | | | | |
| 19 | [K358 × A3-29-305- | Y | Y | Y | Y | Y | 581 | 89.1 | 51.8 |
| | 17-09]-24-6 F4#4 | | | | | | | | |
| 19 | [K358 × A3-29-305- | Y | Y | Y | Y | Y | 607 | 42.0 | 25.5 |
| | 17-09]-24-6 F4#8 | | | | | | | | |
| 21 | [K358 × A3-29-305- | Y | Y | Y | Y | Y | 128 | 134.1 | 172.7 |
| | 17-09]-30-6 F4#1 | | | | | | | | |
| 21 | [K358 × A3-29-305- | Y | Y | N | Y | Y | 1335 | 77.7 | 103.7 |
| | 17-09]-30-6 F4#11 | | | | | | | | |
| 21 | [K358 × A3-29-305- | Y | Y | Y | Y | Y | 307 | 84 .4 | 25.9 |
| | 17-09]-30-6 F4#13 | | | | | | | | |
| 21 | [K358 × A3-29-305- | Y | Y | Y | Y | Y | 192 | 1.9 | 0.4 |
| | 17-09]-30-6 F4#16 | | | | | | | | |
| 21 | [K358 × A3-29-305- | Y | Y | Y | Y | Y | 476 | 56.3 | 26.8 |
| | 17-09]-30-6 F4#19 | | | | | | | | |
| 21 | [K358 × A3-29-305- | Y | Y | Y | Y | Y | 932 | 118.6 | 110.5 |
| | 17-09]-30-6 F4#20 | | | | | | | | |
| 21 | [K358 × A3-29-305- | Y | Y | Y | Y | Y | 1645 | 86.0 | 141.4 |
| | 17-09]-30-6 F4#23 | | | | | | | | |
| 21 | [K358 × A3-29-305- | Y | Y | Y | Y | Y | 823 | 84.5 | 69.5 |
| | 17-09]-30-6 F4#24 | | | | | | | | |
| 21 | [K358 × A3-29-305- | Y | Y | Y | Y | Y | 1077 | 112.0 | 120.6 |
| | 17-09]-30-6 F4#3 | | | | | | | | |
| 21 | [K358 × A3-29-305- | Y | Y | Y | Y | Y | 1007 | 76.1 | 76.7 |
| | 17-09]-30-6 F4#4 | | | | | | | | |
| 21 | [K358 × A3-29-305- | Y | Y | Y | Y | Y | 1023 | 113.2 | 115.8 |
| | 17-09]-30-6 F4#7 | | | | | | | | |
| 21 | [K358 × A3-29-305- | Y | Y | Y | Y | Y | 254 | 52.2 | 13.3 |
| | 17-09]-30-6 F4#8 | | | | | | | | |
| 21 | [K358 × A3-29-305- | Y | Y | Y | N | Y | 870 | 71.1 | 61.9 |
| | 17-09]-30-6 F4#9 | | | | | | | | |
| 28 | [K358 × A3-29-305- | Y | Y | Y | Y | Y | 422 | 72.5 | 30.6 |
| | 17-09]-35-19 F4#13 | | | | | | | | |
| 28 | [K358 × A3-29-305- | Y | Y | Y | Y | Y | 224 | 62.0 | 13.9 |
| | 17-09]-35-19 F4#14 | | | | | | | | |
| 28 | [K358 × A3-29-305- | Y | Y | Y | N | Y | 534 | 74.7 | 39.9 |
| | 17-09]-35-19 F4#15 | | | | | | | | |
| 28 | [K358 × A3-29-305- | Y | Y | Y | Y | Y | 453 | 102.9 | 46.6 |
| | 17-09]-35-19 F4#18 | | | | | | | | |
| 28 | [K358 × A3-29-305- | Y | Y | Y | Y | Y | 450 | 71.1 | 32.0 |
| | 17-09]-35-19 F4#19 | | | | | | | | |
| 28 | [K358 × A3-29-305- | Y | Y | Y | Y | Y | 855 | 90.9 | 77.7 |
| | 17-09]-35-19 F4#20 | | | | | | | | |
| 28 | [K358 × A3-29-305- | Y | Y | Y | Y | Y | 775 | 125.6 | 97.3 |
| | 17-09]-35-19 F4#21 | | | | | | | | |
| 28 | [K358 × A3-29-305- | Y | Y | Y | Y | Y | 285 | 104.6 | 29.8 |
| | 17-09]-35-19 F4#23 | | | | | | | | |
| 28 | [K358 × A3-29-305- | Y | Y | Y | Y | Y | 285 | 113.5 | 32.4 |
| | 17-09]-35-19 F4#24 | | | | | | | | |
| 28 | [K358 × A3-29-305- | Y | Y | Y | Y | Y | 824 | 159.0 | 131.1 |
| | 17-09]-35-19 F4#25 | | | | | | | | |
| 28 | [K358 × A3-29-305- | Y | Y | Y | Y | Y | 420 | 100.6 | 42.2 |
| | 17-09]-35-19 F4#6 | | | | | | | | |
| 28 | [K358 × A3-29-305- | Y | Y | Y | Y | Y | 565 | 95.2 | 53.8 |
| | 17-09]-35-19 F4#9 | | | | | | | | |

### Example 5

### Validation of Genomic inserts events in the generated lines

13 individual plants were grown at the greenhouse. Young buds from each plant were sampled for DNA extraction and PCR analysis. The vegetative pieces (˜0.5cm) were picked into small tubes and shipped in ice for DNA extraction.

DNA was extraction using standard CTAB/chloroform method. DNA quality was assessed using Nanodrop. PCR was conducted according to table below.

TABLE 31

| | Unified | | Expected | | Actual | PCR | |
|---|---|---|---|---|---|---|---|
| Primers | name | Insert | size | | size | condition | Comments |
| Event1- | 1-1F; | P4Hb + LH3 | 800 | | ~750 | 1) 95° C.-5 min; | Right |
| R_Rightjunc | 1-1R | right | | | | 2) 35 cycle of | border |
| Event1- | | junction | | | | 95° C.-1 min, | p4Ha + LH3 |
| F_Rightjunc | | | | | | 60-50° C.- | |
| | | | | | | 1 min, 72° C.- | |
| | | | | | | 1.30 min | |
| | | | | | | 3) Final | |
| | | | | | | extension | |
| | | | | | | 72° C.-8 min | |
| | | | | | | 4) 4° C. hold | |
| 1825R | 2-2F; | P4Ha | 800 | | ~900 | 1) 95° C.-5 min | right |
| FP1 | 2-2R | | | | | 2) 39 cycle of | border |
| | | | | | | 95° C.-1 min, | P4Ha |
| | | | | | | 58° C.-30 sec, | |
| | | | | | | 72° C.-30 sec | |
| | | | | | | 3) Final | |
| | | | | | | extension | |
| | | | | | | 72° C.-5 min | |
| | | | | | | 4) 4° C. hold | |
| 14731F | 3-2F; | cola2 | 800 | | 800 | 1) 95° C.-5min | cola2 left |
| RP1 | 1-3R | | | | | 2) 30 cycle of | border |
| | | | | | | 95° C.-1 min, | |
| | | | | | | 65-40° C.-1.30 | |
| | | | | | | min, 65° C.- | |
| | | | | | | 1.30 min | |
| | | | | | | 3) Final | |
| | | | | | | extension | |
| | | | | | | 65° C.-8 min | |
| | | | | | | 4) 4° C. hold | |
| MP_Col_3R | 5-2F; | Cola1 | 3 | k | 3 kb | 1) 95° C.-5 min | Cola1 left |
| RP2 | 3-3R | | | | | 2) 30 cycle of | border |
| MP_Col_4R | 5-1F; | Cola1 | 2 | kb | ~2 kb | 95° C.-1 min, | |

| | | |
|---|---|---|
| RP2 | 3-3R | 65-40° C.-1.30 |
| | | min, 65° C.- |
| | | 1.30 min |
| | | 3) Final |
| | | extension |
| | | 65° C.-8 min |
| | | 4) ° C. hold |

Results:

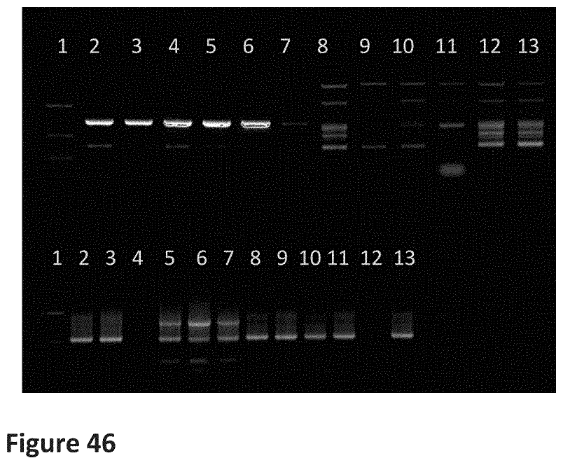

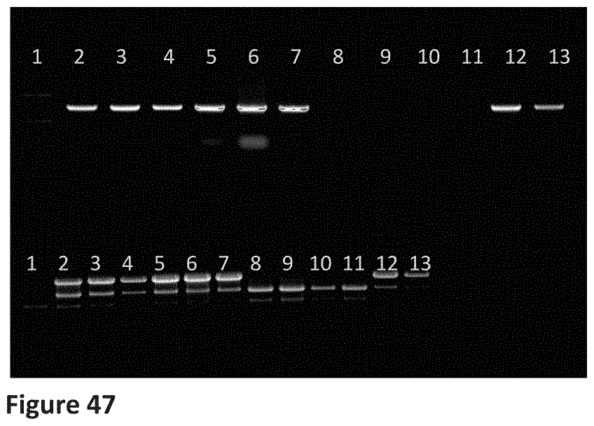

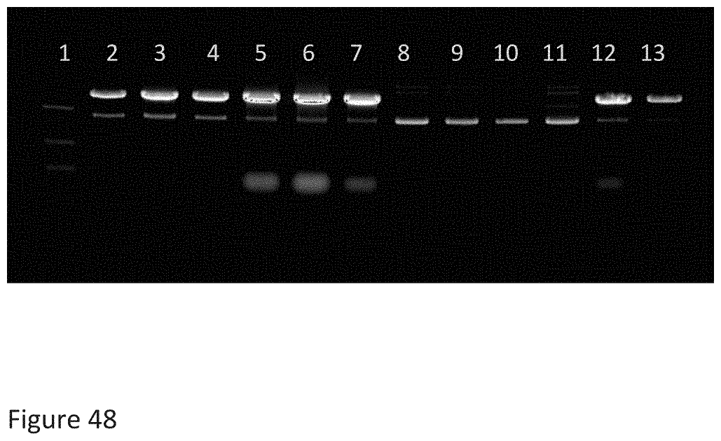

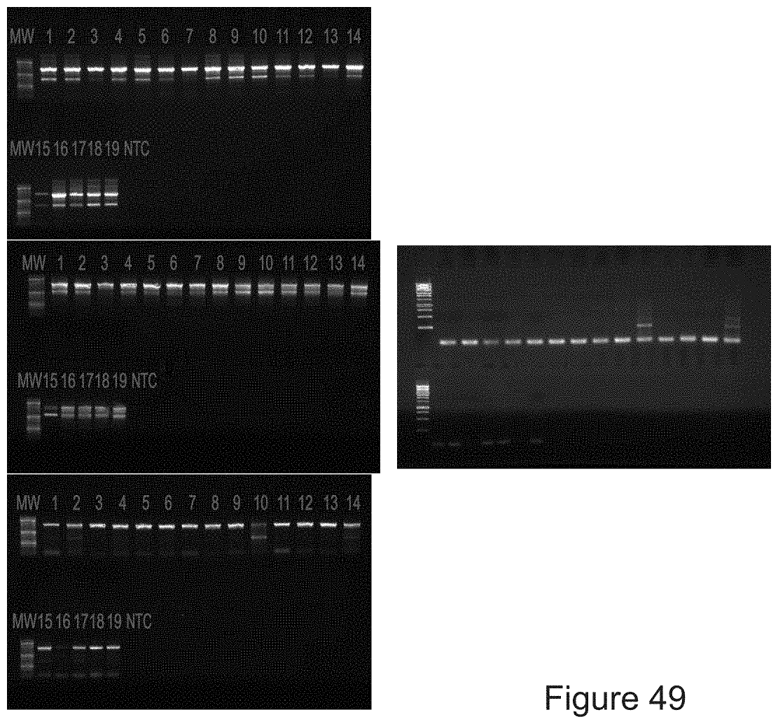

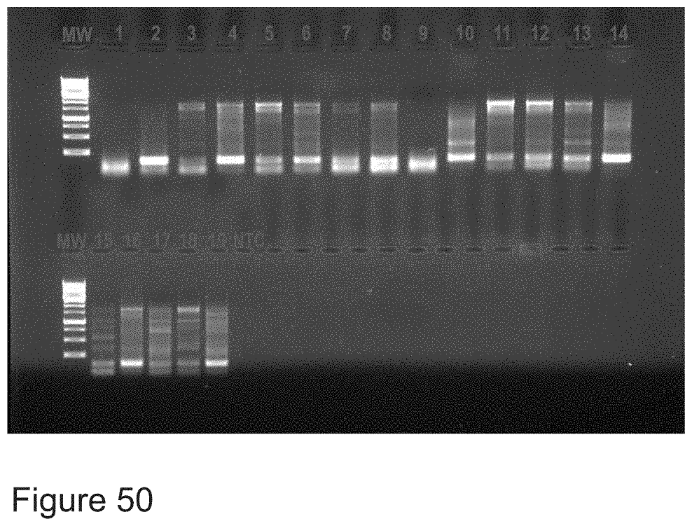

The results are shown in FIGS. 45-48. Note specifically a unique integration site of P4Hα right border shown in bottom panel of FIG. 46 characteristic of A3-29-305-17-09-18 F5 and progeny thereof in lanes 5-7.

More specifically, border PCR was non-specific in all control samples (Samson WT and K358 WT). For P4Hb+LH3 PCR showed expected banding in lines: A3-29 Fl, A3-29-305-17-09 F4, A3-29-305-17-09-F4, A3-29-305-17-09-18 F6*, A3-29-305-17-09-18 F6** and A3-29-305-17-09-18 F6***. For P4Ha expected banding was shown in lines: A3-29-305-17-09-18 F6*, A3-29-305-17-09-18 F6** and A3-29-305-17-09-18 F6***. For Cola2 expected banding was demonstrated in all transgenic lines. For Colal expected banding was demonstrated in all transgenic lines in both primer pairs. Stars represent individual plants originated from seeds.

TABLE 32
List of plant lines tested

| | Sample name | Comments |
|---|---|---|
| 1. | A3-29 F1 (-1c-3)¹ | Pre F5 |
| 2. | A3-29 F1 (-1c-3) | Pre F5 |
| 3. | A3-29-305-17-09 F4 (-2) | Pre F5 |
| 4. | A3-29-305 -17-09-F4(-2-1) | Pre F5 |
| 5. | A3-29-305-17-09-18 F6*² | Post F5 |
| 6. | A3-29-305-17-09-18 F6** | Post F5 |
| 7. | A3-29-305-17-09-18 F6*** | Post F5 |
| 8. | Samson WT* | Wild type control |
| 9. | Samson WT** | Wild type control |
| 10. | Virginia K358 WT* | Wild type control |
| 11. | Virginia K358 WT** | |
| 12. | [K358 × A3-29-305-17- | Pre F5 |
| | 09]-35-19-21-18-13 F6* | |
| 13. | [K358 × A3-29-305-17- | Pre F5 |
| | 09]-35-19-21-18-13 F6** | |

¹Numbers/letters in parenthesis represent specific line originated through cuttings from plants kept at the Master Plant Bank (Yessod);
²Stars represent individual plants originated from seeds.

TABLE 33
list of F1 hybrids tested in the molecular verification PCR assay

| Full Name | VS (Line) | Female | Male |
|---|---|---|---|
| [Virginia K358NN (7) × A3-29-305-17-09-18F5 | 21 | Virginia K358NN | A3-29-305-17-09- |
| (43)] (V521) F1 # 2 | | | 18F5 (43) |
| [Virginia K358NN (7) × A3-29-305-17-09-25-04-19 | 24 | Virginia K358NN | A3-29-305-17-09-25- |
| F7 (46)] (V524)F1 # 2 | | | 04-19 F7 (46) |
| [Burley TN86NN (8) × A3-29-305-17-09-18F5 (43)] | 26 | Burley TN86NN | A3-29-305-17-09-18F5 |
| (V526) F1 # 5 | | | (43) |
| [[Burley TN90NN (9) × A3-29-305-17-09-18F5 (43)] | 31 | Burley TN90NN | A3-29-305-17-09-18F5 |
| ] (Vs-31) (1)″F1 # 1 | | | (43) |
| [[Burley TN90NN (9) × A3-29-305 17 09 18 33 2 F7 | 32 | Burley TN90NN | A3-29-305-17-09-18- |
| (44)] ] (Vs-32) (2)″F1 # 2 | | | 33-2 F7 (44) |
| [Burley TN86NN (8) × A3-29-305-17-09-25-04-19 | 34 | Burley TN86NN | A3-29-305-17-09-25- |
| F7 (46)] (V534) F1 # 3 | | | 04-19 F7 (46) |
| [[Burley TN90NN (9) × A3-29-305 17 09 37 28 31 | 35 | Burley TN90NN | A3-29-305-17-09-37- |
| F7 (47)] ] (Vs-35) (1)″F1 # 1 | | | 28-31 F7 (47) |
| RPG04NN (10) × A3-29-305-17-09-18-33-10F7 | 38 | PG04NN | A3-29-305-17-09-18- |
| (45)] ] (Vs-38) (1)″F1 # 1 | | | 33-10F7 (45) |
| RPG04NN (10) × A3-29-305-17-09-25-04-19 F7 | 39 | PG04NN | A3-29-305-17-09-25- |
| (46)] ] (Vs-39) (1)″F1 # 1 | | | 04-19 F7 (46) |
| [[N tabacum cv. MarylandNN (12) × A3-29-305-17- | 46 | N tabacum cv. | A3-29-305-17-09-18F5 |
| 09-18F5 (43)] ] (Vs-46) (1)″F1 # 1 | | MarylandNN | (43) |
| [[N tabacum cv. MarylandNN (12) × A3-29-305-17- | 47 | N tabacum cv. | A3-29-305-17-09-18- |
| 09-18-33-2 F7 (44)] ] (Vs-47) (1)″F1 # 1 | | MarylandNN | 33-2 F7 (44) |
| [[N tabacum cv. MarylandNN (12) × A3-29-305-17- | 48 | N tabacum cv. | A3-29-305-17-09-18- |
| 09-18-33-10F7 (45)] ] (Vs-48) (1)″F1 # 1 | | MarylandNN | 33-10F7 (45) |

TABLE 34
Summary of the PCR results.
+/− indicate presence/absence of band in the expected size. If a band of
unexpected size is present, then its size is depicted next to the +/− indicator.

| | | | | Cola2 | Cola1 | |
|---|---|---|---|---|---|---|
| # | | P4Hb + LH3 | P4Ha right | left | left | Cola1 left |
| on | | right border | border | border | border | border |
| gel | Line | 800 bp | 800 bp | 800 bp | 3 kb | 2 kb |
| 1 | VS 21 | +/~350 | +/~500 | +/~300 | − | − |
| 2 | VS 24 | +/~350 | +/~500 | +/~300 | − | −/~200 |
| | (1) | | | | | |
| 3 | VS 24 | +/~350 | + | + | − | + |
| | (2) | | | | | |
| 4 | VS 26 | +/~350 | + | + | − | +/~200 |
| | (1) | | | | | |
| 5 | VS 26 | +/~350 | + | + | − | +/~200 |
| | (2) | | | | | |
| 6 | VS 34 | +/~350 | + | + | − | +/~200 |
| | (1) | | | | | |
| 7 | VS 34 | + | + | + | − | +/~200 |
| | (2) | | | | | |
| 8 | VS 35 | +/~350 | + | + | − | +/~200 |
| | (1) | | | | | |
| 9 | VS 35 | +/~350 | +/~500 | + | − | − |
| | (2) | | | | | |
| 10 | VS 38 | +/~350 | +/~500 | +/~300 | − | − |
| | (1) | | | | | |
| 11 | VS 38 | +/~350 | +/~500 | + | − | +/~200 |
| | (2) | | | | | |
| 12 | VS 39 | +/~350 | +/~500 | + | − | +/~200 |
| | (1) | | | | | |
| 13 | VS 46 | +/~350 | + | + | − | +/~200 |
| | (1) | | | | | |
| 14 | VS 46 | +/~350 | +/~500 | + | − | − |
| | (2) | | | | | |
| 15 | VS 47 | +/~350 | +/~500 | +/~300 | − | − |
| | (1) | | | | | |
| 16 | VS 48 | +/~350 | +/~500 | + | − | + |
| | (1) | | | | | |
| 17 | VS 48 | +/~350 | +/~500 | +/~300 | − | − |
| | (2) | | | | | |
| 18 | VS 31 | +/~350 | +/~500 | + | − | + |
| 19 | VS 32 | +/~350 | +/~500 | + | − | − |

All samples are ordered as in Table 34[line number refers “vs” column in Table 33.

TABLE 35
primers ID

| Primer | | | |
|---|---|---|---|
| ID | Name in report | Sequence | Event |
| 1-1F | Event1-F_Rightjunc | GTCTTATCTTCAGCCGACGC/ | #1 |
| | | SEQ ID NO: 27 | |
| 1-1R | Event1-R_Rightjunc | ACACAACAACCACCCCAGAA/ | #1 |
| | | SEQ ID NO: 28 | |
| 1-2F | Event1-F_Leftjunc | CCCCTTCTGATTTTCTTGGTGT/ | #1 |
| | | SEQ ID NO: 29 | |
| 1-2R | Event1-R_LeftRightjunc; | TCCCCTGAAACTTTGGTCCA/ | #1 |
| | E1 R_LF | SEQ ID NO: 30 | |
| 1-3R | RP1 | TGATTTATAAGGGATTTTGCCGAT/ | #1, #2, |
| | | SEQ ID NO: 31 | #3, #4 |
| 2-1F | E2 R_LF, MP_Col_9R | AATTGTTCTGTGAAGGCGGG/ | #2 |
| | | SEQ ID NO: 32 | |
| 2-1R | P4Halpha-F-start | CACCCAGGATTCTTCACTTCV | #2 |
| | | SEQ ID NO: 33 | |
| 2-2F | FP1 | AACCCTGGCGTTACCCAACT/ | #1, #2 |
| | | SEQ ID NO: 34 | |
| 2-2R | 1825R | TGTGTTTGGGGGTTGAGGAT/ | #2 |
| | | SEQ ID NO: 35 | |
| 2-3F | 1825F | GTTTGCATACGCTTGGGTGG/ | #2 |
| | | SEQ ID NO: 36 | |
| 2-4F | MP_Col_:9R | AATTGTTCTGTGAAGGCGGG/ | #2 |
| | | SEQ ID NO: 37 | |
| 2-4R | RP3 | ATTTTGCCGATTTCGGAACC/ | #2 |
| | | SEQ ID NO: 38 | |
| 3-1F | MP_Col_5R | TCATCAAGGACCTGCGTTCAA/ | #3 |
| | | SEQ ID NO: 39 | |
| 3-1R | Colalpha2-r-Start | AGACTCGCCTTTTGATCCAG/ | #3 |
| | | SEQ ID NO: 40 | |
| 3-2F | 14731F | AGGAGTCGTTGTTGTTGGTT/ | #3 |
| | | SEQ ID NO: 41 | |
| 3-3R | RP2 | ATAAGGGATTTTGCCGATTTCG/ | #3 |
| | | SEQ ID NO: 42 | |
| 4-1F | 3815F | TAAGCAGACAACCACGCGAT/ | #4 |
| | | SEQ ID NO: 43 | |
| 4-1R | 3815R | TAAGGTTCGCCGGTGCTATG/ | #4 |
| | | SEQ ID NO: 44 | |
| 5-1F | MP_Col_4R | TGGATCAACTTAGCGGGAGT/ | #5 |
| | | SEQ ID NO: 45 | |
| 5-1R | Colalpha1_R-end | CACATCAAAACCGAACTCTTGA/ | #5 |
| | | SEQ ID NO: 46 | |
| 5-2F | MP_Col_3R | ACGGTTTTAAAGTCTTGCAACC/ | #5 |
| | | SEQ ID NO: 47 | |

TABLE 36

| Primer | | |
|---|---|---|
| set ID | | |
| (ref to | | |
| report) | Event | Comments (ref to report) |
| 1-1F, | #1 | P4H beta, LH3; right junction (FIG. 24A, Sequence |
| 1-1R | | #1) |
| (Table | | |
| 2) | | |
| 1-2F, | #1 | P4H beta, LH3; left junction (FIG. 24B, Sequence |
| 1-2R | | #2) |
| (Table | | |
| 2) | | |
| 2-1F, | #2 | P4H alpha; left junction (FIG. 28A, Sequence #5) |
| 2-1R | | |
| (Table | | |
| 5) | | |
| 2-2F, | #2 | P4H alpha; right junction (FIG. 28B, Sequence #6) |
| 2-1R | | |
| (Table | | |
| 5) | | |
| 3-1F, | #3 | Col alpha2; left junction (FIG. 32, Sequence #10) |
| 3-1R | | |
| (Table | | |
| 8) | | |
| 4-1F, | #4 | P4 beta, LH3; left junction (Sequence #15) |
| 4-1R | | |
| (Table | | |
| 11) | | |
| 5-1F, | #5 | Col alpha 1; left junction (FIG. 39, Sequence #17) |
| 5-1R | | |
| (Table | | |
| 14) | | |

Nanopore PCR sequence
*Primer set 1-2F, 1-2R spans the whole gene

TABLE 37
Sanger validation

| Primer set | | |
|---|---|---|
| ID (ref to | | |
| report) | Event | Comments (ref to report) |
| 1-2F, 1-3R | #1 | P4H beta; LH3; left border (FIG. 25A, Sequence |
| (Table 3) | | #3) |
| 1-4F, 1-2R | #1 | P4H beta, LH3; right border (FIG. 25B, Sequence |
| (Table 3) | | #4) |
| 2-3F, 1-3R | #2 | P4H alpha; left border (FIG. 29A, Sequence #7) |
| (Table 6) | | |
| 2-4F, 2-4R | #2 | P4H alpha; left border (FIG. 29B, Sequence #8) |
| (Tale 6) | | |
| 2-2F, 2-2R | #2 | P4H alpha; right border (FIG. 29C, Sequence #9) |
| (Table 6) | | |
| 3-2F, 1-3R | #3 | Col alpha2; left border (FIG. 33A, Sequence #11, |
| (Table 9) | | #12) |
| 3-1F, 3-3R | #3 | Col alpha2; left border (FIG. 33B, Sequence #13, |
| (Table 9) | | #p14) |
| 4-1F, 1-3R | #4 | P4 beta, LH3; left border (FIG. 36, Sequence #16) |
| (Table 12) | | |
| 5-2F, 3-3R | #5 | Col alpha 1; left border (FIG. 40; #Sequence #18) |
| (Table 15) | | |
| 5-2F, 5-2R | #5 | Col alpha 1; left border (FIG. 40; #Sequence #19) |
| (Table 15) | | |

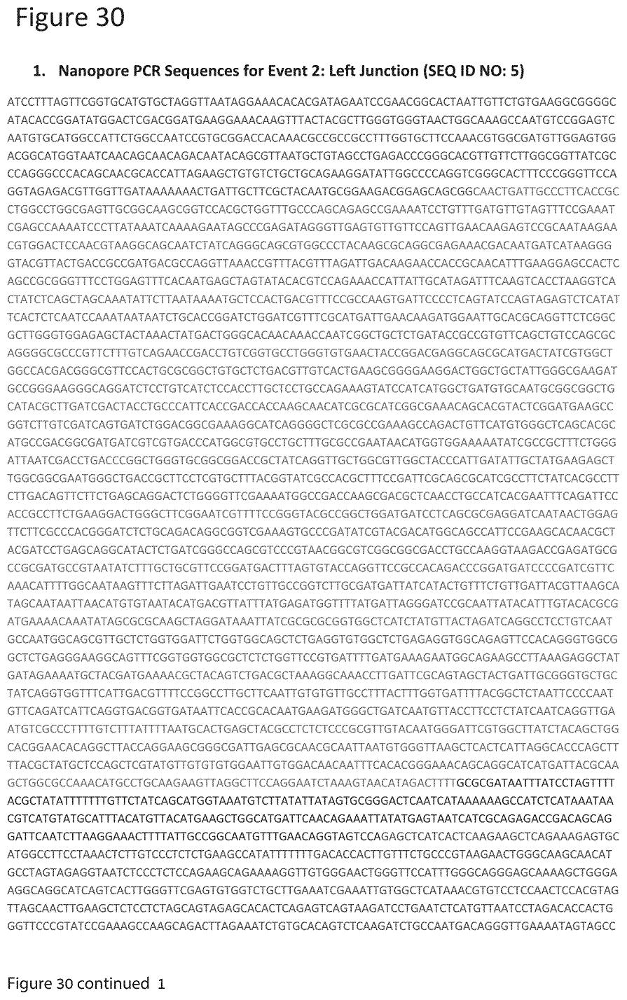

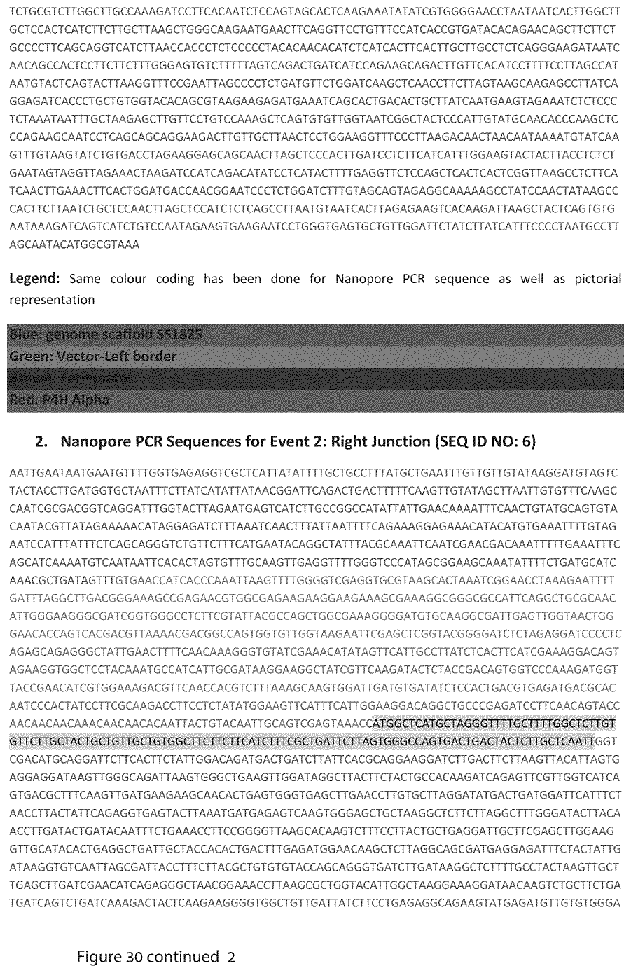

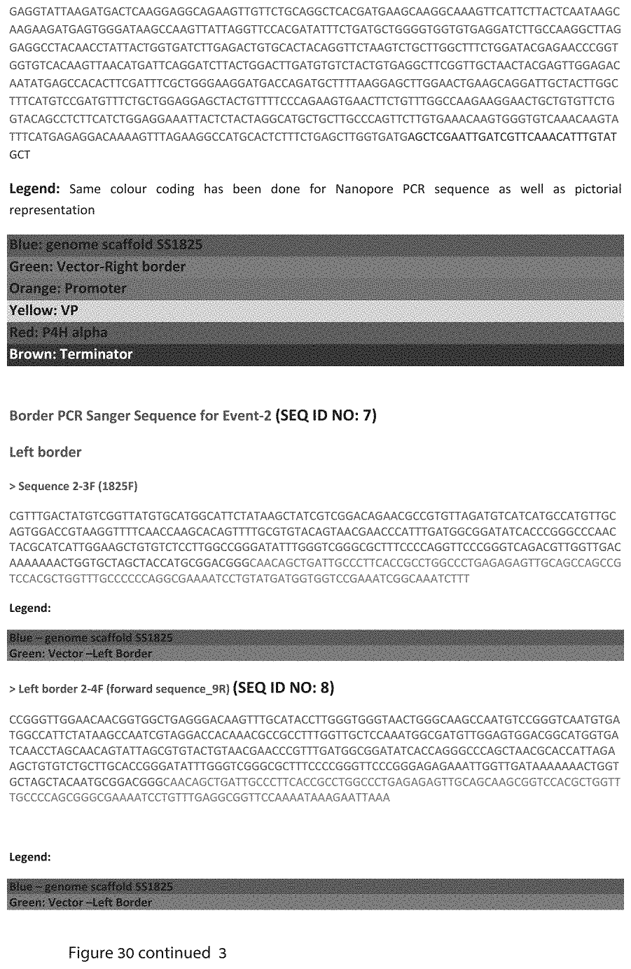

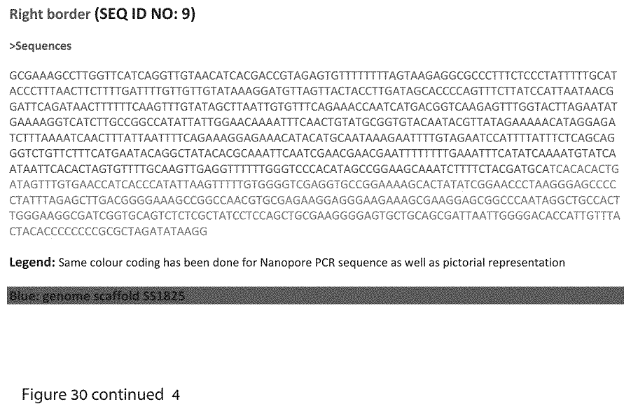

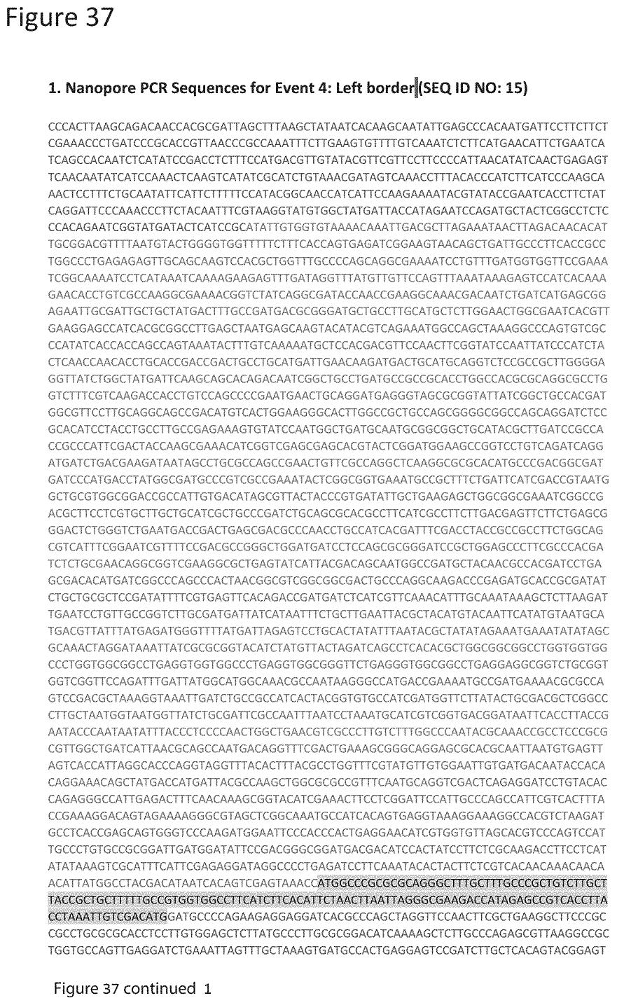

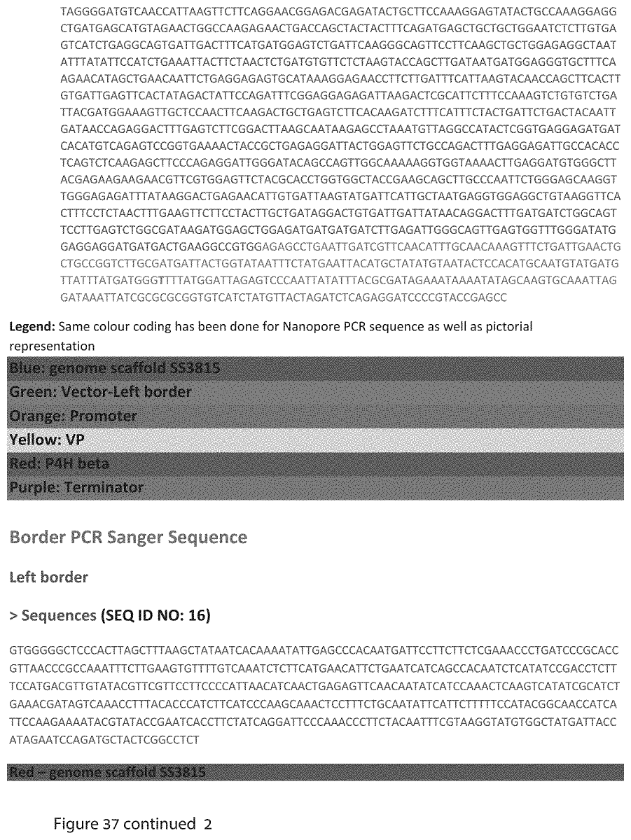

TABLE 38
Sequence list

| Sequence | |
|---|---|
| ID | Description |
| #1 | P4H beta, LH3; right junction; FIG. 26 |
| #2 | P4H beta, LH3; left junction; FIG. 26 |
| #3 | P4H beta; LH3; left border; FIG. 26 |
| #4 | P4H beta, LH3; right border; FIG. 26 |
| #5 | P4H alpha; left junction; FIG. 30 |
| #6 | P4H alpha; right junction; FIG. 30 |
| #7 | P4H alpha; left border; FIG. 30 |
| #8 | P4H alpha; left border; FIG. 30 |
| #9 | P4H alpha; right border: FIG. 30 |
| #10 | Col alpha2; left junction; FIG. 34 |
| #11 | Col alpha2; left border; FIG. 34 |
| #12 | Col alpha2; left border; FIG. 34 |
| #13 | Col alpha2; left border; FIG. 34 |
| #14 | Col alpha2; left border; FIG. 34 |
| #15 | P4 beta, LH3; left junction; FIG. 37 |
| #16 | P4 beta, LH3; left border; FIG. 37 |
| #17 | Col alpha 1; left junction; FIG. 40 |
| #18 | Col alpha 1; left border; FIG. 40 |
| #19 | Col alpha 1; left border; FIG. 40 |
| #20 | Synthetic sequence containing the coding regions of the |
| | vacuolar signal sequence of barley gene for Thiol protease |
| | aleurain precursor fused to the human Collagen alpha 1(I) |
| | chain |
| #21 | Synthetic sequence containing the coding regions of the |
| | vacuolar signal sequence of barley gene for Thiol protease |
| | aleurain precursor fused to the human Collagen alpha 2(I) |
| | chain. |
| #22 | Synthetic sequence containing the coding regions of the |
| | vacuolar signal sequence of barley gene for Thiol protease |
| | aleurain precursor fused to the human Prolyl 4-hydroxylase |
| | alpha-1 subunit |
| #23 | Synthetic sequence containing the coding regions of the |
| | vacuolar signal sequence of barley gene for Thiol protease |
| | aleurain precursor fused to the human Prolyl 4-hydroxylase |
| | beta subunit. |
| #24 | Synthetic sequence containing the coding regions of the |
| | vacuolar signal sequence of barley gene for Thiol protease |
| | aleurain precursor fused to the human Lysyl hydroxylase 3. |
| #25 | AA sequence human procollagen alpha 1(I) chain |
| #26 | AA sequence human procollagen alpha 2(I) chain |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A recombinant DNA molecule detectable in a sample containing tobacco DNA, wherein the nucleotide sequence of said molecule is:
a) at least 99% identical to SEQ ID NO: 6 or 9; or
b) a nucleotide sequence completely complementary to (a),
wherein the presence of the recombinant DNA molecule is diagnostic for tobacco event A3-29-305-17-09-18 DNA or progeny thereof in said sample.

2. A DNA molecule comprising a polynucleotide segment of sufficient length to function as a DNA probe that hybridizes specifically under stringent hybridization conditions with a recombinant DNA of tobacco event A3-29-305-17-09-18 or progeny thereof in a sample, wherein hybridization of said DNA molecule under said hybridization conditions is diagnostic for tobacco event A3-29-305-17-09-18 or progeny thereof in said sample.

3. The DNA molecule of claim 2, wherein said recombinant DNA molecule comprises:
a) a nucleotide sequence at least 99% identical to SEQ ID NO: 6 or 9; or
b) a nucleotide sequence completely complementary to (a).

4. A pair of DNA molecules comprising a first DNA molecule and a second DNA molecule functioning as primers when used together in an amplification reaction with a sample comprising a recombinant DNA of tobacco event A3-29-305-17-09-18 or progeny thereof to produce an amplicon diagnostic for said recombinant DNA of said tobacco event A3-29-305-17-09-18 or progeny thereof in said sample, wherein said amplicon comprises a nucleotide sequence at least 99% identical to SEQ ID NO: 6 or 9.

5. A method of detecting the presence of a recombinant DNA diagnostic for tobacco event A3-29-305-17-09-18 or progeny thereof DNA in a sample, said method comprising:
(a) contacting said sample with the DNA molecule of claim 2 under stringent hybridization conditions; and
(b) detecting hybridization of the DNA molecule to the recombinant DNA, wherein hybridization is diagnostic for the presence of the recombinant DNA of said tobacco event A3-29-305-17-09-18 or progeny thereof in said sample.

6. A method of detecting presence of a recombinant DNA of tobacco event A3-29-305-17-09-18 or progeny thereof in a sample, the method comprising:
(a) contacting said sample with the pair of DNA molecules of claim 4;
(b) performing an amplification reaction sufficient to produce a DNA amplicon using said pair of DNA molecules; and
(c) detecting the presence of said DNA amplicon in said reaction,
wherein said DNA amplicon comprises a nucleotide sequence at least 99% identical to SEQ ID NO: 6 or 9, and wherein presence of said amplicon is diagnostic for the recombinant DNA of tobacco event A3-29-305-17-09-18 or progeny thereof in said sample.

7. The method of claim 5, further comprising detecting at least one of a nucleotide sequence at least 99% identical SEQ ID NOs: 1-5, 7-8, 10-19.

8. A tobacco plant, plant part, or cell thereof comprising a nucleotide sequence at least 99% identical to SEQ ID NOs: 6 or 9.

9. The method of claim 6, further comprising detecting presence and/or orientation of LH3, P4Hb, collagen alpha 1 and/or collagen alpha 2.

10. The method of claim 9, wherein said presence and/or orientation is at least 99% identical to that of event A3-29-305-17-09-18.

11. The method of claim 9, wherein said presence and/or orientation is identical to that of event A3-29-305-17-09-18.

12. The tobacco plant, plant part, or cell thereof of claim 8, wherein said tobacco plant is a progeny of any generation of a tobacco plant comprising said tobacco event A3-29-305-17-09-18.

13. The tobacco plant, plant part, or cell thereof of claim 8, comprising at least one of a nucleotide sequence at least 99% identical SEQ ID NOs: 1-5, 7-8, 10-19.

14. The DNA molecule of claim 1, wherein said progeny is an inbred or a hybrid tobacco plant.

15. The DNA molecule of claim 14, wherein said progeny is listed in any one of Tables 20, 21, 21a and 22.

16. The DNA molecule of claim 1, wherein said recombinant DNA molecule is derived from a tobacco event or progeny thereof listed in any one of Tables 20, 21, 21a and 22.

17. The DNA molecule of claim 1, wherein said nucleotide sequence is as set forth in SEQ ID NOs: 34 and 35.

18. A method of producing procollagen, the method comprising:
(a) growing the plant of claim 8; and
(b) isolating the procollagen from the plant.

19. A procollagen obtainable according to the method of claim 18.

20. A method of processing procollagen, the method comprising:
(a) providing a protein preparation of the plant of claim 8; and
(b) contacting said protein preparation with an effective amount of an enzyme capable of processing to procollagen to collagen.

21. The method of claim 20, wherein said enzyme comprises ficin.

22. A tobacco seed comprising a detectable amount of a nucleotide sequence at least 99% identical to SEQ ID NOs: 6 or 9, or complete complements thereof.

23. The tobacco seed of claim 22 comprising a detectable amount of a nucleotide sequence at least 99% identical to SEQ ID NOs: 1-5, 7-8, 10-19, or complete complements thereof.

24. (canceled)

25. A tobacco plant, tobacco plant part, comprising DNA functional as a template when tested in a DNA amplification method producing an amplicon diagnostic for the presence of event A3-29-305-17-09-18 DNA.

26. (canceled)

27. A method of producing a plant having an improved agricultural trait, the method comprising:
(a) subjecting the plant of claim 8 to a breeding program and/or transgenesis and/or genome editing; and
(b) selecting a plant exhibiting an improved agricultural trait.

28. The DNA molecule of claim 1, wherein said progeny comprises A3-29-305-17-09-18 hybrid with Samsun.

29. A recombinant DNA molecule detectable in a sample containing tobacco DNA, wherein the nucleotide sequence of said molecule is:
a) at least 99% identical to SEQ ID NO: 1-19; or
b) a nucleotide sequence completely complementary to (a),
wherein the presence of the recombinant DNA molecule is diagnostic for tobacco event A3-29-305-17-09 DNA or progeny thereof in said sample.

30-51. (canceled)
